SEQUENCE LISTING (32 sequences)

SEQ ID NO: 1 - protein, 517 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 530 aa, Homo sapiens
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 537 aa, Homo sapiens
SVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 520 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 5 - protein, 541 aa, Homo sapiens
SVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 656 aa, Homo sapiens
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGGGGSGGGGSMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 7 - protein, 692 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 8 - protein, 673 aa, Homo sapiens
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGSGGGGSSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 9 - protein, 668 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 10 - protein, 21 aa, synthetic construct
SGATNFSLLKQAGDVEENPGP

SEQ ID NO: 11 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 219 aa, Homo sapiens
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 13 - protein, 230 aa, Homo sapiens
SVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWP

SEQ ID NO: 14 - protein, 213 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 15 - protein, 677 aa, Homo sapiens
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGSGGGGSSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 16 - protein, 24 aa, Homo sapiens
LLLGVSVSCIVILAVCLLCYVSIT

SEQ ID NO: 17 - protein, 25 aa, Homo sapiens
PILLTISILSFFSVALLVILACVLW

SEQ ID NO: 18 - protein, 21 aa, Homo sapiens
GNTLVAVSIFLLLTGPTYLLF

SEQ ID NO: 19 - protein, 21 aa, Homo sapiens
GWNPHLLLLLLLVIVFIPAFW

SEQ ID NO: 20 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 21 - protein, 25 aa, Homo sapiens
IPWLGHLLVGLSGAFGFIILVYLLI

SEQ ID NO: 22 - protein, 129 aa, Homo sapiens [sequence omitted]

SEQ ID NO: 23 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 24 - protein, 126 aa, Homo sapiens
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 25 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 26 - protein, 22 aa, Homo sapiens
MLLLVTSLLLCELPHPAFLLIP

SEQ ID NO: 27 - protein, 10 aa, synthetic construct
GGGGSGGGGS

SEQ ID NO: 28 - protein, 5 aa, synthetic construct
GSGPR

SEQ ID NO: 29 - protein, 5 aa, synthetic construct
  VARIANT 1..5: note = can be present in repeats of any integer
GGGGS

SEQ ID NO: 30 - protein, 5 aa, synthetic construct
GGGGS

SEQ ID NO: 31 - protein, 15 aa, synthetic construct
GGGGSGGGGSGGGGS

SEQ ID NO: 32 - protein, 26 aa, synthetic construct
SGGGSGGGGSGGGGSGGGGSGGGSLQ

(19) United States
(12) Patent Application Publication
(10) Pub. No.: US 2026/0028388 A1
(43) Pub. Date: Jan. 29, 2026

(54) RECOMBINANT CYTOKINE RECEPTORS AND METHODS OF USE

(71) Applicant: Sonoma Biotherapeutics, Inc., South San Francisco, CA (US)

(72) Inventors: Joshua BEILKE, Seattle, WA (US); James MATTHAEI, South San Francisco, CA (US); Frederic VAN GOOL, South San Francisco, CA (US); Jessica CORTEZ, South San Francisco, CA (US)

(73) Assignee: Sonoma Biotherapeutics, Inc., South San Francisco, CA (US)

(21) Appl. No.: 18/993,216

(22) PCT Filed: Jul. 10, 2023
(86) PCT No.: PCT/US2023/069884

§ 371 Date: Jan. 10, 2025

Related U.S. Application Data
(60) Provisional application No. 63/368,085, filed on Jul. 11, 2022.
(60) Provisional application No. 63/498,802, filed on Apr. 27, 2023.
(60) Provisional application No. 63/499,929, filed on May 3, 2023.

(51) Int. Cl. C07K 14/715; A61K 40/11; A61K 40/30; A61K 40/41; C12N 5/0783

(52) U.S. Cl. CPC C07K 14/7155; A61K 40/11; A61K 40/30; A61K 40/418; C12N 5/0637; C07K 2319/03; C12N 2510/00

(57) ABSTRACT

Provided are recombinant cytokine receptors that comprise an IL-2 intracellular domain and an extracellular domain that binds to a cytokine other than IL-2. Also provided herein are Treg cell comprising recombinant cytokine receptors and methods of use.

### CROSS-REFERENCE TO RELATED APPLICATIONS

This application claims the benefit of, and priority to, U.S. Provisional Application 63/368,085, filed on Jul. 11, 2022, U.S. Provisional Application 63/498,802, filed on Apr. 27, 2023, and U.S. Provisional Application 63/499,929, filed on May 3, 2023, the contents of each of which are hereby incorporated herein by reference in their entirety.

### INCORPORATION BY REFERENCE OF SEQUENCE LISTING

The contents of the electronic sequence listing (237752000241SEQLIST.xml; Size: 36,642 bytes; and Date of Creation: Jul. 7, 2023) is herein incorporated by reference in its entirety.

### BACKGROUND

The immune system plays a critical role in maintaining organismal homeostasis, poised between the elimination of foreign antigens and the self-tolerance of autoantigens. In particular, hyperactive immune dysregulation can lead to various autoimmune disorders (e.g., irritable bowel syndrome, systemic lupus erythematosus, alopecia areata, multiple sclerosis), which is often the result of overactive effector T lymphocytes or underactive regulatory T lymphocytes (i.e., Tregs). Current therapies for autoimmune diseases involve the administration of steroids, which can cause serious side-effects in patients and often offer little relief.

Regulatory T cells are a key player in the maintenance of organismal homeostasis to prevent the destruction of otherwise healthy tissues. Tregs are a unique subset of T cells that inhibit the cytotoxic or pro-inflammatory activity of effector CD4+ or effector CD8+ T cells. Tregs differentiate from the parent T lymphocyte lineage upon the upregulation of key Treg genes, in particular CD25 and FOXP3 (see, e.g., Chen, M L et al. (2005), Proc Natl Acad Sci USA 102 (2): 419-424; and Liu, V C et al. (2007), J Immunol 178 (5): 2883-2892, hereby incorporated by reference in their entirety). Upon T cell receptor (TCR) activation, these Tregs are responsible for directly suppressing effector T cell activity via cytokine production, e.g., TGF-β and IL-10 (see, e.g., Chen, J et al. (2019), Trends Mol Med 25 (11): 1010-1023, hereby incorporated by reference in its entirety), and the engagement of immune checkpoint receptors, e.g., TIGIT- or CTLA-4-engagement (see, e.g., Knochelmann, H M et al. (2018), Cell Mol Immunol; 15 (5): 458-469, hereby incorporated by reference in its entirety). While effector T cells are able to produce the cytokine IL-2 upon TCR activation to support their own expansion, Treg cells are dependent upon exogenous IL-2 to promote Treg survival and maintenance, as Tregs are unable to produce their own IL-2. However, Treg cells require IL-2 signaling for survival and proliferation, like all lineages of T lymphocytes. This biological mechanism ensures that Tregs are maintained in tissue niches that are enriched with active effector T cells, thus creating a cellular negative feedback mechanism whereby the increased IL-2 production by the target effector T cell promotes the expansion of the Tregs that then act to inhibit effector T cell activity and thus downstream inhibit their own expansion and survival (see, e.g., Shevyrev, D & Tereshchenko, V (2020), Front Immunol; 10:3100, 1-13). Thus, targeting of the IL-2 receptor signaling pathway in Tregs is one area of recent interest in the effort to identify novel therapies for autoimmune disorders.

### SUMMARY

In one aspect, the present application relates to recombinant cytokine receptors comprising an intracellular domain of IL-2Rβ and an extracellular domain from a cytokine other than IL-2. Also provided herein are cells that express the recombinant cytokine receptors and methods of use.

### BRIEF DESCRIPTION OF THE DRAWINGS

FIG. 1A shows the organization of a DNA cassette encoding an untethered cytokine receptor. The black arrow indicates the direction of transcription. MND represents the promoter region. The recombinant cytokine receptor comprises the extracellular and transmembrane domain of an IL receptor (indicated by ILXR, where X is 2, 4, 7, 9, or 21; thus, the IL extracellular domain and transmembrane domain can be from any of IL-2, IL-4, IL-7, IL-9, and IL-21), and an IL-2Rβ intracellular domain. The construct also includes the P2A self-cleaving peptide. EGFR is the epidermal growth factor receptor transduction marker. FIG. 1B shows an untethered recombinant receptor protein construct. FIG. 1C shows the organization of a DNA cassette encoding for tethered cytokine receptor. The black arrow indicates the direction of transcription. MND represents the promoter region. FIG. 1D shows a tethered protein construct. FIG. 1E shows schematics of the exemplary recombinant cytokine receptors, which can be untethered or tethered to cytokine.

FIG. 2A shows the degree of secreted embryonic alkaline phosphatase (SEAP) protein production and secretion from HEKBlue cells that express untethered recombinant cytokine receptors. HEKBlue cells express an IL-2 signaling reporter construct. Cells were cultured in the presence or absence of IL-2 or the indicated cytokine at the following doses: IL-4:0.4 ng/ml and 4 ng/ml (“10×”); IL-7:1 ng/ml and 10 ng/ml (“10×”); IL-9:2 ng/ml and 20 ng/ml (“10×”); and IL-21:5 ng/ml and 50 ng/ml (“10×”). FIG. 2B shows the degree of SEAP production and secretion from HEKBlue cells that express tethered recombinant cytokine receptors. FIG. 2C shows a dose response curve for absorbance values based on SEAP production by HEKBlue cells carrying an IL-2 reporter construct when cultured with increasing doses of IL-2. FIG. 2D shows a schematic of the tethered recombinant cytokine receptor transactivation assay. Jurkat T cells that express tethered recombinant cytokine receptors are co-cultured overnight with IL-2 reporter cells that express the untethered version of the recombinant cytokine receptor. FIG. 2E shows the absorbance readout of SEAP production from the IL-2 reporter cells expressing an untethered recombinant cytokine receptor (IL-4Rα/IL-2Rβ, IL-7Rα/IL-2Rβ, IL-9Rα/IL-2Rβ, or IL-21Rα/IL-2Rβ) co-cultured with Jurkat T cells expressing the tethered recombinant cytokine receptor of the same untethered IL-XRα/IL-2Rβ construct in the IL-2 reporter cells, as depicted in FIG. 2D. FIG. 2F shows representative FACS plots of the IL-9Rα levels on control Tregs compared to Tregs expressing the IL-9Rα/IL-2Rβ recombinant cytokine (“IL9/2”).

FIG. 3A shows representative Fluorescence Activated Cell Sorting (FACS) plots of regulatory T cells (Treg cells) successfully expressing one of the tethered or untethered IL-4Rα/IL-2Rβ at day 7 and day 12 post transduction. Cells are stained with EGFR antibodies to identify EGFR+ cells then sorted. Flow Cytometry Standard (FCS) is forward scatter, EGFR is gated on the side scatter. Boxes mark the population of cells that are EGFR+ or EGFR−. The percent of cell population that are EGFR+ or EGFR− are indicated. FIG. 3B shows representative FACS plots of Treg cells successfully expressing one of the tethered or untethered IL-7Rα/IL-2Rβ at day 7 and day 12 post transduction. FIG. 3C shows representative FACS plots of Treg cells successfully expressing either tethered or untethered IL-21Rα/IL-2Rβ or IL-9Rα/IL-2Rβ at day 7 and day 12 post transduction. FIG. 3D shows representative FACS plots of Treg cells successfully expressing either tethered or untethered IL-4Rα/IL-2Rβ at day 7 and day 12 post transduction when cultured in the presence of IL-4 cytokine. FIG. 3E shows FACS plots of Treg cells successfully expressing either tethered or untethered IL-7Rα/IL-2Rβ at day 7 and day 12 post transduction when cultured in the presence of IL-7 cytokine. FIG. 3F shows representative FACS plots of regulatory T cells (Treg cells) successfully expressing either tethered or untethered IL-9Rα/IL-2Rβ at day 7 and day 12 post transduction when cultured in the presence of IL-9 cytokine. FIG. 3G shows representative FACS plots of Treg cell) successfully expressing either tethered or untethered IL-21Rα/IL-2Rβ at day 7 and day 12 post transduction when cultured in the presence of IL-21 cytokine. FIG. 3H shows representative FACS plots of Treg cells successfully expressing either tethered or untethered IL-7Rα/IL-2Rβ at day 7 and day 12 post transduction when cultured in the presence of IL-2 cytokine. FIG. 3I shows representative FACS plots of Treg cells successfully expressing untethered IL-4Rα/IL-2Rβ. IL-9Rα/IL-2Rβ, or IL-21Rα/IL-2Rβ at day 7 and day 12 post transduction when cultured in the presence of IL-2 cytokine.

FIG. 4A shows line graphs representing the percent of EGFR+ Treg cells expressing tethered or untethered IL-7Rα/IL-2Rβ recombinant cytokine receptors at days 7 and 13 when cultured in the absence of cytokine. Constructs transfected with EGFR serve as a positive control (EGFR). UT represents untransduced Tregs. FIG. 4B shows line graphs representing the percent of EGFR+ Treg cells expressing tethered or untethered IL-7Rα/IL-2Rβ recombinant cytokine receptors at days 7 and 13 when cultured in the presence of IL-7 cytokine. FIG. 4C shows line graphs representing the percent of EGFR+ Treg cells expressing tethered or untethered IL-7Rα/IL-2Rβ recombinant cytokine receptors at days 7 and 13 when cultured in the presence of IL-2 cytokine.

FIG. 5A shows a line graph representing the percent of viable Treg cells expressing tethered or untethered IL-7Rα/IL-2Rβ recombinant cytokine receptors at days 2, 5, 7, 9, 12 and 14 when cultured in the absence of cytokine. Constructs transfected with EGFR serve as a positive control (EGFR). UT represents untransduced Treg cells. FIG. 5B shows a line graph representing the percent of viable Treg cells expressing tethered or untethered IL-7Rα/IL-2Rβ recombinant cytokine receptors at days 2, 5, 7, 9, 12 and 14 when cultured in the presence of IL-7 cytokine. Constructs transfected with EGFR serve as a positive control (EGFR). UT represents untransduced Treg cells. FIG. 5C shows a line graph representing the percent of viable Treg cells expressing tethered or untethered IL-7Rα/IL-2Rβ recombinant cytokine receptors at days 2. 5, 7, 9, 12 and 14 when cultured in the presence of IL-2 cytokine. FIG. 5D shows a line graph representing the percent of viable Treg cells expressing tethered or untethered IL-4Rα/IL-2Rβ, IL-9Rα/IL-2Rβ, or IL-21Rα/IL-2Rβ recombinant cytokine receptors at days 5, 7, 9, 12 and 14 when cultured in the absence of cytokine. FIG. 5E shows a line graph representing the percent of viable Treg cells expressing tethered or untethered IL-4Rα/IL-2Rβ, IL-9Rα/IL-2Rβ, or IL-21Rα/IL-2Rβ recombinant cytokine receptors at days 5, 7, 9, 12 and 14 when cultured in the presence of IL-2 cytokine. FIG. 5F shows a line graph representing the percent of viable Treg cells expressing tethered or untethered IL-4Rα/IL-2Rβ recombinant cytokine receptors at days 5, 7, 9, 12 and 14 when cultured in the presence of IL-4 cytokine. FIG. 5G shows a line graph representing the percent of viable Treg cells expressing tethered or untethered IL-9Rα/IL-2Rβ recombinant cytokine receptors at days 5, 7, 9, 12 and 14 when cultured in the presence of IL-9 cytokine. FIG. 5H shows a line graph representing the percent of viable Treg cells expressing tethered or untethered IL-21Rα/IL-2Rβ recombinant cytokine receptors at days 5, 7, 9, 12 and 14 when cultured in the presence of IL-21 cytokine.

FIG. 6A shows a line graph representing the number of Treg cells expressing tethered or untethered IL-7Rα/IL-2Rβ recombinant cytokine receptors at days 2, 5, 7, 9, 12 and 14 when cultured in the absence of cytokine. Constructs transfected with EGFR serve as a positive control (EGFR). UT represents untransduced Treg cells. FIG. 6B shows a line graph representing the number of Treg cells expressing tethered or untethered IL-7Rα/IL-2Rβ recombinant cytokine receptors at days 2, 5, 7, 9, 12 and 14 when cultured in the presence of IL-7 cytokine. FIG. 6C shows a line graph representing the number of Treg cells expressing tethered or untethered IL-7Rα/IL-2Rβ recombinant cytokine receptors at days 2, 5, 7, 9, 12 and 14 when cultured in the presence of IL-2 cytokine. FIG. 6D shows a line graph representing the number of Treg cells expressing tethered or untethered IL-4Rα/IL-2Rβ, IL-9Rα/IL-2Rβ, or IL-21Rα/IL-2Rβ recombinant cytokine receptors at days 2, 5, 7, 9, 12 and 14 when cultured in the absence of cytokine. FIG. 6E shows a line graph representing the number of Treg cells expressing tethered or untethered IL-4Rα/IL-2Rβ, IL-9Rα/IL-2Rβ, or IL-21Rα/IL-2Rβ recombinant cytokine receptors at days 2, 5, 7, 9, 12 and 14 when cultured in the presence of IL-2 cytokine. FIG. 6F shows a line graph representing the number of Treg cells expressing tethered or untethered IL-4Rα/IL-2Rβ recombinant cytokine receptors at days 2, 5, 7, 9, 12 and 14 when cultured in the presence of IL-4 cytokine. FIG. 6G shows a line graph representing the number of Treg cells expressing tethered or untethered IL-9Rα/IL-2Rβ recombinant cytokine receptors at days 2, 5, 7, 9, 12 and 14 when cultured in the presence of IL-9 cytokine. FIG. 6H shows a line graph representing the number of Treg cells expressing tethered or untethered IL-21Rα/IL-2Rβ recombinant cytokine receptors at days 2, 5, 7, 9, 12 and 14 when cultured in the presence of IL-21 cytokine. FIG. 6I shows a bar graph representing the cell fold expansion in vitro for control Tregs cultured in the presence of IL-2 cytokine, Tregs expressing tethered IL-9Rα/IL-2Rβ recombinant cytokine receptors (“IL-9 SR”) cultured in the presence of IL-2, and Tregs expressing tethered IL-9Rα/IL-2Rβ recombinant cytokine receptors cultured without IL-2.

FIG. 6J provides line graph representation of the fold expansion over time of control Tregs and Tregs expressing tethered IL-9Rα/IL-2Rβ recombinant cytokine receptors (“IL-9STR”) either cultured in the presence of IL-2 (left panel) or without IL-2 (right panel).

FIGS. 7A-7R show the expression levels of Treg markers, in particular as identified by flow cytometry. FIGS. 7A-C show FOXP3 expression in Tregs that express IL-7Rα/IL-2Rβ recombinant cytokine receptors, wherein mean FOXP3 expression is analyzed by FACS in control and recombinant Tregs cultured without cytokine (FIG. 7A), with IL-7 (FIG. 7B), or with IL-2 (FIG. 7C). FIGS. 7D-F show CD25 expression in Tregs that express IL-7Rα/IL-2Rβ recombinant cytokine receptors, wherein mean CD25 expression is analyzed by FACS in control and recombinant Tregs cultured without cytokine (FIG. 7D), with IL-7 (FIG. 7E), or with IL-2 (FIG. 7F). The dotted line indicates the baseline mean FOXP3 or CD25 expression level in control Tregs cultured with IL-2. FIG. 7G shows CD71 expression in Tregs that express tethered or untethered IL-7Rα/IL-2Rβ recombinant cytokine receptors cultured without cytokine, with IL-7, or with IL-2. FIG. 7H shows HELIOS and FOXP3 co-expression at days 7 and 12 in Tregs that express tethered IL-7Rα/IL-2Rβ recombinant cytokine receptors when Tregs are cultured without cytokine. FIG. 7I shows HELIOS and FOXP3 co-expression at days 7 and 12 in Tregs that express tethered IL-7Rα/IL-2Rβ recombinant cytokine receptors when Tregs are cultured with IL-2. FIGS. 7J-7K show HELIOS and FOXP3 co-expression at days 7 and 12 in Tregs that express tethered and untethered IL-4Rα/IL-2Rβ (FIG. 7J) or express tethered and untethered IL-9Rα/IL-2Rβ or tethered and untethered IL-21Rα/IL-2Rβ (FIG. 7K) recombinant cytokine receptors when Tregs are cultured without cytokine. FIG. 7L-7M show HELIOS and FOXP3 co-expression at days 7 and 12 in Tregs that express tethered or untethered IL-4Rα/IL-2Rβ (FIG. 7L) or express tethered or untethered IL-9Rα/IL-2Rβ or tethered or untethered IL-21Rα/IL-2Rβ (FIG. 7M) recombinant cytokine receptors when Tregs are cultured with IL-2. FIG. 7N shows HELIOS and FOXP3 co-expression at days 7 and 12 in Tregs that express tethered or untethered IL-4Rα/IL-2Rβ recombinant cytokine receptors and are cultured with IL-4. FIG. 7O shows HELIOS and FOXP3 co-expression at days 7 and 12 in Tregs that express tethered or untethered IL-9Rα/IL-2Rβ recombinant cytokine receptors and are cultured with IL-9. FIG. 7P shows HELIOS and FOXP3 co-expression at days 7 and 12 in Tregs that express tethered or untethered IL-21Rα/IL-2Rβ recombinant cytokine receptors and are cultured with IL-21. FIG. 7Q shows a bar graph representation of FOXP3 mean fluorescence intensity (MFI) as measured by flow cytometry in EGFR− negative and EGFR− positive cells from control Tregs and Tregs expressing tethered IL-9Rα/IL-2Rβ recombinant cytokine receptors (“IL-9TSR”) cultured in the presence of or without IL-2. FIG. 7R provides a bar graph representing the quantification of the percent methylation at the TSDR FOXP3 locus in conventional CD4+ T cells, control Tregs, and Tregs expressing tethered IL-9Rα/IL-2Rβ recombinant cytokine receptors (“Tether SR”).

FIGS. 8A-8M show representative histograms and bar graphs displaying the degree of STAT5 phosphorylation (pSTAT5) in Tregs expressing recombinant cytokine receptors. Phospho-STAT5 levels were analyzed by phosphoflow FACS analysis in control Tregs and Tregs expressing tethered or untethered IL-7Rα/IL-2Rβ recombinant cytokine receptors cultured without cytokine (FIG. 8A), with IL-7 (FIG. 8B), or with IL-2 (FIG. 8C) as well as presented as pSTAT5 levels in control Tregs (FIG. 8D), in Tregs expressing IL-7-tethered IL-7Rα/IL-2Rβ recombinant cytokine receptors in the presence of no cytokine (FIG. 8E), or in Tregs expressing IL-7Rα/IL-2Rβ recombinant cytokine receptors in the presence of IL-7 (FIG. 8F). FIG. 8G demonstrates representative histograms of pSTAT5 levels in response to cognate cytokine stimulation of control Tregs compared to Tregs expressing untethered IL-4Rα/IL-2Rβ, IL-9Rα/IL-2Rβ, or IL-21Rα/IL-2Rβ cytokine receptors. FIG. 8H demonstrates representative histograms of pSTAT5 levels in the presence of no cytokine stimulation for control Tregs compared to Tregs expressing tethered IL-4Rα/IL-2Rβ. IL-9Rα/IL-2Rβ, or IL-21Rα/IL-2Rβ cytokine receptors. FIG. 8I-8K provide representative histograms (FIGS. 81 and 8K) and bar graph (FIG. 8J) of pSTAT5 levels in the presence of or without IL-2 for EGFR− positive cells from control Tregs and Tregs expressing tethered IL-9Rα/IL-2Rβ cytokine receptors (“IL-9 SR”). FIG. 8L-8M provide representative histogram (FIG. 8L) and bar graph (FIG. 8M) of pSTAT5 levels in the presence of or without IL-2 for EGFR− negative cells from control Tregs and Tregs expressing tethered IL-9Rα/IL-2Rβ cytokine receptors (“IL-9 SR”).

FIGS. 9A-D show the cognate cytokine dose dependency of pSTAT5 levels in Tregs expressing IL-4Rα/IL-2Rβ (FIG. 9A), IL-7Rα/IL-2Rβ (FIG. 9B), IL-9Rα/IL-2Rβ (FIG. 9C), and IL-21Rα/IL-2Rβ (FIG. 9D) recombinant cytokine receptors in the presence of IL-4, IL-7, IL-9, or IL-21 cytokine at doses of 0 ng/mL, 0.156 ng/ml, 0.625 ng/ml, 2.5 ng/mL, 10 ng/mL, or 100 ng/mL for 40 min; or IL-2 stimulation performed at 300 U for 40 min.

FIGS. 10A-D show the IL-2 dose dependency of pSTAT5 levels in control Tregs (EGFR− Tregs) and Tregs expressing IL-7-tethered IL-7Rα/IL-2Rβ recombinant cytokine receptors (EGFR+ Tregs) upon 40 min stimulation with no cytokine (FIG. 10A), 0.156 U IL-2 (FIG. 10B), 2.5 U IL-2 (FIG. 10C), or 10 U IL-2 (FIG. 10D).

FIGS. 11A and 11B show the suppressive activity of Tregs expressing IL-7-tethered IL-7Rα/IL-2Rβ recombinant cytokine receptors on effector CD4+ T cells and effector CD8+ T cells, respectively. FIGS. 11C and 11D show the suppressive activity of Tregs expressing IL-9-tethered IL-9Rα/IL-2Rβ recombinant cytokine receptors (“IL-9TSR”) on effector CD4+ T cells and effector CD8+ T cells, respectively. FIGS. 11E and 11F show a comparison of the suppressive activity of Tregs expressing (i) IL-4-tethered IL-4Rα/IL-2Rβ recombinant cytokine receptor (“Tether 4/2”), (ii) IL-7-tethered IL-7Rα/IL-2Rβ recombinant cytokine receptor (“Tether 7/2”), or (iii) IL-9-tethered IL-9Rα/IL-2Rβ recombinant cytokine receptors (“Tether 9/2”) on effector CD4+ T cells and effector CD8+ T cells, respectively. Suppressive activity was calculated as the percent of undivided effector T cells in the PBMC/Treg co-cultures at the indicated cell ratios. Control Tregs were incubated with IL-2; Tregs expressing tethered IL-7Rα/IL-2Rβ or IL-9Rα/IL-2Rβ recombinant cytokine receptors were incubated without cytokine.

FIGS. 12A-12D show the levels of Treg lineage and activation markers on control and tethered IL-9Rα/IL-2Rβ recombinant cytokine receptor-expressing Tregs (“IL-9TSR”) cultured with PBMCs at PBMC: Treg ratios of 1:1, 1:2, 1:4 or without PBMCs, including FOXP3 (FIG. 12A), CD25 (FIG. 12B), CTLA4 (FIG. 12C), and GARP (FIG. 12D).

FIG. 13A provides a schematic overview of the Treg (re) stimulation experiment testing Treg response to anti-CD3/anti-CD28 antibody versus CD19 antigen stimulation in vitro.

FIG. 13B shows the proliferation over time of control and tethered IL-9Rα/IL-2Rβ recombinant cytokine receptor-expressing Tregs (“IL-9SR Treg”) cultured with or without IL-2 in vitro.

FIGS. 14A-14C show the average mean fluorescence intensity (MFI) over time of markers of Treg proliferation, activation, or exhaustion such as CD71 (FIG. 14A), ICOS (FIG. 14B), and PD-1 (FIG. 14C) for control and tethered IL-9Rα/IL-2Rβ recombinant cytokine receptor-expressing Tregs (“IL9 SR”) cultured with or without IL-2 in vitro using the stimulation conditions indicated in FIG. 13A.

FIGS. 15A-15C show the percent of Tregs producing cytokines, such as IL-10 (FIG. 15A), IFN-γ (FIG. 15B), and GrB (FIG. 15C) for control and tethered IL-9Rα/IL-2Rβ recombinant cytokine receptor-expressing Tregs (“IL9 SR”) cultured with IL-2 in vitro at Days 2 and 19.

FIG. 16A shows the number of Tregs that engrafted and expanding in vivo in an immunocompromised NSG mouse model within the spleen, lung, and liver for (i) control Tregs, (ii) Tregs expressing tethered IL-4Rα/IL-2Rβ recombinant cytokine receptor (“Tether 4/2”), (iii) Tregs expressing tethered IL-7Rα/IL-2Rβ recombinant cytokine receptor (“Tether 7/2”), or (iv) Tregs expressing tethered IL-9Rα/IL-2Rβ recombinant cytokine receptor (“Tether 9/2”). FIG. 16B shows the number of Tregs that engrafted and expanding in vivo in an immunocompromised NSG mouse model within the blood, spleen, lung, and liver for control and tethered IL-9Rα/IL-2Rβ recombinant cytokine receptor-expressing Tregs (“IL-9TSR”).

FIG. 16C shows the cell count over time of control and tethered IL-9Rα/IL-2Rβ recombinant cytokine receptor-expressing Tregs (IL-9TSR″) cultured with (left panel) or without (right panel) IL-2, where the cells are stimulated on Days 0, 7, and 14 as shown in FIG. 13A. FIG. 16D (graphical representation) and FIG. 16E (FACS plot) show the number of human immune cells and percent of those immune cells that are Treg cells (e.g., “CD19 CAR+IL9SR” or “CAR+IL-9SR”) circulating in the blood at 15 days post adoptive cell transfer, with versus without exogenous IL-2.

FIG. 17 shows the in vivo suppressive activity of Tregs expressing IL-9-tethered IL-9Rα/IL-2Rβ recombinant cytokine receptors (“IL-9SR Tregs”) in a graft versus host disease (GVHD) humanized murine model. PBMC, peripheral blood mononuclear cells; 5M, 5 million (5×10⁶).

FIG. 18 shows the progression of EAE score over time as an indicator of neurological autoimmunity severity (i.e., multiple sclerosis) in control mice and mice with constitutively active STAT5b (CA-STAT5b) in cells that express FOXP3 (i.e., Tregs).

FIGS. 19A-19B show the fold expansion as bar graph (FIG. 19A) and FACS plot (FIG. 19B) for wildtype (WT) Tregs with or without IL-2 and CA-STAT5b Tregs without IL-2.

FIGS. 19C-19D show the suppressive activity of WT Tregs and CA-STAT5b Tregs in vitro (FIG. 19C) and in an in vivo graft versus host disease (GVHD) murine model (FIG. 19D).

FIGS. 20A-20B show the persistence and expansion of IL-2 secreting Tregs. FIG. 20A shows the percent of IL-2 secreting Tregs cultured with or without IL-2 that persist over time. FIG. 20B shows the percent of IL-2 secreting Tregs that have expanded when cultured with (bottom panel) or without (top panel) IL-2.

FIG. 21A shows the cell count over time for control Tregs and Tregs expressing IL-2 tethered protein tags cultured with or without IL-2. FIG. 21B shows FACS analysis over time of IL-2 and EGFR protein tag levels in Tregs expressing IL-2 tethered EGFR that were grown without IL-2 in vitro. FIG. 21C provides a bar graph showing the amount of soluble IL-2 accumulating over time in the media of Tregs expressing IL-2 tethered protein tags that were grown without IL-2 in vitro.

FIG. 22 shows a heatmap of RNA sequencing analysis performed on wildtype Tregs and CA-STAT5b Tregs. 278 differentially expressed genes (DEGs) were identified, of which 99 were downregulated and 179 were upregulated in the CA-STAT5b Tregs compared to wildtype Tregs.

FIG. 23A shows a schematic of an additional exemplary recombinant cytokine receptor, wherein the recombinant cytokine receptor comprises an IL-9 cytokine tethered to a receptor with an IL-9Rα extracellular domain (ED) and IL-2Rβ transmembrane (TM) and intracellular (ID) domains (“IL9TSR (IL2RTM)”; right side), which is compared to an exemplary recombinant cytokine receptor with an IL-9Rα ED and TD and an IL-2Rβ ID (“IL9TSR (IL9RTM)”, left side, previously shown in FIG. 1E). FIG. 23B shows line graphs representing the fold expansion over time of Treg cells expressing EGFR only (control Tregs), the IL9TSR (IL9RTM) construct (also referred to as “T 9/2 (OG)” or “IL9TSR”), and the IL9TSR (IL2RTM) (also referred to as “IL-2TM”, “IL9TSR (IL2TM)”, or “T9/2 TSR (IL2RTM)”). Fold expansion was measured over time in cells cultured with (top panel) or without (bottom panel) exogenous IL-2. FIG. 23C shows FACS plots comparing cell size (i.e., SCC-H, side scatter-height) and EGFR to assess the survival at day 7 and day 14 post-transduction of Treg cells expressing EGFR as a readout for the recombinant cytokine receptor construct for Treg cells expressing EGFR only, the IL9TSR (IL9RTM) construct (i.e., “IL9TSR”), and the IL9TSR (IL2RTM) construct (i.e., “IL9TSR (IL2TM)”). FIG. 23D shows bar graphs representing the mean fluorescence intensity (MFI) of the Treg marker, FoxP3, as calculated and analyzed by FACS and flow cytometry on day 7 (top panel) and day 14 (bottom panel) post-transduction for Treg cells expressing EGFR only (both cultured with and without exogenous IL-2), the IL9TSR (IL9RTM) construct (i.e., “OG”; cultured without exogenous IL-2), and the IL9TSR (IL2RTM) construct (i.e., “IL-2TM”; cultured without exogenous IL-2).

FIG. 23E shows the percentage and distribution of Treg cells expressing phosphorylated STAT5 on day 15 in culture for Treg cells expressing EGFR only (both pulsed with and without exogenous IL-2 as pSTAT5 positive and negative control, respectively), the IL9TSR (IL9RTM) construct (without pulsing with exogenous IL-2), and the IL9TSR (IL2RTM) construct (without pulsing with exogenous IL-2).

### DETAILED DESCRIPTION

Successful adoptive cell therapy requires a robust expansion and persistence of administered cells, and the environmental signals received by the cell contribute heavily to these behaviors. Provided herein in some embodiments are systems in which the proliferation of immune cells (e.g., T cells) along with their persistence in vivo, production of immune activating cytokines, and immune function can be enhanced by the introduction of recombinant cytokine receptors. These recombinant cells may be used as a cell therapy, for example, autoimmune disorders. The present invention relates generally to the field of immunology and relates in part to compositions, and methods for growing, modifying, and expanding cells, including for example, recombinant cytokine receptors that allow immune cells such as Treg cells to proliferate in the absence of IL-2. Provided herein are recombinant cytokine receptors that activate IL-2Rβ signaling using alternative cytokines, e.g., other than IL-2. Also provided herein are recombinant cytokine receptors that are not dependent on exogenous cytokines for activation. Thus, cells transduced with the recombinant cytokine receptors described herein have certain advantages as compared to non-transduced cells, including IL-2 independence. In some embodiments of the methods and compositions provided herein, a cell is engineered to include a recombinant cytokine receptor as described herein. The recombinant cytokine receptors described herein provide stimulatory cytokine signals to the cell and improves the efficacy of cell therapy. In some embodiments, the cell is a T cell, for example a Treg.

The cells, e.g., the Tregs, that comprise these recombinant cytokine receptors can still be activated, e.g., by canonical TCR activation in the presence of target antigens, in order to suppress conventional T cells. Thus, the Tregs are not able to suppress off-target T cells or to produce suppressive cytokines in the absence of their target T cells. Such off-target activity could lead to an increased risk of chronic infection or cancer progression due to the constant suppression of conventional T cells. Thus, in some embodiments, the engineered Treg cells have fewer off-target effects and side effects compared to alternatives.

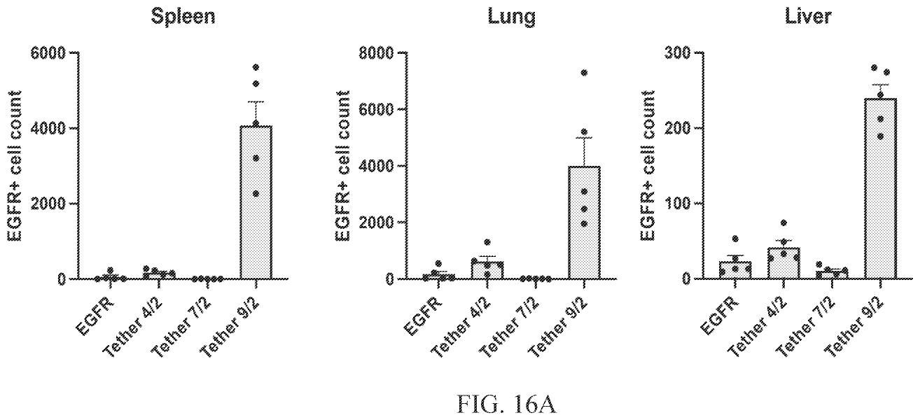

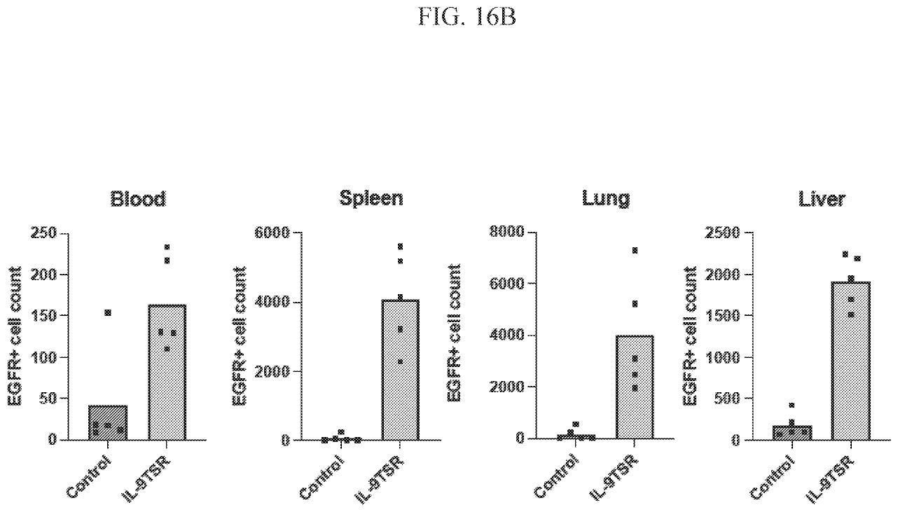

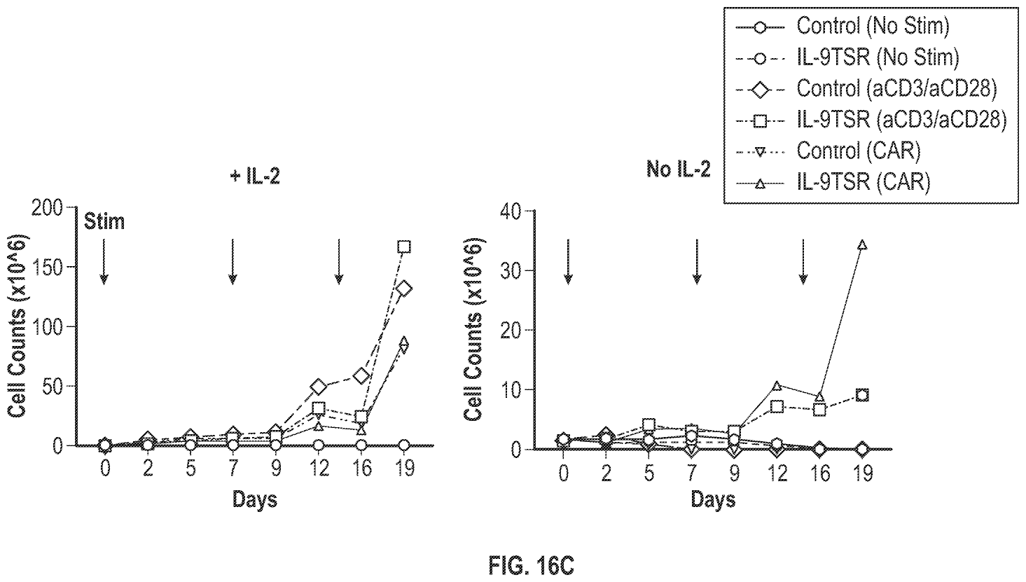

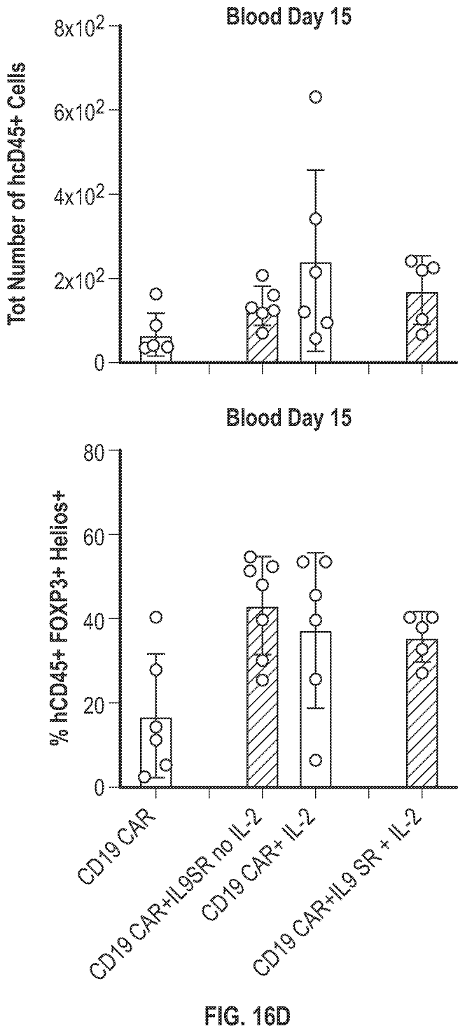

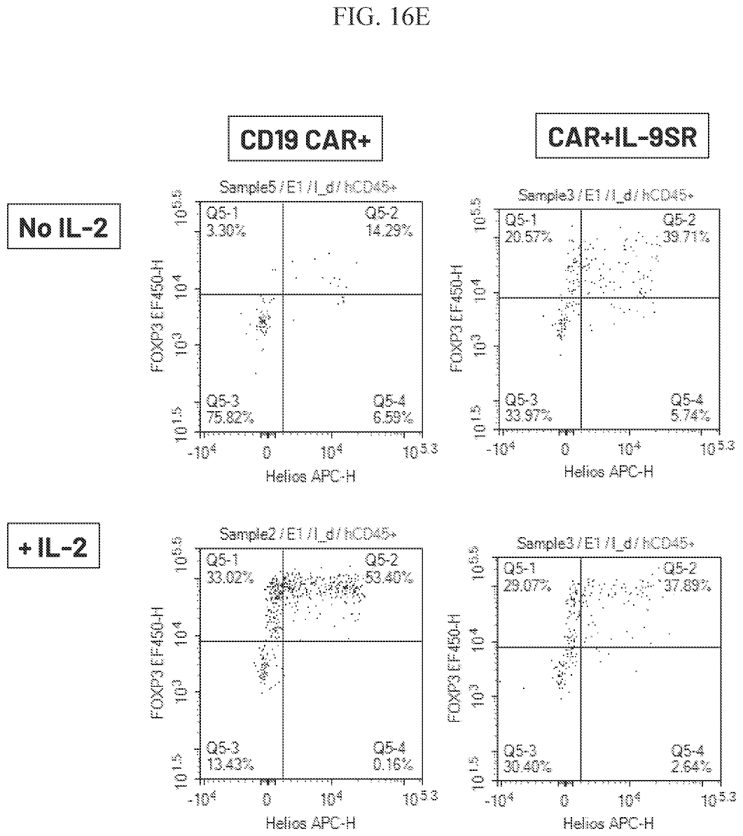

Each recombinant cytokine receptor provided herein demonstrates similar trends, albeit to varying degrees. For example, exemplary recombinant cytokine receptors tested herein include: untethered IL-4Rα/IL-2Rβ, IL-7Rα/IL-2Rβ, IL-9Rα/IL-2Rβ, and IL-21Rα/IL-2Rβ recombinant cytokine receptors, which each turns on intracellular IL-2 signaling (e.g., when transduced into a Treg) upon receptor binding to the respective cognate ligand (i.e., IL-4, IL-7, IL-9, or IL-21, respectively); and cytokine-tethered IL-4Rα/IL-2Rβ, IL-7Rα/IL-2Rβ, IL-9Rα/IL-2Rβ, and IL-21Rα/IL-2Rβ recombinant cytokine receptors, wherein intracellular IL-2 signaling is constitutively activated. Analyses in vitro demonstrated varying degrees of Treg survival, proliferation, or function (e.g., both tethered and untethered IL-21Rα/IL-2Rβ recombinant cytokine receptors showed the lowest rate of proliferation and the earliest decline in Treg survival in vitro of the eight receptors tested). Further in vivo experiments demonstrated that the tethered IL-9Rα/IL-2Rβ recombinant cytokine receptors conferred the highest degree of Treg engraftment, survival, and proliferation, even as both tethered IL-4Rα/IL-2Rβ and IL-7Rα/IL-2Rβ recombinant cytokine receptors showed in vivo engraftment, survival, and proliferation to varying degrees across the tested receptors and also across the tested tissues (see FIG. 16A). Accordingly, the in vitro and in vivo functional features of the recombinant cytokine receptors were surprising, and these differences can be leveraged to meet the biological constraints of different diseases where Treg targeting may prove beneficial for therapeutic purposes.

Furthermore, various proposed methods of activating the IL-2 signaling pathway within Tregs failed to show functional success. See Example 9, wherein three separate approaches each failed to support Treg function: a constitutively active STAT-5 Treg model, an IL-2 secreting Treg model, and an IL-2 tethered tag protein Treg model each failed to promote Treg activity against conventional T cells, thereby demonstrating that not every approach to facilitate IL-2 independence is successful when applied into Treg cells. Moreover, Treg cells are known to be quite different in terms of function, molecular profile, and genetic profile compared to conventional T cells (for example, see Grinberg-Bleyer et al. Cell 170 (6): 1096-1108, 2017; Grinberg-Bleyer et al. J Immunol 200 (7): 2362-71, 2018, both hereby incorporated by reference in their entirety), thereby necessitating a Treg-specific tailored approach, as described herein.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. Recombinant Cytokine Receptors

The present invention provides recombinant cytokine receptors that transduce intracellular interleukin-2 (IL-2) signaling in the absence of exogenous IL-2. In some embodiments, recombinant cytokine receptors are engineered molecules comprising an extracellular cytokine-binding domain (ED; also called an “extracellular cytokine receptor domain”), a transmembrane domain (TD), and an intracellular cytokine signaling domain (ID; also called an “intracellular domain”). In some embodiments, provided herein are receptors comprising an ED tethered to its cognate cytokine, which is therefore able to be activated in the absence of any exogenous cytokine. Recombinant cytokine receptors described herein are derived from interleukin cytokine receptors, wherein the ID comprises the ID of the Interleukin-2 Receptor beta (IL-2Rβ) chain of the IL-2R. Canonical IL-2 signaling causes STAT5 phosphorylation, thereby activating STAT5 nuclear translocation and initiation of target gene transcription. In some cells, e.g., T cells, including Treg cells, IL-2 signaling is required for cell survival and cell proliferation. Therefore, recombinant cytokine receptors comprising an IL-2Rβ intracellular domain that bind to a cytokine other than IL-2 are able to activate cell survival and proliferation in the absence of IL-2 via binding to another cytokine.

In some embodiments recombinant cytokine receptors described herein are described with respect to the extracellular domain and intracellular domain. Thus, for example, an “IL-4Rα/IL-2Rβ” receptor comprises an IL-4 receptor extracellular domain linked to an IL-2Rβ intracellular domain. In some embodiments, the receptor comprises a transmembrane domain that is from the same cytokine receptor as the extracellular domain. In some embodiments, the receptor comprises a transmembrane domain from a different cytokine from the extracellular domain.

In some embodiments, the recombinant cytokine receptors are more effective for maintaining immune cell persistence. In some embodiments, the immune cell is a T cell. In some embodiments, the immune cell is a Treg.

In some embodiments, the recombinant cytokine receptors are more effective for maintaining Treg persistence compared to other strategies for producing IL-2 independent Tregs, such as, for example, Tregs that are engineered to secrete IL-2. In some embodiments, cells transduced with the recombinant cytokine receptors provided herein are able to maintain a Treg phenotype and/or have increased persistence compared to Treg cells that secrete IL-2.

In some embodiments, the recombinant cytokine receptors provided herein have significant advantages including, but not limited to: (1) expression in regulatory T cells (Tregs); (2) increasing Treg survival and proliferation in the absence of IL-2; (3) increased IL-2 receptor signaling via STAT5 phosphorylation; and (4) ability to suppress effector T cells in the absence of IL-2 to about or approximately the same degree as wild-type Tregs grown in the presence of IL-2.

TABLE 1
Untethered recombinant cytokine receptor sequences

| Recombinant | Full | Extracellular | Transmembrane | Intracellular |
|---|---|---|---|---|
| Cytokine | receptor | domain | domain | domain |
| Receptor | sequence | sequence | sequence | sequence |
| IL-4Rα/ | (SEQ ID | (SEQ ID | (SEQ ID | (SEQ ID |
| IL-2Rβ | NO: 1) | NO: 11) | NO: 16) | NO: 20) |
| IL-7Rα/ | (SEQ ID | (SEQ ID | (SEQ ID | (SEQ ID |
| IL-2Rβ | NO: 2) | NO: 12) | NO: 17) | NO: 20) |
| IL-9Rα/ | (SEQ ID | (SEQ ID | (SEQ ID | (SEQ ID |
| IL-2Rβ | NO: 3) | NO: 13) | NO: 18) | NO: 20) |
| IL-21Rα/ | (SEQ ID | (SEQ ID | (SEQ ID | (SEQ ID |
| IL-2Rβ | NO: 4) | NO: 14) | NO: 19) | NO: 20) |
| IL-9RαED/ | (SEQ ID | (SEQ ID | (SEQ ID | (SEQ ID |
| IL-2RβTD + | NO: 5) | NO: 13) | NO: 21) | NO: 20) |
| ID | | | | |

TABLE 2
Tethered recombinant cytokine receptor sequences

| | | | Trans- | | | |
|---|---|---|---|---|---|---|
| Recombinant | Full | Extracellular | membrane | Intracellular | | |
| Cytokine | receptor | domain | domain | domain | Linker | Cytokine |
| Receptor | sequence | sequence | sequence | sequence | sequence | sequence |
| IL-4 + IL-4Rα/IL-2Rβ | (SEQ ID | (SEQ ID | (SEQ ID | (SEQ ID | (SEQ ID | (SEQ ID |
| | NO: 6) | NO: 11) | NO: 16) | NO: 20) | NO: 27) | NO: 22) |
| IL-7 + IL-7Rα/IL-2Rβ | (SEQ ID | (SEQ ID | (SEQ ID | (SEQ ID | (SEQ ID | (SEQ ID |
| | NO: 7) | NO: 12) | NO: 17) | NO: 20) | NO: 27) | NO: 23) |
| IL-9 + IL-9Rα/IL-2Rβ | (SEQ ID | (SEQ ID | (SEQ ID | (SEQ ID | (SEQ ID | (SEQ ID |
| | NO: 8) | NO: 13) | NO: 18) | NO: 20) | NO: 27) | NO: 24) |
| IL-21 + IL-21Rα/IL- | (SEQ ID | (SEQ ID | (SEQ ID | (SEQ ID | (SEQ ID | (SEQ ID |
| 2Rβ | NO: 9) | NO: 14) | NO: 19) | NO: 20) | NO: 27) | NO: 25) |
| IL-9 + IL-9RαED/IL- | (SEQ ID | (SEQ ID | (SEQ ID | (SEQ ID | (SEQ ID | (SEQ ID |
| 2RβTD + ID | NO: 15) | NO: 13) | NO: 21) | NO: 20) | NO: 27) | NO: 24) |

Provided herein are recombinant cytokine receptors comprising a cytokine receptor ED and TD, and the ID of Interleukin-2 Receptor beta (IL-2Rβ), wherein the ED does not bind to exogenous IL-2. In some embodiments, the recombinant cytokine receptor is able to signal in the absence of exogenous IL-2. In some embodiments, the recombinant cytokine receptor comprises an interleukin ED, a TD, and an ID. In some embodiments, the ED binds to a cytokine other than IL-2. Thus, in some embodiments, there is provided a recombinant cytokine receptor comprising: (I) the ED of IL-4Rα, IL-7Rα, IL-9Rα, or IL-21Rα; (II) the TD of IL-2Rβ, IL-4Rα, IL-7Rα, IL-9Rα, or IL-21Rα; (III) the ID of IL-2Rβ. In some embodiments, the recombinant cytokine receptor comprises the amino acid sequence set forth in SEQ ID NOs: 1-5. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NOs: 1-5. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NOs: 11-14.

In some embodiments, the recombinant cytokine receptor comprises (I) the ED of IL-4Rα; (II) the TD of IL-4Rα; and (III) the ID of IL-2Rβ. In some embodiments, the recombinant cytokine receptor comprises the amino acid sequence set forth in SEQ ID NO:1. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:1. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 11. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:11; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 16; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) an ED comprising the amino acid sequence of SEQ ID NO: 11; (II) a TD comprising the amino acid sequence of SEQ ID NO: 16; and (III) an ID comprising the amino acid sequence of SEQ ID NO:20.

In some embodiments, the recombinant cytokine receptor comprises (I) the ED of IL-4Rα; (II) the TD of IL-2Rβ; and (III) the ID of IL-2Rβ. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:11. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:11; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) an ED comprising the amino acid sequence of SEQ ID NO: 11; (II) a TD comprising the amino acid sequence of SEQ ID NO:21; and (III) an ID comprising the amino acid sequence of SEQ ID NO:20.

In some embodiments, the recombinant cytokine receptor comprises (I) the ED of IL-7Rα; (II) the TD of IL-7Rα; and (III) the ID of IL-2Rβ. In some embodiments, the recombinant cytokine receptor comprises the amino acid sequence set forth in SEQ ID NO:2. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:2. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 12. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:12; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 17; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) an ED comprising the amino acid sequence of SEQ ID NO: 12; (II) a TD comprising the amino acid sequence of SEQ ID NO:17; and (III) an ID comprising the amino acid sequence of SEQ ID NO:20.

In some embodiments, the recombinant cytokine receptor comprises (I) the ED of IL-7Rα; (II) the TD of IL-2Rβ; and (III) the ID of IL-2Rβ. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 12. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 12; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) an ED comprising the amino acid sequence of SEQ ID NO: 12; (II) a TD comprising the amino acid sequence of SEQ ID NO:21; and (III) an ID comprising the amino acid sequence of SEQ ID NO:20.

In some embodiments, the recombinant cytokine receptor comprises (I) the ED of IL-9Rα; (II) the TD of IL-9Rα; and (III) the ID of IL-2Rβ. In some embodiments, the recombinant cytokine receptor comprises the amino acid sequence set forth in SEQ ID NO:3. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:3. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 13. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:13; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 18; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) an ED comprising the amino acid sequence of SEQ ID NO: 13; (II) a TD comprising the amino acid sequence of SEQ ID NO:18; and (III) an ID comprising the amino acid sequence of SEQ ID NO:20.

In some embodiments, the recombinant cytokine receptor comprises (I) the ED of IL-9Rα; (II) the TD of IL-2Rβ; and (III) the ID of IL-2Rβ. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:13. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 13; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) an ED comprising the amino acid sequence of SEQ ID NO: 13; (II) a TD comprising the amino acid sequence of SEQ ID NO:21; and (III) an ID comprising the amino acid sequence of SEQ ID NO:20. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:5. In some embodiments, the recombinant cytokine receptor comprises the amino acid sequence set forth in SEQ ID NO:5.

In some embodiments, the recombinant cytokine receptor comprises (I) the ED of IL-21Rα; (II) the TD of IL-21Rα; and (III) the ID of IL-2Rβ. In some embodiments, the recombinant cytokine receptor comprises the amino acid sequence set forth in SEQ ID NO:4. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:4. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:14. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 14; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 19; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) the ED comprising the amino acid sequence of SEQ ID NO: 14; (II) a TD comprising the amino acid sequence of SEQ ID NO:19; and (III) an ID comprising the amino acid sequence of SEQ ID NO: 20.

In some embodiments, the recombinant cytokine receptor comprises (I) the ED of IL-21Rα; (II) the TD of IL-2Rβ; and (III) the ID of IL-2Rβ. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 14. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 14; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; and (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:20. In some embodiments, the recombinant cytokine receptor comprises (I) the ED comprising the amino acid sequence of SEQ ID NO: 14; (II) a TD comprising the amino acid sequence of SEQ ID NO: 21; and (III) an ID comprising the amino acid sequence of SEQ ID NO:20.

In some embodiments, the recombinant cytokine receptor comprises (I) the ED of IL-4Rα, IL-7Rα, IL-9Rα, or IL-21Rα; (II) the TD of IL-2Rβ, IL-4Rα, IL-7Rα, IL-9Rα, or IL-21Rα; (III) the ID of IL-2Rβ; and (IV) a cognate cytokine tethered by a polypeptide linker to the ED of the recombinant cytokine receptor. In some embodiments, the ED is from the same cytokine receptor as the TD (e.g., an ED from IL-4Rα and a TD from IL-4Rα). In some embodiments, the cognate cytokine is IL-4, IL-7, IL-9, or IL-21. In some embodiments, the cognate cytokine is IL-4. In some embodiments, the cognate cytokine is IL-7. In some embodiments, the cognate cytokine is IL-9. In some embodiments, the cognate cytokine is IL-21. In some embodiments, the recombinant cytokine receptor comprises the amino acid sequence set forth in SEQ ID NOs: 6-9 and 15.

In some embodiments, the recombinant cytokine receptor comprises (I) the ED of IL-4Rα; (II) the TD of IL-4Rα; (III) the ID of IL-2Rβ; and (IV) the cognate cytokine IL-4 tethered by a polypeptide linker to the ED of the recombinant cytokine receptor. In some embodiments, the recombinant cytokine receptor comprises the amino acid sequence set forth in SEQ ID NO:6.

In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:6. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:11. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 11; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 16; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:22 tethered by a polypeptide linker to the ED of the recombinant cytokine receptor. In some embodiments, the recombinant cytokine receptor comprises (I) an ED comprising the amino acid sequence of SEQ ID NO: 11; (II) a TD comprising the amino acid sequence of SEQ ID NO: 16; (III) an ID comprising the amino acid sequence of SEQ ID NO:20; and (IV) a cytokine comprising the amino acid sequence of SEQ ID NO:22 tethered by a polypeptide linker to the ED of the recombinant cytokine receptor.

In some embodiments, the recombinant cytokine receptor comprises (I) the ED of IL-4Rα; (II) the TD of IL-2Rβ; (III) the ID of IL-2Rβ; and (IV) the cognate cytokine IL-4 tethered by a polypeptide linker to the ED of the recombinant cytokine receptor. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:11. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:11; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:22 tethered by a polypeptide linker to the ED of the recombinant cytokine receptor. In some embodiments, the recombinant cytokine receptor comprises (I) an ED comprising the amino acid sequence of SEQ ID NO: 11; (II) a TD comprising the amino acid sequence of SEQ ID NO:21; (III) an ID comprising the amino acid sequence of SEQ ID NO:20; and (IV) a cytokine comprising the amino acid sequence of SEQ ID NO:22 tethered by a polypeptide linker to the ED of the recombinant cytokine receptor.

In some embodiments, the recombinant cytokine receptor comprises (I) the ED of IL-7Rα; (II) the TD of IL-7Rα; (III) the ID of ILR-2B; and (IV) the cognate cytokine IL-7 tethered by a polypeptide linker to the ED of the recombinant cytokine receptor. In some embodiments, the recombinant cytokine receptor comprises the amino acid sequence set forth in SEQ ID NO:7. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:7. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 12. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 12; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 17; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:23 tethered by a polypeptide linker to the ED of the recombinant cytokine receptor. In some embodiments, the recombinant cytokine receptor comprises (I) an ED comprising the amino acid sequence of SEQ ID NO: 12; (II) a TD comprising the amino acid sequence of SEQ ID NO: 17; (III) an ID comprising the amino acid sequence of SEQ ID NO:20; and (IV) a cytokine comprising the amino acid sequence of SEQ ID NO:23 tethered by a polypeptide linker to the ED of the recombinant cytokine receptor.

In some embodiments, the recombinant cytokine receptor comprises (I) the ED of IL-7Rα; (II) the TD of IL-2Rβ; (III) the ID of IL-2Rβ; and (IV) the cognate cytokine IL-7 tethered by a polypeptide linker to the ED of the recombinant cytokine receptor. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 12. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 12; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:23 tethered by a polypeptide linker to the ED of the recombinant cytokine receptor. In some embodiments, the recombinant cytokine receptor comprises (I) an ED comprising the amino acid sequence of SEQ ID NO: 12; (II) a TD comprising the amino acid sequence of SEQ ID NO:21; (III) an ID comprising the amino acid sequence of SEQ ID NO:20; and (IV) a cytokine comprising the amino acid sequence of SEQ ID NO:23 tethered by a polypeptide linker to the ED of the recombinant cytokine receptor.

In some embodiments, the recombinant cytokine receptor comprises (I) the ED of IL-9Rα; (II) the TD of IL-9Rα; (III) the ID of IL-2Rβ; and (IV) the cognate cytokine IL-9 tethered by a polypeptide linker to the ED of the recombinant cytokine receptor. In some embodiments, the recombinant cytokine receptor comprises the amino acid sequence set forth in SEQ ID NO:8. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:8. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 13. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 13; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 18; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:24 tethered by a polypeptide linker to the ED of the recombinant cytokine receptor. In some embodiments, the recombinant cytokine receptor comprises (I) an ED comprising the amino acid sequence of SEQ ID NO: 13; (II) a TD comprising the amino acid sequence of SEQ ID NO: 18; (III) an ID comprising the amino acid sequence of SEQ ID NO:20; and (IV) a cytokine comprising the amino acid sequence of SEQ ID NO:24 tethered by a polypeptide linker to the ED of the recombinant cytokine receptor.

In some embodiments, the recombinant cytokine receptor comprises (I) the ED of IL-9Rα; (II) the TD of IL-2Rβ; (III) the ID of IL-2Rβ; and (IV) the cognate cytokine IL-9 tethered by a polypeptide linker to the ED of the recombinant cytokine receptor. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 13. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 13; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:24 tethered by a polypeptide linker to the ED of the recombinant cytokine receptor. In some embodiments, the recombinant cytokine receptor comprises (I) an ED comprising the amino acid sequence of SEQ ID NO: 13; (II) a TD comprising the amino acid sequence of SEQ ID NO:21; (III) an ID comprising the amino acid sequence of SEQ ID NO:20; and (IV) a cytokine comprising the amino acid sequence of SEQ ID NO:24 tethered by a polypeptide linker to the ED of the recombinant cytokine receptor. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 15. In some embodiments, the recombinant cytokine receptor comprises the amino acid sequence set forth in SEQ ID NO:15.

In some embodiments, the recombinant cytokine receptor comprises (I) the ED of IL-21Rα; (II) the TD of IL-21Rα; (III) the ID of IL-2Rβ; and (IV) the cognate cytokine IL-21 tethered by a polypeptide linker to the ED of the recombinant cytokine receptor. In some embodiments, the recombinant cytokine receptor comprises the amino acid sequence set forth in SEQ ID NO:9. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:9. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:14. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 14; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 19; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:25 tethered by a polypeptide linker to the ED of the recombinant cytokine receptor. In some embodiments, the recombinant cytokine receptor comprises (I) an ED comprising the amino acid sequence of SEQ ID NO: 14; (II) a TD comprising the amino acid sequence of SEQ ID NO: 19; (III) an ID comprising the amino acid sequence of SEQ ID NO:20; and (IV) a cytokine comprising the amino acid sequence of SEQ ID NO:25 tethered by a polypeptide linker to the ED of the recombinant cytokine receptor.

In some embodiments, the recombinant cytokine receptor comprises (I) the ED of IL-21Rα; (II) the TD of IL-2Rβ; (III) the ID of IL-2Rβ; and (IV) the cognate cytokine IL-21 tethered by a polypeptide linker to the ED of the recombinant cytokine receptor. In some embodiments, the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:14. In some embodiments, the recombinant cytokine receptor comprises (I) an ED comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 14; (II) a TD comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:21; (III) an ID comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO: 20; and (IV) a cytokine comprising an amino acid sequence comprising at least about 80%, at least about 85%, at least about 90%, at least about 95%, at least about 96%, at least about 97%, at least about 98%, at least about 99%, or about 100% sequence identity with the amino acid sequence of SEQ ID NO:25 tethered by a polypeptide linker to the ED of the recombinant cytokine receptor. In some embodiments, the recombinant cytokine receptor comprises (I) an ED comprising the amino acid sequence of SEQ ID NO: 14; (II) a TD comprising the amino acid sequence of SEQ ID NO:21; (III) an ID comprising the amino acid sequence of SEQ ID NO:20; and (IV) a cytokine comprising the amino acid sequence of SEQ ID NO:25 tethered by a polypeptide linker to the ED of the recombinant cytokine receptor.

In some embodiments, the cells expressing the recombinant cytokine receptor can survive and proliferate in the absence of IL-2. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the cells transduced with the recombinant cytokine receptor remain viable in the absence of IL-2. In some embodiments, the cells are T cells. In some embodiments, the cells are Treg cells.

In some embodiments, the Tregs expressing the recombinant cytokine receptor can survive and proliferate in vitro in the absence of IL-2 in a suitable medium that can maintain Tregs. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor remain viable in vitro about 2 days to about 30 days, such as about 10 to about 30 days, about 20 to about 30 days, or about 23 to about 30 days after transduction. In some embodiments, at least about 60%-99% of the Tregs transduced with the recombinant cytokine receptor remain viable in vitro about 7 to about 20 days after transduction. In some embodiments, at least about 60-90% of the Tregs transduced with the recombinant cytokine receptor remain viable in vitro about 7 to about 20 days after transduction. In some embodiments, at least about 60-80% of the Tregs transduced with the recombinant cytokine receptor remain viable in vitro about 7 to about 20 days after transduction. In some embodiments, at least about 60%-99% of the Tregs transduced with the recombinant cytokine receptor remain viable in vitro about 7 days after transduction. In some embodiments, at least about 60-90% of the Tregs transduced with the recombinant cytokine receptor remain viable in vitro about 7 after days after transduction. In some embodiments, at least about 60-80% of the Tregs transduced with the recombinant cytokine receptor remain viable in vitro about 7 days after transduction. In some embodiments, at least about 60%-99% of the Tregs transduced with the recombinant cytokine receptor remain viable in vitro about 12 days after transduction. In some embodiments, at least about 60-90% of the Tregs transduced with the recombinant cytokine receptor remain viable in vitro about 12 after days after transduction. In some embodiments, at least about 60-80% of the Tregs transduced with the recombinant cytokine receptor remain viable in vitro about 12 days after transduction. In some embodiments, at least about 60%-99% of the Tregs transduced with the recombinant cytokine receptor remain viable in vitro about 14 days after transduction. In some embodiments, at least about 60-90% of the Tregs transduced with the recombinant cytokine receptor remain viable in vitro about 14 after days after transduction. In some embodiments, at least about 60-80% of the Tregs transduced with the recombinant cytokine receptor remain viable in vitro about 14 days after transduction. In some embodiments, the viability of Tregs transduced with the recombinant cytokine receptor is increased compared to Treg cells that are not transduced with the recombinant cytokine receptor. In some embodiments, the viability of Tregs transduced with the recombinant cytokine receptor is increased compared to Treg cells that are not transduced with the recombinant cytokine receptor when cultured without the cognate receptor cytokine. In some embodiments, the viability of Tregs is increased at least about 1.1-fold, at least about 1.5-fold, at least about 2-fold, at least about 3-fold, at least about 3.2-fold, at least about 4-fold, at least about 10-fold, about 20-fold, at least about 25-fold, at least about 30-fold, at least about 35-fold, at least about 45-fold, at least about 50-fold, or more compared to untransduced T cells.

In some embodiments, the Tregs expressing the recombinant cytokine receptor can survive and proliferate in vivo in the absence of IL-2 in the host organism. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor can persist in vivo for at least about 3 days or longer. In some embodiments, at least about 60%-99% of the Tregs transduced with the recombinant cytokine receptor can persist in vivo for at least about 3 days or longer. In some embodiments, at least about 60-90% of the Tregs transduced with the recombinant cytokine receptor can persist in vivo for at least about 3 days or longer. In some embodiments, at least about 60-80% of the Tregs transduced with the recombinant cytokine receptor can persist in vivo for at least about 3 days or longer.

In some embodiments, the cells transduced with the recombinant cytokine receptor have increased or sustained proliferation post-transduction and in vivo. In some embodiments, the cells transduced with the recombinant cytokine receptor have more cells in an expanded in vitro culture compared to the number of cells before transduction. In some embodiments, the cells are T cells. In some embodiments, the cells are Treg cells.

In some embodiments, the Tregs transduced with the recombinant cytokine receptor have increased or sustained proliferation post-transduction. In some embodiments, the Tregs transduced with the recombinant cytokine receptor have more cells after expansion compared to the number of cells before transduction. In some embodiments, Tregs transduced with the recombinant cytokine receptor have a higher rate of proliferation compared to untransduced Treg cells. In some embodiments, the number of cells post-transduction is increased at least about 1.1-fold, at least about 1.5-fold, at least about 2-fold, at least about 5-fold, at least about 10-fold, at least about 20-fold, at least about 50-fold, at least about 75-fold, or more compared to untransduced cells. In some embodiments, the cells are cultured post-transduction in the presence of cytokine. In some embodiments, the cells are cultured post-transduction without cytokine.

In some embodiments, cell viability, transduction of intracellular signaling, activation of intracellular signaling, or the ability to proliferate is increased upon cell transduction with the recombinant cytokine receptor.

In some embodiments, cell viability, transduction of intracellular signaling, activation of intracellular signaling, or the ability to proliferate is increased upon cell transduction with the recombinant cytokine receptor. In some embodiments, the recombinant cytokine receptor expressed in cells transduces intracellular IL-2 signaling within the cells. In some embodiments, the recombinant cytokine receptor activates intracellular IL-2 signaling in cells. In some embodiments, the recombinant cytokine receptor promotes intracellular IL-2 signaling in cells. In some embodiments, the recombinant cytokine receptor increases intracellular IL-2 signaling in cells compared to cells without the recombinant cytokine receptor. In some embodiments, the intracellular domain of the recombinant cytokine receptors provided herein comprise an active IL-2Rβ intracellular domain. In some embodiments, the recombinant cytokine receptors comprise an active IL-2Rβ intracellular domain. In some embodiments, the intracellular domain of the recombinant cytokine receptors provided herein are able to engage in downstream signal transduction. In some embodiments, the recombinant cytokine receptor comprises an IL-2Rβ intracellular domain which phosphorylates STAT5 through its activation of the JAK1 kinase upon recombinant cytokine receptor activation. In some embodiments, the IL-2Rβ intracellular domain phosphorylates STAT5 through its activation of the JAK1 kinase upon recombinant cytokine receptor activation. In some embodiments, the recombinant cytokine receptor phosphorylates Shc upon recombinant cytokine receptor activation, thereby activating the downstream PI3K-AKT pathway. In some embodiments, the recombinant cytokine receptor phosphorylates Shc upon recombinant cytokine receptor activation, thereby activating the downstream Ras/MAPK pathway. In some embodiments, the recombinant cytokine receptor comprises IL-4Rα/IL-2Rβ, IL-7Rα/IL-2Rβ, IL-9Rα/IL-2Rβ, or IL-21Rα/IL-2Rβ extracellular/intracellular receptor domains. In some embodiments, the cells are T cells. In some embodiments, the cells are Treg cells.

In some embodiments, the recombinant cytokine receptor phosphorylates STAT5 through its activation of the JAK1 kinase upon recombinant cytokine receptor activation. In some embodiments, the recombinant cytokine receptor phosphorylates Shc upon recombinant cytokine receptor activation, thereby activating the downstream PI3K-AKT pathway. In some embodiments, the recombinant cytokine receptor phosphorylates Shc upon recombinant cytokine receptor activation, thereby activating the downstream Ras/MAPK pathway. In some embodiments, the recombinant cytokine receptor comprises IL-4Rα/IL-2Rβ extracellular/intracellular receptor domains. In some embodiments, the recombinant cytokine receptor is IL-4Rα/IL-2Rβ.

In some embodiments, the recombinant cytokine receptor has a capability to stimulate STAT5 phosphorylation in cells (e.g., Tregs). STAT5 signaling can be measured, for example, by phosphorylation of STAT5 using any suitable method known in the art. For example, STAT5 phosphorylation can be measured using antibodies specific for the phosphorylated version of these molecules in combination with flow cytometry analysis as described herein.

### 1. Extracellular Domain

IL-2 is a class I cytokine. Class I cytokine receptors generally have large extracellular domains (EDs) that include multiple all-β Ig-like domains and Fn3 domains (see, e.g., Metcalfe, R D et al. (2020), Front Immunol; 11:1424). These domains possess a β-sandwich structure with two anti-parallel β sheets. Two Fn3 domains form the cytokine binding homology region at the domain juncture. Class I cytokine receptor EDs contain a conserved WSXWS (Trp-Ser-X-Trp-Ser, where X is any amino acid) motif that may act to stabilize the receptor, undergo conformational change upon cytokine binding, and can be extensively glycosylated. Class I cytokine receptor chains, including IL-2Rβ, IL-4Rα, IL-7Rα, IL-9Rα, and IL-21Rα are most often found in heterodimers or heterotrimers. For example, IL-2Rβ can be found in a heterodimer with IL-2Rγ_{c} or in a heterotrimer with IL-2Rα and IL-2Rγ_{c}, wherein the IL-2 binding affinity for IL-2Rβ alone is K_{d}˜100 nm, in heterodimeric form K_{d}˜1 nM, and in heterotrimeric form K_{d}˜10 pM (see, e.g., Wang, X et al. (2009), Annu Rev Immunol; 27:29-60, hereby incorporated by reference in its entirety). Reconstitution studies have identified the cognate cytokine binding affinities for IL-4Rα, IL-7Rα, IL-9Rα, and IL-21Rα are the following: K_{d}˜266 pM, K_{d}˜250 pM, K_{d}˜100 pM (see, e.g., Lin, J X & Leonard, W J (2018), Cold Spring Harb Perspect Biol; 10 (9): a028449, hereby incorporated by reference in its entirety), and K_{d}˜70 pM (see, e.g., Kang, L et al. (2010), J Biol Chem; 285 (16): 12223-12231, hereby incorporated by reference in its entirety), respectively. These binding affinities can become higher-affinity upon each cytokine receptor complexing with ye chains. The EDs of IL-4Rα, IL-7Rα, IL-9Rα, and IL-21Rα are 232 residues, 239 residues, 270 residues, and 232 residues, respectively, in length. IL-2, IL-4, IL-7, IL-9, and IL-21 signaling pathways each have their own distinct impact on T cell biology: T cell survival and proliferation; T helper 2 (T_{H}2) differentiation from naïve T cells and anti-inflammatory actions via suppression of T helper 1 (T_{H}1) and induced differentiation of Tregs; survival of naïve and memory T cells; enhancement of Treg suppressive activity and differentiation of T_{H}17 cells; and promotion of CD8+ T cell clonal expansion and cytolytic activity, respectively.

In some embodiments, the recombinant cytokine receptor is able to signal in the absence of exogenous IL-2. In some embodiments, the recombinant cytokine receptor comprises an interleukin ED, a TD, and an ID. Thus, in some embodiments, there is provided a recombinant cytokine receptor comprising: (I) the ED of IL-4Rα, IL-7Rα, IL-9Rα, or IL-21Rα; (II) the TD of IL-2Rβ, IL-4Rα, IL-7Rα, IL-9Rα, or IL-21Rα; (III) the ID of IL-2Rβ; and (IV) optionally, a cognate cytokine tethered by a polypeptide linker to the ED of the recombinant cytokine receptor. In some embodiments, the ED comprises the amino acid sequence set forth in SEQ ID NOs: 11-14. In some embodiments, the ED is from the same cytokine receptor as the TD (e.g., an ED from IL-4Rα and a TD from IL-4Rα). In some embodiments, the ED is from a different cytokine receptor from the TD.

In some embodiments, the recombinant cytokine receptor comprises the ED of IL-4Rα. In some embodiments ED comprises the amino acid sequence set forth in SEQ ID NO:11. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 11. In some embodiments, the recombinant cytokine receptor comprises in N-terminal to C-terminal direction: (I) the ED of IL-4Rα; (II) the TD of IL-2Rβ, IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-4Rα; (II) the TD of IL-4Rα; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 11; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:16; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO: 11; (II) a TD comprising the amino acid sequence of SEQ ID NO: 16; and (III) an ID comprising the amino acid sequence of SEQ ID NO: 20. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:1.

In some embodiments, the recombinant cytokine receptor comprises the ED of IL-4Rα. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 11. In some embodiments ED comprises the amino acid sequence set forth in SEQ ID NO:11. In some embodiments, the recombinant cytokine receptor comprises in N-terminal to C-terminal direction: (I) the ED of IL-4Rα; (II) the TD of IL-2Rβ, IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-4Rα; (II) the TD of IL-2Rβ; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:11; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 21; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO:11; (II) a TD comprising the amino acid sequence of SEQ ID NO:21; and (III) an ID comprising the amino acid sequence of SEQ ID NO:20.

In some embodiments, the recombinant cytokine receptor comprises the ED of IL-7Rα. In some embodiments, the IL-7Rα ED comprises the amino acid sequence set forth in SEQ ID NO: 12. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:12. In some embodiments, the recombinant cytokine receptor comprises in N-terminal to C-terminal direction: (I) the ED of IL-7Rα; (II) the TD of IL-2Rβ, IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-7Rα; (II) the TD of IL-7Rα; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 12; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 17; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO: 12; (II) a TD comprising the amino acid sequence of SEQ ID NO: 17; and (III) an ID comprising the amino acid sequence of SEQ ID NO:20. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 2.

In some embodiments, the recombinant cytokine receptor comprises the ED of IL-7Rα. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 12. In some embodiments ED comprises the amino acid sequence set forth in SEQ ID NO:12. In some embodiments, the recombinant cytokine receptor comprises in N-terminal to C-terminal direction: (I) the ED of IL-7Rα; (II) the TD of IL-2Rβ. IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-7Rα; (II) the TD of IL-2Rβ; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 12; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 21; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO:12; (II) a TD comprising the amino acid sequence of SEQ ID NO: 21; and (III) an ID comprising the amino acid sequence of SEQ ID NO:20.

In some embodiments, the recombinant cytokine receptor comprises the ED of IL-9Rα. In some embodiments, the ED comprises the amino acid sequence set forth in SEQ ID NO: 13. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:13. In some embodiments, the recombinant cytokine receptor comprises in N-terminal to C-terminal direction: (I) the ED of IL-9Rα; (II) the TD of IL-2Rβ, IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-9Rα; (II) the TD of IL-9Rα; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:13; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:18; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO: 13; (II) a TD comprising the amino acid sequence of SEQ ID NO:18; and (III) an ID comprising the amino acid sequence of SEQ ID NO:20. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 3.

In some embodiments, the recombinant cytokine receptor comprises the ED of IL-9Rα. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 13. In some embodiments ED comprises the amino acid sequence set forth in SEQ ID NO: 13. In some embodiments, the recombinant cytokine receptor comprises in N-terminal to C-terminal direction: (I) the ED of IL-9Rα; (II) the TD of IL-2Rβ. IL-4Rα. IL-7Rα. IL-9Rα, or IL-21Rα; and (III) the ID of IL-2Rβ. In some embodiments, the recombinant cytokine receptor comprises in N-terminal to C-terminal direction: (I) the ED of IL-9Rα; (II) the TD of IL-2Rβ; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 13; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 21; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO:13; (II) a TD comprising the amino acid sequence of SEQ ID NO:21; and (III) an ID comprising the amino acid sequence of SEQ ID NO:20. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:5.

In some embodiments, the IL-21Rα ED comprises the amino acid sequence set forth in SEQ ID NO:14. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 14. In some embodiments, the recombinant cytokine receptor comprises in N-terminal to C-terminal direction: (I) the ED of IL-21Rα; (II) the TD of IL-2Rβ, IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-21Rα; (II) the TD of IL-21Rα; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:14; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 19; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO:14; (II) a TD comprising the amino acid sequence of SEQ ID NO:19; and (III) an ID comprising the amino acid sequence of SEQ ID NO: 20. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:4.

In some embodiments, the recombinant cytokine receptor comprises the ED of IL-21Rα. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 14. In some embodiments ED comprises the amino acid sequence set forth in SEQ ID NO:14. In some embodiments, the recombinant cytokine receptor comprises in N-terminal to C-terminal direction: (I) the ED of IL-21Rα; (II) the TD of IL-2Rβ. IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-21Rα; (II) the TD of IL-2Rβ; and (III) the ID of IL-2Rβ. In some embodiments, the recombinant cytokine receptor comprises in N-terminal to C-terminal direction: (I) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 14; (II) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 21; and (III) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) an ED comprising the amino acid sequence of SEQ ID NO: 14; (II) a TD comprising the amino acid sequence of SEQ ID NO:21; and (III) an ID comprising the amino acid sequence of SEQ ID NO:20.

In some embodiments, the recombinant cytokine receptor comprises the ED of IL-4Rα. In some embodiments ED comprises the amino acid sequence set forth in SEQ ID NO:11. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:11. In some embodiments, the recombinant cytokine receptor comprises in N-terminal to C-terminal direction: (I) IL-4 cytokine; (II) the ED of IL-4Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-4 cytokine; (II) the ED of IL-4Rα; (III) the TD of IL-4Rα or IL-2Rβ; and (IV) the ID of IL-2Rβ. In some embodiments, the recombinant cytokine receptor comprises in N-terminal to C-terminal direction: (I) a cytokine comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:22; (II) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:11; (III) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 16 or SEQ ID NO: 21; and (IV) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) a cytokine comprising the amino acid sequence of SEQ ID NO:22; (II) an ED comprising the amino acid sequence of SEQ ID NO: 11; (III) a TD comprising the amino acid sequence of SEQ ID NO:16 or SEQ ID NO:21; and (IV) an ID comprising the amino acid sequence of SEQ ID NO:20. In some embodiments the cytokine is tethered to the ED through a linker. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:6. In some embodiments, the recombinant cytokine receptor comprises a cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 22.

In some embodiments, the recombinant cytokine receptor comprises the ED of IL-7Rα. In some embodiments, the IL-7Rα ED comprises the amino acid sequence set forth in SEQ ID NO: 12. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 12. In some embodiments, the recombinant cytokine receptor comprises in N-terminal to C-terminal direction: (I) IL-7 cytokine; (II) the ED of IL-7Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-7 cytokine; (II) the ED of IL-7Rα or IL-2Rβ; (III) the TD of IL-7Rα; and (IV) the ID of IL-2Rβ. In some embodiments, the recombinant cytokine receptor comprises in N-terminal to C-terminal direction: (I) a cytokine comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:23; (II) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 12; (III) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 17 or SEQ ID NO: 21; and (IV) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) a cytokine comprising the amino acid sequence of SEQ ID NO:23; (II) an ED comprising the amino acid sequence of SEQ ID NO: 12; (III) a TD comprising the amino acid sequence of SEQ ID NO:17 or SEQ ID NO:21; and (IV) an ID comprising the amino acid sequence of SEQ ID NO:20. In some embodiments the cytokine is tethered to the ED through a linker. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:7. In some embodiments, the recombinant cytokine receptor comprises a cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 23.

In some embodiments, the IL-9Rα ED comprises the amino acid sequence set forth in SEQ ID NO:13. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 13. In some embodiments, the recombinant cytokine receptor comprises in N-terminal to C-terminal direction: (I) IL-9 cytokine; (II) the ED of IL-9Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-9 cytokine; (II) the ED of IL-9Rα; (III) the TD of IL-9Rα; and (IV) the ID of IL-2Rβ. In some embodiments, the recombinant cytokine receptor comprises in N-terminal to C-terminal direction: (I) a cytokine comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:24; (II) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:13; (III) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 18; and (IV) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 20. In some embodiments, the recombinant cytokine receptor comprises in N-terminal to C-terminal direction: (I) a cytokine comprising the amino acid sequence of SEQ ID NO:24; (II) an ED comprising the amino acid sequence of SEQ ID NO:13; (III) a comprising the amino acid sequence of SEQ ID NO: 18; and (IV) an ID comprising the amino acid sequence of SEQ ID NO: 20. In some embodiments the cytokine is tethered to the ED through a linker. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:8. In some embodiments, the recombinant cytokine receptor comprises a cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:24.

In some embodiments, the recombinant cytokine receptor comprises the ED of IL-9Rα. In some embodiments ED comprises the amino acid sequence set forth in SEQ ID NO:13. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:13. In some embodiments, the recombinant cytokine receptor comprises in N-terminal to C-terminal direction: (I) IL-9 cytokine; (II) the ED of IL-9Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-9 cytokine; (II) the ED of IL-9Rα; (III) the TD of IL-2Rβ; and (IV) the ID of IL-2Rβ. In some embodiments, the recombinant cytokine receptor comprises in N-terminal to C-terminal direction: (I) a cytokine comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:24; (II) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:13; (III) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:21; and (IV) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO: 20. In some embodiments, the recombinant cytokine receptor comprises in N-terminal to C-terminal direction: (I) a cytokine comprising the amino acid sequence of SEQ ID NO:24; (II) an ED comprising the amino acid sequence of SEQ ID NO: 13; (III) a TD comprising the amino acid sequence of SEQ ID NO:21; and (IV) an ID comprising the amino acid sequence of SEQ ID NO: 20. In some embodiments the cytokine is tethered to the ED through a linker. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:15. In some embodiments, the recombinant cytokine receptor comprises a cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:24.

In some embodiments, the IL-21Rα ED comprises the amino acid sequence set forth in SEQ ID NO:14. In some embodiments, the ED comprises an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:14. In some embodiments, the recombinant cytokine receptor comprises in N-terminal to C-terminal direction: (I) IL-21 cytokine; (II) the ED of IL-21Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-21 cytokine; (II) the ED of IL-21Rα; (III) the TD of IL-21Rα or IL-2Rβ; and (IV) the ID of IL-2Rβ. In some embodiments, the recombinant cytokine receptor comprises in N-terminal to C-terminal direction: (I) a cytokine comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:25; (II) an ED comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:14; (III) a TD comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:19 or SEQ ID NO:21; and (IV) an ID comprising an amino acid sequence comprising at least about 80%, at least about 85%, at least about 90%, at least about 95%, at least about 96%, at least about 97%, at least about 98%, at least about 99%, or about 100% sequence identity to the amino acid sequence of SEQ ID NO:20. In some embodiments, the recombinant cytokine receptor comprises in N-terminal to C-terminal direction: (I) a cytokine comprising the amino acid sequence of SEQ ID NO:25; (II) an ED comprising the amino acid sequence of SEQ ID NO: 14; (III) a TD comprising the amino acid sequence of SEQ ID NO: 19 or SEQ ID NO:21; and (IV) an ID comprising the amino acid sequence of SEQ ID NO:20. In some embodiments the cytokine is tethered to the ED through a linker. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:9. In some embodiments, the recombinant cytokine receptor comprises a cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:25.

In some embodiments, the recombinant cytokine receptor comprising the aforementioned ED is expressed in cells. In some embodiments, the recombinant cytokine receptor comprising the aforementioned ED is expressed in immune cells. In some embodiments, the immune cells are T cells. In some embodiments, the immune cells are Treg cells.

In some embodiments, the recombinant cytokine receptor comprising the aforementioned ED is expressed in Tregs. In some embodiments, the Treg cells are CD4+, CD25+, and CD127lo. In some embodiments, the Tregs that express the recombinant cytokine receptor comprising the aforementioned ED also express FOXP3 and HELIOS. In some embodiments, the Tregs that express the recombinant cytokine receptor comprising the aforementioned ED also express high levels of FOXP3 and HELIOS. In some embodiments, the Tregs expressing the recombinant cytokine receptor comprising the aforementioned ED can survive and proliferate in the absence of IL-2. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned ED remain viable in vitro 2-20 days after transduction. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned ED can persist in vivo for at least about 3 days or longer. In some embodiments, the recombinant cytokine receptor comprising the aforementioned ED expressed in Tregs transduces intracellular IL-2 signaling within the Tregs. In some embodiments, signaling through the recombinant cytokine receptor comprising the aforementioned ED expressed in the Tregs induces phosphorylation of STAT5. In some embodiments, the Tregs expressing the recombinant cytokine receptor comprising the aforementioned ED are capable of suppressing the activity of effector T cells in the absence of IL-2 to at least the same degree as or more than wild-type Tregs cultured with exogenous IL-2. In some embodiments, the suppressed effector T cells are CD4+ effector T cells. In some embodiments, the suppressed effector T cells are CD8+ effector T cells. In some embodiments, Tregs that express the recombinant cytokine receptor comprising the aforementioned ED also express chimeric antigen receptors (CARs).

### 2. Intracellular Domains

The IL-2Rβ is primarily expressed in hematopoietic cells, and its involvement in immune cell-mediated immune responses is well described. IL-2Rβ polypeptide interacts with JAK1 through its C-terminal cytoplasmic domain, which comprises amino acids 240-525. The IL-2Rβ intracellular domain (ID) lacks intrinsic catalytic activity, thus relying upon its constitutive interaction with JAK1 at box 1 and box 3 motifs in its membrane-proximal region to promote IL-2 signal transduction (see, e.g., Wang, X et al. (2009), Annu Rev Immunol; 27:29-60). In activated T cells, IL-2 signal transduction through IL-2Rβ leads to the activation of three pathways: (1) STAT5 phosphorylation via JAK1 kinase, (2) PI3K-AKT pathway activation via Shc phosphorylation, and (3) Ras/MAPK pathway activation via Shc phosphorylation (see, e.g., Ye, C et al. (2018), Signal Transduct Target Ther; 3:2, hereby incorporated by reference in its entirety). Of these three pathways, JAK1/STAT5 signaling is predominant. Activation of the IL-2 pathways across all T cell lineages has been shown to induce T cell proliferation and survival. However, within CD4+CD25+ Treg cells, IL-2Rβ signaling only engages the JAK1/STAT5 pathway (see, e.g., Bensinger, S J et al. (2010), J Immunol; 172 (9): 5287-5296, hereby incorporated by reference in its entirety), which is required for Treg suppressive activity (see, e.g., Ye, C et al. (2018), Signal Transduct Target Ther; 3:2). While CD4+, and to a lesser degree CD8+ effector T cells are capable of producing IL-2 to signal their own proliferation and survival after activation, Tregs are not capable of producing IL-2. Therefore, Treg survival relies upon IL-2 produced by effector T cells.

In some embodiments, the intracellular domain of the recombinant cytokine receptors provided herein comprises an active IL-2Rβ intracellular domain. In some embodiments, the intracellular domain of the recombinant cytokine receptors is an active IL-2Rβ intracellular domain. In some embodiments, the intracellular domain of the recombinant cytokine receptors provided herein is able to engage in downstream signal transduction. In some embodiments, the IL-2Rβ intracellular domain phosphorylates STAT5 through its activation of the JAK1 kinase upon IL-2Rβ ID activation. In some embodiments, the IL-2 intracellular domain phosphorylates Shc upon IL-2Rβ receptor activation, thereby activating the downstream PI3K-AKT pathway. In some embodiments, the IL-2 intracellular domain phosphorylates Shc upon IL-2Rβ ID activation, thereby activating the downstream Ras/MAPK pathway. In some embodiments, the IL-2Rβ intracellular domain phosphorylates STAT5 through its activation of the JAK1 kinase upon recombinant cytokine receptor activation. In some embodiments, the IL-2 intracellular domain phosphorylates Shc upon recombinant cytokine receptor activation, thereby activating the downstream PI3K-AKT pathway. In some embodiments, the IL-2 intracellular domain phosphorylates Shc upon recombinant cytokine receptor activation, thereby activating the downstream Ras/MAPK pathway. In some embodiments, the recombinant cytokine receptor comprises IL-4Rα/IL-2Rβ, IL-7Rα/IL-2Rβ, IL-9Rα/IL-2Rβ, or IL-21Rα/IL-2Rβ extracellular/intracellular receptor domains. In some embodiments, the recombinant cytokine receptor is IL-4Rα/IL-2Rβ, IL-7Rα/IL-2Rβ, IL-9Rα/IL-2Rβ, or IL-21Rα/IL-2Rβ.

In some embodiments, the IL-2Rβ intracellular domain phosphorylates STAT5 through its activation of the JAK1 kinase upon recombinant cytokine receptor activation. In some embodiments, the IL-2Rβ intracellular domain phosphorylates Shc upon recombinant cytokine receptor activation, thereby activating the downstream PI3K-AKT pathway. In some embodiments, the IL-2Rβ intracellular domain phosphorylates Shc upon recombinant cytokine receptor activation, thereby activating the downstream Ras/MAPK pathway. In some embodiments, the recombinant cytokine receptor comprises IL-4Rα/IL-2Rβ extracellular/intracellular receptor domains. In some embodiments, the recombinant cytokine receptor is IL-4Rα/IL-2Rβ.

In some embodiments, the IL-2Rβ intracellular domain phosphorylates STAT5 through its activation of the JAK1 kinase upon recombinant cytokine receptor activation. In some embodiments, the IL-2Rβ intracellular domain phosphorylates Shc upon recombinant cytokine receptor activation, thereby activating the downstream PI3K-AKT pathway. In some embodiments, the IL-2Rβ intracellular domain phosphorylates Shc upon recombinant cytokine receptor activation, thereby activating the downstream Ras/MAPK pathway. In some embodiments, the recombinant cytokine receptor comprises IL-7Rα/IL-2Rβ extracellular/intracellular receptor domains. In some embodiments, the recombinant cytokine receptor is IL-7Rα/IL-2Rβ.

In some embodiments, the IL-2Rβ intracellular domain phosphorylates STAT5 through its activation of the JAK1 kinase upon recombinant cytokine receptor activation. In some embodiments, the IL-2Rβ intracellular domain phosphorylates Shc upon recombinant cytokine receptor activation, thereby activating the downstream PI3K-AKT pathway. In some embodiments, the IL-2Rβ intracellular domain phosphorylates Shc upon recombinant cytokine receptor activation, thereby activating the downstream Ras/MAPK pathway. In some embodiments, the recombinant cytokine receptor comprises IL-9Rα/IL-2Rβ extracellular/intracellular receptor domains. In some embodiments, the recombinant cytokine receptor is IL-9Rα/IL-2Rβ.

In some embodiments, the IL-2Rβ intracellular domain phosphorylates STAT5 through its activation of the JAK1 kinase upon recombinant cytokine receptor activation. In some embodiments, the IL-2Rβ intracellular domain phosphorylates Shc upon recombinant cytokine receptor activation, thereby activating the downstream PI3K-AKT pathway. In some embodiments, the IL-2Rβ intracellular domain phosphorylates Shc upon recombinant cytokine receptor activation, thereby activating the downstream Ras/MAPK pathway. In some embodiments, the recombinant cytokine receptor comprises IL-21Rα/IL-2Rβ extracellular/intracellular receptor domains. In some embodiments, the recombinant cytokine receptor is IL-21Rα/IL-2Rβ.

In some embodiments, the recombinant cytokine receptor is able to signal in the absence of exogenous IL-2. In some embodiments, the recombinant cytokine receptor is able to signal in the presence of a cytokine other than IL-2. In some embodiments, the IL-2Rβ ID comprises the amino acid sequence set forth in SEQ ID NO:20. In some embodiments, the ID comprises an amino acid sequence comprising at least about 80%, at least about 85%, at least about 90%, at least about 95%, at least about 96%, at least about 97%, at least about 98%, at least about 99%, or about 100% to SEQ ID NO:20. In some embodiments, the recombinant cytokine receptor comprises (I) the ED of IL-4Rα, IL-7Rα, IL-9Rα, or IL-21Rα; (II) the TD of IL-2Rβ, IL-4Rα, IL-7Rα, IL-9Rα, or IL-21Rα; (III) the ID of IL-2Rβ; and (IV) optionally, a cognate cytokine tethered by a polypeptide linker to the ED of the recombinant cytokine receptor. In some embodiments, the recombinant cytokine receptor comprises in N-terminal to C-terminal direction: (I) the ED of IL-4Rα; (II) the TD of IL-4Rα; (III) the ID of IL-2Rβ; and (IV) optionally, an IL-4 cytokine tethered by a polypeptide linker to the ED of the recombinant cytokine receptor. In some embodiments, the recombinant cytokine receptor comprises in N-terminal to C-terminal direction: (I) the ED of IL-7Rα; (II) the TD of IL-7Rα; (III) the ID of IL-2Rβ; and (IV) optionally, an IL-7 cytokine tethered by a polypeptide linker to the ED of the recombinant cytokine receptor. In some embodiments, the recombinant cytokine receptor comprises in N-terminal to C-terminal direction: (I) the ED of IL-9Rα; (II) the TD of IL-9Rα; (III) the ID of IL-2Rβ; and (IV) optionally, an IL-9 cytokine tethered by a polypeptide linker to the ED of the recombinant cytokine receptor. In some embodiments, the recombinant cytokine receptor comprises in N-terminal to C-terminal direction: (I) the ED of IL-21Rα; (II) the TD of IL-21Rα; (III) the ID of IL-2Rβ; and (IV) optionally, an IL-21 cytokine tethered by a polypeptide linker to the ED of the recombinant cytokine receptor. In some embodiments, the recombinant cytokine receptor comprises (I) optionally, a cognate cytokine tethered by a polypeptide linker to the ED of the recombinant cytokine receptor; (II) the ED of IL-4Rα, IL-7Rα, IL-9Rα, or IL-21Rα; (III) the TD of IL-2Rβ, IL-4Rα, IL-7Rα, IL-9Rα, or IL-21Rα; and (IV) the ID of IL-2Rβ. In some embodiments, the ED is from the same cytokine receptor as the TD (e.g., an ED from IL-4Rα and a TD from IL-4Rα). In some embodiments, the recombinant cytokine receptor comprises in N-terminal to C-terminal direction: (I) optionally, an IL-4 cytokine tethered by a polypeptide linker to the ED of the recombinant cytokine receptor; (II) the ED of IL-4Rα; (III) the TD of IL-4Rα; and (IV) the ID of IL-2Rβ. In some embodiments, the recombinant cytokine receptor comprises in N-terminal to C-terminal direction: (I) optionally, an IL-4 cytokine tethered by a polypeptide linker to the ED of the recombinant cytokine receptor; (II) the ED of IL-4Rα; (III) the TD of IL-2Rβ; and (IV) the ID of IL-2Rβ. In some embodiments, the recombinant cytokine receptor comprises in N-terminal to C-terminal direction: (I) optionally, an IL-7 cytokine tethered by a polypeptide linker to the ED of the recombinant cytokine receptor; (II) the ED of IL-7Rα; (III) the TD of IL-7Rα; and (IV) the ID of IL-2Rβ. In some embodiments, the recombinant cytokine receptor comprises in N-terminal to C-terminal direction: (I) optionally, an IL-7 cytokine tethered by a polypeptide linker to the ED of the recombinant cytokine receptor; (II) the ED of IL-7Rα; (III) the TD of IL-2Rβ; and (IV) the ID of IL-2Rβ. In some embodiments, the recombinant cytokine receptor comprises in N-terminal to C-terminal direction: (I) optionally, an IL-9 cytokine tethered by a polypeptide linker to the ED of the recombinant cytokine receptor; (II) the ED of IL-9Rα; (III) the TD of IL-9Rα; and (IV) the ID of IL-2Rβ. In some embodiments, the recombinant cytokine receptor comprises in N-terminal to C-terminal direction: (I) optionally, an IL-9 cytokine tethered by a polypeptide linker to the ED of the recombinant cytokine receptor; (II) the ED of IL-9Rα; (III) the TD of IL-2Rβ; and (IV) the ID of IL-2Rβ. In some embodiments, the recombinant cytokine receptor comprises in N-terminal to C-terminal direction: (I) optionally, an IL-21 cytokine tethered by a polypeptide linker to the ED of the recombinant cytokine receptor; (II) the ED of IL-21Rα; (III) the TD of IL-21Rα; and (IV) the ID of IL-2Rβ. In some embodiments, the recombinant cytokine receptor comprises in N-terminal to C-terminal direction: (I) optionally, an IL-21 cytokine tethered by a polypeptide linker to the ED of the recombinant cytokine receptor; (II) the ED of IL-21Rα; (III) the TD of IL-2Rβ; and (IV) the ID of IL-2Rβ. In some embodiments, the recombinant cytokine receptor comprises (I) the ED of IL-4Rα, IL-7Rα, IL-9Rα, or IL-21Rα; (II) the TD of IL-2Rβ, IL-4Rα, IL-7Rα, IL-9Rα, or IL-21Rα; and (III) the ID of IL-2Rβ. In some embodiments, the recombinant cytokine receptor comprises in N-terminal to C-terminal direction: (I) the ED of IL-4Rα; (II) the TD of IL-4Rα; and (III) the ID of IL-2Rβ. In some embodiments, the recombinant cytokine receptor comprises in N-terminal to C-terminal direction: (I) the ED of IL-4Rα; (II) the TD of IL-2Rβ; and (III) the ID of IL-2Rβ. In some embodiments, the recombinant cytokine receptor comprises in N-terminal to C-terminal direction: (I) the ED of IL-7Rα; (II) the TD of IL-7Rα; and (III) the ID of IL-2Rβ. In some embodiments, the recombinant cytokine receptor comprises in N-terminal to C-terminal direction: (I) the ED of IL-7Rα; (II) the TD of IL-2Rβ; and (III) the ID of IL-2Rβ. In some embodiments, the recombinant cytokine receptor comprises in N-terminal to C-terminal direction: (I) the ED of IL-9Rα; (II) the TD of IL-9Rα; and (III) the ID of IL-2Rβ. In some embodiments, the recombinant cytokine receptor comprises in N-terminal to C-terminal direction: (I) the ED of IL-9Rα; (II) the TD of IL-2Rβ; and (III) the ID of IL-2Rβ. In some embodiments, the recombinant cytokine receptor comprises in N-terminal to C-terminal direction: (I) the ED of IL-21Rα; (II) the TD of IL-21Rα; and (III) the ID of IL-2Rβ. In some embodiments, the recombinant cytokine receptor comprises in N-terminal to C-terminal direction: (I) the ED of IL-21Rα; (II) the TD of IL-2Rβ; and (III) the ID of IL-2Rβ

### 3. Transmembrane Domain

The TDs of IL-2Rβ, IL-4Rα, IL-7Rα, IL-9Rα, and IL-21Rα receptors as members of the class I cytokine receptors comprise a modular, single-pass TD. The TDs of IL-2Rβ, IL-4Rα, IL-7Rα, IL-9Rα, and IL-21Rα are 25 residues, 25 residues, 24 residues, 25 residues, 21 residues, and 21 residues long.

In some embodiments, the recombinant cytokine receptor comprises a transmembrane domain. In some embodiments, the transmembrane domain is located between the extracellular domain and the intracellular domain of the recombinant cytokine. In some embodiments, the transmembrane domain is from a cytokine receptor. In some embodiments, the transmembrane domain is from a class I cytokine receptor. In some embodiments, the transmembrane domain is from a class II cytokine receptor. In some embodiments, the transmembrane domain is a transmembrane domain selected from the group consisting of IL-2Rβ, IL-4Rα, IL-7Rα, IL-9Rα, and IL-21Rα. In some embodiments, the transmembrane domain is a transmembrane domain from IL-2Rβ. In some embodiments, the transmembrane domain is a transmembrane domain from IL-4Rα. In some embodiments, the transmembrane domain is a transmembrane domain from IL-7Rα. In some embodiments, the transmembrane domain is a transmembrane domain from IL-9Rα. In some embodiments, the transmembrane domain is a transmembrane domain from IL-21Rα. In some embodiments, the TD is selected from the same cytokine receptor as the ED. In some embodiments, the TD is selected from a different cytokine receptor than the ED. In some embodiments, the TD comprises any one of the amino acid sequences set forth in SEQ ID NOs: 16-19 and 21.

In some embodiments, the IL-2Rβ TD comprises the amino acid sequence set forth in SEQ ID NO:21. In some embodiments, the TD comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:21. In some embodiments, the recombinant cytokine receptor comprises a TD that is about 20-30 amino acids in length. In some embodiments, the recombinant cytokine receptor comprises a TD that is 21, 22, 23, 24, 25, 26, 27, 28, 29, or 30 amino acids in length. In some embodiments, the recombinant cytokine receptor comprises: (I) the ED of IL-4Rα, IL-7Rα, IL-9Rα, or IL-21Rα; (II) the TD of IL-2Rβ; and (III) the ID of IL-2Rβ. In some embodiments, the recombinant cytokine receptor comprises in N-terminal to C-terminal direction: (I) the ED of IL-4Rα; (II) the TD of IL-2Rβ; and (III) the ID of IL-2Rβ. In some embodiments, the recombinant cytokine receptor comprises in N-terminal to C-terminal direction: (I) IL-4 cytokine; (II) the ED of IL-4Rα; (III) the TD of IL-2Rβ; and (IV) the ID of IL-2Rβ. In some embodiments IL-4 cytokine is tethered to the ED of IL-4Rα by a polypeptide linker. In some embodiments, the recombinant cytokine receptor comprises in N-terminal to C-terminal direction: (I) the ED of IL-7Rα; (II) the TD of IL-2Rβ; and (III) the ID of IL-2Rβ. In some embodiments, the recombinant cytokine receptor comprises in N-terminal to C-terminal direction: (I) IL-7 cytokine; (II) the ED of IL-7Rα; (III) the TD of IL-2Rβ; and (IV) the ID of IL-2Rβ. In some embodiments IL-7 cytokine is tethered to the ED of IL-7Rα by a polypeptide linker. In some embodiments, the recombinant cytokine receptor comprises in N-terminal to C-terminal direction: (I) the ED of IL-9Rα; (II) the TD of IL-2Rβ; and (III) the ID of IL-2Rβ. In some embodiments, the recombinant cytokine receptor comprises in N-terminal to C-terminal direction: (I) IL-9 cytokine; (II) the ED of IL-9Rα; (III) the TD of IL-2Rβ; and (IV) the ID of IL-2Rβ. In some embodiments IL-9 cytokine is tethered to the ED of IL-9Rα by a polypeptide linker. In some embodiments, the recombinant cytokine receptor comprises in N-terminal to C-terminal direction: (I) the ED of IL-21Rα; (II) the TD of IL-2Rβ; and (III) the ID of IL-2Rβ. In some embodiments, the recombinant cytokine receptor comprises in N-terminal to C-terminal direction: (I) IL-21 cytokine; (II) the ED of IL-21Rα; (III) the TD of IL-2Rβ; and (IV) the ID of IL-2Rβ. In some embodiments IL-21 cytokine is tethered to the ED of IL-21Rα by a polypeptide linker.

In some embodiments, the IL-4Rα TD comprises the amino acid sequence set forth in SEQ ID NO:16. In some embodiments, the TD comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 16. In some embodiments, the recombinant cytokine receptor comprises a TD that is about 20-30 amino acids in length. In some embodiments, the recombinant cytokine receptor comprises a TD that is 21, 22, 23, 24, 25, 26, 27, 28, 29, or 30 amino acids in length. In some embodiments, the recombinant cytokine receptor comprises: (I) the ED of IL-4Rα, IL-7Rα, IL-9Rα, or IL-21Rα; (II) the TD of IL-4Rα; and (III) the ID of IL-2Rβ. In some embodiments, the recombinant cytokine receptor comprises in N-terminal to C-terminal direction: (I) the ED of IL-4Rα; (II) the TD of IL-4Rα; and (III) the ID of IL-2Rβ. In some embodiments, the recombinant cytokine receptor comprises in N-terminal to C-terminal direction: (I) IL-4 cytokine; (II) the ED of IL-4Rα; (III) the TD of IL-4Rα; and (IV) the ID of IL-2Rβ. In some embodiments IL-4 cytokine is tethered to the ED of IL-4Rα by a polypeptide linker.

In some embodiments, the IL-7Rα TD comprises the amino acid sequence set forth in SEQ ID NO:17. In some embodiments, the TD comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 17. In some embodiments, the recombinant cytokine receptor comprises a TD that is about 20-30 amino acids in length. In some embodiments, the recombinant cytokine receptor comprises a TD that is 21, 22, 23, 24, 25, 26, 27, 28, 29, or 30 amino acids in length. In some embodiments, the recombinant cytokine receptor comprises: (I) the ED of IL-4Rα, IL-7Rα, IL-9Rα, or IL-21Rα; (II) the TD of IL-7Rα; and (III) the ID of IL-2Rβ. In some embodiments, the recombinant cytokine receptor comprises in N-terminal to C-terminal direction: (I) the ED of IL-7Rα; (II) the TD of IL-7Rα; and (III) the ID of IL-2Rβ. In some embodiments, the recombinant cytokine receptor comprises in N-terminal to C-terminal direction: (I) IL-7 cytokine; (II) the ED of IL-7Rα; (III) the TD of IL-7Rα; and (IV) the ID of IL-2Rβ. In some embodiments IL-7 cytokine is tethered to the ED of IL-7Rα by a polypeptide linker.

In some embodiments, the IL-9Rα TD comprises the amino acid sequence set forth in SEQ ID NO:18. In some embodiments, the TD comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 18. In some embodiments, the recombinant cytokine receptor comprises a TD that is about 20-30 amino acids in length. In some embodiments, the recombinant cytokine receptor comprises a TD that is 21, 22, 23, 24, 25, 26, 27, 28, 29, or 30 amino acids in length. In some embodiments, the recombinant cytokine receptor comprises: (I) the ED of IL-4Rα, IL-7Rα, IL-9Rα, or IL-21Rα; (II) the TD of IL-9Rα; and (III) the ID of IL-2Rβ. In some embodiments, the recombinant cytokine receptor comprises in N-terminal to C-terminal direction: (I) the ED of IL-9Rα; (II) the TD of IL-9Rα; and (III) the ID of IL-2Rβ. In some embodiments, the recombinant cytokine receptor comprises in N-terminal to C-terminal direction: (I) IL-9 cytokine; (II) the ED of IL-9Rα; (III) the TD of IL-9Rα; and (IV) the ID of IL-2Rβ. In some embodiments IL-9 cytokine is tethered to the ED of IL-9Rα by a polypeptide linker.

In some embodiments, the IL-21Rα TD comprises the amino acid sequence set forth in SEQ ID NO:19. In some embodiments, the TD comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 19. In some embodiments, the recombinant cytokine receptor comprises a TD that is about 20-30 amino acids in length. In some embodiments, the recombinant cytokine receptor comprises a TD that is 21, 22, 23, 24, 25, 26, 27, 28, 29, or 30 amino acids in length. In some embodiments, the recombinant cytokine receptor comprises: (I) the ED of IL-4Rα, IL-7Rα, IL-9Rα, or IL-21Rα; (II) the TD of IL-21Rα; and (III) the ID of IL-2Rβ. In some embodiments, the recombinant cytokine receptor comprises in N-terminal to C-terminal direction: (I) the ED of IL-21Rα; (II) the TD of IL-21Rα; and (III) the ID of IL-2Rβ. In some embodiments, the recombinant cytokine receptor comprises in N-terminal to C-terminal direction: (I) IL-21 cytokine; (II) the ED of IL-21Rα; (III) the TD of IL-21Rα; and (IV) the ID of IL-2Rβ. In some embodiments IL-21 cytokine is tethered to the ED of IL-21Rα by a polypeptide linker.

In some embodiments, the TD comprises an amino acid sequence comprising at least about 80%, at least about 85%, at least about 90%, at least about 95%, at least about 96%, at least about 97%, at least about 98%, at least about 99%, or about 100% sequence identity with any one of the amino acid sequences set forth in SEQ ID NOs: 16-19 and 21. In some embodiments, the TD comprises an amino acid sequence selected from the group consisting of SEQ ID NOs: 16-19 and 21.

In some embodiments, the recombinant cytokine receptor comprising the aforementioned TD is expressed in immune cells. In some embodiments, the recombinant cytokine receptor comprising the aforementioned TD is expressed in T cells. In some embodiments, the recombinant cytokine receptor comprising the aforementioned TD is expressed in regulatory T cells (Tregs). In some embodiments, the Treg cells are CD4+, CD25+, and CD127lo (i.e., CD127low). In some embodiments, the Tregs that express the recombinant cytokine receptor comprising the aforementioned TD express FOXP3 and/or HELIOS. In some embodiments, the Tregs that express the recombinant cytokine receptor comprising the aforementioned TD also express high levels of FOXP3 and/or HELIOS. In some embodiments, the Tregs expressing the recombinant cytokine receptor comprising the aforementioned TD can survive and proliferate in the absence of IL-2. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned TD remain viable in vitro 12-20 days after transduction. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned TD can persist in vivo for at least about 3 days or longer. In some embodiments, the recombinant cytokine receptor comprising the aforementioned TD expressed in Tregs transduces intracellular IL-2 signaling within the Tregs. In some embodiments, signaling through the recombinant cytokine receptor comprising the aforementioned TD expressed in the Tregs induces phosphorylation of STAT5. In some embodiments, the Tregs expressing the recombinant cytokine receptor comprising the aforementioned TD are capable of suppressing the activity of effector T cells in the absence of IL-2 to at least the same degree as or more than wild-type Tregs cultured with exogenous IL-2. In some embodiments, the suppressed effector T cells are CD4+ effector T cells. In some embodiments, the suppressed effector T cells are CD8+ effector T cells. In some embodiments, Tregs that express the recombinant cytokine receptor comprising the aforementioned TD also express chimeric antigen receptors (CARs).

### 4. Tethered Cytokines and Linkers

The aforementioned recombinant cytokine receptors can be optionally tethered by a polypeptide linker to their cognate cytokine. In some embodiments, IL-4 can be tethered to the recombinant cytokine receptor IL-4Rα ED. In some embodiments, the IL-4 tethered recombinant cytokine receptor comprises an amino acid sequence as set forth in SEQ ID NO:6. In some embodiments, the IL-4 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:6. In some embodiments, IL-7 can be tethered to the recombinant cytokine receptor IL-7Rα ED. In some embodiments, the IL-7 tethered recombinant cytokine receptor comprises an amino acid sequence as set forth in SEQ ID NO:7. In some embodiments, the IL-7 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:7. In some embodiments, IL-9 can be tethered to the recombinant cytokine receptor IL-9Rα ED. In some embodiments, the IL-9 tethered recombinant cytokine receptor comprises an amino acid sequence as set forth in SEQ ID NO:8. In some embodiments, the IL-9 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:8. In some embodiments, the IL-9 tethered recombinant cytokine receptor comprises an amino acid sequence as set forth in SEQ ID NO: 15. In some embodiments, the IL-9 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 15. In some embodiments, IL-21 can be tethered to the recombinant cytokine receptor IL-21Rα ED. In some embodiments, the IL-21 tethered recombinant cytokine receptor comprises an amino acid sequence as set forth in SEQ ID NO:9. In some embodiments, the IL-21 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:9. In some embodiments, the recombinant cytokine receptor is able to signal in the absence of exogenous IL-2. In some embodiments, the recombinant cytokine receptor is able to signal in the absence of exogenous interleukin cytokine.

In some embodiments, the cognate cytokine tethered by a polypeptide linker to the recombinant cytokine receptor comprises any of the SEQ ID NOs: 22-25. In some embodiments, the cognate cytokine comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NOs: 22-25.

In some embodiments, the recombinant cytokine receptor comprises an IL-4 cytokine tethered to the extracellular domain of IL-4Rα. In some embodiments, IL-4 comprises the amino acid sequence set forth in SEQ ID NO:22. In some embodiments, IL-4 comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:22. In some embodiments, the IL-4 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:6, wherein the recombinant cytokine receptor comprises an IL-4 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:22, and wherein the recombinant cytokine receptor comprises a ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 11. In some embodiments, the IL-4 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO: 6, wherein the recombinant cytokine receptor comprises an IL-4 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:22, and wherein the recombinant cytokine receptor comprises a ED having an amino acid sequence comprising at least about 90%, at least about 95%, at least about 96%, at least about 97%, at least about 98%, at least about 99%, or about 100% sequence identity with the amino acid sequence set forth in SEQ ID NO:11.

In some embodiments, the recombinant cytokine receptor comprises an IL-7 cytokine tethered to the extracellular domain of IL-7Rα. In some embodiments, IL-7 comprises the amino acid sequence set forth in SEQ ID NO:23. In some embodiments, IL-7 comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:23. In some embodiments, the IL-7 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:7, wherein the recombinant cytokine receptor comprises an IL-7 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:23, and wherein the recombinant cytokine receptor comprises a ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 12. In some embodiments, the IL-7 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO: 7, wherein the recombinant cytokine receptor comprises an IL-7 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:23, and wherein the recombinant cytokine receptor comprises a ED having an amino acid sequence comprising at least about 90%, at least about 95%, at least about 96%, at least about 97%, at least about 98%, at least about 99%, or about 100% sequence identity with the amino acid sequence set forth in SEQ ID NO: 12.

In some embodiments, the recombinant cytokine receptor comprises an IL-9 cytokine tethered to the extracellular domain of IL-9Rα. In some embodiments, IL-9 comprises the amino acid sequence set forth in SEQ ID NO:24. In some embodiments, IL-9 comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:24. In some embodiments, the IL-9 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:8, wherein the recombinant cytokine receptor comprises an IL-9 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:24, and wherein the recombinant cytokine receptor comprises a ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 13. In some embodiments, the IL-9 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO: 8, wherein the recombinant cytokine receptor comprises an IL-9 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:24, and wherein the recombinant cytokine receptor comprises a ED having an amino acid sequence comprising at least about 90%, at least about 95%, at least about 96%, at least about 97%, at least about 98%, at least about 99%, or about 100% sequence identity with the amino acid sequence set forth in SEQ ID NO: 13. In some embodiments, the IL-9 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 15, wherein the recombinant cytokine receptor comprises an IL-9 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:24, and wherein the recombinant cytokine receptor comprises a ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 13. In some embodiments, the IL-9 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO: 15, wherein the recombinant cytokine receptor comprises an IL-9 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:24, and wherein the recombinant cytokine receptor comprises a ED having an amino acid sequence comprising at least about 90%, at least about 95%, at least about 96%, at least about 97%, at least about 98%, at least about 99%, or about 100% sequence identity with the amino acid sequence set forth in SEQ ID NO:13.

In some embodiments, the recombinant cytokine receptor comprises an IL-21 cytokine tethered to the extracellular domain of IL-21Rα. In some embodiments, IL-21 comprises the amino acid sequence set forth in SEQ ID NO:25. In some embodiments, IL-21 comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 25. In some embodiments, the IL-21 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:9, wherein the recombinant cytokine receptor comprises an IL-21 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:25, and wherein the recombinant cytokine receptor comprises a ED having an amino acid sequence comprising at least 80%, at least about 85%, at least about 90%, at least about 95%, at least about 96%, at least about 97%, at least about 98%, at least about 99%, or about 100% sequence identity with the amino acid sequence set forth in SEQ ID NO: 14. In some embodiments, the IL-21 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO: 9, wherein the recombinant cytokine receptor comprises an IL-21 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:25, and wherein the recombinant cytokine receptor comprises a ED having an amino acid sequence comprising at least about 90%, at least about 95%, at least about 96%, at least about 97%, at least about 98%, at least about 99%, or about 100% sequence identity with the amino acid sequence set forth in SEQ ID NO: 14.

The linkers can be polypeptide linkers of any length. In some embodiments, the polypeptide linker is from about 1 amino acid to about 10 amino acids long, from about 2 amino acids to about 15 amino acids long, from about 3 amino acids to about 12 amino acids long, from about 4 amino acids to about 10 amino acids long, from about 5 amino acids to about 9 amino acids long, from about 6 amino acids to about 8 amino acids long, from about 1 amino acid to about 20 amino acids long, from about 21 amino acids to about 30 amino acids long, from about 1 amino acid to about 30 amino acids long, from about 2 amino acids to about 20 amino acids long, from about 10 amino acids to about 30 amino acids long, from about 2 amino acids to about 19 amino acids long, from about 2 amino acids to about 18 amino acids long, from about 2 amino acids to about 17 amino acids long, from about 2 amino acids to about 16 amino acids long, from about 2 amino acids to about 10 amino acids long, from about 2 amino acids to about 14 amino acids long, from about 2 amino acids to about 13 amino acids long, from about 2 amino acids to about 12 amino acids long, from about 2 amino acids to about 11 amino acids long, from about 2 amino acids to about 9 amino acids long, from about 2 amino acids to about 8 amino acids long, from about 2 amino acids to about 7 amino acids long, from about 2 amino acids to about 6 amino acids long, from about 2 amino acids to about 5 amino acids long, from about 2 amino acids to about 4 amino acids long, or from about 2 amino acids to about 3 amino acids long. In some embodiments, the polypeptide linker is any of 1, 2, 3, 4, 5, 6, 7, 8, 9, 10, 11, 12, 13, 14, 15, 16, 17, 18, 19, or 20 amino acids long. In some embodiments, the polypeptide linker is any of 21, 22, 23, 24, 25, 26, 27, 28, 29, or 30 amino acids long. For example, in some embodiments, the polypeptide linker is about 5 amino acids long. In some embodiments, the N-terminus of the polypeptide linker is covalently linked to the C-terminus of the interleukin cytokine, and the C-terminus of the polypeptide linker is covalently linked to the N-terminus of the ED.

A polypeptide linker can have a naturally occurring sequence or a non-naturally occurring sequence. For example, a sequence derived from the hinge region of a heavy chain only antibody can be used as a linker. See, for example, WO1996/34103. In some embodiments, the linker is a flexible linker. In some embodiments, the exemplary flexible linker is (GGGGS) n where n is an integer of at least one (SEQ ID NO:29; SEQ ID NO:27 when n=2). Glycine and glycine-serine polymers are relatively unstructured, and therefore may be able to serve as a neutral tether between components. Glycine accesses significantly more phi-psi space than even alanine and is much less restricted than residues with longer side chains (see Scheraga, Rev. Computational Chem. 11 173-142 (1992)). Thus, an exemplary flexible linker includes, but is not limited to, Gly-Gly-Gly-Gly-Ser-Gly-Gly-Gly-Gly-Ser (SEQ ID NO:27), Gly-Gly-Gly-Gly-Ser (SEQ ID NO:30), Gly-Gly-Gly-Gly-Ser-Gly-Gly-Gly-Gly-Ser-Gly-Gly-Gly-Gly-Ser (SEQ ID NO: 31), and Ser-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Ser-Leu-Gln (SEQ ID NO:32). The ordinarily skilled artisan will recognize that design of a recombinant cytokine receptor tethered to its cognate cytokine can include linkers that are all or partially flexible, such that the linker can include a flexible linker portion as well as one or more portions that confer less flexible structure to provide a desired recombinant cytokine receptor structure.

In some embodiments, the linker between the cytokine and the recombinant cytokine receptor is a stable linker. In some embodiments, the linker between the cytokine and the recombinant cytokine receptor is not cleavable by a protease.

### II. Cells

The disclosure provides for an adoptive cellular immunotherapy composition comprising a genetically modified cell preparation as described herein, e.g., genetically modified immune cells preparation, e.g., lymphocytes. These cells are, for example, multipotent cells such as hematopoietic stem cells, various progenitor or precursor cells of hematopoietic lineages, and various immune cells (e.g., human autologous or allogeneic T, natural killer (NK), dendritic, or B cells). These cells may also be pluripotent stem cells (PSCs) such as human embryonic stem cells and induced PSCs, which can be used to generate therapeutic cell populations. In some embodiments, pluripotent and multipotent cells are differentiated into a desired cell type in vitro before being implanted, injected, or infused into the patient.

In some alternatives, the genetically modified cell preparation is a T lymphocyte cell preparation. In some embodiments the T lymphocyte cell preparation comprises CD4+ T cells that have (i) a chimeric receptor comprising an extracellular antibody domain specific for a ligand associated with a disease or disorder, a spacer region, a transmembrane domain, and an intracellular signaling domain of a T cell receptor, and (ii) a recombinant cytokine receptor as described herein. In other alternatives, an adoptive cellular immunotherapy composition comprises a chimeric receptor modified CD8+ cytotoxic T lymphocyte cell preparation that provides a cellular immune response, wherein the cytotoxic T lymphocyte cell preparation comprises CD8+ T cells that have (i) a chimeric receptor comprising an extracellular antibody domain specific for a ligand associated with a disease or disorder, a spacer region, a transmembrane domain, and an intracellular signaling domain of a T cell receptor, and (ii) a recombinant cytokine receptor as described herein. In some alternatives, the chimeric receptor modified T cell population of the disclosure can persist in vivo for at least about 3 days or longer. In some alternatives, each of these populations can be combined with one another or with one or more other cell types to provide a composition. In some alternatives, the genetically modified cells are Treg cells.

Regulatory T (Treg) cells are involved in the maintenance of immunological self-tolerance and in mitigating deleterious immune responses to both self and non-self (allo) antigens. Tregs comprise both natural and induced subtypes. Natural Tregs (nTregs) are cells which originate as a separate cell lineage during development. Peripheral or induced Tregs (iTregs) differentiate from conventional T cells. In some embodiments, CD4+ T cells that are not nTregs and not iTregs can be engineered into Tregs using the methods and compositions of the disclosure. In some embodiments, the CD4+ T cells are used to make Treg cells expressing recombinant cytokine receptors using the methods and compositions of the disclosure.

In some embodiments, the cell expressing the recombinant cytokine receptor of the disclosure is not naturally occurring. In some embodiments, the cell is an immune cell. In some embodiments, the cell is a lymphocyte. In some embodiments, the cell is a T cell. In some embodiment, the cell is a regulatory T cell (Treg).

In some embodiments, the Treg expressing the recombinant cytokine receptor of the disclosure is not naturally occurring (not an nTreg and/or not an iTreg). In some embodiments, the cell expresses one or markers characteristic of a Treg. Treg markers include high levels of CD25 (CD25+), low levels of CD127 (CD127lo), or both high CD25 and low CD127. The levels of CD25 and CD127 are compared, for example to a CD4+ T cell that is not a Treg. In some embodiments, the Treg cell expressing the recombinant cytokine receptor has a high CD25, high CD4, and low CD127 phenotype.

In some embodiments, the Treg expressing the tethered IL9Rα/IL-2Rβ recombinant cytokine receptor is not naturally occurring (not an nTreg and/or not an iTreg). In some embodiments, the cell expresses one or markers characteristic of a Treg. Treg markers include high levels of CD25 (CD25+), low levels of CD127 (CD127lo or CD127low), or both high CD25 and low CD127. In some embodiments, the Treg cell expressing the recombinant cytokine receptor has a high CD25, high CD4, and low CD127 phenotype.

In some embodiments, the Tregs expressing the tethered IL9Rα/IL-2Rβ recombinant cytokine receptor survive in the absence of exogenous cytokine, e.g., IL-2. In some embodiments, the Tregs expressing the tethered IL9Rα/IL-2Rβ recombinant cytokine receptor proliferate in the absence of exogenous cytokine, e.g., IL-2. In some embodiments, the Tregs expressing the tethered IL9Rα/IL-2Rβ recombinant cytokine receptor proliferate in the absence of exogenous cytokine. In some embodiments, the Tregs expressing the tethered IL9Rα/IL-2Rβ recombinant cytokine receptor engraft into an individual, e.g., a rodent (such as a mouse) or a human. In some embodiments, the individual expresses IL-2. In some embodiments, the Tregs expressing the tethered IL9Rα/IL-2Rβ recombinant cytokine receptor survive and proliferate upon adoptive cellular transfer into an individual, e.g., a rodent (such as a mouse) or a human, for example an individual expressing IL-2. In some embodiments, the adoptively transferred Tregs expressing the tethered IL9Rα/IL-2Rβ recombinant cytokine receptor prevent, treat, ameliorate, or otherwise reduce the severity of a disease, such as an autoimmune disease (e.g., graft versus host disease).

Also provided herein is a cell that expresses a recombinant cytokine receptor. In some embodiments, the recombinant cytokine receptor comprises an intracellular IL-2 receptor beta chain domain, a transmembrane domain, and an extracellular domain that binds to a cytokine other than IL-2. In some embodiments, the recombinant cytokine receptor comprise an extracellular domain selected from the group consisting of an IL-4 extracellular domain, an IL-7 extracellular domain, an IL-9 extracellular domain, and an IL-21 extracellular domain. In some embodiments, the cytokine is tethered to the recombinant cytokine receptor. In some embodiments, the cytokine is not IL-2.

In some embodiments, the cell expresses one or more proteins associated with a Treg phenotype. In some embodiments, the cell expresses FOXP3. FOXP3 plays a crucial role in development and function of Treg cells (see Yagi et al. 2004, Int Immunol. 16 (11): 1643-56; Sadlon et al. 2018, Clin Transl Immunology 7 (2): e1011). FOXP3 is initially expressed during the expansion of T cells for a first time but is followed by the loss of FOXP3 expression after polyclonal stimulation. This is in contrast to Tregs, where FOXP3 expression rises and is maintained over time. Expression levels of FOXP3 may be assessed by conventional methods such as Western blotting, flow cytometry, or ELISA. Expression levels may also be assessed by analyzing mRNA using techniques such as RT-qPCR. In some embodiments, expression of FOXP3 increases compared to untransduced cells. In some embodiments, expression of FOXP3 is increased compared to cells without recombinant cytokine receptors.

In some embodiments, FOXP3 expression increases at least about 2-fold, at least about 3-fold, at least about 4-fold, at least about 5-fold, or more compared to cells without recombinant cytokine receptors. In some embodiments, FOXP3 expression increases at least about 1.1-fold, at least about 1.5-fold, at least about 2-fold, at least about 3-fold, at least about 4-fold, at least about 5-fold, or more compared to cells that have not been transduced with a recombinant receptor. In some embodiments, the expression of FOXP3 is increased in Tregs transduced with recombinant cytokine receptor compared to untransduced cells cultured without one or more cytokines. In some embodiments, the expression of FOXP3 is increased in the Tregs transduced with recombinant cytokine receptor compared to untransduced cells cultured with a cytokine that is not IL-2. In some embodiments, FOXP3 expression is maintained at approximately the same level as the day of highest FOXP3 expression following transduction with the recombinant cytokine receptor.

In some embodiments, the Treg cell is CD4 positive (CD4+). In some embodiments, the cell is CD4+/CD25+. In some embodiments, the cell is CD4+/CD127 low (i.e., CD4+/CD127lo). In some embodiments, the cell is CD4+/CD25+/CD127 low (i.e., CD4+/CD25+/CD127lo).

Tregs can be characterized by expression of CD25+. In some embodiments, the cell is Treg cell is CD25+. In some embodiments, the cell is CD4+/CD25+. In some embodiments, the cell is CD25+/CD127 low (i.e., CD25+/CD127lo). In some embodiments, the cell is CD4+/CD25+/CD127 low (i.e., CD4+/CD25+/CD127lo).

In some embodiments, the cell expresses a low level of CD127 (CD127lo). In some embodiments, the cell is CD4+/CD127 low (i.e., CD4+/CD127lo). In some embodiments, the cell is CD25+/CD127 low (i.e., CD25+/CD127lo). In some embodiments, the cell is CD4+/CD25+/CD127 low (i.e., CD4+/CD25+/CD127lo). In some embodiments, cells transduced with the recombinant cytokine receptors provided herein maintain CD127 low (CD127lo) status upon transduction.

HELIOS is a transcription factor that is expressed in Tregs. In some embodiments, the Treg cells of the present disclosure express HELIOS. In some embodiments, the expression of HELIOS is increased compared to conventional T cells. In some embodiments, the expression of HELIOS is detectable compared to conventional T cells. Expression levels of HELIOS may be assessed by conventional methods such as Western blotting, flow cytometry, or ELISA. Expression levels may also be assessed by analyzing mRNA using techniques such as RT-qPCR. In some embodiments, HELIOS expression increases at least 2-fold, at least 3-fold, at least 4-fold, at least 5-fold, at least 6-fold, at least 7-fold, at least 8-fold, at least 9-fold, at least 10-fold, at least 20-fold, or more compared to conventional T cells. In some embodiments, the expression of HELIOS is increased in the Tregs transduced with recombinant cytokine receptor compared to untransduced cells cultured without cytokine. In some embodiments, Tregs transduced with a recombinant cytokine receptor maintain HELIOS expression following transduction.

In some embodiments, the cell expresses or secretes a low level of IL-2. In some embodiments, expression or secretion of IL-2 in transduced Treg cells is decreased compared to conventional T cells. In some embodiments, the expression or secretion IL-2 is decreased in the Tregs transduced with recombinant cytokine receptor compared to untransduced cells cultured without cytokine. In some embodiments, the expression or secretion IL-2 is decreased in the Tregs transduced with recombinant cytokine receptor compared to untransduced cells.

In some embodiments, the cell has an immunosuppressive phenotype. In one embodiment, the cell generates an immunosuppressive effect in an individual having an immune-related disorder. In some embodiments, the individual is human. In some embodiments, the individual expresses IL-2. In some embodiments, the cell is autologous to the individual.

In some embodiment, the cell suppresses, blocks, or inhibits graft-vs-host disease in the individual. In one embodiment, the cell suppresses, blocks, or inhibits an immune-related disorder in the individual. In some embodiments, the cell is administered prior to onset of an immune-related disorder. In some embodiments, the individual is human. In some embodiments, the individual expresses IL-2. In some embodiments, the cell is autologous to the individual. In some embodiments, the cell is allogeneic to the individual.

Methods of measuring markers used to characterize Treg cells will be readily apparent to the person of ordinary skill in the art. For example, Tregs or populations of T cells comprising Tregs can be cultured using the methods described herein. Following culturing. Tregs can be collected and stained using antibodies against Treg markers such as FOXP3 (e.g., labeled by PE), CD25 (e.g., labeled by APC), and CD127 (e.g., labeled by BV421) and expression analyzed using fluorescence activated flow cytometry (FACS) or fluorescence microscopy. Gene expression may be measured by methods such as RT-qPCR.

### III. Methods of Preparation

The recombinant cytokine receptors described herein may be prepared and transduced into host cells, e.g., Treg cells, by any of the known protein expression methods in the art. For example, see Examples 1-3. DNA sequences encoding the recombinant cytokine receptors can be synthesized. After obtaining such sequence, it is cloned into a suitable expression vector, then transduced into a suitable host cell. The transduced host cells are recovered and cultured to obtain viable host cells that stably express the recombinant cytokine receptors of the present invention.

In some embodiments, the present application provides isolated nucleic acids encoding one or more of the polypeptides of any one of the recombinant cytokine receptors. The isolated nucleic acids may be DNA. In some embodiments, the isolated nucleic acid is inserted into a vector, such as an expression vector, a viral vector, or a cloning vector. For expression of the nucleic acids, the vector may be introduced into a host cell to allow expression of the nucleic acids within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, T7 promoter, T3 promoter, SP6 promoter, beta-actin promoter. EFLa promoter, CMV promoter, and SV40 promoter. Enhancer sequences may be selected to enhance the transcription of the nucleic acids. Selectable markers may be chosen to allow selection of the host cells inserted with the vector from those not, for example, a selectable marker may be a transmembrane gene such as EGFR that can be identified by flow cytometry and FACS analysis.

In some embodiments, the nucleic acid (e.g., vector, such as an expression vector, a viral vector, or a cloning vector) expresses an antigen receptor and/or another additional polypeptide. In some embodiments, the nucleic acid (e.g., vector, such as an expression vector, a viral vector, or a cloning vector) encodes an antigen receptor and/or another additional polypeptide. The antigen receptor may be, for example, an antibody, an engineered antibody such as an scFv, a CAR, an engineered TCR, a TCR mimic or a chimeric antibody-T cell receptor, or a chimeric signaling receptor. The antigen receptor may target an antigen of interest (e.g., a tumor antigen, an antigen of a pathogen, or a target in an inflammation site). The antigens may include, without limitation, AFP (alpha-fetoprotein), αvβ6 or another integrin, BCMA, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66c, CD70, CD74, CD79a, CD79b, CD98, CD123, CD138, CD171, CD352, CEA (carcinoembryonic antigen), Claudin, c-MET, DLL3 (delta-like protein 3). DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein a), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPa (signal-regulatory protein alpha), SLIT, SLITRK6 (NTRK-like protein 6), STEAPI (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

In some embodiments, the construct comprising the recombinant cytokine receptors described herein comprises a cleavable linker. In some embodiments, the cleavable linker is a 2A polypeptide. In some embodiments, the construct comprising the recombinant cytokine receptor comprises a nucleotide sequence encoding a P2A linker between a nucleotide sequence encoding the recombinant cytokine receptor and a marker (such as EGFR). In some embodiments, 2A-like sequences, or “peptide bond-skipping” 2A sequences, are derived from, for example, many different viruses, including, for example, from Thosea asigna. These sequences are sometimes also known as “peptide skipping sequences.” When this type of sequence is placed within a cistron between two polypeptides that are intended to be separated, the ribosome appears to skip a peptide bond; in the case of the Thosea asigna sequence, the bond between the Gly and Pro amino acids at the carboxy terminal “P-G-P” is omitted. This may leave two to three polypeptides, for example, an inducible chimeric pro-apoptotic polypeptide and a chimeric antigen receptor, or, for example, a marker polypeptide and an inducible chimeric pro-apoptotic polypeptide. When this sequence is used, the polypeptide that is encoded 5′ of the 2A sequence may end up with additional amino acids at the carboxy terminus, including the Gly residue and any upstream residues in the 2A sequence. The peptide that is encoded 3′ of the 2A sequence may end up with additional amino acids at the amino terminus, including the Pro residue and any downstream residues following the 2A sequence. In some embodiments, the cleavable linker is a 2A polypeptide derived from porcine teschovirus-1 (P2A). In some embodiments, the 2A cotranslational sequence is a 2A-like sequence. In some embodiments, the 2A cotranslational sequence is T2A (Thosea asigna virus 2A), F2A (foot and mouth disease virus 2A), P2A (porcine teschovirus-1 2A), BmCPV 2A (cytoplasmic polyhedrosis virus 2A), BmIFV 2A (flacherie virus of B, mori 2A), or E2A (equine rhinitis A virus 2A). In some embodiments, the 2A cotranslational sequence is T2A-GSG, F2A-GSG, P2A-GSG, or E2A-GSG. In some embodiments, the 2A cotranslational sequence is selected from the group consisting of T2A, P2A, and F2A. By “cleavable linker”, it is meant that the linker is cleaved by any means, including, for example, nonenzymatic means, such as peptide skipping, or enzymatic means (see, e.g., Donnelly, M L 2001, J. Gen. Virol. 82:1013-25, hereby incorporated by reference in its entirety). In a specific embodiment, a P2A comprises (or consists of) a sequence disclosed herein. In a specific embodiment, a P2A comprises (or consists of) a sequence disclosed herein (e.g., a sequence disclosed in the Examples below).

In certain embodiments, a 2A linker includes the amino acid sequence of SEQ ID NO: 10 (SGATNFSLLKQAGDVEENPGP). In certain embodiments, the 2A linker further includes a GSG amino acid sequence at the amino terminus of the polypeptide; in other embodiments, the 2A linker includes a GSGPR (SEQ ID NO:28) amino acid sequence at the amino terminus of the polypeptide. Thus, by a “2A” sequence, the term may refer to a 2A sequence in an example described herein or may also refer to a 2A sequence as listed herein further comprising a GSG or GSGPR (SEQ ID NO:28) sequence at the amino terminus of the linker.

In some embodiments, the host cell (e.g., Treg cell) contains the vector described above. The vector can be introduced to the cell using any suitable methods known in the art, including, but not limited to, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transduction of cells for expression of a vector of interest are well known in the art. In some embodiments, the host cells comprise a vector comprising the isolated nucleic acid encoding the recombinant cytokine receptor.

In some embodiments, the present application provides methods of expressing any of the recombinant cytokine receptors described herein, comprising culturing the isolated host cell containing the vector and recovering the recombinant cytokine receptor from the cell culture. The isolated host cells are cultured under conditions that allow expression of the isolated nucleic acids inserted in the vectors. Suitable conditions for expression of polynucleotides may include, without limitation, suitable medium, suitable density of host cells in the culture medium, presence of necessary nutrients, presence of supplemental factors, suitable temperatures and humidity, and absence of microorganism contaminants. A person with ordinary skill in the art can select the suitable conditions as appropriate for the purpose of the expression.

### 1. Vectors

In certain aspects, the present disclosure provides nucleic acid molecules that encode any one or more of the recombinant cytokine receptors described herein. Such nucleic acid molecules can be inserted into an appropriate vector (e.g., viral vector or non-viral plasmid vector) for introduction in a host regulatory T cell (Treg) of interest.

As used herein, the term “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, fusion proteins, enzymes, other nucleic acid molecule additions, deletions, substitutions, or other functional disruption of a cell's genetic material. Additional modifications include, for example, non-coding regulatory regions in which the modifications alter expression of a gene or operon. In some embodiments, a cell, such as a regulatory T cell (Treg), obtained from a subject may be converted into a non-natural or recombinant regulatory T cell (Treg) (e.g., a non-natural or recombinant Treg) by introducing a nucleic acid that encodes a recombinant cytokine receptor as described herein and whereby the cell expresses a cell surface located recombinant cytokine receptor.

A vector that encodes a core virus is referred to herein as a “viral vector.” There are a large number of available viral vectors suitable for use with the compositions of the instant disclosure, including those identified for human gene therapy applications (see Pfeifer and Verma, Ann. Rev. Genomics Hum. Genet. 2:177, 2001). Suitable viral vectors include vectors based on RNA viruses, such as retrovirus-derived vectors, e.g., Maloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host Tregs with viral particles containing chimeric antigen receptor transgenes are known in the art and have been previously described, for example, in U.S. Pat. No. 8,119,772; Walchli et al., pLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available.

In some embodiments, a viral vector is used to introduce a non-endogenous nucleic acid sequence encoding a recombinant cytokine receptor. A viral vector may be a retroviral vector or a lentiviral vector. A viral vector may also include nucleic acid sequences encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising green fluorescent protein, an extracellular domain of human CD2, or a truncated human EGFR (huEGFRt; see Wang et al., Blood 118:1255, 2011). When a viral vector genome comprises a plurality of nucleic acid sequences to be expressed in a host cell (e.g., T cell such as Treg) as separate transcripts, the viral vector may also comprise additional sequences between the two (or more) transcripts allowing bicistronic or multicistronic expression. Examples of such sequences used in viral vectors include internal ribosome entry sites (IRES), furin cleavage sites, viral 2A peptide, or any combination thereof.

Other vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors recently developed for gene therapy uses can also be used with the compositions and methods of this disclosure. Such vectors include those derived from baculoviruses and α-viruses. (Jolly, D J. 1999. Emerging Viral Vectors, pp 209-40 in Friedmann T, ed. The Development of Human Gene Therapy. New York: Cold Spring Harbor Lab), or plasmid vectors (such as sleeping beauty or other transposon vectors).

In certain embodiments, hematopoietic progenitor cells or embryonic stem cells are modified to comprise a non-endogenous nucleic acid molecule that encodes a recombinant cytokine receptor of this disclosure. Hematopoietic progenitor cells may comprise induced pluripotent stem cells, which may be derived or originate from fetal liver tissue, bone marrow, cord blood, or peripheral blood. The hematopoietic progenitor cells may be from human, mouse, rat, or other mammals.

In certain embodiments, the host cell transfected to express a recombinant cytokine receptor of this disclosure is a functional Treg.

One or more growth factor cytokines that promote proliferation of Tregs expressing a recombinant cytokine receptor of this disclosure may be added to the culture. The cytokines may be human or non-human. Exemplary growth factor cytokines that may be used to promote Treg proliferation include IL-2, TGFβ, or similar.

### IV. Methods of Expanding and Culturing Cells

In some embodiments, the cell transfected to express a recombinant cytokine receptor of this disclosure is a eukaryotic cell. In some embodiments, the cell transfected to express a recombinant cytokine receptor of this disclosure is a human cell. In some embodiments, the cell transfected to express a recombinant cytokine receptor of this disclosure is a human embryonic kidney (HEK) cell. In certain embodiments, the cell transfected to express a recombinant cytokine receptor of this disclosure is an immune cell. In certain embodiments, the cell transfected to express a recombinant cytokine receptor of this disclosure is a T cell. In certain embodiments, the cell transfected to express a recombinant cytokine receptor of this disclosure is a functional Treg.

In some embodiments the Treg cell is isolated from peripheral blood mononuclear (PBMC) cells. In some embodiments, the Treg cell is isolated from PBMCs using density gradient centrifugation. In some embodiments, the Treg cells are enriched by positive selection. In some embodiments, the Treg cells are enriched by positive selection for CD25+. In some embodiments, the Treg cells are enriched by positive selection for CD4+CD25+CD127lo cells. In some embodiments, the enrichment occurs by FACS. In some embodiments, the Tregs are stimulated by anti-CD3 antibody and/or anti-CD28 antibody on day 0 after positive selection. In some embodiments, the Tregs are re-stimulated by anti-CD3 antibody and/or anti-CD28 antibody on day 9 of culture after positive selection.

One or more growth factor cytokines that promote proliferation of Tregs expressing a recombinant cytokine receptor of this disclosure may be added to the culture. The cytokines may be human or non-human cytokines. Exemplary growth factor cytokines that may be used to promote Treg proliferation include IL-4, IL-7, IL-9, IL-21, or the like. In some embodiments, the cytokines are added to the media prior to selection. In some embodiments, the cytokines are added to the media after cell isolation. In some embodiments, the cytokines are added to the media for approximately 15-60 minutes. In some embodiments, the cytokines are added to the media for approximately 40 minutes. In some embodiments, cytokines are added to the cell culture approximately every 12 to 60, such as about 24 to about 48 hours. In some embodiments, the cytokines are added to the media for the duration of the culture. In some embodiments, the cells are treated with cytokines wherein the cells are cultured at a cell density of approximately 0.25-0.3 million cells/mL. In some embodiments, the concentration of cytokines during cytokine treatment is approximately 0.15 ng/ml-100 ng/ml. In some embodiments, the concentration of IL-2 during cytokine treatment is approximately 300 IU/mL. In some embodiments, the concentration of IL-2 during cytokine treatment is 300 IU/mL.

In some embodiments, cells expressing the recombinant cytokine receptor of this disclosure are cultured for a sufficient time to induce proliferation or differentiation. The cells are maintained in culture generally for about 3 days to about 5 days, about 4 to about 10 days, about 5 to about 20 days, about 10 to about 23 days, about 15 to about 30 days, or about 23 to about 30 days. It will be appreciated that the cells may be maintained for an appropriate amount of time required to achieve a desired result, i.e., a desired cellular composition or level of proliferation. For example, to generate a cellular composition comprising primarily Tregs, cells may be maintained in culture for about 30 days.

In some embodiments, the method further comprises detecting one or more Treg markers provided herein. In some embodiments, the method further comprises detecting IL-2 signaling. In some embodiments, the method further comprises detecting phosphorylated STAT-5.

In some embodiments, the suppressive activity of the transduced Treg is increased upon transduction with a recombinant cytokine receptor provided herein. In some embodiments, the rate of division of conventional (e.g., cytotoxic) T cells is decreased by the transduced Treg cells provided herein. In some embodiments, the rate of division of CD4+ T cells is decreased by the transduced Treg cells provided herein compared to CD4+ T cells cultured without Tregs. In some embodiments, the rate of division of CD8+ T cells is decreased by the transduced Treg cells provided herein compared to CD8+ T cells cultured without Tregs. In some embodiments, the activity of cytotoxic T cells is decreased.

In some embodiments, provided herein is a method of expanding a transduced Treg cell in the absence of IL-2 comprising culturing a cell expressing a recombinant receptor provided herein. In some embodiments, the cell is cultured in a culture medium comprising a cytokine other than IL-2. In some embodiments, the cell is cultured in a cell medium comprising a cytokine that binds to the ED of the recombinant cytokine receptor. In some embodiments, the cell is cultured in a medium comprising two, three, four, five, or more cytokines.

### V. Pharmaceutical Compositions, Articles of Manufacture, and Kits

Further provided by the present application are pharmaceutical compositions comprising a cell, e.g., a T cell such as a Treg cell, comprising a recombinant cytokine receptor described herein.

The pharmaceutical compositions may be suitable for a variety of modes of administration described herein, including, for example, systemic or localized administration. In some embodiments, the pharmaceutical composition is formulated for intravenous administration.

The pharmaceutical compositions to be used for in vivo administration are generally formulated as sterile, substantially isotonic, and in full compliance with all Good Manufacturing Practice (GMP) regulations of the U.S. Food and Drug Administration. Sterility is readily accomplished by filtration through sterile filtration membranes, for example the filtration of any liquid solutions as described herein used for reconstituting, storing, etc, the Treg cells or pharmaceutical composition described herein. In some embodiments, the composition is free of pathogens. For injection, the pharmaceutical composition can be in the form of liquid solutions, for example in physiologically compatible buffers such as Hank's solution or Ringer's solution.

In some embodiments, the pharmaceutical composition is suitable for administration to a human. In some embodiments, the pharmaceutical composition is suitable for administration to a rodent (e.g., mice, rats) or non-human primates (e.g., Cynomolgus monkey). In some embodiments, the pharmaceutical composition is cryopreserved.

The present application also provides kits comprising compositions (such as pharmaceutical compositions) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### VI. Methods of Treatment

In some embodiments, provided herein is a method of treating an immune-related disorder comprising administering a cell (e.g., a Treg cell) comprising a recombinant cytokine receptor provided herein. In some embodiments, the recombinant cytokine receptor comprises an ED and TD, and the ID of Interleukin-2 Receptor beta (IL-2Rβ), wherein the ED does not bind to exogenous IL-2. In some embodiments, the recombinant cytokine receptor used in the method of treating an immune-related disorder is able to signal in the absence of exogenous IL-2. In some embodiments, the recombinant cytokine receptor used in the method of treating an immune-related disorder comprises an interleukin ED, a TD, and an ID. In some embodiments, the ED binds to a cytokine other than IL-2. Thus, in some embodiments, there is provided a method of treating an immune-related disorder comprising administering a cell (e.g., a Treg cell) comprising a recombinant cytokine receptor comprising: (I) the ED of IL-4Rα, IL-7Rα, IL-9Rα, or IL-21Rα; (II) the IL-2Rβ, IL-4Rα, IL-7Rα, IL-9Rα, or IL-21Rα; and (III) the ID of IL-2Rβ. In some embodiments, the recombinant cytokine receptor comprises the amino acid sequence set forth in SEQ ID NOs: 1-5. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NOs: 1-5. In some embodiments, the cell is an immune cell. In some embodiments, the cell is a lymphocyte. In some embodiments, the cell is a T-cell. In some embodiment, the cell is a Treg.

In some embodiments, the method of treating an immune-related disorder comprises administering a cell (e.g., a Treg cell, such as any of the Treg cells described herein) comprising a recombinant cytokine receptor comprising the ED of IL-4Rα. In some embodiments, the method comprises transducing the cell (e.g., the Treg cell) with a vector encoding the recombinant cytokine receptor. In some embodiments, the cells are autologous to the individual to be treated. In some embodiments, the individual to be treated expresses IL-2. In some embodiments ED comprises the amino acid sequence set forth in SEQ ID NO: 11. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:11. In some embodiments, the method of treating an immune disorder comprises transducing a cell (e.g., a Treg cell) with a recombinant cytokine receptor comprising in N-terminal to C-terminal direction: (I) the ED of IL-4Rα; (II) the TD of IL-2Rβ, IL-4Rα, IL-7Rα, IL-9Rα, or IL-21Rα; (III) the ID of IL-2Rβ. In some embodiments, the recombinant cytokine receptor comprises in N-terminal to C-terminal direction: (I) the ED of IL-4Rα; (II) the TD of IL-4Rα or IL-2Rβ; (III) the ID of IL-2Rβ. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 1, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:11. In some embodiments, the recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO:1, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO:11. In some embodiments, the cell is an immune cell. In some embodiments, the cell is a lymphocyte. In some embodiments, the cell is a T-cell. In some embodiment, the cell is a Treg.

In some embodiments, the method of an immune-related disorder comprises administering a cell (e.g., a Treg cell, such as any of the Treg cells described herein) comprising a recombinant cytokine receptor comprising the ED of IL-7Rα. In some embodiments, the method comprises transducing the cell (e.g., Treg cell) with a vector encoding the recombinant cytokine receptor. In some embodiments, the cells are autologous to the individual to be treated. In some embodiments, the individual to be treated expresses IL-2. In some embodiments, the IL-7Rα ED comprises the amino acid sequence set forth in SEQ ID NO: 12. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO: 12. In some embodiments, the method of treating an immune-related disorder comprising administering a cell (e.g., a Treg cell) comprising a recombinant cytokine receptor comprises in N-terminal to C-terminal direction: (I) the ED of IL-7Rα; (II) the TD of IL-2Rβ, IL-4Rα, IL-7Rα, IL-9Rα, or IL-21Rα; (III) the ID of IL-2Rβ. In some embodiments, the recombinant cytokine receptor comprises in N-terminal to C-terminal direction: (I) the ED of IL-7Rα; (II) the TD of IL-7Rα or IL-2Rβ; (III) the ID of IL-2Rβ. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:2, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 12. In some embodiments, the recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO: 2, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO:12. In some embodiments, the cell is an immune cell. In some embodiments, the cell is a lymphocyte. In some embodiments, the cell is a T-cell. In some embodiment, the cell is a Treg.

In some embodiments, the method of treating an immune-related disorder comprises administering a cell (e.g., a Treg cell, such as any of the Treg cells described herein) comprising a recombinant cytokine receptor comprising the ED of IL-9Rα. In some embodiments, the method comprises transducing the cell (e.g., Treg cell) with a vector encoding the recombinant cytokine receptor. In some embodiments, the cells are autologous to the individual to be treated. In some embodiments, the individual to be treated expresses IL-2. In some embodiments, the IL-9Rα ED comprises the amino acid sequence set forth in SEQ ID NO:13. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:13. In some embodiments, the method of treating an immune-related disorder comprises administering a cell (e.g., a Treg cell) comprising a recombinant cytokine receptor comprising in N-terminal to C-terminal direction: (I) the ED of IL-9Rα; (II) the TD of IL-2Rβ, IL-4Rα, IL-7Rα, IL-9Rα, or IL-21Rα; (III) the ID of IL-2Rβ. In some embodiments, the recombinant cytokine receptor comprises in N-terminal to C-terminal direction: (I) the ED of IL-9Rα; (II) the TD of IL-9Rα or IL-2Rβ; (III) the ID of IL-2Rβ. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:3 or 5, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 13. In some embodiments, the recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO:3 or 5, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO:13. In some embodiments, the cell is an immune cell. In some embodiments, the cell is a lymphocyte. In some embodiments, the cell is a T-cell. In some embodiment, the cell is a Treg.

In some embodiments, the method of treating an immune-related disorder comprises using a recombinant cytokine receptor comprising the ED of IL-21Rα. In some embodiments, the method comprises transducing the cell (e.g., Treg cell, such as any of the Treg cells described herein) with a vector encoding the recombinant cytokine receptor. In some embodiments, the cells are autologous to the individual to be treated. In some embodiments, the individual to be treated expresses IL-2. In some embodiments, the IL-21Rα ED comprises the amino acid sequence set forth in SEQ ID NO:14. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:14. In some embodiments, the method of treating an immune-related disorder comprises using a recombinant cytokine receptor comprising in N-terminal to

C-terminal direction: (I) the ED of IL-21Rα; (II) the TD of IL-2Rβ, IL-4Rα, IL-7Rα, IL-9Rα, or IL-21Rα; (III) the ID of IL-2Rβ. In some embodiments, the recombinant cytokine receptor comprises in N-terminal to C-terminal direction: (I) the ED of IL-21Rα; (II) the TD of IL-21Rα or IL-2Rβ; (III) the ID of IL-2Rβ. In some embodiments, the recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 4, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:14. In some embodiments, the recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO:4, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO: 14. In some embodiments, the cell is an immune cell. In some embodiments, the cell is a lymphocyte. In some embodiments, the cell is a T-cell. In some embodiment, the cell is a Treg.

In some embodiments, the method of treating an immune-related disorder comprises administering a cell (e.g., a Treg cell, such as any of the Treg cells described herein) comprising a recombinant cytokine receptor comprising the ED of IL-4Rα. In some embodiments, the method comprises transducing the cell (e.g., Treg cell) with a vector encoding the recombinant cytokine receptor. In some embodiments, the cells are autologous to the individual to be treated. In some embodiments, the individual to be treated expresses IL-2. In some embodiments ED comprises the amino acid sequence set forth in SEQ ID NO:11. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:11. In some embodiments, the recombinant cytokine receptor comprises in N-terminal to C-terminal direction: (I) IL-4 cytokine, (II) the ED of IL-4Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-4 cytokine; (II) the ED of IL-4Rα; (III) the TD of IL-4Rα or IL-2Rβ; and (IV) the ID of IL-2Rβ. In some embodiments IL-4 cytokine is tethered to the ED of IL-4Rα by a polypeptide linker. In some embodiments, the IL-4 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 6, wherein the recombinant cytokine receptor comprises an IL-4 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:22, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:11. In some embodiments, the IL-4 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO:6, wherein the recombinant cytokine receptor comprises an IL-4 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:22, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO:11. In some embodiments, the cell is an immune cell. In some embodiments, the cell is a lymphocyte. In some embodiments, the cell is a T-cell. In some embodiment, the cell is a Treg.

In some embodiments, the method of treating an immune-related disorder comprises administering a cell (e.g., a Treg cell, such as any of the Treg cells described herein) comprising a recombinant cytokine receptor comprising the ED of IL-7Rα. In some embodiments, the method comprises transducing the cell (e.g., Treg cell) with a vector encoding the recombinant cytokine receptor. In some embodiments, the cells are autologous to the individual to be treated. In some embodiments, the individual to be treated expresses IL-2. In some embodiments, the IL-7Rα ED comprises the amino acid sequence set forth in SEQ ID NO: 12. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:12. In some embodiments, the recombinant cytokine receptor comprises in N-terminal to C-terminal direction: (I) IL-7 cytokine, (II) the ED of IL-7Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-7 cytokine; (II) the ED of IL-7Rα; (III) the TD of IL-7Rα or IL-2Rβ; and (IV) the ID of IL-2Rβ. In some embodiments, IL-7 cytokine is tethered to the ED of IL-7Rα by a polypeptide linker. In some embodiments, the IL-7 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 7, wherein the recombinant cytokine receptor comprises an IL-7 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:23, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:12. In some embodiments, the IL-7 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO:7, wherein the recombinant cytokine receptor comprises an IL-7 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:23, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO: 12. In some embodiments, the cell is an immune cell. In some embodiments, the cell is a lymphocyte. In some embodiments, the cell is a T-cell. In some embodiment, the cell is a Treg.

In some embodiments, the method of treating an immune-related disorder comprises administering a cell (e.g., a Treg cell, such as any of the Treg cells described herein) comprising a recombinant cytokine receptor comprising the ED of IL-9Rα. In some embodiments, the method comprises transducing the cell (e.g., Treg cell) with a vector encoding the recombinant cytokine receptor. In some embodiments, the cells are autologous to the individual to be treated. In some embodiments, the individual to be treated expresses IL-2. In some embodiments, the IL-9Rα ED comprises the amino acid sequence set forth in SEQ ID NO: 13. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:13. In some embodiments, the recombinant cytokine receptor comprises in N-terminal to C-terminal direction: (I) IL-9 cytokine, (II) the ED of IL-9Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-9 cytokine; (II) the ED of IL-9Rα; (III) the TD of IL-9Rα or IL-2Rβ; and (IV) the ID of IL-2Rβ. In some embodiments IL-9 cytokine is tethered to the ED of IL-9Rα by a polypeptide linker. In some embodiments, the IL-9 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 8 or 15, wherein the recombinant cytokine receptor comprises an IL-9 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:24, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:13. In some embodiments, the IL-9 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO:8 or 15, wherein the recombinant cytokine receptor comprises an IL-9 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:24, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO:13. In some embodiments, the cell is an immune cell. In some embodiments, the cell is a lymphocyte. In some embodiments, the cell is a T-cell. In some embodiment, the cell is a Treg.

In some embodiments, the method of treating an immune-related disorder comprises administering a cell (e.g., a Treg cell, such as any of the Treg cells described herein) comprising a recombinant cytokine receptor comprising the ED of IL-21Rα. In some embodiments, the method comprises transducing the cell (e.g., Treg cell) with a vector encoding the recombinant cytokine receptor. In some embodiments, the cells are autologous to the individual to be treated. In some embodiments, the individual to be treated expresses IL-2. In some embodiments, the IL-21Rα ED comprises the amino acid sequence set forth in SEQ ID NO: 14. In some embodiments, the ED comprises an amino acid sequence comprising at least about 80%, at least about 85%, at least about 90%, at least about 95%, at least about 96%, at least about 97%, at least about 98%, at least about 99%, or about 100% to SEQ ID NO:14. In some embodiments, the recombinant cytokine receptor comprises in N-terminal to C-terminal direction: (I) IL-21 cytokine, (II) the ED of IL-21Rα; (III) the TD of IL-2Rβ, IL-4Rα, IL-7Rα, IL-9Rα, or IL-21Rα; and (IV) the ID of IL-2Rβ. In some embodiments, the recombinant cytokine receptor comprises in N-terminal to C-terminal direction: (I) IL-21 cytokine; (II) the ED of IL-21Rα; (III) the TD of IL-21Rα or IL-2Rβ; and (IV) the ID of IL-2Rβ. In some embodiments, the IL-21 tethered recombinant cytokine receptor comprises an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:9, wherein the recombinant cytokine receptor comprises an IL-21 cytokine having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO:25, and wherein the recombinant cytokine receptor comprises an ED having an amino acid sequence comprising at least about 80%, at least about 85%, at least about 90%, at least about 95%, at least about 96%, at least about 97%, at least about 98%, at least about 99%, or about 100% sequence identity with the amino acid sequence set forth in SEQ ID NO: 14. In some embodiments, the IL-21 tethered recombinant cytokine receptor comprises an amino acid sequence comprising at least about 90%, at least about 95%, at least about 96%, at least about 97%, at least about 98%, at least about 99%, or about 100% sequence identity with the amino acid sequence set forth in SEQ ID NO:9, wherein the recombinant cytokine receptor comprises an IL-21 cytokine having an amino acid sequence comprising at least about 90%, at least about 95%, at least about 96%, at least about 97%, at least about 98%, at least about 99%, or about 100% sequence identity with the amino acid sequence set forth in SEQ ID NO:25, and wherein the recombinant cytokine receptor comprises an ED having an amino acid sequence comprising at least about 90%, at least about 95%, at least about 96%, at least about 97%, at least about 98%, at least about 99%, or about 100% sequence identity with the amino acid sequence set forth in SEQ ID NO: 14. In some embodiments IL-21 cytokine is tethered to the ED of IL-21Rα by a polypeptide linker. In some embodiments, the cell is an immune cell. In some embodiments, the cell is a lymphocyte. In some embodiments, the cell is a T-cell. In some embodiment, the cell is a Treg.

In some embodiments, the recombinant cytokine receptor of the present disclosure is expressed in a T cell. In some embodiments, one or more recombinant cytokine receptors are expressed in a T cell. In some embodiments, the recombinant cytokine receptor can be co-expressed with a chimeric antigen receptor (CAR). In some embodiments, the T cell is a regulatory T cell (Treg). In some embodiments, Treg is CD4+, CD25+, CD127lo. In some embodiments, the Treg expresses FOXP3 and/or HELIOS. In some embodiments, the recombinant cytokine receptor is expressed in a Treg cell.

Immune conditions, diseases, disorders and reactions or responses to be treated according to the methods and compositions of the invention means a disease in which the immune system contributes to pathogenesis or can be part of the treatment. These reactions include, but are not limited to, cancers, inflammation, autoimmune conditions, disorders or diseases and persistent and progressive immune reactions to infectious non-self-antigens from bacterial, viral (e.g., HCV), fungal, or parasitic organisms which invade and persist within mammals and humans. Such conditions and disorders include allergies and/or asthma. The allergies and asthma may be due to sensitization with foreign or non-self-antigens as pollen, animal dander and food proteins. The source of the provoking foreign antigen can be plant, fungal, mold, or other environmental contaminants.

Autoimmunity is defined as persistent and progressive immune reactions to noninfectious self-antigens, as distinct from infectious non-self-antigens from bacterial, viral, fungal, or parasitic organisms which invade and persist within mammals and humans. Autoimmune conditions include graft-versus-host disease, autoimmune polyendocrinopathy syndromes, Type I diabetes mellitus (TIDM), autoimmune gastritis, autoimmune uveoretinitis, autoimmune vasculitis, colitis, thyroiditis, Addison's disease, agammaglobulinemia, alopecia arcata, amyloidosis, ankylosing spondylitis, anti-GBM/anti-TBM nephritis, antiphospholipid syndrome, autoimmune hepatitis, autoimmune inner ear disease, axonal & neuronal neuropathy, Behqet's disease, bullous pemphigold, Castleman disease, celiac disease, Chagas disease, chronic inflammatory demyelinating polyneuropathy (CIDP), chronic recurrent multifocal osteomyelitis, Churg-Strauss, cicatrical pemphigold/benign mucosal pemphigold, Cogan's syndrome, cold agglutinin disease, congenital heart block, coxsackie myocarditis, CREST syndrome, Crohn's disease, dermatitis herpetiformis, dermatomyositis, Devic's disease, discoid lupus, Dressler's syndrome, endometriosis, cosinophilic esophagitis, cosinophilic fasciitis, crythema nodosum, essential mixed cryoglobulinemia, Evans syndrome, fibromyalgia, fibrosing alveolitits, giant cell arteritis (temporal arteritis), giant cell myocarditis, glomerulonephritis, Goodpasture's syndrome, granulomatosis with polyangitis, Grave's disease, Guillian-Barre syndrome, Hashimoto's thyroiditis, hemolytic anemia, Henoch-Schonnlien purpura, herpes gestationis or pemphigold gestationis, hypogammaglobulinemia, IgA nephropathy, IgG4-related sclerosing disease, inclusion body myositis, interstitial cystitis, juvenile arthritis, juvenile diabetes (Type1diabetes), juvenile myositis, Kawasaki disease, Lambert-Eaton syndrome, leukocyteclastic vasculitis, lichen planus, lichen sclerosis, ligneous conjunctivitis, linear IgA disease, lupus, Lyme disease, chronic Meniere's disease, microscopic polyanglitis, mixed connective tissue disease, Mooren's ulcer, Mucha-Habermann disease, multiple sclerosis, myasthenia gravis, myositis, narcolepsy, neuromyelitis opica, neutropenia, ocular cicatrical pemphigold, optic neuritis, palindromic rheumatism, PANDAS (pediatric autoimmune neuropsychiatric disorders associated with streptococcus), parancoplastic cerebellar degeneration, paroxysmal nocturnal hemoglobinuria, Parry Romber syndrome, pars planitis (peripheral uveitis), Parsonnage-Turner syndrome, pemphigus, peripheral neuropathy, perivenous encephalomyelitis, pernicious anemia, POEMS syndrome, polyarteritis nodosa, polymyalgia rheumatic, polymyositis, postmycoardial infarction syndrome, postpericadiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, Reiter's syndrome, relapsing polychondritis, restless leg syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophtalmia, Takayasu's arteritis, temporal arteritis/giant cell arteritis, thrombocytopenia purpura. Tolosa-Hunt syndrome, transverse myelitis, ulcerative colitis, undifferentiated connective tissue disease, uveitis, vasculitis, vitiligo, and Wegner's granulomatosis (granulomatosis with polyangitis). “Autoantigen” or “self-antigen” as used herein refers to an antigen or epitope which is native to the mammal, and which is immunogenic in said mammalian disease. One aspect of the present application provides a method of treating an immune-related disorder.

The cells that may be “allogeneic cells” are those isolated from one individual (the donor) and infused into another; whereas “autologous cells” refer to those cells that are isolated and infused back into the same individual. In some embodiments, the cells are autologous to the individual. In some embodiments, the individual is a human. In some embodiments, the individual expresses IL-2. In some embodiments, the cells are T cells. In some embodiments, the cells are Tregs. In some embodiments, the Treg cells are isolated from human peripheral blood mononuclear cells (PBMCs). In some embodiments, the Treg cells are modified ex vivo with a vector encoding for one or more recombinant cytokine receptors. In some embodiments, the Treg cells are expanded ex vivo in the absence of IL-2. In some embodiments, the Treg cells are modified with a nucleic acid or vector coding for the recombinant cytokine receptors. In some embodiments, the Treg cells are modified to proliferate in the absence of IL-2. In some embodiments, the Treg cells have one or more markers of IL-2 signaling detected. In some embodiments, the detected marker of IL-2 signaling is phosphorylated STAT-5. In some embodiments, the Treg is CD4+, CD25+, CD127lo. In some embodiments, the Treg expresses FOXP3 and/or HELIOS. In some embodiments, the Treg cells are administered to the same individual to treat an immune-related disorder.

In some embodiments, the method of treating an immune-related disorder comprises administering Treg cells to an individual in need thereof. In some embodiments, the individual expresses IL-2. In some embodiments, the recombinant cytokine receptor can be co-expressed with a chimeric antigen receptor (CAR). In some embodiments, the Treg is CD4+, CD25+, CD127lo. In some embodiments, the Treg expresses FOXP3 and/or HELIOS.

In some embodiments, the method of treating an immune-related disorder comprises administering Treg cells that are autologous to the individual. In some embodiments, the individual is a human. In some embodiments, the individual expresses IL-2. In some embodiments, the method of treating an immune-related disorder comprises administering Treg cells that are isolated from human peripheral blood mononuclear cells (PBMCs). In some embodiments, the Treg cells are modified ex vivo with a vector encoding for one or more recombinant cytokine receptors. In some embodiments, the Treg cells are expanded ex vivo in the absence of IL-2. In some embodiments, the method of treating an immune-related disorder comprises administering Treg cells that are modified ex vivo with a vector encoding for one or more recombinant cytokine receptors. In some embodiments, the method of treating an immune-related disorder comprises administering Treg cells that are expanded ex vivo in the absence of IL-2. In some embodiments, the method of treating an immune-related disorder comprises administering Treg cells that are modified with a nucleic acid or vector coding for the recombinant cytokine receptors. In some embodiments, the method of treating an immune-related disorder comprises administering the Treg cells that are modified to proliferate in the absence of IL-2. In some embodiments, the method of treating an immune-related disorder comprises administering the Treg cells wherein one or more markers of IL-2 signaling are detected. In some embodiments, the method of treating an immune-related disorder comprises administering the Treg cells wherein the detected marker of IL-2 signaling is phosphorylated STAT-5. In some embodiments, the method of treating an immune-related disorder comprises administering the Treg cells which are CD4+, CD25+, CD127lo. In some embodiments, the method of treating an immune-related disorder comprises administering the Treg cells that express FOXP3 and/or HELIOS. In some embodiments, the method of treating an immune-related disorder comprises administering the Treg cells described herein.

In some embodiments, the method of treating an immune-related disorder comprises administering the Treg cells comprising the recombinant cytokine receptors of the present disclosure. In some embodiments, the suppressive activity of the Tregs on CD8+ and/or CD4+ T cells is increased compared to a wild-type Treg. In some embodiments, the suppressive activity of the Tregs on CD8+ and/or CD4+ T cells is increased compared to a wild-type Treg when cultured without IL-2. In some embodiments, the suppressive activity of the Tregs on CD8+ and/or CD4+ T cells is increased compared to unmodified cells cultured without IL-2. In some embodiments, the suppressive activity of the Tregs on CD8+ and/or CD4+ T cells cultured without IL-2 is approximately equivalent or about the same as unmodified Treg cells cultured with IL-2. In some embodiments, the suppressive activity of the Tregs decreases the rate of division of CD4+ and/or CD8+ T cells. In some embodiments, the method further comprises culturing the Treg cells in a composition comprising cytokine. In some embodiments, the method further comprises culturing the Treg cells in a composition comprising the cognate cytokine. In some embodiments, the method further comprises culturing the Treg cells in a composition comprising one or more additional cytokines other than IL-2.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor. In some embodiments, the viability of the cells is increased compared to an untransduced Treg. In some embodiments, at least about 60-99% of the cells in the population of Treg cells transduced with the recombinant cytokine remain viable in vitro approximately 7-20 days after transduction. In some embodiments, at least about 60-99% of the cells in the population of Treg cells transduced with the recombinant cytokine remain viable in vitro approximately 7-14 days after transduction. In some embodiments, at least about 60% of the cells in the population of Treg cells transduced with the recombinant cytokine remain viable in vitro approximately 7 days after transduction. In some embodiments, the method further comprises culturing the Treg cells in a composition comprising cytokine. In some embodiments, the method further comprises culturing the Treg cells in a composition comprising the cognate cytokine. In some embodiments, the method further comprises culturing the Treg cells in a composition comprising one or more cytokines additional cytokines other than IL-2.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor. In some embodiments, the viability of the cells is increased compared to an untransduced Treg. In some embodiments, at least about 60-99% of the cells in the population of Treg cells transduced with the recombinant cytokine can persist in vivo for at least about 3 days or longer. In some embodiments, at least about 60-99% of the cells in the population of Treg cells transduced with the recombinant cytokine can persist in vivo for at least about 3 days or longer. In some embodiments, at least about 60% of the cells in the population of Treg cells transduced with the recombinant cytokine can persist in vivo for at least about 3 days or longer. In some embodiments, the population of Treg cells transduced with the recombinant cytokine can persist in vivo for at least about 1, 2, 3, 5, 6, 8, 10, or 11 months, or at least a year or more.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor, which results in a population of cells that contains more viable cells compared to a composition comprising a population of the same Treg cells not transduced with the recombinant cytokine receptor. In some embodiments, at least about 60-99% of the cells in the population of Treg cells transduced with the recombinant cytokine remain viable in vitro approximately 7-20 days after transduction. In some embodiments, at least about 60-99% of the cells in the population of Treg cells transduced with the recombinant cytokine remain viable in vitro approximately 7-14 days after transduction. In some embodiments, at least about 60% of the cells in the population of Treg cells transduced with the recombinant cytokine remain viable in vitro approximately 7 days after transduction. In some embodiments, the method further comprises culturing the Treg cells in a composition comprising cytokine. In some embodiments, the method further comprises culturing the Treg cells in a composition comprising the cognate cytokine.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor, which results in a population of cells that contains more viable cells compared to a composition comprising a population of the same Treg cells not transduced with the recombinant cytokine receptor. In some embodiments, at least about 60-99% of the cells in the population of Treg cells transduced with the recombinant cytokine can persist in vivo for at least about 3 days or longer. In some embodiments, at least about 60-99% of the cells in the population of Treg cells transduced with the recombinant cytokine can persist in vivo for at least about 3 days or longer. In some embodiments, at least about 60% of the cells in the population of Treg cells transduced with the recombinant cytokine can persist in vivo for at least about 3 days or longer. In some embodiments, the population of Treg cells transduced with the recombinant cytokine can persist in vivo for at least about 1, 2, 3, 5, 6, 8, 10, or 11 months, or at least a year or more.

In some embodiments, the method of treating an immune-related disorder comprises transducing a population of Treg cells transduced with the recombinant cytokine receptor, wherein the population of transduced Treg cells expands at least two-fold more than a population of the same Treg cells not transduced with the recombinant cytokine receptor. In some embodiments, the method of treating an immune-related disorder comprises a composition wherein the population of Treg cells transduced with the recombinant cytokine receptor expands at least two-fold, and wherein the population of Treg cells transduced with the recombinant cytokine receptor maintains expression of least one Treg marker selected from the group consisting of CD4+, CD25+, and CD127lo.

In some embodiments, the recombinant cytokine receptors of the present disclosure may also be used in combination with a chimeric antigen receptor (CAR). In some embodiments, the modified Treg comprises a recombinant cytokine receptor and a chimeric antigen

### VII. Definitions

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely.” “only.” and the like in connection with the recitation of claim elements, or use of a “negative” limitation.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X”.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising.” “consisting.” and “consisting essentially of” aspects and embodiments.

As used herein, the terms “including.” “containing,” and “comprising” are used in their open, non-limiting sense.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc, and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, preferably digital UNIX V4.0D. All sequence comparison parameters are set by the ALIGN-2 program and do not vary.

An “individual” or “subject” is a mammal. Mammals include, but are not limited to, domesticated animals (e.g., cows, sheep, cats, dogs, and horses), primates (e.g., humans and non-human primates such as rhesus and cynomolgus monkeys), rabbits, and rodents (e.g., mice and rats). In some embodiments, the individual or subject is a human.

Treatment” or “therapy” of a subject refers to any type of intervention or process performed on, or the administration of an active agent to, the subject with the objective of curing, reversing, alleviating, ameliorating, inhibiting, slowing down, or preventing the onset, progression, development, severity, or recurrence of a symptom, complication, condition, or biochemical indicia associated with a disease.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The phrase “pharmaceutically acceptable” indicates that the substance or composition must be compatible chemically and/or toxicologically, with the other ingredients comprising a formulation, and/or the mammal being treated therewith.

As described herein, the term “tethered” includes fused, linked, connected, attached, etc, and can include any method known to one of skill in the art for fusing, linking, connecting, attaching, etc. For example, two polypeptide sequences can be “tethered” or otherwise fused, linked, connected, attached, etc, using a polypeptide or peptide linker.

As described herein, any concentration range, percentage range, ratio range, or integer range is to be understood to include the value of any integer within the recited range and, when appropriate, fractions thereof (such as one tenth and one hundredth of an integer), unless otherwise indicated. Description of endpoints includes ranges between all endpoints disclosed. For example, description of 1, 2, or 3 includes the ranges 1-2, 2-3, and 1-3.

### Embodiments

1. A recombinant cytokine receptor comprising an extracellular cytokine receptor domain, a transmembrane domain, and an intracellular IL-2 receptor beta chain domain, wherein the extracellular cytokine receptor domain binds to a cytokine other than IL-2.
2. A recombinant cytokine receptor comprising an extracellular cytokine receptor domain, a transmembrane domain, and an intracellular IL-2 receptor beta domain, wherein the extracellular cytokine receptor domain binds to and is tethered to a cytokine other than IL-2.
3. The recombinant cytokine receptor of embodiment 1 or 2, wherein the extracellular cytokine receptor domain is selected from the group consisting of an IL-4 extracellular domain, an IL-7 extracellular domain, an IL-9 extracellular domain, and an IL-21 extracellular domain.
4. The recombinant cytokine receptor of any one of embodiments 2-3, wherein the extracellular cytokine receptor domain is tethered to the cytokine by a polypeptide linker.
5. The recombinant cytokine receptor of embodiment 4, wherein the peptide linker comprises glycine and serine residues.
6. The recombinant cytokine receptor of embodiment 1, wherein the extracellular cytokine receptor domain is not tethered to the cytokine.
7. The recombinant cytokine receptor of any one of embodiments 1-6, wherein the cytokine receptor engages in IL-2 signaling in the absence of IL-2.
8. The cytokine receptor of any one of embodiments 1-7, wherein the transmembrane domain is a transmembrane domain of a cytokine receptor.
9. The recombinant cytokine receptor of embodiment 8, wherein the transmembrane domain is a transmembrane domain of an IL-9 receptor, an IL-2 receptor, an IL-4 receptor, an IL-7 receptor, or an IL-21 receptor.
10. The recombinant cytokine receptor of any one of embodiments 1-9, wherein the intracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:20.
11. The recombinant cytokine receptor of any one of embodiments 1-10, wherein the extracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:11-14.
12. The recombinant cytokine receptor of any one of embodiments 1-11, wherein the transmembrane domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:16-19.
13. The recombinant cytokine receptor of any one of embodiments 1-12, wherein the cytokine receptor comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:6-9.
14. A nucleic acid encoding the recombinant cytokine receptor of any one of embodiments 1-13.
15. A vector comprising the nucleic acid of embodiment 14.
16. The vector of embodiment 15, wherein the vector is a lentiviral vector.
17. The vector of embodiment 15 or 16, further comprising a marker gene.
18. The vector of embodiment 17, wherein the marker gene is a transmembrane protein.
19. The vector of embodiment 18, wherein the transmembrane protein is EGFR.
20. A T cell comprising the recombinant cytokine receptor of any one of embodiments 1-13, the nucleic acid of embodiment 14, or the vector of any one of embodiments 15-19.
21. The T cell of embodiment 20, wherein the T cell is a regulatory T cell (Treg), wherein the Treg is CD4+, CD25+, and CD127lo.
22. The T cell of embodiment 21, wherein the Treg expresses FOX3P and/or HELIOS.
23. The T cell of any one of embodiments 20-22, further comprising a chimeric antigen receptor (CAR).
24. A composition comprising the nucleic acid of embodiment 14, the vector of any one of embodiments 15-20, or the T cell of any one of embodiments 21-23.
25. A method of treating an immune-related disorder comprising administering the T cell of any one of embodiments 20-23 or the composition of embodiment 24, to an individual in need thereof.
26. The method of embodiment 25, wherein the cells are autologous to the individual.
27. The method of any one of embodiments 25-26, wherein the individual is human.
28. A method of expanding a transduced Treg cell in the absence of IL-2 comprising introducing the nucleic acid of embodiment 14, or the vector of any one of embodiments 15-19 into the Treg cell and culturing the cell in the absence of IL-2.
29. The method of embodiment 28, further comprising detecting at least one Treg marker selected from the group consisting of CD4+, CD25+, and CD127.
30. The method of embodiment 29, further comprising detecting FOXP3 and/or HELIOS.
31. The method of any one of embodiments 28-30, wherein the Treg cell is able to proliferate in the absence of IL-2.
32. The method of any one of embodiments 28-31, wherein one or more markers of IL-2 signaling is detected.
33. The method of any one of embodiments 28-32, wherein the one or more markers of IL-2 signaling comprises phosphorylated STAT5.
34. The method of any one of embodiments 28-33, wherein the suppressive activity of the Treg on CD8+ and/or CD4+ T cells is increased.
35. The method of embodiment 34, wherein the rate of division of CD4+ and/or CD8+ T cells is decreased.
36. The method of any one of embodiments 28-35, wherein the relative amount of Treg cells in a composition comprising a population of Treg cells transduced with a recombinant cytokine receptor increases over time.
37. The method of any one of embodiments 28-36, wherein a composition comprising a population of Treg cells transduced with the recombinant cytokine receptor contains more viable cells compared to a composition comprising a population of the same Treg cells not transduced with the recombinant cytokine receptor.
38. The method of embodiment 36 or embodiment 37, wherein at least 60% of the cells in the population of Treg cells transduced with the recombinant cytokine receptor remain viable 7 days after transduction.
39. The method of any one of embodiments 28-38, further comprising culturing the Treg cell in a composition comprising the cytokine.
40. The method of any one of embodiments 28-39, further comprising, further comprising culturing the Treg cell in a composition comprising one or more cytokines additional cytokines other than IL-2.
41. The method of any one of embodiments 28-40, wherein the population of Treg cells transduced with the recombinant cytokine receptor expands at least two-fold more than a population of the same Treg cells not transduced with the recombinant cytokine receptor.
42. The method of any one of embodiments 28-41, wherein the population of Treg cells transduced with the recombinant cytokine receptor expands at least two-fold, and wherein the population of Treg cells transduced with the recombinant cytokine receptor maintains expression of least one Treg marker selected from the group consisting of CD4+, CD25+, and CD127 lo.
43. A recombinant cytokine receptor comprising an extracellular cytokine receptor domain, a transmembrane domain, and an intracellular IL-2 receptor beta chain domain, wherein the extracellular cytokine receptor domain is able to bind to a cytokine other than IL-2.
44. A recombinant cytokine receptor comprising an extracellular cytokine receptor domain, a transmembrane domain, and an intracellular IL-2 receptor beta domain, wherein the extracellular cytokine receptor domain is able to bind to and is tethered to a cytokine other than IL-2.
45. The recombinant cytokine receptor of embodiment 43 or embodiment 44, wherein the extracellular cytokine receptor domain is selected from the group consisting of an IL-4 extracellular domain, an IL-7 extracellular domain, an IL-9 extracellular domain, and an IL-21 extracellular domain.
46. The recombinant cytokine receptor of embodiment 44 or embodiment 45, wherein the extracellular cytokine receptor domain is tethered to the cytokine by a polypeptide linker.
47. The recombinant cytokine receptor of embodiment 46, wherein the polypeptide linker comprises glycine and serine residues.
48. The recombinant cytokine receptor of embodiment 43 or embodiment 45, wherein the extracellular cytokine receptor domain is not tethered to the cytokine.
49. The recombinant cytokine receptor of any one of embodiments 43-48, wherein the recombinant cytokine receptor engages in IL-2 signaling in the absence of IL-2.
50. The cytokine receptor of any one of embodiments 43-49, wherein the transmembrane domain is a transmembrane domain of a cytokine receptor.
51. The recombinant cytokine receptor of embodiment 50, wherein the transmembrane domain is a transmembrane domain of an IL-9 receptor, an IL-2 receptor, an IL-4 receptor, an IL-7 receptor, or an IL-21 receptor.
52. The recombinant cytokine receptor of any one of embodiments 43-51, wherein the TM and the ED are from the same cytokine receptor.
53. The recombinant cytokine receptor of any one of embodiments 43-5, wherein the intracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:20.
54. The recombinant cytokine receptor of any one of embodiments 43-53, wherein the extracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:11-14 or 21.
55. The recombinant cytokine receptor of any one of embodiments 43-54, wherein the transmembrane domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO: 16-19.
56. The recombinant cytokine receptor of any one of embodiments 43-55, wherein
   the intracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:20;
   the extracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO: 11-14; and/or
   the transmembrane domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO: 16-19.
57. The recombinant cytokine receptor of any one of embodiments 43-56, wherein the recombinant cytokine receptor comprises
   a) an IL-4Rα extracellular domain, an IL-4Rα transmembrane domain, and an IL-2Rβ intracellular domain;
   b) an IL-4Rα extracellular domain, an IL-2Rβ transmembrane domain, and an IL-2Rβ intracellular domain;
   c) an IL-7Rα extracellular domain, an IL-7Rα transmembrane domain, and an IL-2Rβ intracellular domain;
   d) an IL-7Rα extracellular domain, an IL-2Rβ transmembrane domain, and an IL-2Rβ intracellular domain;
   e) an IL-9Rα extracellular domain, an IL-9Rα transmembrane domain, and an IL-2Rβ intracellular domain;
   f) an IL-9Rα extracellular domain, an IL-2Rβ transmembrane domain, and an IL-2Rβ intracellular domain;
   g) an IL-21Rα extracellular domain, an IL-21Rα transmembrane domain, and an IL-2Rβ intracellular domain; or
   h) an IL-21Rα extracellular domain, an IL-2Rβ transmembrane domain, and an IL-2Rβ intracellular domain.
58. The recombinant cytokine receptor of any one of embodiments 43-57, wherein:
   a) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:16, and the extracellular domain comprises an amino acid sequence comprising at least 80%, at least 90%, at least 95%, at least 99%, or 100% sequence identity with the amino acid sequence set forth in SEQ ID NO:11;
   b) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:17, and the extracellular domain comprises an amino acid sequence comprising at least 80%, at least 90%, at least 95%, at least 99%, or 100% sequence identity with the amino acid sequence set forth in SEQ ID NO: 12;
   c) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 18, and the extracellular domain comprises an amino acid sequence comprising at least 80%, at least 90%, at least 95%, at least 99%, or 100% sequence identity with the amino acid sequence set forth in SEQ ID NO:13;
   d) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO: 13;
   e) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO: 14;
   f) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 16, and the extracellular domain comprises an amino acid sequence comprising at least 80%, at least 90%, at least 95%, at least 99%, or 100% sequence identity with the amino acid sequence set forth in SEQ ID NO:11, and the cytokine comprises an amino acid sequence comprising at least 80%, at least 90%, at least 95%, at least 99%, or 100% sequence identity with the amino acid sequence set forth in SEQ ID NO: 22;
   g) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 17, and the extracellular domain comprises an amino acid sequence comprising at least 80%, at least 90%, at least 95%, at least 99%, or 100% sequence identity with the amino acid sequence set forth in SEQ ID NO:12, and the cytokine comprises an amino acid sequence comprising at least 80%, at least 90%, at least 95%, at least 99%, or 100% sequence identity with the amino acid sequence set forth in SEQ ID NO: 23;
   h) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 18, and the extracellular domain comprises an amino acid sequence comprising at least 80%, at least 90%, at least 95%, at least 99%, or 100% sequence identity with the amino acid sequence set forth in SEQ ID NO:13, and the cytokine comprises an amino acid sequence comprising at least 80%, at least 90%, at least 95%, at least 99%, or 100% sequence identity with the amino acid sequence set forth in SEQ ID NO: 24;
   i) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO: 13, and the cytokine comprises an amino acid sequence comprising at least 80%, at least 90%, at least 95%, at least 99%, or 100% sequence identity with the amino acid sequence set forth in SEQ ID NO: 24; or
   j) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO:14, and the cytokine comprises an amino acid sequence comprising at least 80%, at least 90%, at least 95%, at least 99%, or 100% sequence identity with the amino acid sequence set forth in SEQ ID NO: 25.
59. The recombinant cytokine receptor of any one of embodiments 43, 45, and 48-58, wherein the recombinant cytokine receptor comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:1-5.
60. The recombinant cytokine receptor of any one of embodiments 43-47 and 49-59, wherein the recombinant cytokine receptor comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:6-9 or 15.
61. A nucleic acid encoding the recombinant cytokine receptor of any one of embodiments 43-60.
62. A vector comprising the nucleic acid of embodiment 61.
63. The vector of embodiment 62, wherein the vector is a lentiviral vector.
64. The vector of embodiment 62 or 63, further comprising a marker gene.
65. The vector of embodiment 64, wherein the marker gene is a transmembrane protein.
66. The vector of embodiment 65, wherein the transmembrane protein is EGFR.
67. A T cell comprising the recombinant cytokine receptor of any one of embodiments 43-60, the nucleic acid of embodiment 61, or the vector of any one of embodiments 62-66.
68. The T cell of embodiment 67, wherein the T cell is a regulatory T cell (Treg), wherein the Treg is CD4+, CD25+, and CD127lo.
69. The T cell of embodiment 68, wherein the Treg expresses FOX3P and/or HELIOS.
70. The T cell of any one of embodiments 67-69, further comprising a chimeric antigen receptor (CAR).
71. A composition comprising the nucleic acid of embodiment 61, the vector of any one of embodiments 62-66, or the T cell of any one of embodiments 67-70.
72. A method of expanding a Treg cell in the absence of IL-2 comprising introducing the nucleic acid of embodiment 61, or the vector of any one of embodiments 62-66 into the Treg cell and culturing the cell in vitro in the absence of IL-2.
73. The method of embodiment 72, further comprising detecting at least one Treg marker selected from the group consisting of CD4+, CD25+, and CD127.
74. The method of embodiment 73, further comprising detecting FOXP3 and/or HELIOS.
75. The method of any one of embodiments 72-74, wherein the Treg cell is able to proliferate in the absence of IL-2.
76. The method of any one of embodiments 72-75, wherein one or more markers of IL-2 signaling is detected.
77. The method of any one of embodiments 72-76, wherein the one or more markers of IL-2 signaling comprises phosphorylated STAT5.
78. The method of any one of embodiments 72-77, wherein the suppressive activity of the Treg on CD8+ and/or CD4+ T cells is increased.
79. The method of embodiment 78, wherein the rate of division of CD4+ and/or CD8+ T cells is decreased.
80. The method of any one of embodiments 72-79, wherein the relative amount of Treg cells in a composition comprising a population of Treg cells transduced with a recombinant cytokine receptor increases over time.
81. The method of any one of embodiments 72-80, wherein a composition comprising a population of Treg cells transduced with the recombinant cytokine receptor contains more viable cells compared to a composition comprising a population of the same Treg cells not transduced with the recombinant cytokine receptor.
82. The method of embodiment 80 or embodiment 81, wherein at least 60% of the cells in the population of Treg cells transduced with the recombinant cytokine receptor remain viable 7 days after transduction.
83. The method of any one of embodiments 72-82, further comprising culturing the Treg cell in a composition comprising the cytokine.
84. The method of any one of embodiments 72-83, further comprising, further comprising culturing the Treg cell in a composition comprising one or more cytokines additional cytokines other than IL-2.
85. The method of any one of embodiments 72-84 wherein the population of Treg cells transduced with the recombinant cytokine receptor expands at least two-fold more than a population of the same Treg cells not transduced with the recombinant cytokine receptor.
86. The method of any one of embodiments 72-85, wherein the population of Treg cells transduced with the recombinant cytokine receptor expands at least two-fold, and wherein the population of Treg cells transduced with the recombinant cytokine receptor maintains expression of least one Treg marker selected from the group consisting of CD4+, CD25+, and CD127 lo.
87. A regulatory T cell (Treg) comprising a recombinant cytokine receptor, wherein the recombinant cytokine receptor comprises an extracellular cytokine receptor domain, a transmembrane domain, and an intracellular IL-2 receptor beta chain domain, wherein the extracellular cytokine receptor domain binds to a cytokine other than IL-2.
88. The Treg of embodiment 87, wherein the extracellular cytokine receptor domain is tethered to the cytokine.
89. The Treg of embodiment 87 or embodiment 88, wherein the extracellular cytokine receptor domain is selected from the group consisting of an IL-4 extracellular domain, an IL-7 extracellular domain, an IL-9 extracellular domain, and an IL-21 extracellular domain.
90. The Treg of embodiment 88 or embodiment 89, wherein the extracellular cytokine receptor domain is tethered to the cytokine by a polypeptide linker, optionally wherein the polypeptide linker comprises glycine and serine residues.
91. The Treg of embodiment 87 or embodiment 89, wherein the extracellular cytokine receptor domain is not tethered to the cytokine.
92. The Treg of any one of embodiments 87-91, wherein the recombinant cytokine receptor engages in IL-2 signaling in the absence of IL-2.
93. The Treg of any one of embodiments 87-92, wherein the transmembrane domain is a transmembrane domain of a cytokine receptor.
94. The Treg of embodiment 93, wherein the transmembrane domain is a transmembrane domain of an IL-9 receptor, an IL-2 receptor, an IL-4 receptor, an IL-7 receptor, or an IL-21 receptor.
95. The Treg of any one of embodiments 87-94, wherein the TM and the ED are from the same cytokine receptor.
96. The Treg of any one of embodiments 87-95, wherein the intracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:20; and/or the extracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:11-14; and/or the transmembrane domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO: 16-19 or 21.
97. The Treg of any one of embodiments 87-96, wherein the recombinant cytokine receptor comprises
   a) an IL-4Rα extracellular domain, an IL-4Rα transmembrane domain, and an IL-2Rβ intracellular domain;
   b) an IL-4Rα extracellular domain, an IL-2Rβ transmembrane domain, and an IL-2Rβ intracellular domain;
   c) an IL-7Rα extracellular domain, an IL-7Rα transmembrane domain, and an IL-2Rβ intracellular domain;
   d) an IL-7Rα extracellular domain, an IL-2Rβ transmembrane domain, and an IL-2Rβ intracellular domain;
   e) an IL-9Rα extracellular domain, an IL-9Rα transmembrane domain, and an IL-2Rβ intracellular domain;
   f) an IL-9Rα extracellular domain, an IL-2Rβ transmembrane domain, and an IL-2Rβ intracellular domain;
   g) an IL-21Rα extracellular domain, an IL-21Rα transmembrane domain, and an IL-2Rβ intracellular domain; or
   h) an IL-21Rα extracellular domain, an IL-2Rβ transmembrane domain, and an IL-2Rβ intracellular domain.
98. The Treg of any one of embodiments 87-97, wherein:
   a) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 16, and the extracellular domain comprises an amino acid sequence comprising at least 80%, at least 90%, at least 95%, at least 99%, or 100% sequence identity with the amino acid sequence set forth in SEQ ID NO:11;
   b) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 17, and the extracellular domain comprises an amino acid sequence comprising at least 80%, at least 90%, at least 95%, at least 99%, or 100% sequence identity with the amino acid sequence set forth in SEQ ID NO:12;
   c) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 18, and the extracellular domain comprises an amino acid sequence comprising at least 80%, at least 90%, at least 95%, at least 99%, or 100% sequence identity with the amino acid sequence set forth in SEQ ID NO:13;
   d) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO:13;
   e) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO:14;
   f) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 16, and the extracellular domain comprises an amino acid sequence comprising at least 80%, at least 90%, at least 95%, at least 99%, or 100% sequence identity with the amino acid sequence set forth in SEQ ID NO: 11, and the cytokine comprises an amino acid sequence comprising at least 80%, at least 90%, at least 95%, at least 99%, or 100% sequence identity with the amino acid sequence set forth in SEQ ID NO: 22;
   g) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:17, and the extracellular domain comprises an amino acid sequence comprising at least 80%, at least 90%, at least 95%, at least 99%, or 100% sequence identity with the amino acid sequence set forth in SEQ ID NO:12, and the cytokine comprises an amino acid sequence comprising at least 80%, at least 90%, at least 95%, at least 99%, or 100% sequence identity with the amino acid sequence set forth in SEQ ID NO: 23;
   h) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 18, and the extracellular domain comprises an amino acid sequence comprising at least 80%, at least 90%, at least 95%, at least 99%, or 100% sequence identity with the amino acid sequence set forth in SEQ ID NO: 13, and the cytokine comprises an amino acid sequence comprising at least 80%, at least 90%, at least 95%, at least 99%, or 100% sequence identity with the amino acid sequence set forth in SEQ ID NO: 24;
   i) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO:13, and the cytokine comprises an amino acid sequence comprising at least 80%, at least 90%, at least 95%, at least 99%, or 100% sequence identity with the amino acid sequence set forth in SEQ ID NO: 24; or
   j) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO: 14, and the cytokine comprises an amino acid sequence comprising at least 80%, at least 90%, at least 95%, at least 99%, or 100% sequence identity with the amino acid sequence set forth in SEQ ID NO: 25.
99. The Treg of any one of embodiments 87, 89, and 91-98, wherein the recombinant cytokine receptor comprises an amino acid sequence comprising at least 80%, at least 85%, at least 90%, at least 95%, at least 96%, at least 97%, at least 98%, at least 99%, or 100% sequence identity with an amino acid sequence selected from the group consisting of SEQ ID NOs: 1-5.
100. The Treg of any one of embodiments 87-90 and 92-99, wherein the recombinant cytokine receptor comprises an amino acid sequence comprising at least 80%, at least 85%, at least 90%, at least 95%, at least 96%, at least 97%, at least 98%, at least 99%, or 100% sequence identity with an amino acid sequence selected from the group consisting of SEQ ID NOs: 6-9 and 15.
101. The Treg of any one of embodiments 87-100, wherein the Treg is CD4+, CD25+, and CD127lo, optionally, wherein the Treg expresses FOX3P and/or HELIOS.
102. The Treg of any one of embodiments 87-101, further comprising a chimeric antigen receptor (CAR).
103. The T cell of any one of embodiments 20-23 and 67-70 or the Treg of any one of embodiments 86-100 for use in treating an immune-related disorder in an individual.
104. The T cell of embodiment 103, wherein the T cell or the Treg is autologous to the individual.
105. The T cell of embodiment 103 or embodiment 104, wherein the individual is human.
106. The use of the T cell of any one of embodiments 20-23 and 67-70 or the Treg of any one of embodiments 87-102 in the manufacture of a medicament for treating an immune-related disorder in an individual.
107. The use of embodiment 106, wherein the T cell or the Treg is autologous to the individual.
108. The use of embodiment 106 or embodiment 107, wherein the individual is human.
109. A method of treating an immune-related disorder comprising administering the T cell of any one of embodiments 67-70, the Treg of any one of embodiments 87-102, or the composition of embodiment 71, to an individual in need thereof.
110. The method of embodiment 109, wherein the cells are autologous to the individual.
111. The method of embodiment 109 or embodiment 110, wherein the individual is human.
112. The T cell or Treg of any one of embodiments 103-105, the use of any one of embodiments 106-108, or the methods of any one of embodiments 25-27 and 109-111, wherein the individual expresses IL-2.
113. The method of any one of embodiments 28-32 and 72-76, wherein one or more markers of IL-2 signaling is detected, optionally wherein the one or more markers of IL-2 signaling comprises phosphorylated STAT-5.
114. The method of any one of embodiments 28-32 and 72-76, wherein one or more markers of endogenous IL-2 signaling is detected, optionally wherein the one or more markers of endogenous IL-2 signaling comprises phosphorylated STAT-5.
115. The method of any one of embodiments 28-33, 72-77, 99, and 100, wherein:
   i, the suppressive activity of the Treg on CD8+ and/or CD4+ T cells is increased compared to a control Treg;
   ii, the rate of division of CD4+ and/or CD8+ T cells is decreased when cultured in the presence of the Treg compared to the rate of division of CD4+ and/or CD8+ T cells when cultured without the Treg;
   iii, the relative amount of Treg cells in a composition comprising a population of Treg cells transduced with a recombinant cytokine receptor increases over time;
   iv, a composition comprising a population of Treg cells transduced with the recombinant cytokine receptor contains more viable cells compared to a composition comprising a population of the same Treg cells not transduced with the recombinant cytokine receptor;
   v, at least 60% of the cells in the population of Treg cells transduced with the recombinant cytokine receptor remain viable 7 days after transduction;
   vi, the level of Treg proliferation increases from re-stimulation one or more times;
   vii, the IL-10 cytokine levels produced by the Treg are increased compared to a control Treg;
   viii, the IFN-γ cytokine levels produced by the Treg are increased compared to a control Treg; and/or
   ix, the Gr-B cytokine levels produced by the Treg are increased compared to a control Treg.
116. The method of any one of embodiments 28-33, 72-77, 99, and 100, wherein the method generates Treg cells comprising a recombinant cytokine receptor, wherein:
   i, the in vitro and/or in vivo suppressive activity of the Treg on CD8+ and/or CD4+ T cells is increased compared to a control Treg;
   ii, the rate of division of CD4+ and/or CD8+ T cells is decreased when cultured in the presence of the Treg compared to the rate of division of CD4+ and/or CD8+ T cells when cultured without the Treg;
   iii, the relative amount of Treg cells in a composition comprising a population of Treg cells transduced with a recombinant cytokine receptor increases over time;
   iv, a composition comprising a population of Treg cells transduced with the recombinant cytokine receptor contains more viable cells compared to a composition comprising a population of the same Treg cells not transduced with the recombinant cytokine receptor;
   v, at least 60% of the cells in the population of Treg cells transduced with the recombinant cytokine receptor remain viable in vitro 7 days after transduction;
   vi, the level of Treg proliferation increases from re-stimulation in vitro and/or in vivo one or more times;
   vii, the IL-10 cytokine levels produced by the Treg in vitro and/or in vivo are increased compared to a control Treg;
   viii, the IFN-γ cytokine levels produced by the Treg in vitro and/or in vivo are increased compared to a control Treg; and/or
   ix, the Gr-B cytokine levels produced by the Treg in vitro and/or in vivo are increased compared to a control Treg.

### EXAMPLES

The following examples are included for illustrative purposes only and are not intended to limit the scope of the invention.

### Example 1: IL-2 Receptors are Engineered to Form Recombinant Cytokine Receptors that Bind to Alternative Ligands

Human regulatory T cells were engineered to express chimeric IL-2 recombinant cytokine receptors that bind to alternative interleukins (e.g., IL-4, IL-7, IL-9, or IL-21) to induce IL-2 signaling. IL-2 signaling is required for regulatory T cells (hereafter referred to as regulatory T cells, Tregs, or Treg cells interchangeably) to survive and proliferate, thus the recombinant cytokine receptors described in detail below permit IL-2 independent survival and function of Tregs.

Vectors encoding all untethered and tethered recombinant IL-4Rα/IL-2Rβ, IL-7Rα/IL-2Rβ, IL-9Rα/IL-2Rβ, IL-21Rα/IL-2Rβ receptor DNA fragments were similarly constructed. Sequencing results indicated correct gene insertion. Amino acid sequences of untethered recombinant cytokine receptors and the domains comprised therein are shown in Table 1. Amino acid sequences of tethered recombinant cytokine receptors and the domains comprised therein are shown in Table 2.

Lentiviral construct production: Recombinant cytokine receptor constructs were packaged as lentiviral particles for transduction into cells of interest. HEK 293 FT suspension cells (Invitrogen) were seeded at 4.7×10⁶/mL and transfected with transfer and packaging plasmids (Aldevron pALD-VSV-G, pALD-GagPol, pALD-Rev) using LV_MAX transfection reagent as per manufacturer's protocol (Gibco). Viral supernatant was collected and filtered through a 0.45 μm polyvinylidene difluoride filter to remove cell debris. The viral supernatant was centrifuged at 10,000×g overnight to concentrate viral vectors. After 12-16 h, supernatant was removed, and the viral vector pellet was resuspended in Opti-MEM media. Resuspended viral vector was aliquoted and stored at −80° C.

Functional titer assay: Functional titers were assessed to identify ideal transduction conditions for experiments performed in Examples 3-11. SupT1 cells (ATCC) were cultured in RPMI medium supplemented with 10% fetal bovine serum. On the day of transduction, 50 μL of SupT1 cells at 4×10⁵ cells/mL were added to each well of a 96-well plate. Lentivirus stock was thawed at room temperature and diluted 1:20 in culture medium. Subsequently, a three-fold serial dilution was performed from 60 to 393,660-fold in culture medium. 50 μL of diluted lentivirus were added to a 96-well plate containing 50 μL of cells. On day 2 of transduction, 100 μL of culture media was added. On day 3, transduced cells were harvested and stained with appropriate antibody to detect surface markers. Labeled cells were analyzed by flow cytometry. Untransduced SupT1 cells were used as negative control to set a gate for flow cytometry analysis. Viral dilution that produced between 5-20% maker positive cells were used for the calculation of functional titer, based on the following formula:

Titer(TU/mL)=[Cell #]_{transduced}×[(%)cells]_{transduced}×[dilution factor]/[(mL)volume]_{transduced}.

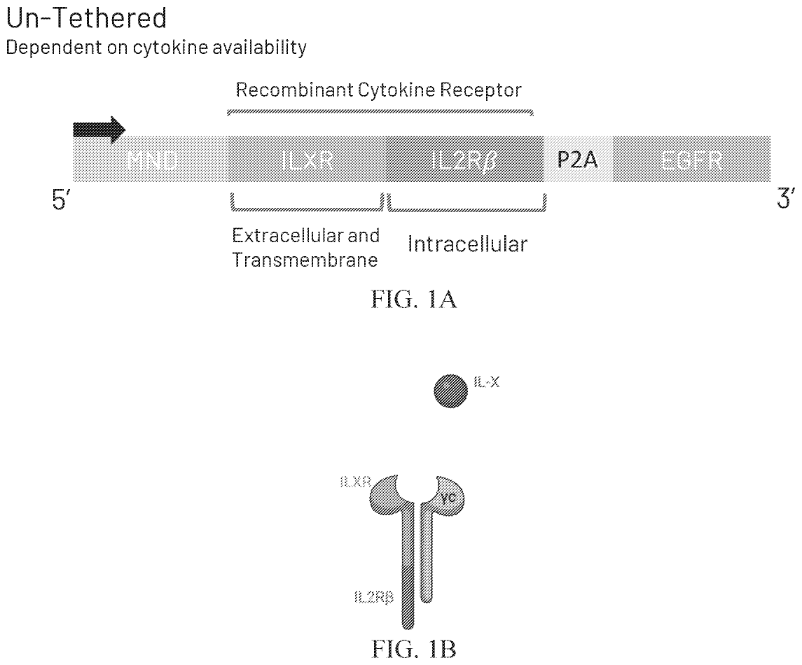

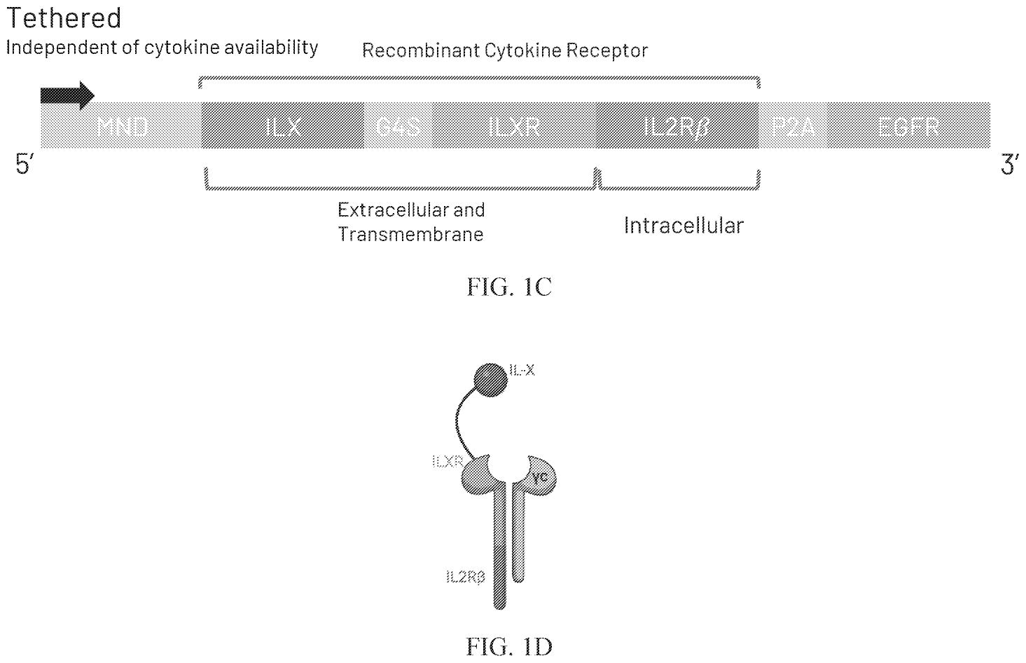

FIG. 1A shows a schematic of the general untethered recombinant cytokine receptor DNA construct wherein the extracellular cytokine binding domain and transmembrane domain of IL-4Rα, IL-7Rα, IL-9Rα, or IL-21Rα fused to the intracellular domain of the IL-2Rβ chain. FIG. 1B shows a schematic of the general untethered recombinant cytokine receptor. FIG. 1C shows a schematic of the general tethered recombinant cytokine receptor DNA construct wherein the cognate cytokine is tethered to the N-terminus extracellular cytokine binding domain and transmembrane domain of IL-4Rα. IL-7Rα, IL-9Rα, or IL-21Rα fused to the intracellular domain of the IL-2Rβ chain. FIG. 1D shows a schematic of the general tethered recombinant cytokine receptor covalently linked to its cognate cytokine. FIG. 1E shows a schematic of the exemplary recombinant cytokine receptors: cytokine-bound IL-4Rα/IL-2Rβ, IL-7Rα/IL-2Rβ, IL-9Rα/IL-2Rβ, and IL-21Rα/IL-2Rβ recombinant receptors and the IL-2 receptor.

### Example 2: Recombinant Cytokine Receptors are Expressed in HEKBlue IL-2 Reporter Cells

Example 2 shows that cells express the recombinant cytokine receptor constructs and that these constructs engage intracellular IL-2 signaling cascade independent of exogenous IL-2. The recombinant cytokine receptors were expressed in the IL-2 reporter HEKBlue cell line, in which the detectable reporter protein, secreted embryonic alkaline phosphatase (SEAP), is located downstream of an IL-2 target promoter. As a result, IL-2 signaling through the recombinant cytokine receptors produces SEAP, which is secreted into the cell culture medium. Example 2 demonstrates that each recombinant cytokine receptor transduces IL-2 signaling in response to exogenous cognate cytokine stimulus (i.e., IL-4, IL-7, IL-9, or IL-21) in the absence of exogenous IL-2.

IL-2 reporter cell assay: HEKBlue IL-2 reporter cells (Invivogen) were cultured as directed by manufacturer in DMEM media supplemented with 4.5 g/L glucose, 2 mM L-glutamine, and 10% FBS. Reporter cells were transduced with lentivirus corresponding to recombinant cytokine receptor constructs at moiety of infection (MOI) of 8. Two days post-transduction, marker positivity (EGFR) was assessed by flow cytometry. Three days post-transduction, 50,000 marker positive cells were plated in triplicate and incubated overnight at 37° C. Untethered recombinant IL-4Rα/IL-2Rβ, IL-7Rα/IL-2Rβ, IL-9Rα/IL-2Rβ, and IL-21Rα/IL-2Rβ receptors were incubated with their corresponding cytokine at the following concentrations: 0.4 ng/ml and 4.0 ng/ml (“10×”) IL-4, 1 ng/mL and 10 ng/ml (“10×”) IL-7, 2 ng/ml and 20 ng/mL (“10×”) IL-9, and 5 ng/mL and 50 ng/ml (“10×”) IL-21. Control cells were incubated with vehicle or with 300 IU/mL IL-2. SEAP was detected in culture supernatant using the QUANTI-Blue colorimetric assay and spectrophotometric analysis at 650 nM wavelength.

Tethered receptor transactivation assay: Reporter cells expressing untethered recombinant cytokine receptors were co-cultured overnight at a ratio of 1:1 with Jurkat T cells that were transduced with tethered recombinant cytokine receptors. SEAP was detected in culture supernatant using the QUANTI-Blue colorimetric assay and spectrophotometric analysis at 650 nM wavelength. Production of SEAP indicated that the tethered cytokine from the transduced Jurkat T cells was able to bind to the untethered receptors in the IL-2 reporter cells and activate IL-2 signaling cascade. Results from this assay indicated that recombinant cytokine receptor transactivation did not occur with any of these receptor constructs.

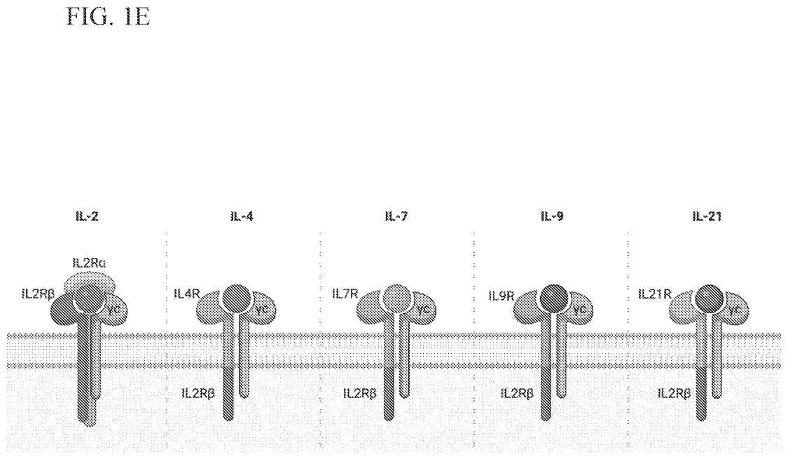

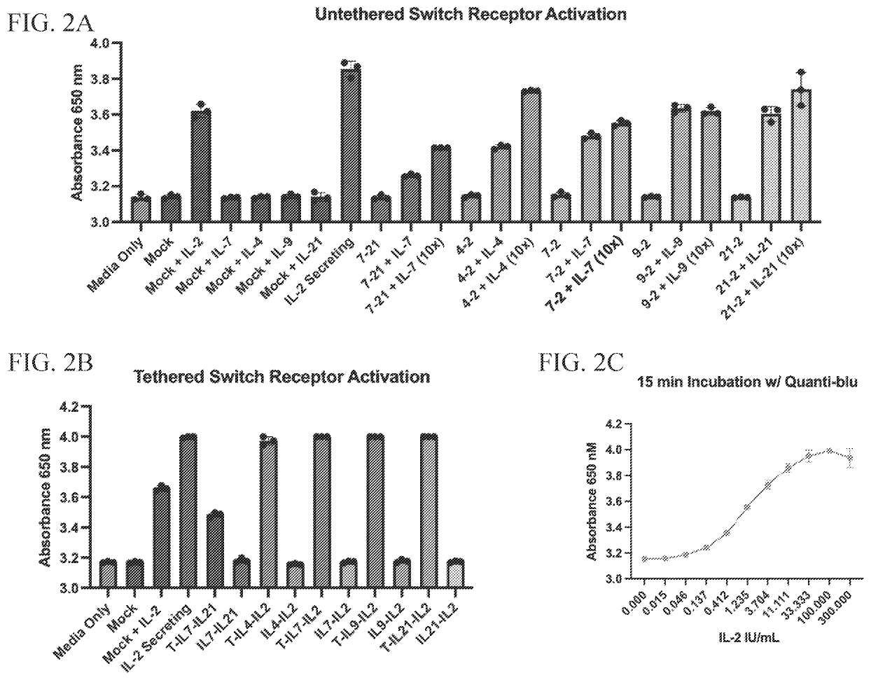

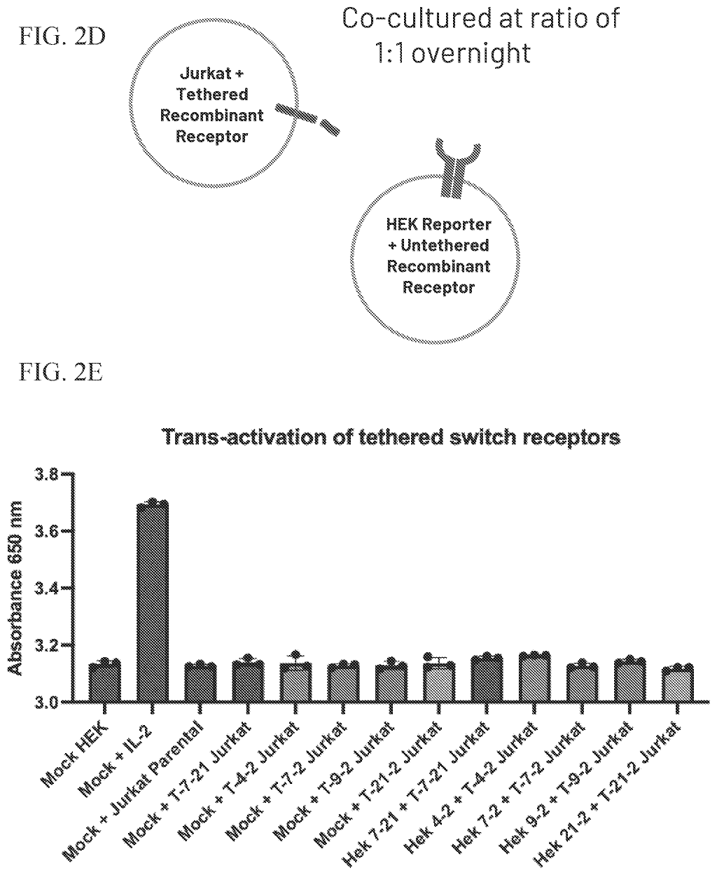

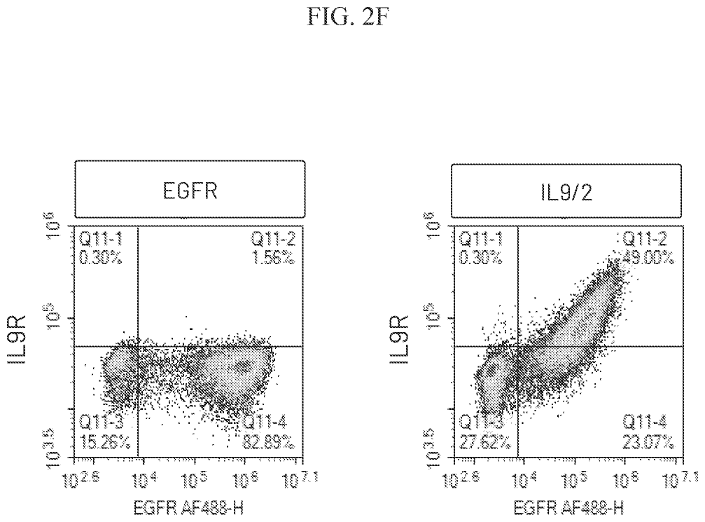

FIG. 2A depicts the degree to which the IL-2 signaling pathway was activated in IL-2 reporter cells expressing untethered recombinant cytokine receptors. Untethered constructs all demonstrated induction of IL-2 pathway activation when cultured in the presence of the respective cognate cytokine.

FIG. 2B depicts the degree to which cytokine-tethered recombinant receptor constructs were able to induce IL-2 pathway activation. All cytokine-tethered constructs equally induced IL-2 signaling in the absence of cytokine to a greater degree than the signal transduction in control cells treated with IL-2.

FIG. 2C depicts the absorbance values from SEAP detection using the QUANTI-Blue colorimetric assay and spectrophotometric analysis at 650 nM wavelength in IL-2 reporter cells treated with escalating doses of IL-2.

Tethered recombinant cytokine receptors were tested for the ability to transactivate untethered recombinant cytokine receptors as described above. FIG. 2D presents a schematic of the transduced cells that were co-cultured together in this example. FIG. 2E shows that no conditions other than the IL-2 reporter cell cultured with IL-2 as a positive control showed SEAP production. Furthermore, FIG. 2F demonstrates that IL-9Rα is not expressed on control Tregs, and therefore Tregs that express IL-9-tethered IL-9Rα/IL-2Rβ recombinant cytokine receptors are not at risk of activating control Tregs in cis. These results indicate that none of the tethered recombinant cytokine receptors can activate receptors on a neighboring cell via the tethered cytokine binding to the neighboring receptor.

### Example 3: Recombinant Cytokine Receptors are Expressed in Regulatory T Cells

The following example demonstrates that tethered and untethered recombinant IL-4Rα/IL-2Rβ, IL-7Rα/IL-2Rβ, IL-9Rα/IL-2Rβ, or IL-21Rα/IL-2Rβ receptors can be expressed in Treg cells, and these Treg cells can be distinguished by flow cytometry. The vector comprising each recombinant cytokine receptor also expresses the EGFR protein such that Treg cells expressing a recombinant cytokine receptor likewise express EGFR to become EGFR− positive (+). Thus, this Treg population can be distinguished from the untransduced Tregs from the parent population by antibody staining for co-expressed EGFR, as described in further detail below.

Treg isolation and expansion: Primary human Treg cells were sourced from healthy donors from leukoreduction chamber residuals or leukopaks. Peripheral blood mononuclear cells (PBMC) were isolated by density gradient centrifugation using Ficoll-Paque Plus. CD25+ cells were enriched by positive selection. Treg cells were next isolated using FACS by gating for CD4+/CD25+/CD127lo cells. After isolation, cells were stimulated with CTS Dynabeads Treg Xpander (Gibco) at a 1:1 bead to cell ratio and expanded for 14 days, with restimulation on day 9. Cells were cultured in RPMI medium supplemented with 10% FBS, non-essential amino acids, sodium pyruvate, and penicillin/streptomycin with recombinant human IL-2 at 300 IU/mL at a density of 0.25-0.3 million cells/mL.

Treg Transduction: Recombinant IL-4Rα/IL-2Rβ, IL-7Rα/IL-2Rβ, IL-9Rα/IL-2Rβ, IL-21Rα/IL-2Rβ receptors or EGFR− only controls were expressed in Treg cells by transducing Tregs with vectors containing the recombinant cytokine receptors described in Example 1. Treg cells were plated in a 24-well plate on Day 0 at 2.5×10⁵ cells/mL in 1 mL of media per well. On Day 2. Tregs were transduced via spinfection by adding the appropriate volume of virus to achieve an MOI of 5-10 to each well and protamine sulfate at a final concentration of 100 μg/mL. Cells were centrifuged with lentivirus at 1200× g for 30 min at 30° C. Shortly or immediately after spin, fresh media was added with 2× recombinant human IL-2 at 1:1 ratio with conditioned media.

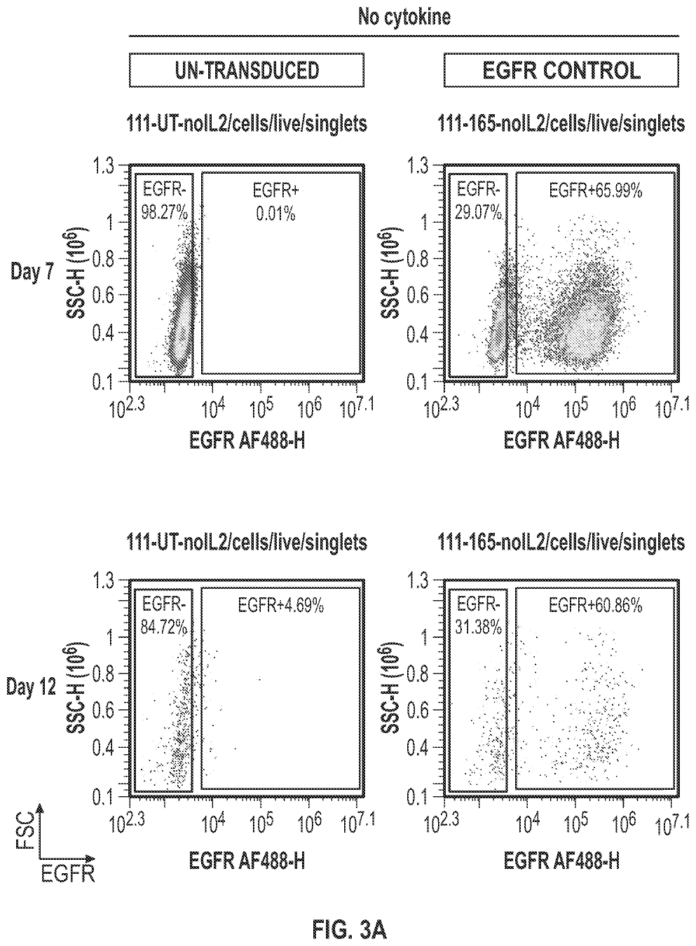

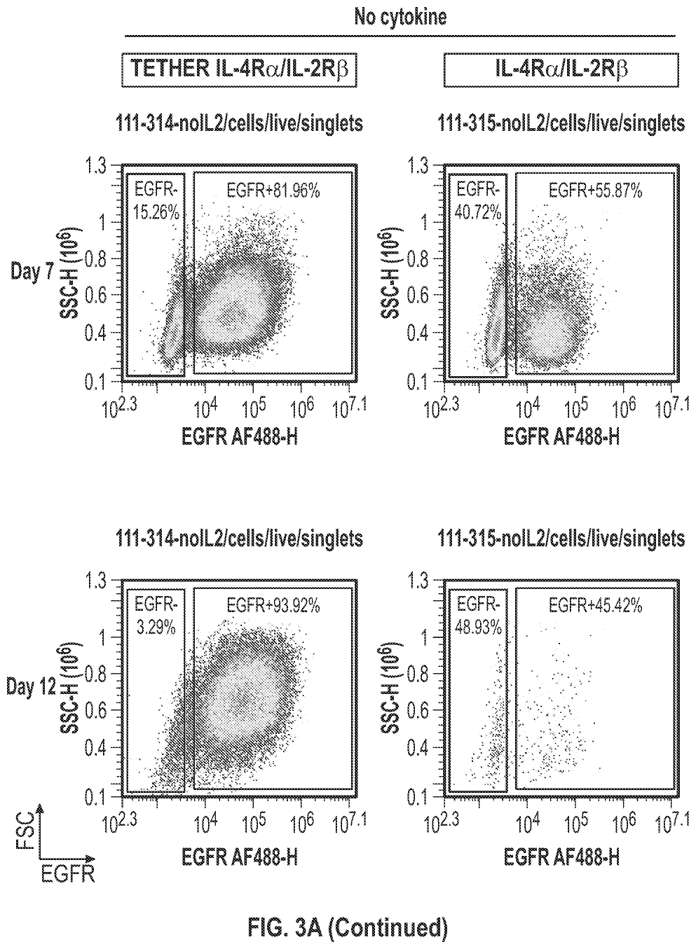

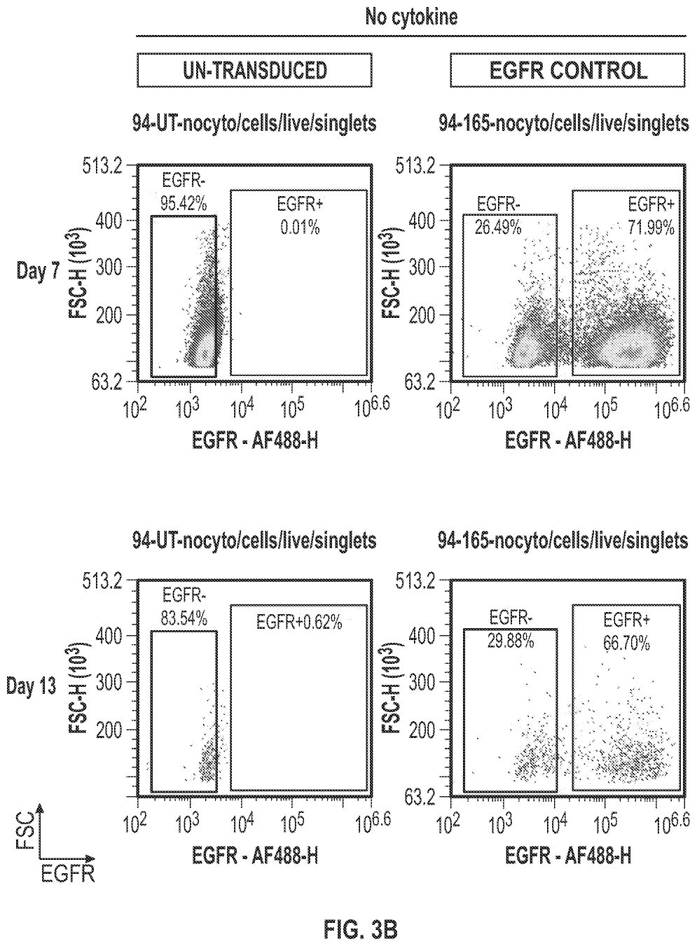

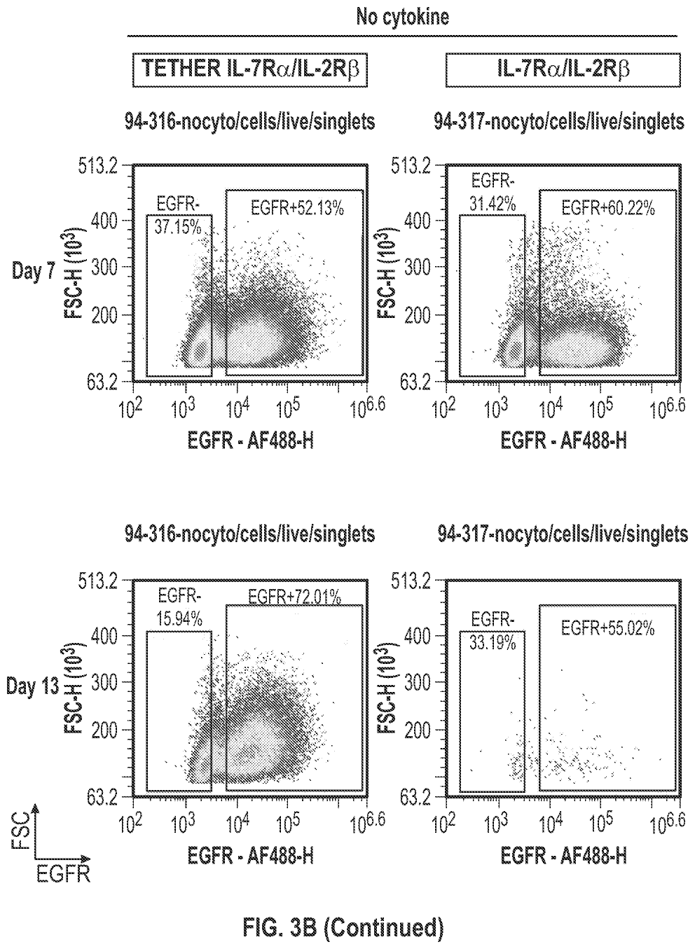

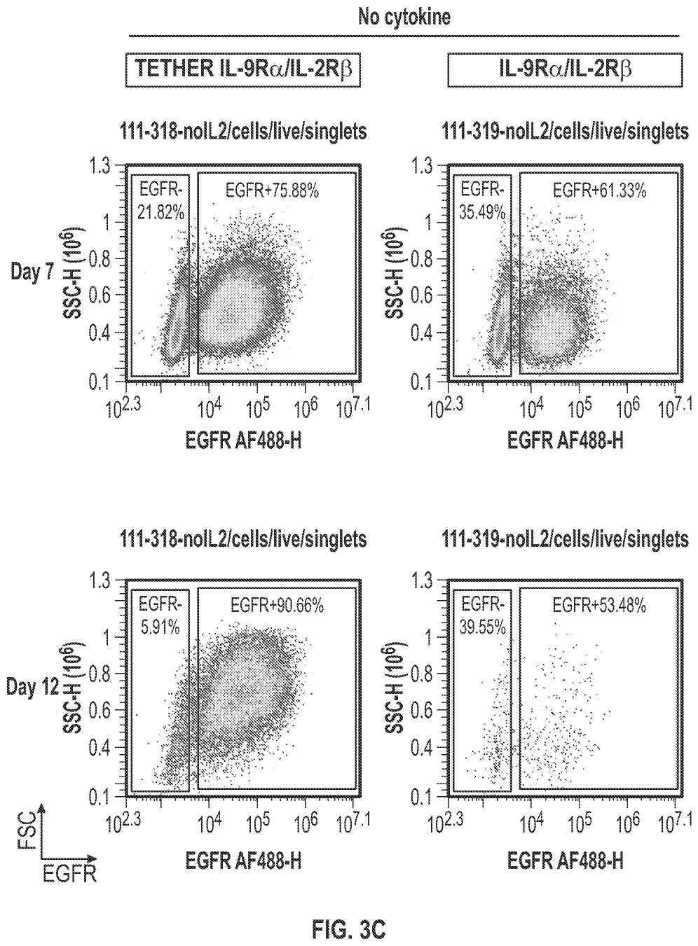

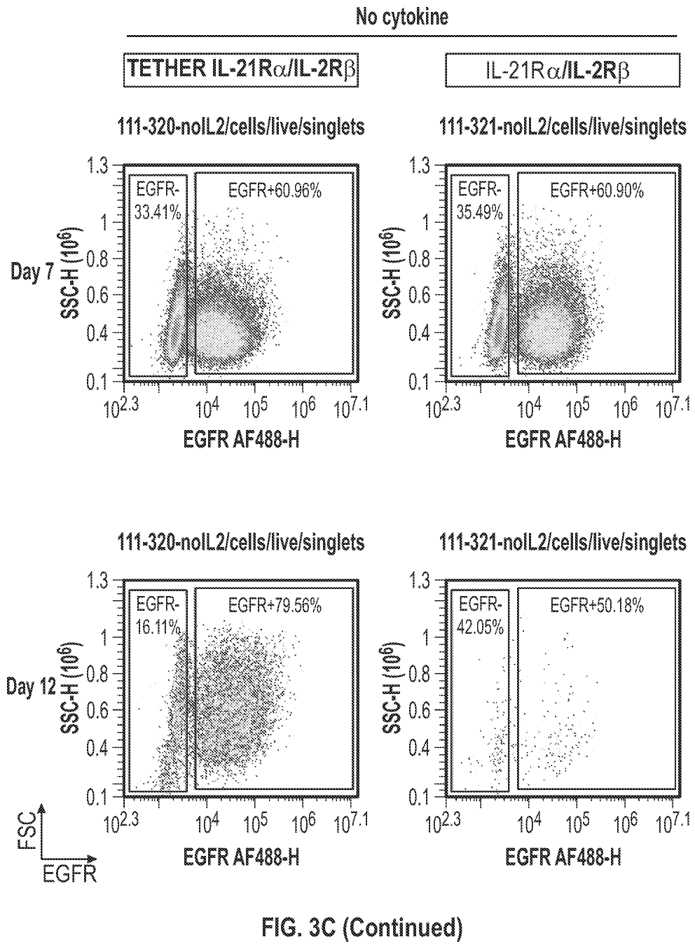

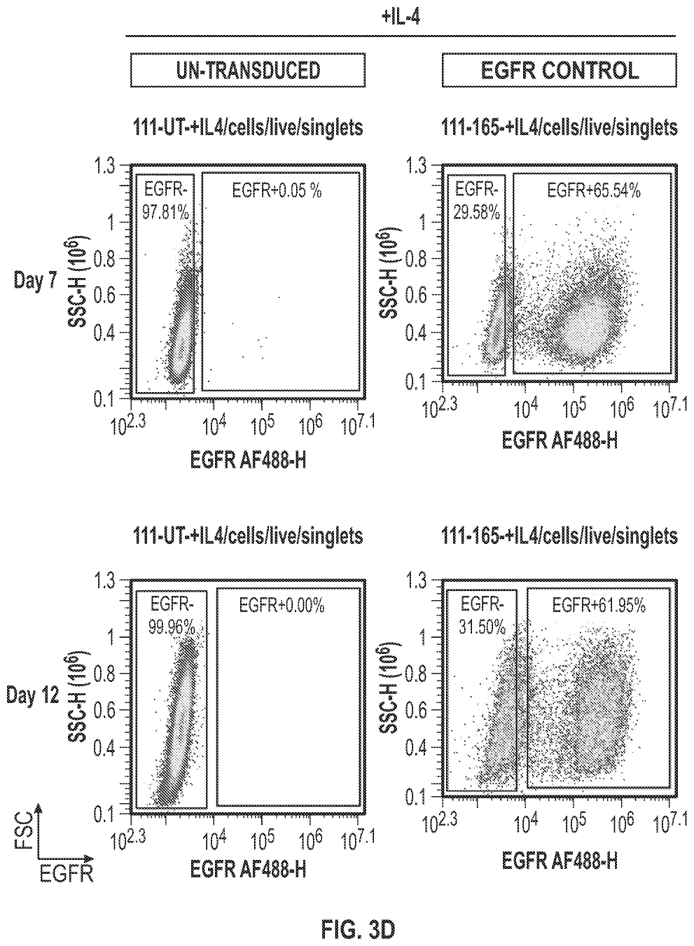

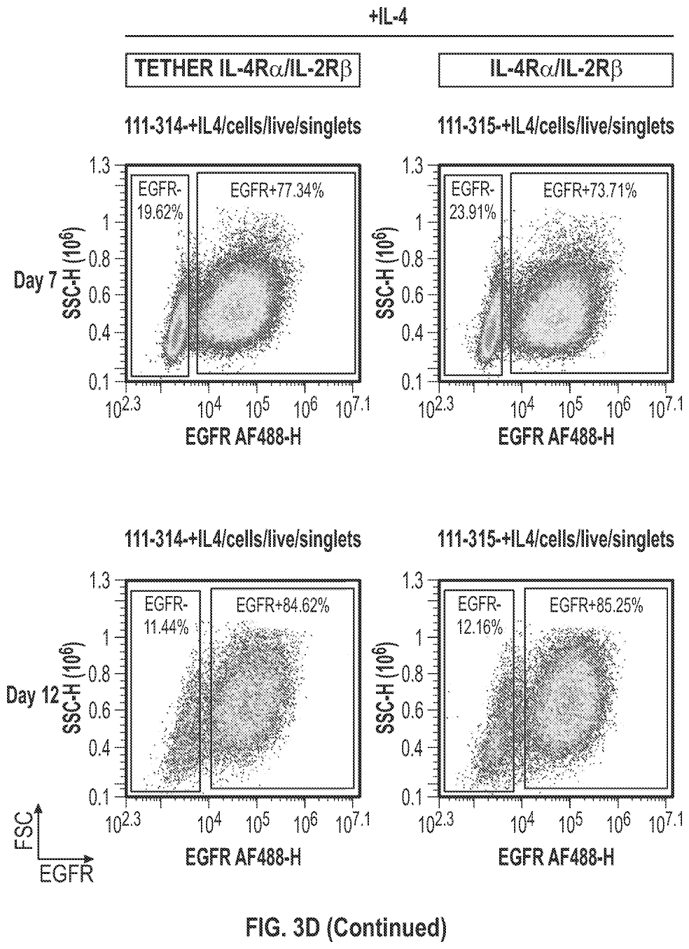

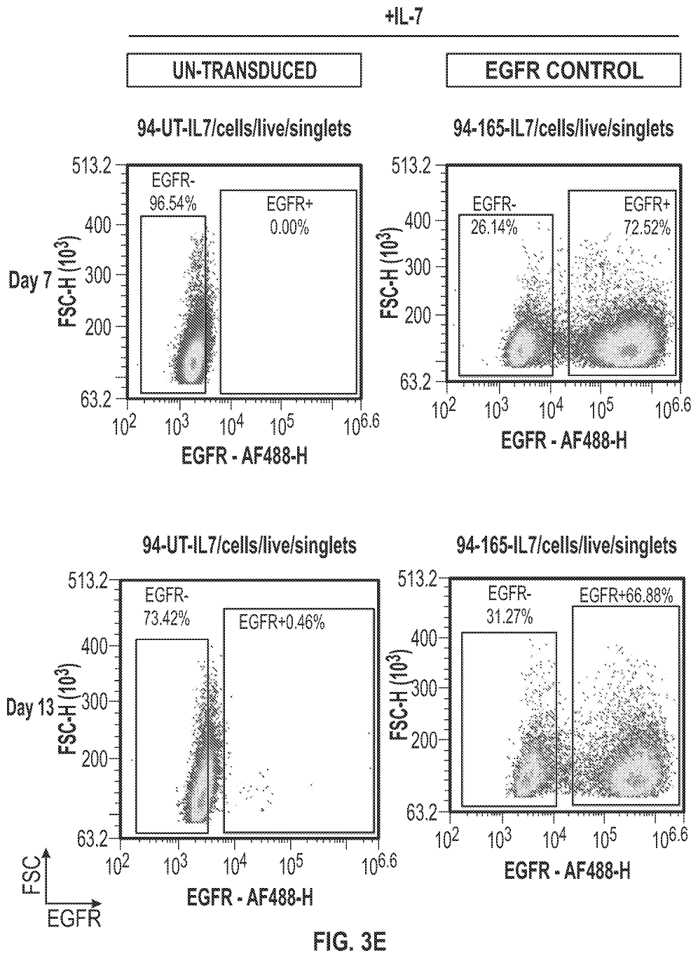

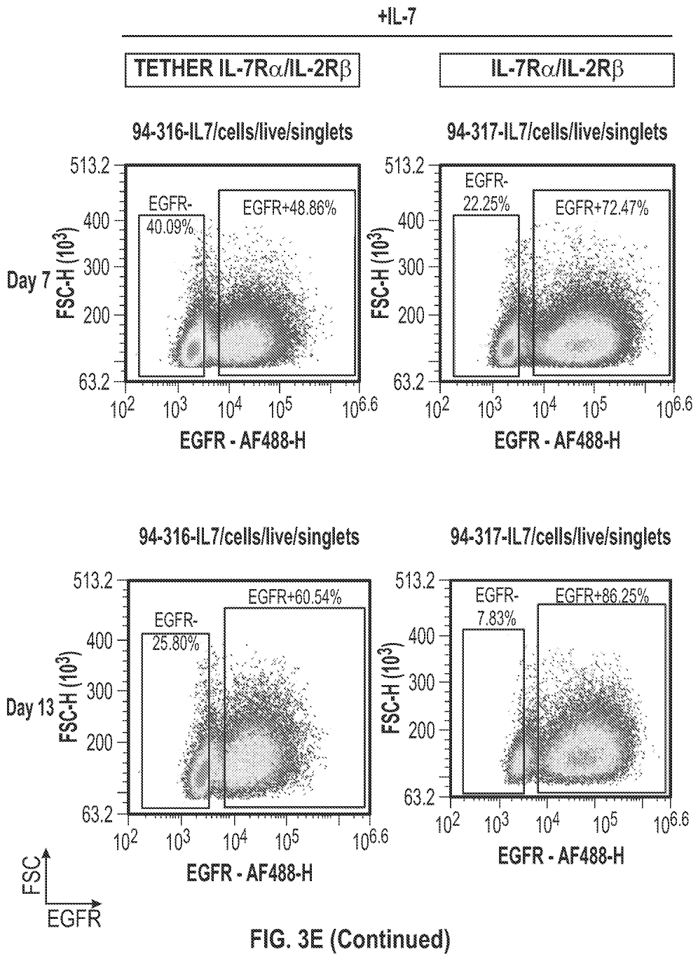

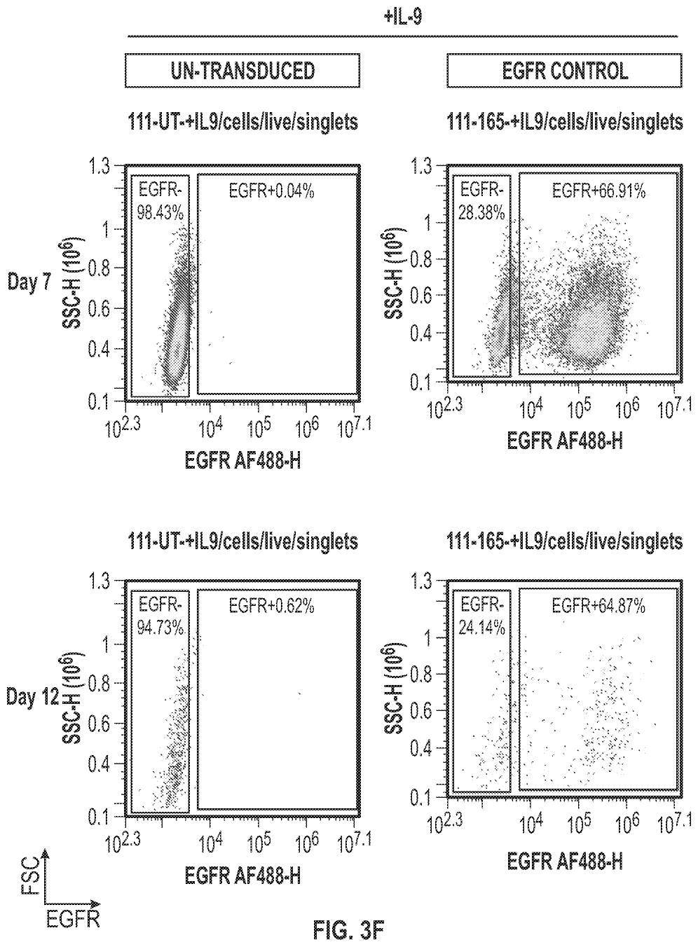

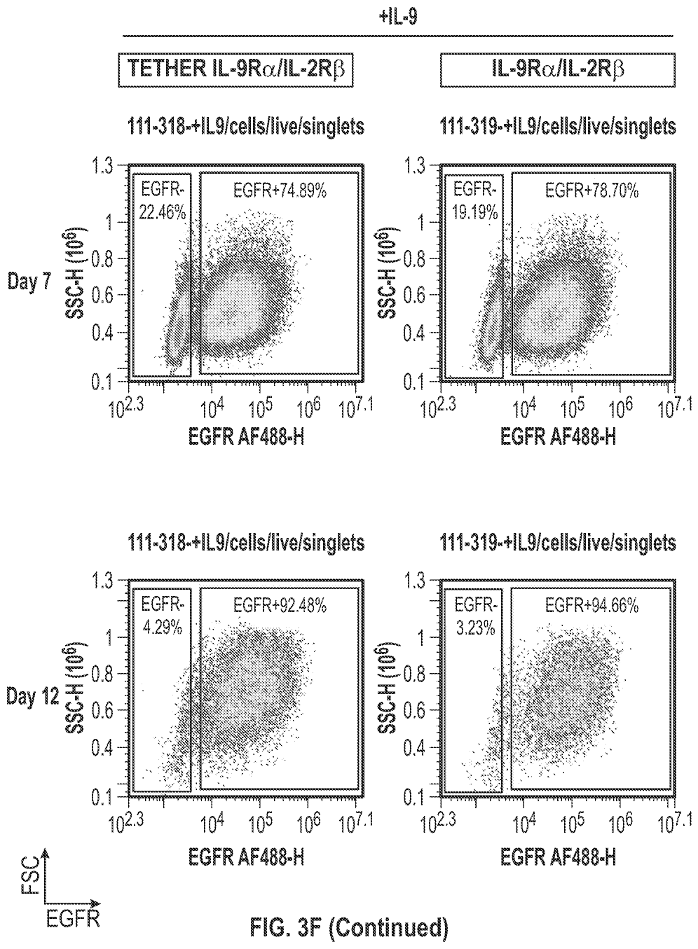

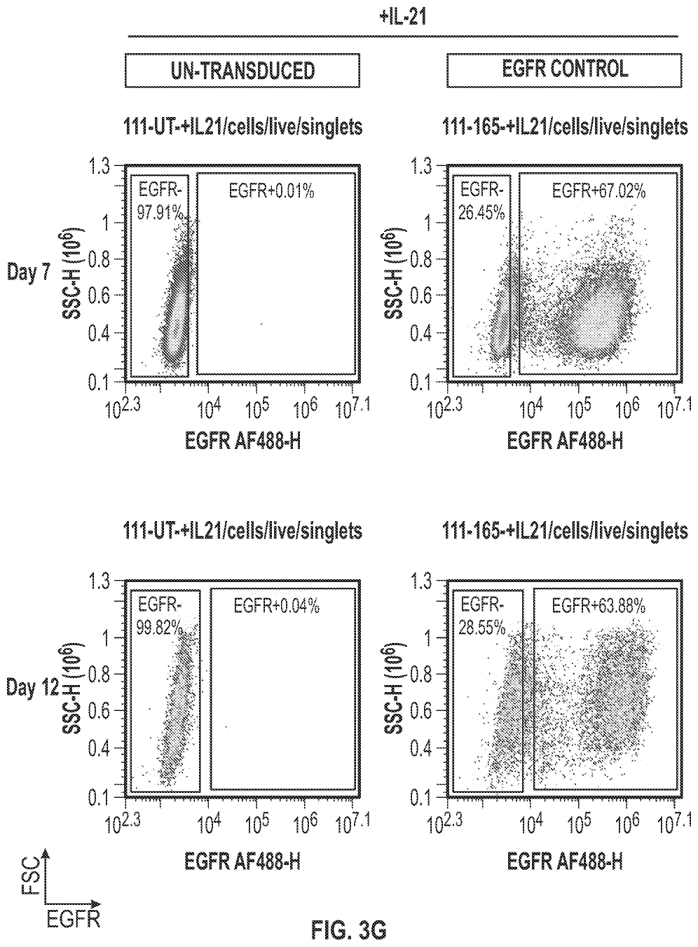

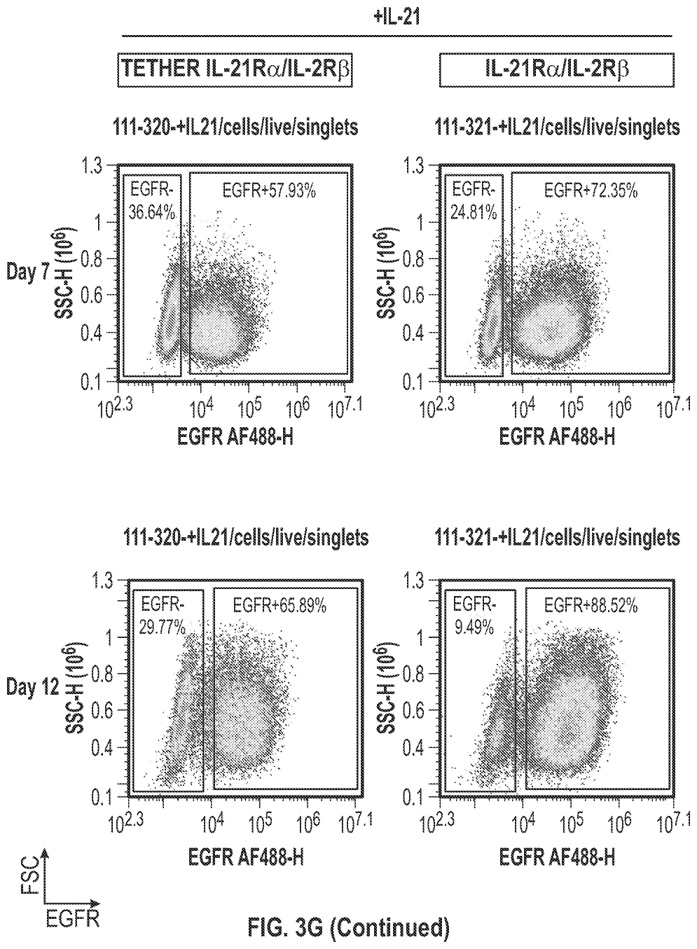

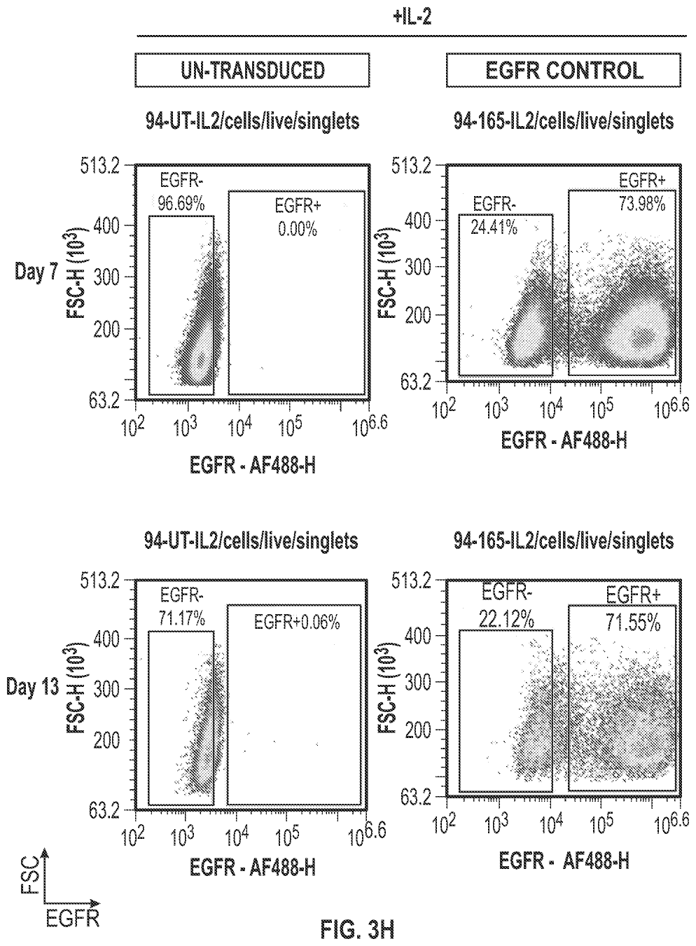

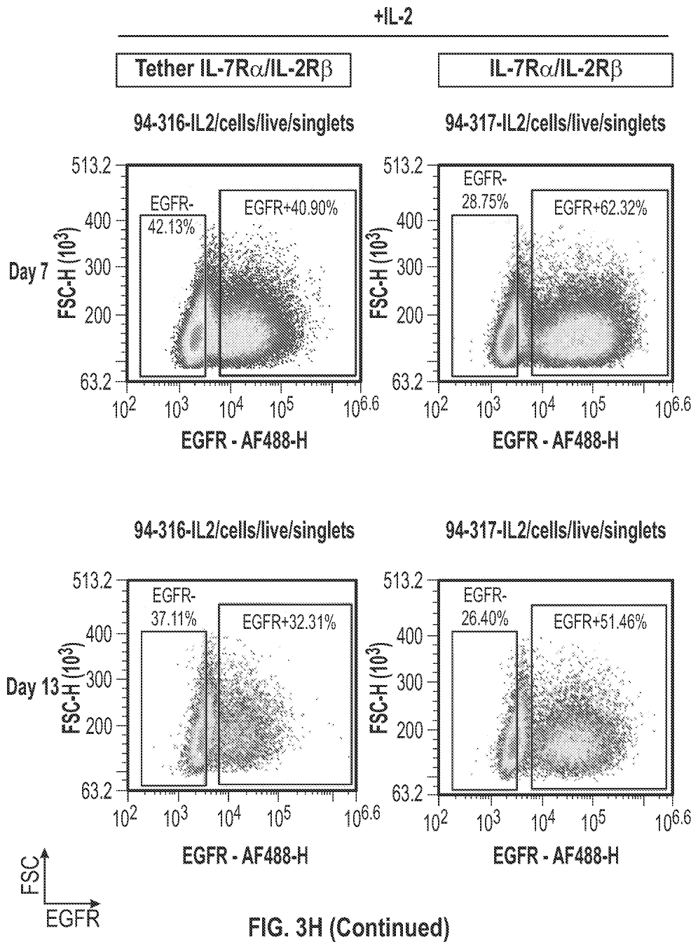

Flow cytometry and FACS analysis: Presence of EGFR within Tregs was assessed at days 7 and 13 post-transduction by FACS analysis. Treg cells were collected and centrifuged at 300×g for 5 min and then resuspended in 1× RoboSep Buffer (StemCell Technologies) with a surface staining antibody and viability dye cocktail. Tregs were then incubated for 30 min at 4° C., then centrifuged and washed with 1× RoboSep Buffer. For analysis of intracellular markers, Tregs were fixed and permeabilized, then stained for intracellular factors using the eBioscience™ FOXP3/Transcription Factor Staining Buffer Set (ThermoFisher) according to manufacturer's instructions. Stained cells were then analyzed by FACS and the percentage of successfully transduced Treg cells were assessed and reported in FIGS. 3A-3I.

FIGS. 3A-3I depict the successful transduction and durable expression of recombinant IL-4Rα/IL-2Rβ. IL-7Rα/IL-2Rβ. IL-9Rα/IL-2Rβ, or IL-21Rα/IL-2Rβ receptors into Tregs in the absence or presence of IL-2 or cognate cytokine. Experimental transduction groups showed equal or higher percentage EGFR+ Treg cells at day 7 and day 13 compared to untransduced controls, indicating increased transduced Treg survival and enrichment over time. FIGS. 3A-3C depict the percentage of EGFR+ control and recombinant cytokine receptor-expressing Treg cells cultured without cytokine at day 7 and day 13 (FIG. 3A: IL-4Rα/IL-2Rβ receptor; FIG. 3B: IL-7Rα/IL-2Rβ receptor; FIG. 3C: IL-9Rα/IL-2Rβ receptor and IL-21Rα/IL-2Rβ receptor). FIGS. 3D-3G depict the percentage of EGFR+ control and recombinant cytokine receptor-expressing Treg cells cultured with cognate cytokine at day 7 and day 12 or 13 (FIG. 3D: IL-4Rα/IL-2Rβ receptor; FIG. 3E: IL-7Rα/IL-2Rβ receptor; FIG. 3F: IL-9Rα/IL-2Rβ receptor; FIG. 3G: IL-21Rα/IL-2Rβ receptor). FIGS. 3H-3I depict the percentage of EGFR+ control and recombinant cytokine receptor-expressing Treg cells cultured with IL-2 at day 7 and day 13, demonstrating that cells express and maintain recombinant cytokine receptor expression in the presence of IL-2. (FIG. 3H: IL-7Rα/IL-2Rβ receptor; FIG. 3I: IL-4Rα/IL-2Rβ receptor, IL-9Rα/IL-2Rβ receptor, and IL-21Rα/IL-2Rβ receptor).

### Example 4: Recombinant Cytokine Receptor Expression is Enriched Among Regulatory T Cells

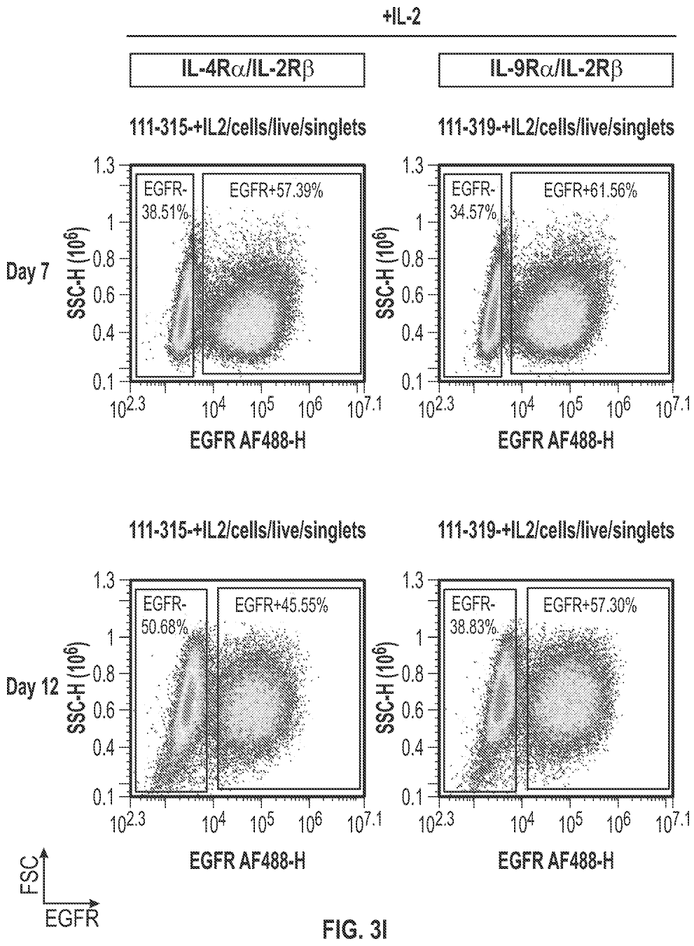

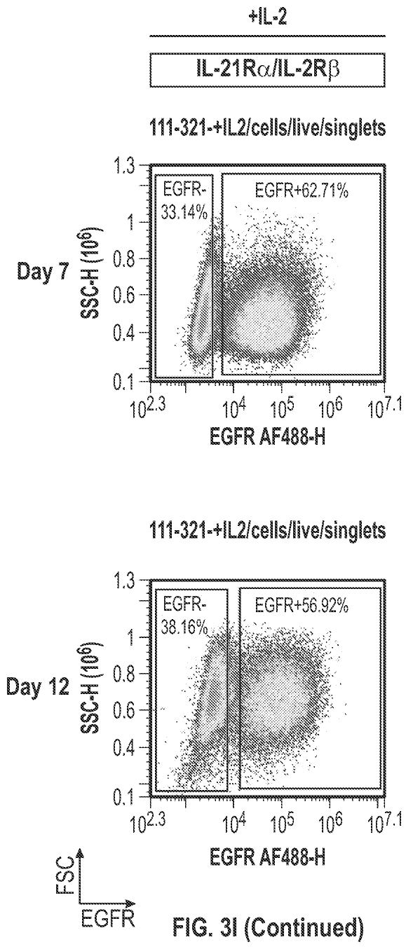

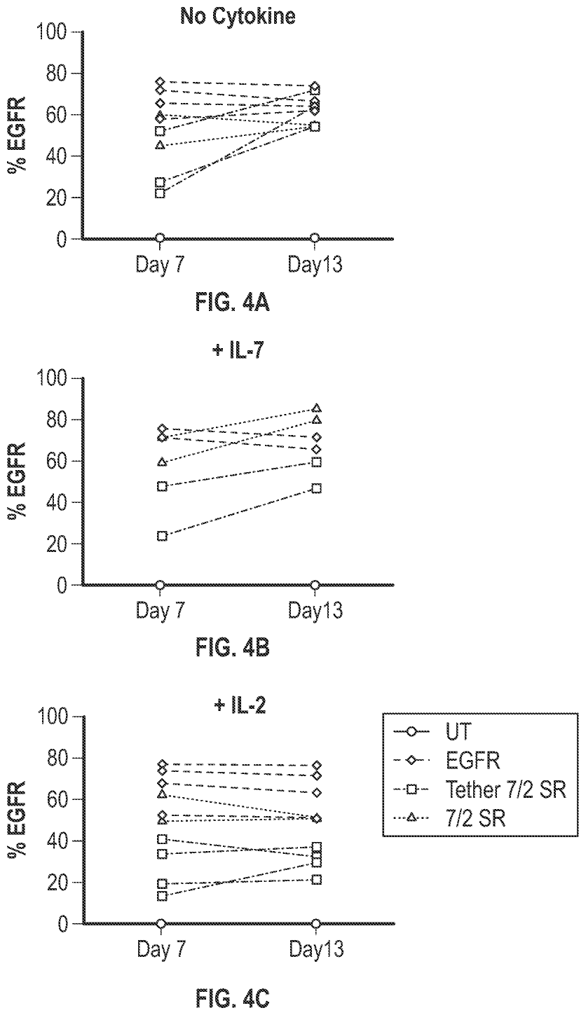

FACS results from the recombinant IL-7Rα/IL-2Rβ receptor-transduced and mock-transduced Treg experiment described in Example 3 were further collated, statistically analyzed, and presented in FIGS. 4A-4C.

FIGS. 4A-4C depict the mean of the percentage (%) of EGFR+ cells for each group that is presented in FIG. 3. FIG. 4A depicts the enrichment of Tregs expressing tethered recombinant IL-7Rα/IL-2Rβ receptors in the absence of cytokine. FIG. 4B depicts the enrichment of Tregs expressing both tethered and untethered recombinant IL-7Rα/IL-2Rβ receptors in the presence of IL-7. FIG. 4C depicts the maintenance of untransduced, mock transduced, tethered recombinant IL-7Rα/IL-2Rβ receptor-expressing, and untethered recombinant IL-7Rα/IL-2Rβ receptor-expressing Treg cells in the presence of IL-2 and shows that Treg cell percentages at day 13 are equivalent to day 7, indicating Treg persistence over time.

Tregs expressing tethered and untethered recombinant IL-7Rα/IL-2Rβ receptors became an enriched population when cultured over time in the absence of IL-2, as these Tregs survived and expanded whereas the untransduced cells underwent T cell contraction and apoptosis. These biological processes can effectively be sorted for and analyzed by FACS, as shown in Example 3.

FACS data from Treg cells that are transduced with recombinant IL-4Rα/IL-2Rβ, IL-9Rα/IL-2Rβ, and IL-21Rα/IL-2Rβ receptors are processed and analyzed as described above.

### Example 5: Recombinant Cytokine Receptors Promote Regulatory T Cell Survival

Treg cells require IL-2 signaling to survive but are unable to produce IL-2; this example demonstrates that Treg cells expressing tethered or untethered IL-4Rα/IL-2Rβ, IL-7Rα/IL-2Rβ, IL-9Rα/IL-2Rβ, or IL-21Rα/IL-2Rβ receptors are able to survive in the absence of exogenous IL-2. As described below, tethered recombinant cytokine receptors survived in the absence of any cytokine, and untethered recombinant cytokine receptors survived in the presence of cognate cytokine alone. Thus, when expressed in Tregs, the recombinant cytokine receptors were able to sustain Treg survival independent of exogenous IL-2.

Untethered and tethered recombinant IL-4Rα/IL-2Rβ, IL-7Rα/IL-2Rβ, IL-9Rα/IL-2Rβ, or IL-21Rα/IL-2Rβ receptor-transduced and control Treg cells were cultured for up to 14 days post-transduction with vehicle control or with 300 IU/mL IL-2, 0.4 ng/ml IL-4, 1 ng/ml IL-7. 2 ng/mL IL-9, or 5 ng/ml IL-21. At specific intervals, i.e., day 2, day 5, day 7, day 9, day 12, and day 14, Treg cells were sampled from each condition and assessed for the percentage of viable Treg cells using live/dead FACS analysis with a cell viability dye and following the methods described in Example 3 above.

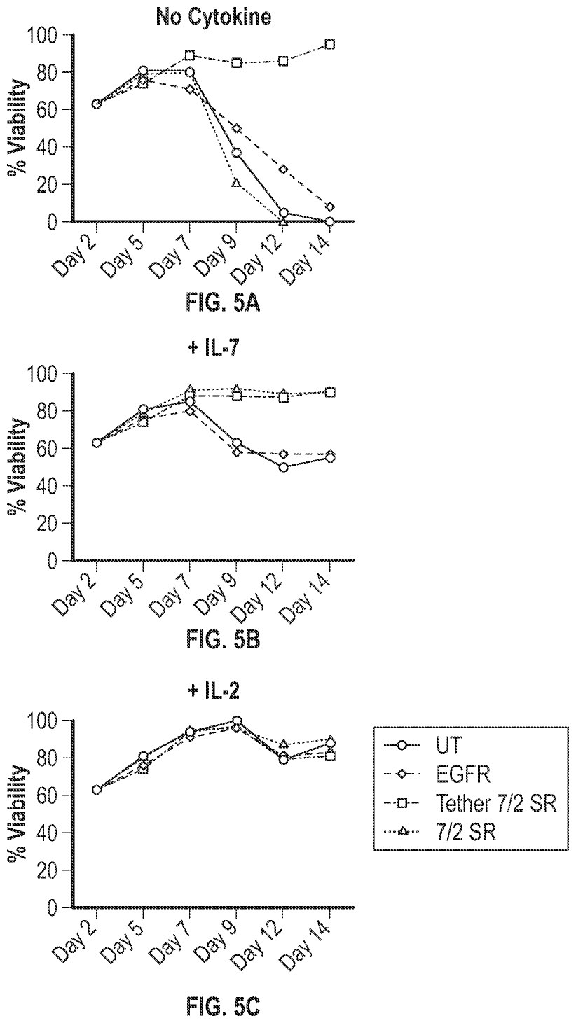

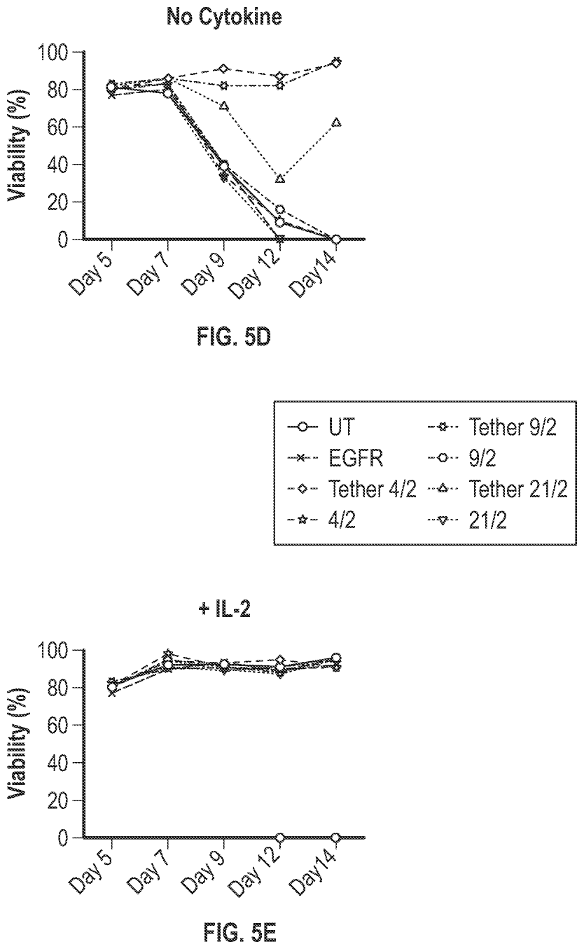

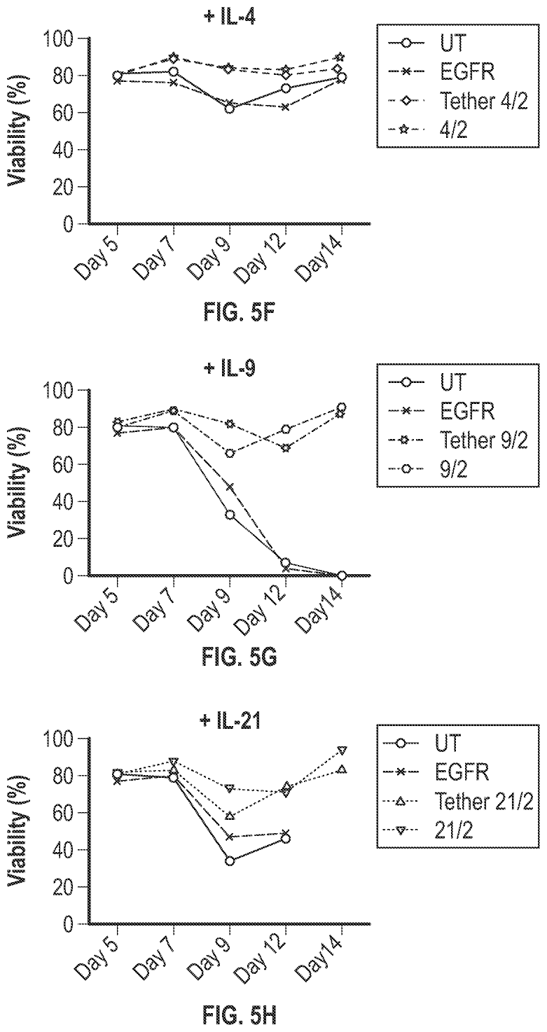

FIGS. 5A-5H depicts the means of the percentage (%) of EGFR+ cells for Treg cells from each experimental condition over time. FIG. 5A depicts control and recombinant IL-7Rα/IL-2Rβ receptor-expressing Treg cells cultured in the absence of cytokine. FIG. 5B depicts control and recombinant IL-7Rα/IL-2Rβ receptor-expressing Treg cells cultured in the presence of IL-7. FIG. 5C depicts control and recombinant IL-7Rα/IL-2Rβ receptor-expressing Treg cells cultured in the presence of IL-2. FIG. 5D depicts control and recombinant IL-4Rα/IL-2Rβ, IL-9Rα/IL-2Rβ, and IL-21Rα/IL-2Rβ receptor-expressing Treg cells cultured in the absence of cytokine. FIG. 5E depicts control and recombinant IL-4Rα/IL-2Rβ, IL-9Rα/IL-2Rβ, and IL-21Rα/IL-2Rβ receptor-expressing Treg cells cultured with IL-2. FIG. 5F depicts control and recombinant IL-4Rα/IL-2Rβ receptor-expressing Treg cells cultured in the presence of IL-4. FIG. 5G depicts control and recombinant IL-9Rα/IL-2Rβ receptor-expressing Treg cells cultured in the presence of IL-9. FIG. 5H depicts control and recombinant IL-21Rα/IL-2Rβ receptor-expressing Treg cells cultured in the presence of IL-21.

Tregs expressing tethered recombinant IL-4Rα/IL-2Rβ. IL-7Rα/IL-2Rβ, IL-9Rα/IL-2Rβ, or IL-21Rα/IL-2Rβ receptors demonstrated constant cell viability and persistence over time both in the absence and presence of either cognate cytokine or IL-2. Tregs expressing untethered recombinant IL-4Rα/IL-2Rβ. IL-7Rα/IL-2Rβ. IL-9Rα/IL-2Rβ, or IL-21Rα/IL-2Rβ receptors demonstrated a constant cell viability and persistence over time when cultured in the presence of either cognate cytokine or IL-2. Control Tregs demonstrated cell viability and persistence only when cultured with IL-2. Thus, upon expression in transduced Tregs, the recombinant cytokine receptors were able to sustain Treg survival independent of exogenous IL-2 in vitro.

### Example 6: Recombinant Cytokine Receptors Promote Regulatory T Cell Proliferation

IL-2 signals to T cells, including Treg cells, to proliferate. This example demonstrates that Treg cells expressing tethered or untethered recombinant IL-4Rα/IL-2Rβ, IL-7Rα/IL-2Rβ, IL-9Rα/IL-2Rβ, or IL-21Rα/IL-2Rβ receptors are able to proliferate independent of exogenous IL-2. As described herein. Tregs expressing tethered recombinant IL-4Rα/IL-2Rβ. IL-7Rα/IL-2Rβ. IL-9Rα/IL-2Rβ, or IL-21Rα/IL-2Rβ receptors proliferated in the absence of any cytokine, and Tregs expressing untethered recombinant IL-4Rα/IL-2Rβ, IL-7Rα/IL-2Rβ, IL-9Rα/IL-2Rβ, or IL-21Rα/IL-2Rβ receptors proliferated in the presence of cognate cytokine alone.

Treg cells expressing recombinant IL-4Rα/IL-2Rβ, IL-7Rα/IL-2Rβ, IL-9Rα/IL-2Rβ, or IL-21Rα/IL-2Rβ receptors and control Treg cells were prepared using the same method as described in Example 4. At specific intervals, i.e., day 2, day 5, day 7, day 9, day 12, and day 14, the number of Treg cells from each condition were counted, averaged within each condition, and plotted over time in order to quantify the level of Treg proliferation.

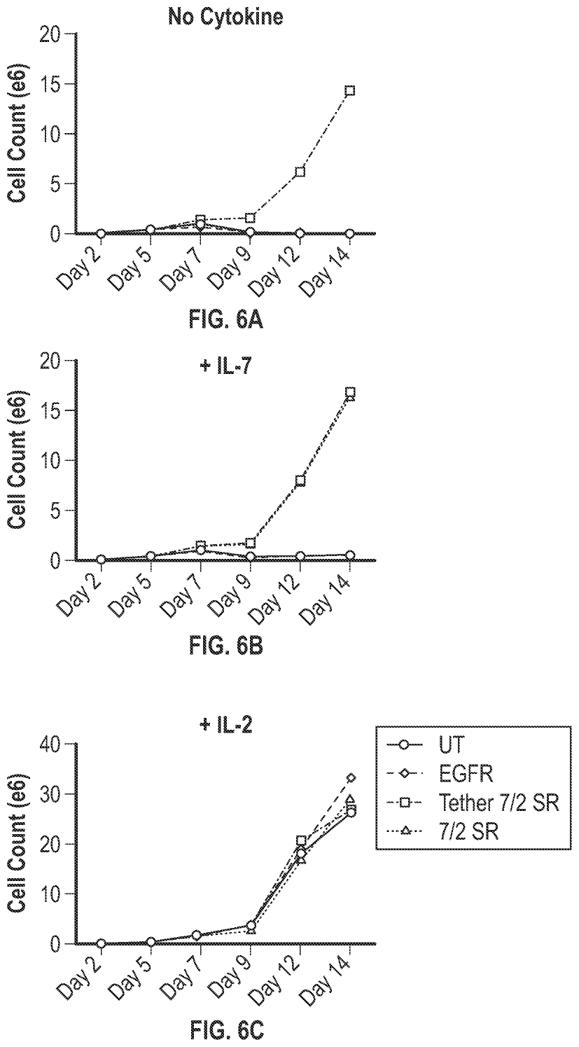

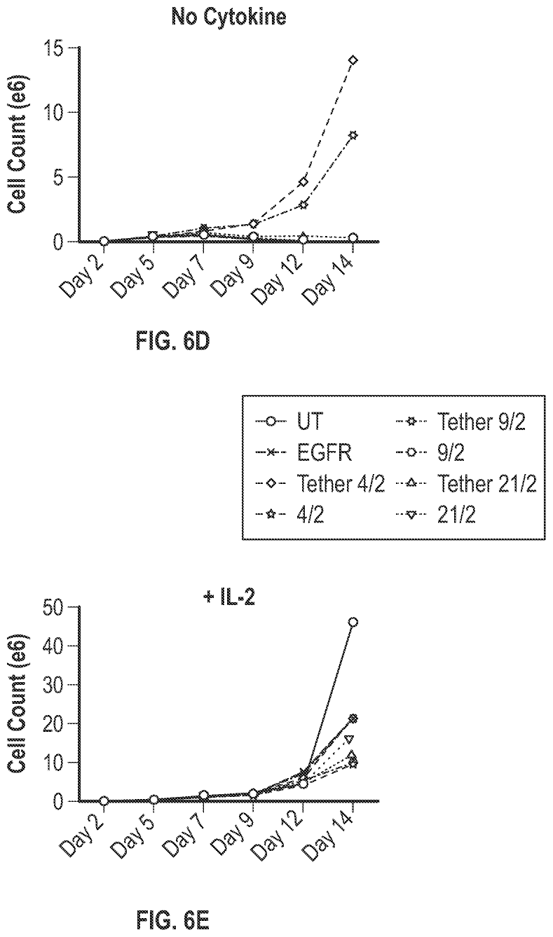

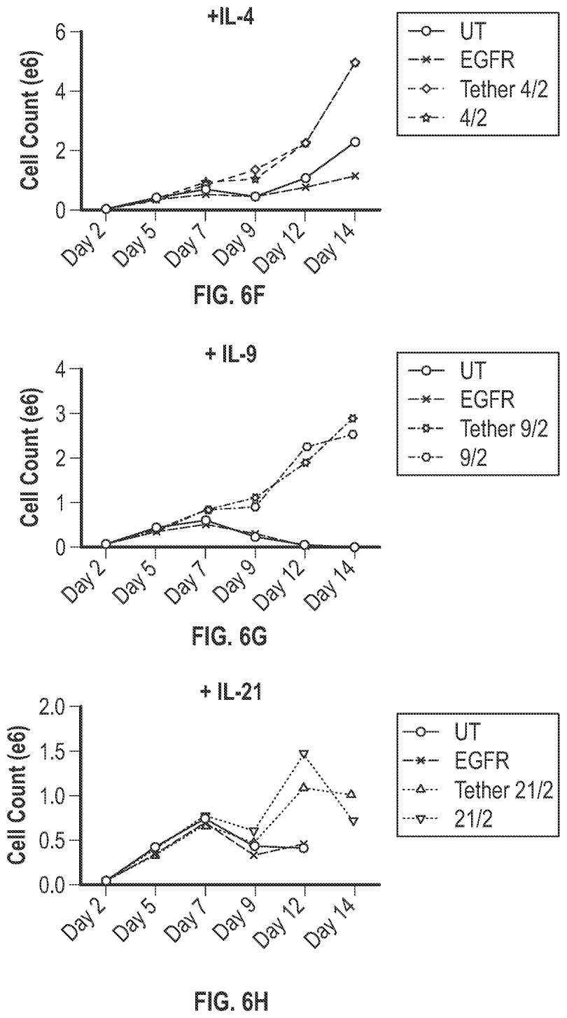

FIGS. 6A-6J shows the means of the total number of cultured Treg cells from each experimental and control condition over time. FIG. 6A depicts control and recombinant IL-7Rα/IL-2Rβ receptor-expressing Treg cells cultured in the absence of cytokine. FIG. 6B depicts control and recombinant IL-7Rα/IL-2Rβ receptor-expressing Treg cells cultured in the presence of IL-7. FIG. 6C depicts control and recombinant IL-7Rα/IL-2Rβ receptor-expressing Treg cells cultured in the presence of IL-2. FIG. 6D depicts control and recombinant IL-4Rα/IL-2Rβ, IL-9Rα/IL-2Rβ, and IL-21Rα/IL-2Rβ receptor-expressing Treg cells cultured in the absence of cytokine. FIG. 6E depicts control and recombinant IL-4Rα/IL-2Rβ. IL-9Rα/IL-2Rβ, and IL-21Rα/IL-2Rβ receptor-expressing Treg cells cultured with IL-2. FIG. 6F depicts control and recombinant IL-4Rα/IL-2Rβ receptor-expressing Treg cells cultured in the presence of IL-4. FIG. 6G depicts control and recombinant IL-9Rα/IL-2Rβ receptor-expressing Treg cells cultured in the presence of IL-9. FIG. 6H depicts control and recombinant IL-21Rα/IL-2Rβ receptor-expressing Treg cells cultured in the presence of IL-21. FIG. 6I depicts the fold expansion of control and IL-9-tethered recombinant IL-9Rα/IL-2Rβ receptor-expressing Treg cells cultured in the presence or absence of IL-2. FIG. 6J shows the line graph representation of fold expansion over time between control Tregs and Tregs expressing recombinant IL-9Rα/IL-2Rβ receptors in the absence or presence of IL-2.

Tregs expressing tethered recombinant cytokine receptors demonstrated an exponential increase in the number of cells as a readout of Treg proliferative capacity in the absence and presence of either cognate cytokine or IL-2. Tregs expressing untethered recombinant cytokine receptors demonstrated an exponential increase in the number of cells over time when cultured in the presence of either cognate cytokine or IL-2. Control Tregs only demonstrated an exponential increase in the number of Treg cells in the presence of IL-2.

### Example 7: Recombinant Cytokine Receptors Promote the Expression of Markers of the Regulatory T Cell Profile

Treg cells are a unique lineage within CD4+ and CD8+ T cells and thus express a unique gene profile and protein markers. Traditionally. Tregs have been identified from broader T cell populations by CD25 (also known as IL-2 receptor) and the FOXP3 transcription factor that both play key roles in Treg differentiation. More recently, the CD71 (also known as transferrin receptor) and HELIOS transcription factor likewise were identified as relevant markers of the Treg profile. Using FACS analysis, tethered recombinant IL-4Rα/IL-2Rβ, IL-7Rα/IL-2Rβ. IL-9Rα/IL-2Rβ, or IL-21Rα/IL-2Rβ cytokine receptors displayed increased Treg-specific lineage markers in the absence of any cytokine, and untethered recombinant IL-4Rα/IL-2Rβ. IL-7Rα/IL-2Rβ. IL-9Rα/IL-2Rβ, or IL-21Rα/IL-2Rβ cytokine receptors displayed increased Treg-specific lineage markers in the presence of cognate cytokine alone. Additionally, Tregs expressing recombinant cytokine receptors also displayed greater homogeneity across all Treg markers. These data indicate that the expression of the recombinant cytokine receptors supports the Treg phenotype in environments that are limited or lacking in IL-2.

Treg cells expressing recombinant IL-4Rα/IL-2Rβ. IL-7Rα/IL-2Rβ, IL-9Rα/IL-2Rβ, or IL-21Rα/IL-2Rβ receptors and control Treg cells were prepared using the same method as described in Example 4. At day 7 and day 12, Treg cells from each condition were processed for FACS analysis of multiple Treg lineage markers. Staining for surface markers, such as CD25 and CD71, and intracellular markers, such as FOXP3 and HELIOS, and FACS analysis were performed as described in Example 3. Mean fluorescence intensity (MFI) for each marker was assessed such that higher MFI for each marker was indicative of a Treg phenotype.

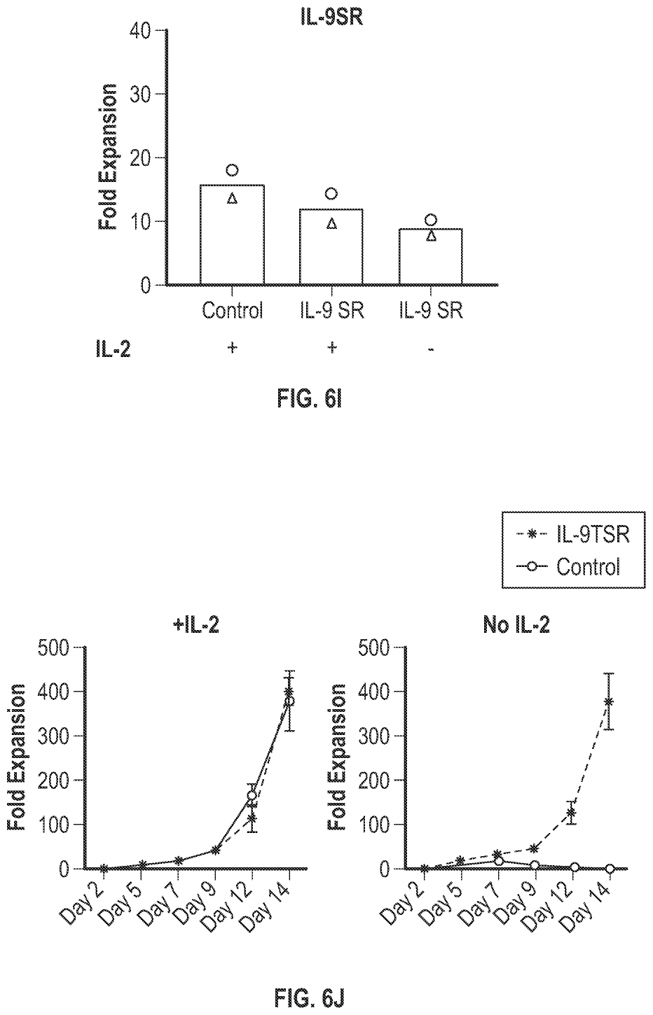

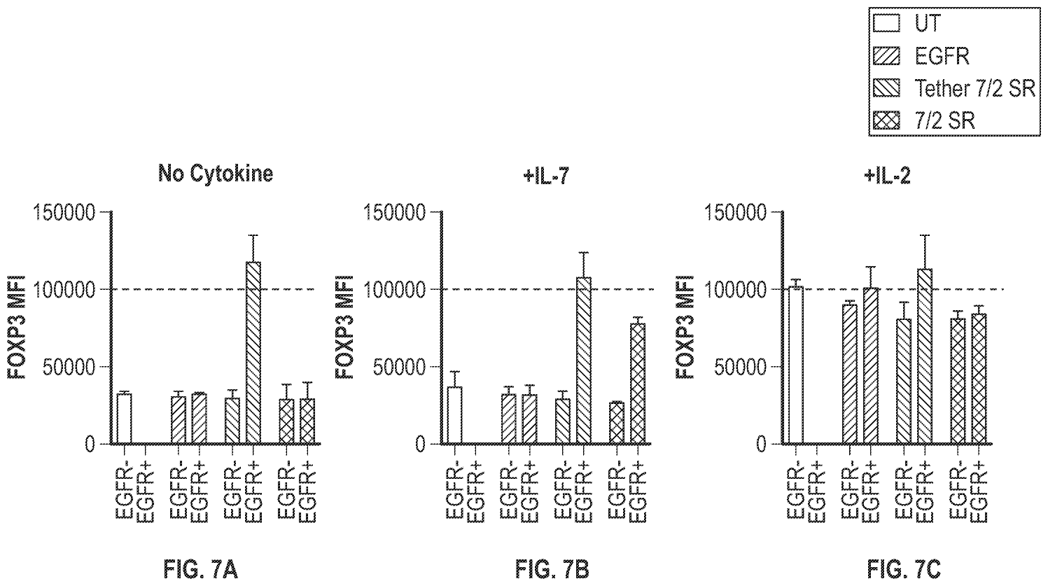

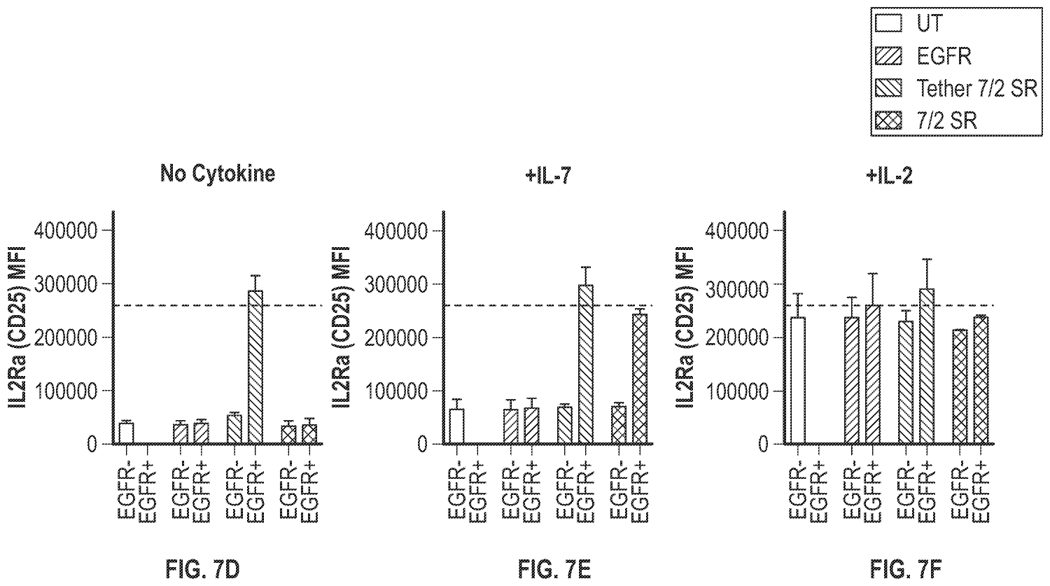

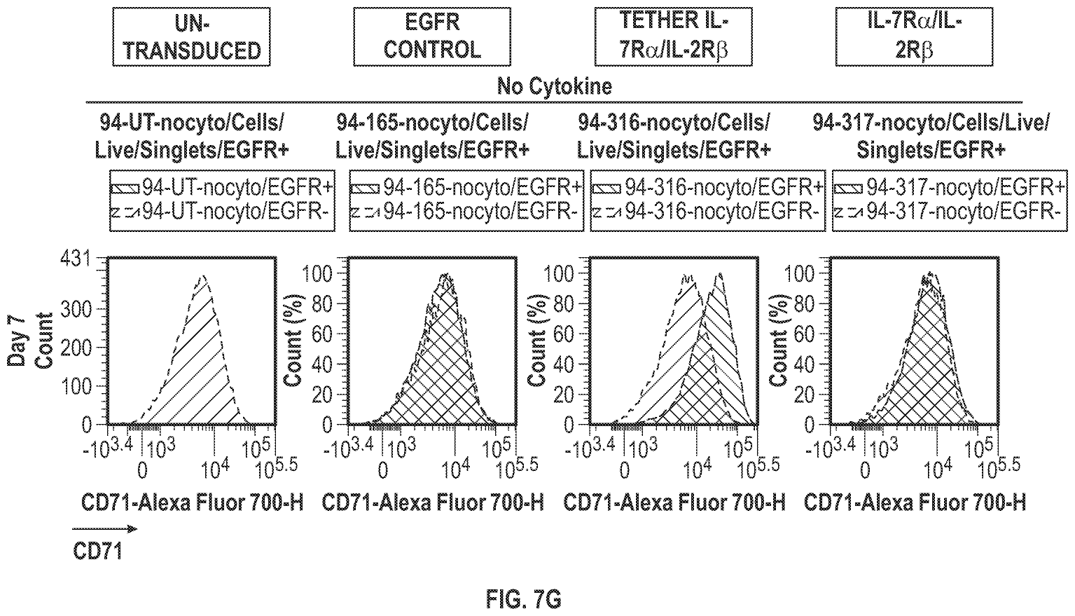

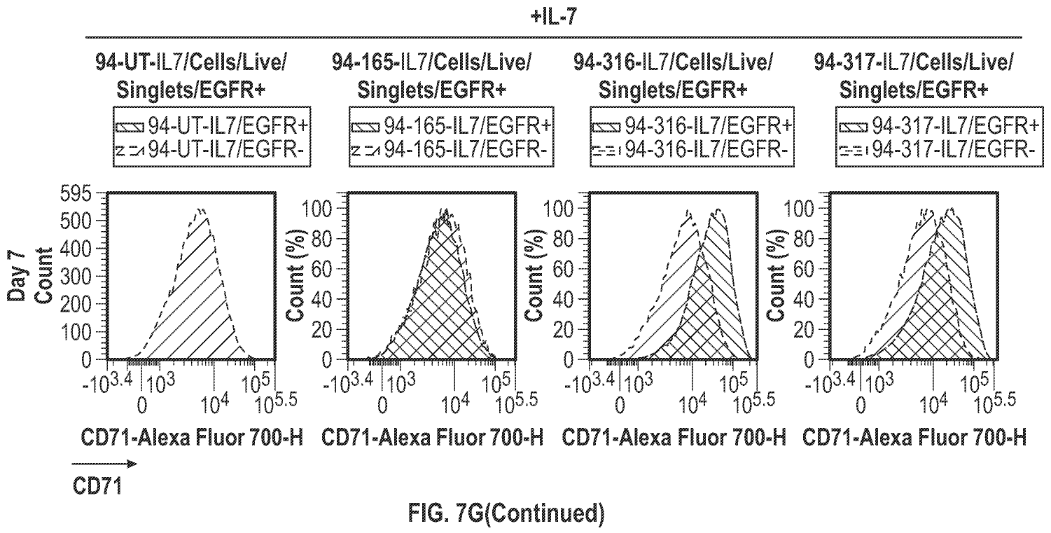

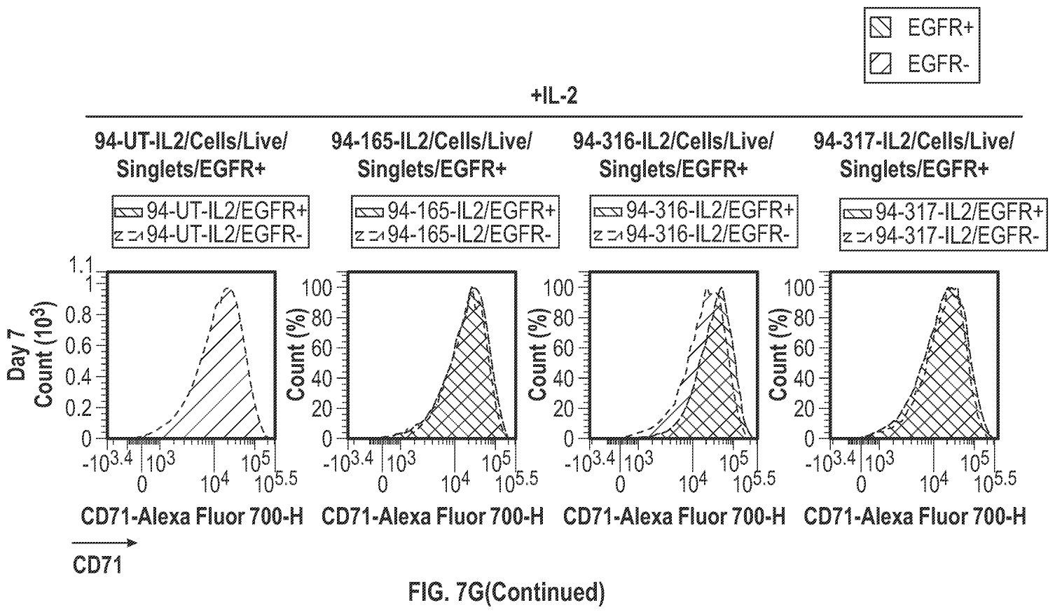

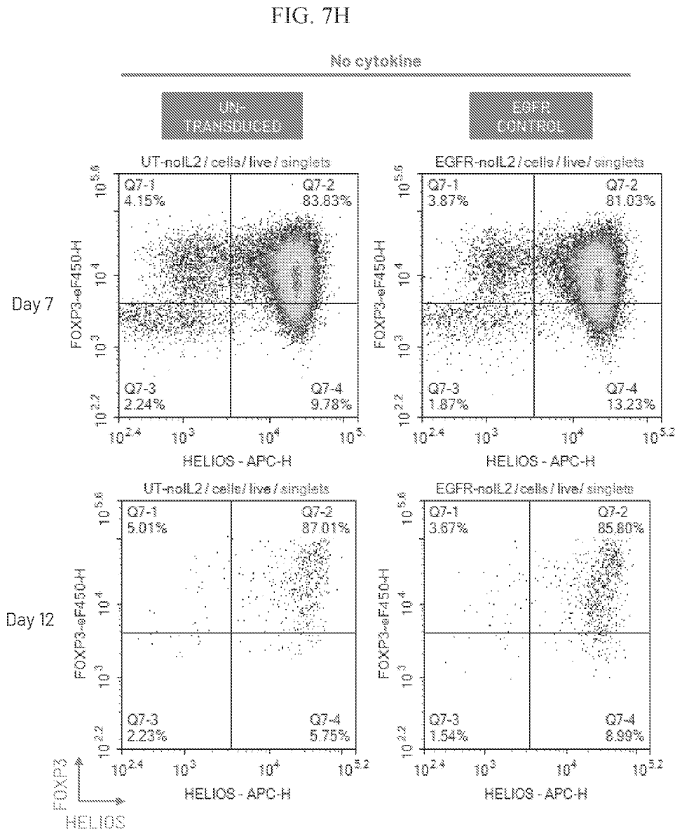

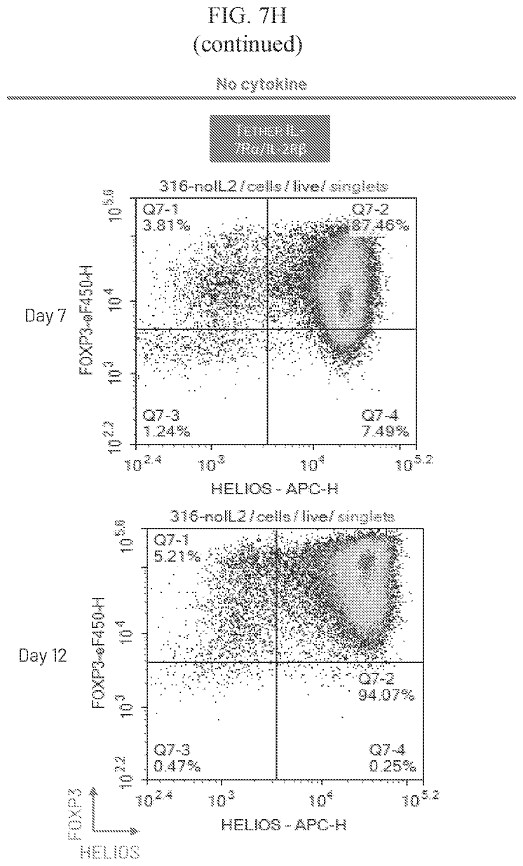

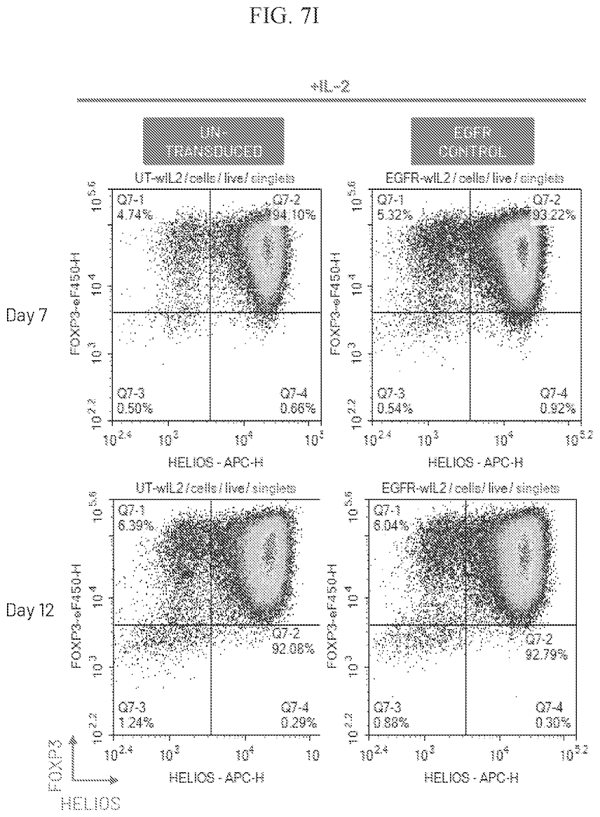

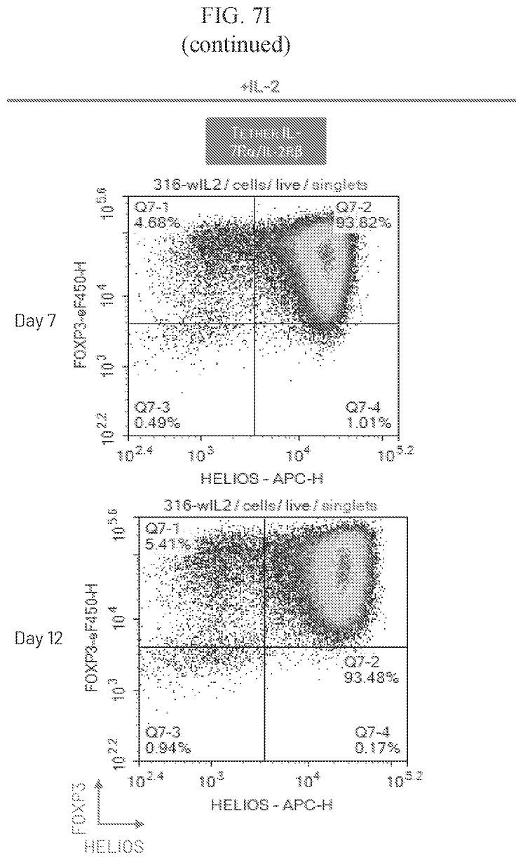

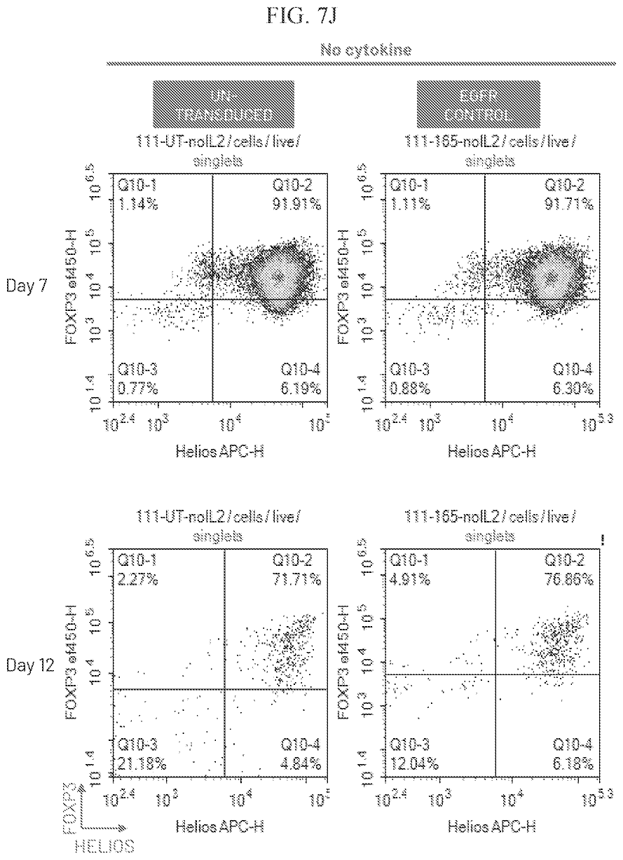

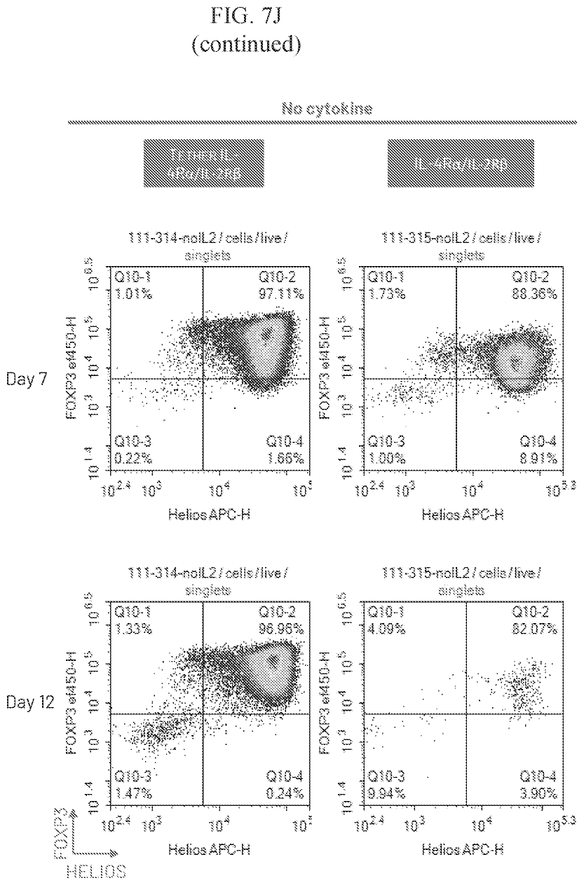

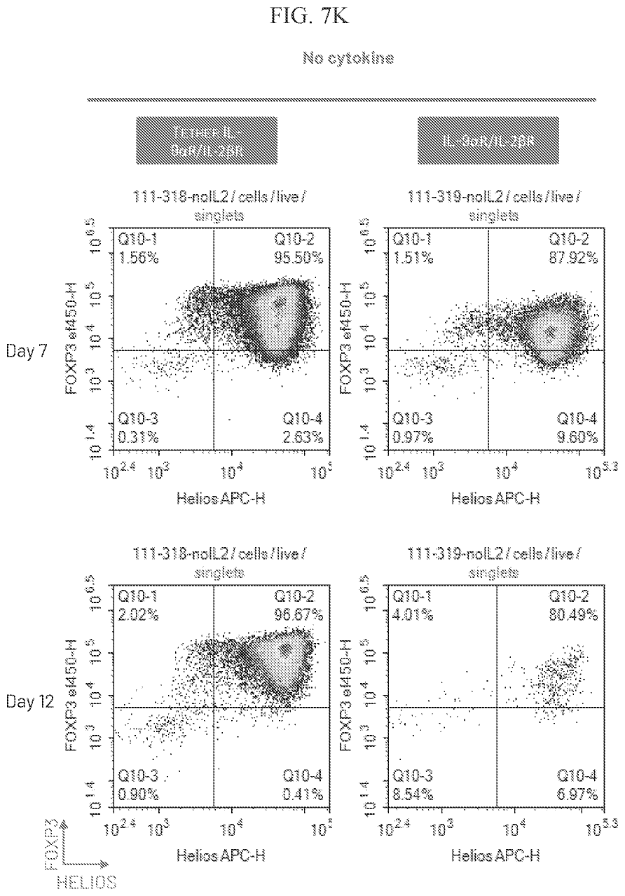

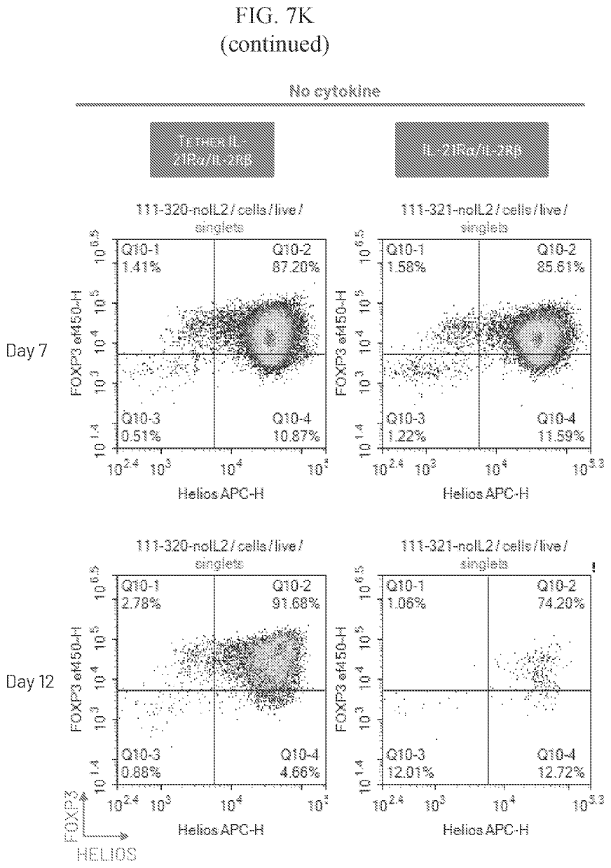

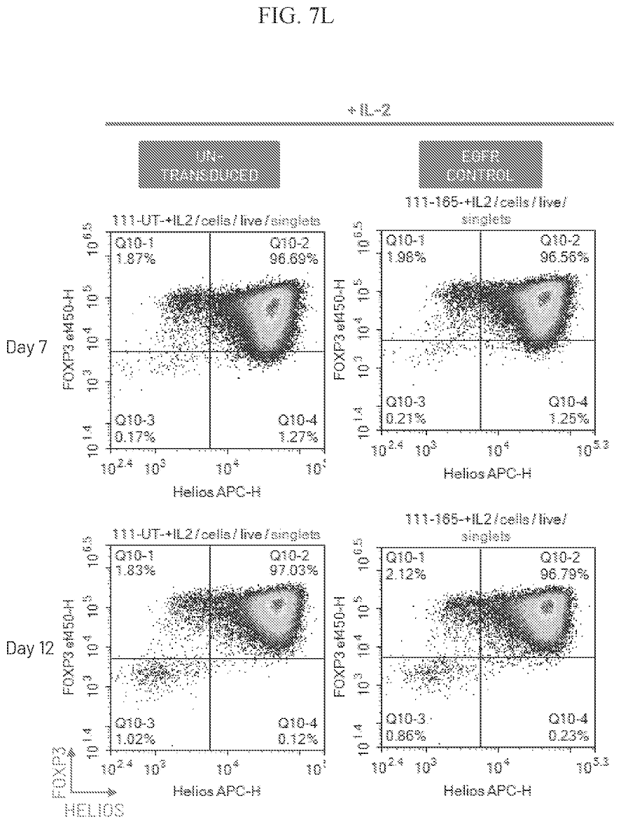

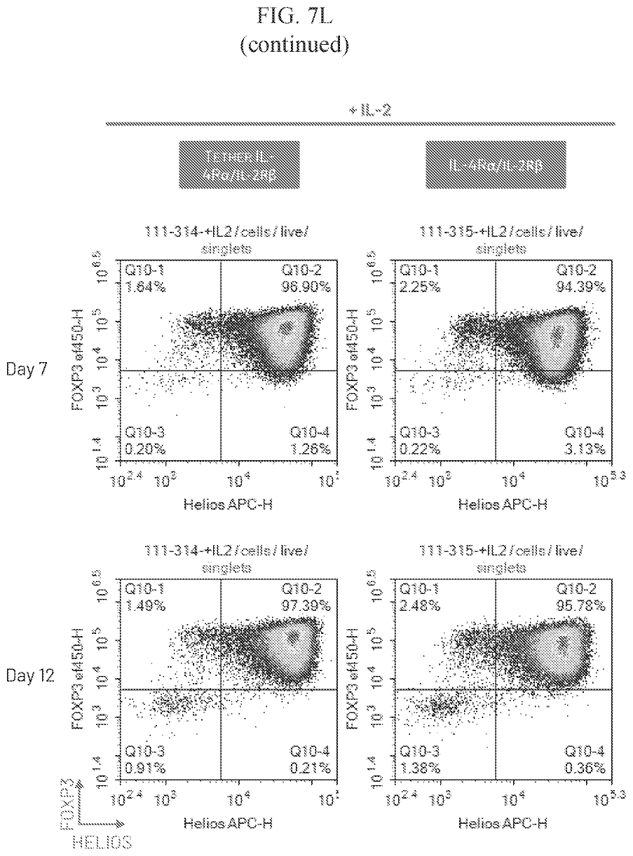

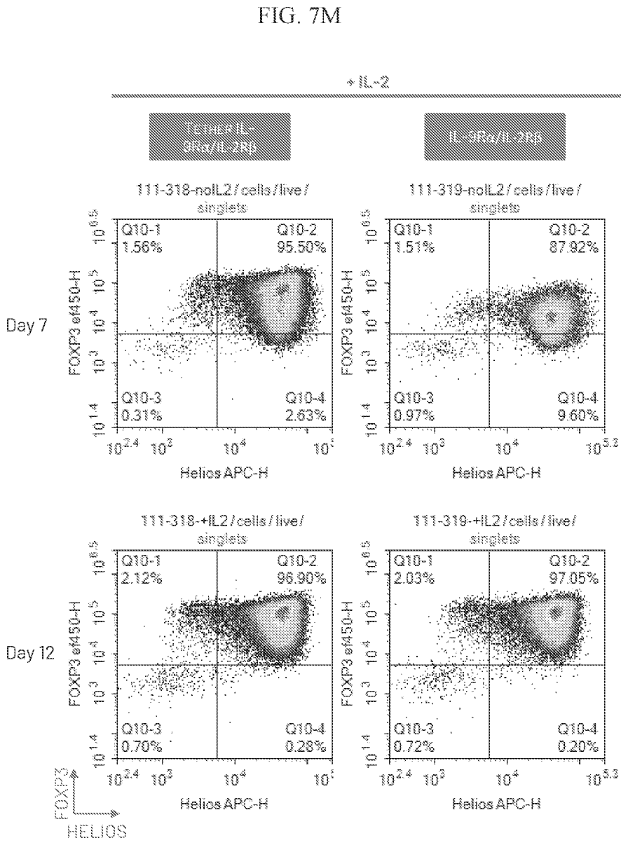

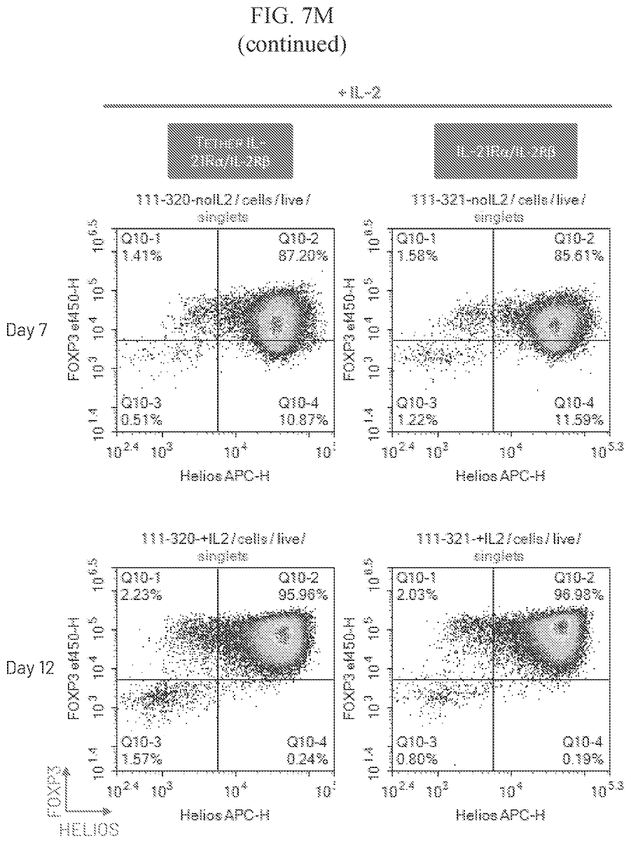

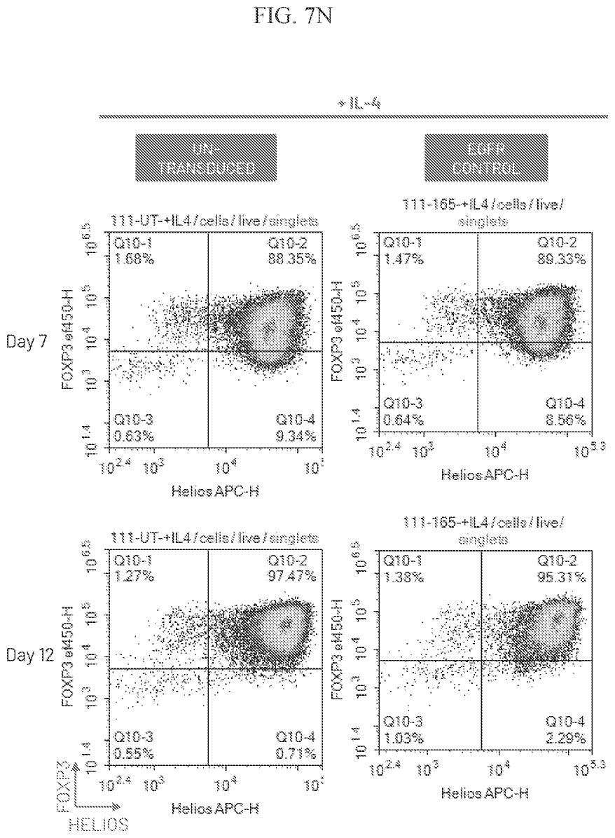

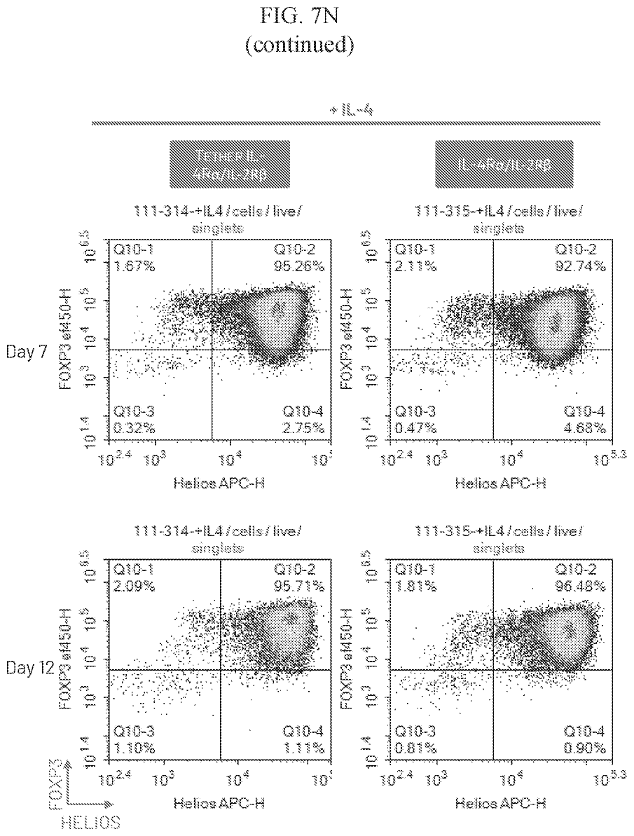

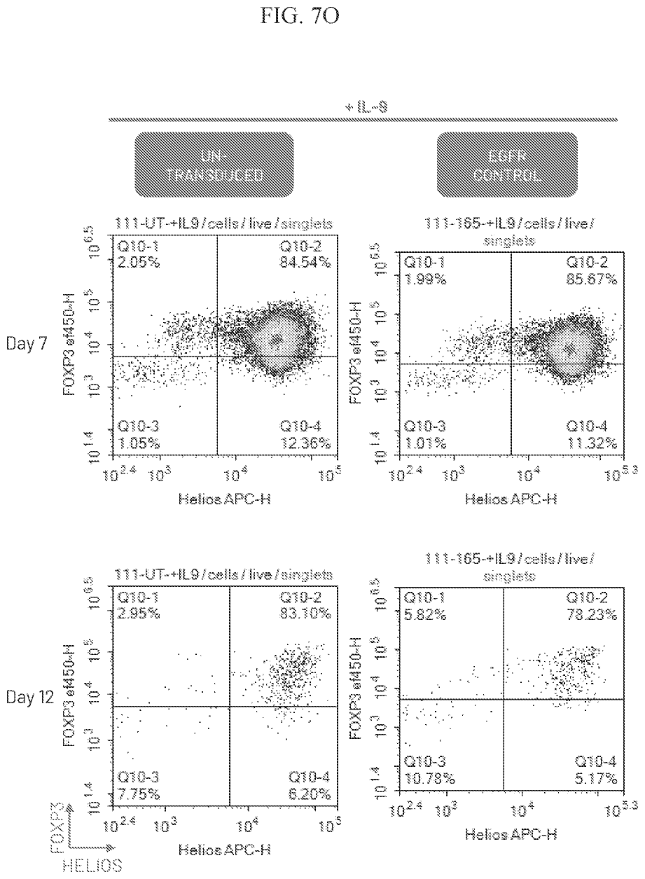

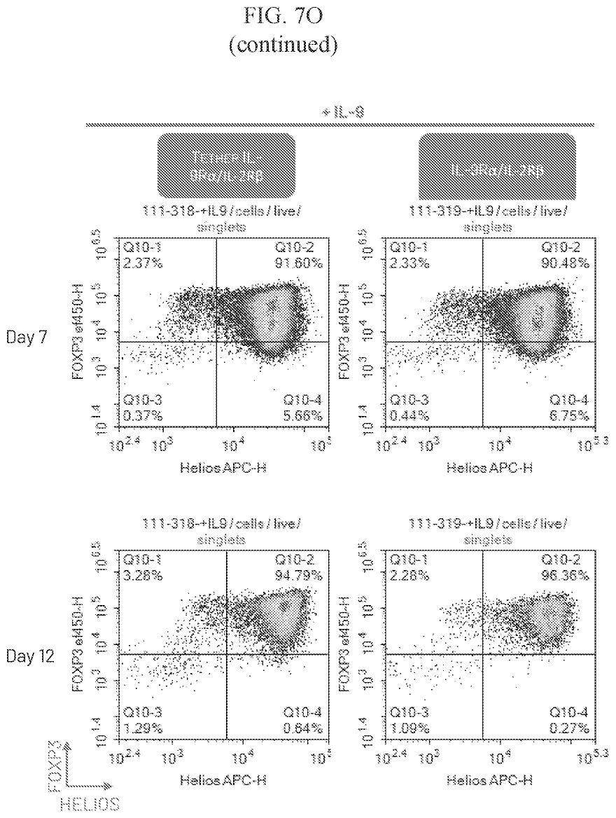

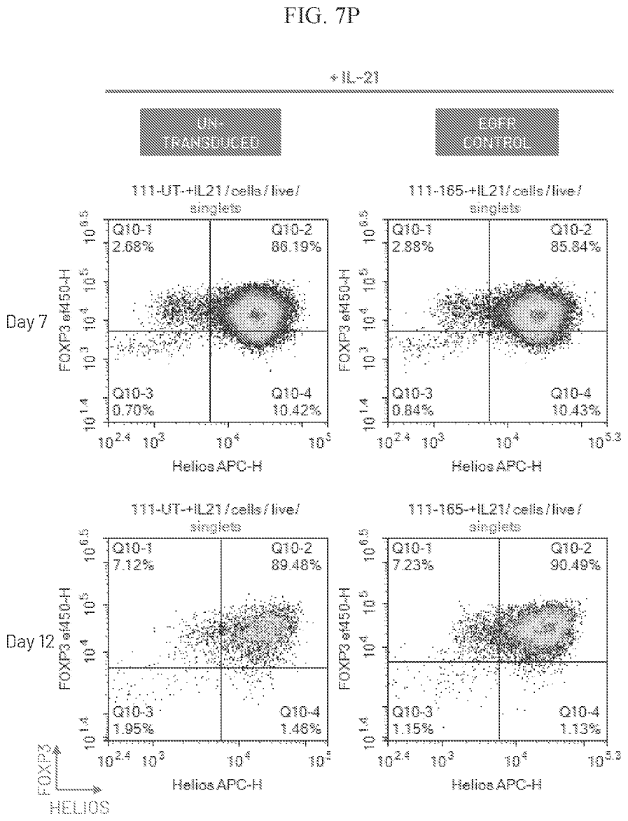

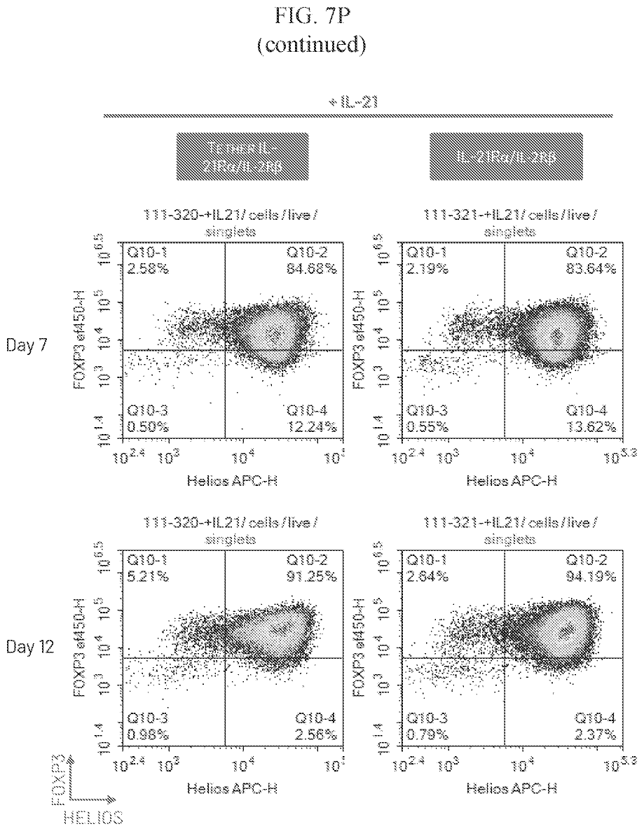

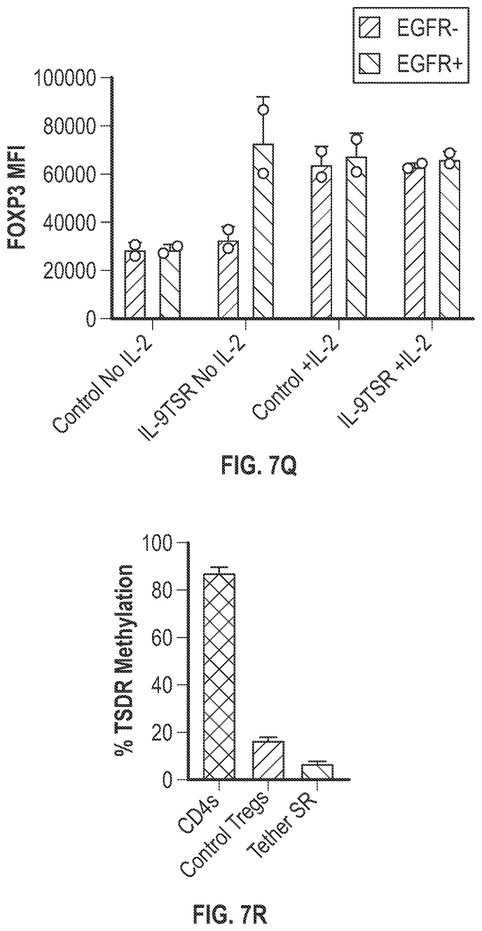

FIGS. 7A-7R depicts the expression of Treg lineage-specific markers by representative FACS plots, MFI histograms, and graphical representations from each experimental and control condition over time. Dotted lines in FIGS. 7A-7F indicate the expected baseline expression levels of FOXP3 and CD25 in IL-2-treated control Tregs for comparing the Treg profile of recombinant IL-7Rα/IL-2Rβ receptor-expressing Tregs. FIG. 7A depicts FOXP3 MFI for control and recombinant IL-7Rα/IL-2Rβ receptor-expressing Treg cells cultured in the absence of cytokine. FIG. 7B depicts FOXP3 MFI for control and recombinant IL-7Rα/IL-2Rβ receptor-expressing Treg cells cultured in the presence of IL-7. FIG. 7C depicts FOXP3 MFI for control and recombinant IL-7Rα/IL-2Rβ receptor-expressing Treg cells cultured in the presence of IL-2. Tregs expressing tethered recombinant IL-7Rα/IL-2Rβ receptors displayed higher FOXP3 MFI in both the absence and presence of IL-7 or IL-2 than at the baseline established by control Tregs cultured with IL-2. Tregs expressing untethered recombinant IL-7Rα/IL-2Rβ receptors displayed an increase in FOXP3 MFI when cultured in the presence of IL-7 or IL-2 compared to no cytokine. FIG. 7Q depicts FOXP3 MFI for control and recombinant IL-9Rα/IL-2Rβ receptor-expressing Treg cells cultured in the presence or absence of IL-2. Tregs expressing tethered recombinant IL-9Rα/IL-2Rβ receptors displayed higher FOXP3 MFI in the absence of IL-2 than, and on par in the presence of IL-2 with, the baseline established by control Tregs cultured with IL-2. FIG. 7R depicts the graphical representation of the percent TSDR methylation in effector CD4+ T cells, control Tregs, and recombinant IL-9Rα/IL-2Rβ receptor-expressing Tregs. Tregs display reduced TSDR methylation profile, or increased demethylation, compared to effector CD4+ T cells, which is sufficient to induce FOXP3 protein expression. TSDR is a CpG dinucleotide-rich and highly conserved region within the conserved non-coding sequences 2 (CNS2), located in the first intron of the FOXP3 gene. Demethylation in TSDR is thought to contribute to both the stability of FOXP3 expression and the maintenance of the suppressive phenotype for Tregs (see, e.g., Zhuo, et al. Mol Cancer 2014; 13 (153), hereby incorporated by reference in its entirety). The recombinant IL-9Rα/IL-2Rβ receptor-expressing Tregs show a 50% decrease in TSDR methylation compared to control Tregs, suggesting increased FOXP3, and therefore increased Treg, stability.

FIG. 7D depicts CD25 MFI for control and recombinant IL-7Rα/IL-2Rβ receptor-expressing Treg cells cultured in the absence of any cytokine. FIG. 7E depicts CD25 MFI for control and recombinant IL-7Rα/IL-2Rβ receptor-expressing Treg cells cultured in the presence of IL-7. FIG. 7F depicts CD25 MFI for control and recombinant IL-7Rα/IL-2Rβ receptor-expressing Treg cells cultured with IL-2. Tregs expressing tethered recombinant IL-7Rα/IL-2Rβ receptors displayed higher CD25 MFI both with and without either IL-7 or IL-2 than at the baseline established by control Tregs cultured with IL-2. Tregs expressing untethered recombinant IL-7Rα/IL-2Rβ receptors displayed an increase in CD25 MFI when cultured with either IL-7 or IL-2 compared to no cytokine.

FIG. 7G depicts representative histograms of CD71 signal intensity for control and recombinant IL-7Rα/IL-2Rβ receptor-expressing Treg cells cultured without cytokine, with IL-7, or with IL-2 at day 7. Tregs expressing tethered recombinant IL-7Rα/IL-2Rβ receptors displayed high CD71 signal intensity both with and without either IL-7 or IL-2 treatment. Tregs expressing untethered recombinant IL-7Rα/IL-2Rβ receptors displayed high CD71 signal intensity when cultured with IL-7 or IL-2. Control Tregs lacking recombinant cytokine receptors displayed high CD71 signal intensity only when cultured with IL-2.

FIG. 7H depicts representative dot plots of HELIOS and FOXP3 signal intensity for control and recombinant IL-7Rα/IL-2Rβ receptor-expressing Treg cells cultured without any cytokine at day 7 and day 12. FIG. 7I depicts representative dot plots of HELIOS and FOXP3 signal intensity for Treg cells cultured with IL-2 at day 7 and day 13. Tregs expressing tethered recombinant IL-7Rα/IL-2Rβ receptors displayed high HELIOS and FOXP3 signal intensity both with and without IL-2 treatment. Control Tregs lacking recombinant cytokine receptors displayed high HELIOS and FOXP3 signal intensity only when cultured with IL-2.

Representative dot plots show the levels of HELIOS and FOXP3 in control Tregs and Tregs expressing recombinant IL-4α/IL-2Rβ receptors (FIG. 7J), IL-9α/IL-2Rβ receptors (FIG. 7K), and IL-21α/IL-2Rβ receptors (FIG. 7K) cultured without any cytokine at day 7 and day 12. Representative dot plots show HELIOS and FOXP3 levels in control Tregs and Tregs expressing recombinant IL-4α/IL-2Rβ receptors (FIG. 7L), IL-9α/IL-2Rβ receptors (FIG. 7M), and IL-21α/IL-2Rβ receptors (FIG. 7M) cultured with IL-2 at day 7 and day 12. Representative dot plots show HELIOS and FOXP3 levels in control Tregs and Tregs expressing recombinant IL-4α/IL-2Rβ receptors (FIG. 7N), IL-9α/IL-2Rβ receptors (FIG. 7O), and IL-21α/IL-2Rβ receptors (FIG. 7P) cultured with cognate cytokine at day 7 and day 12. These results show that Tregs expressing recombinant cytokine receptors can maintain a homogenous Treg profile in the absence of IL-2.

### Example 8: Recombinant Cytokine Receptors Activate the IL-2 Signaling Pathway in Regulatory T Cells

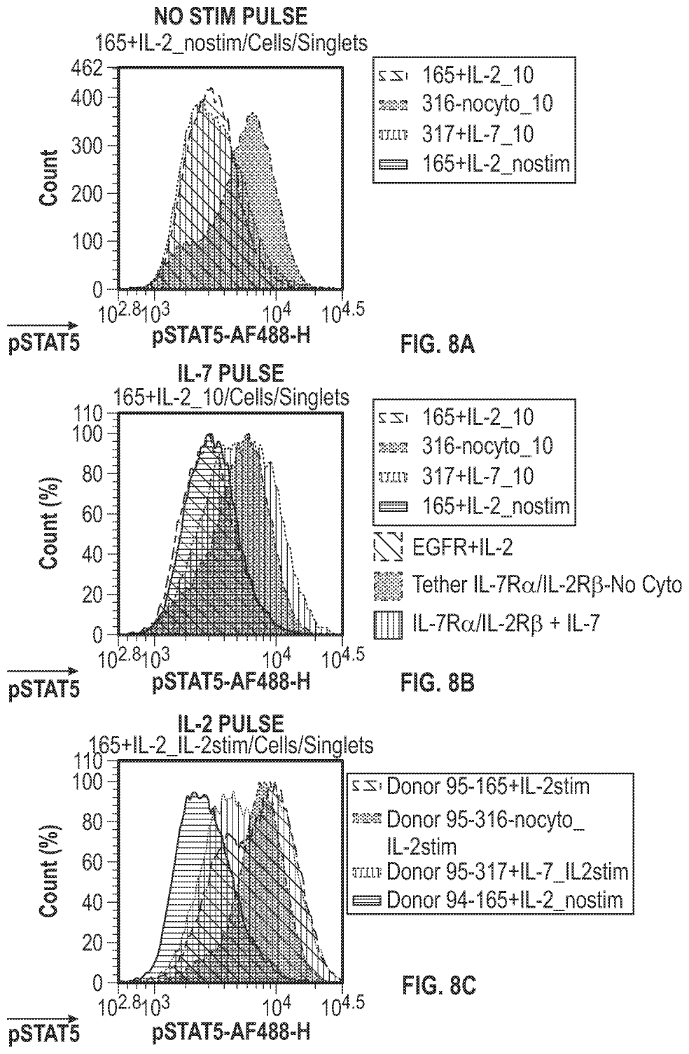

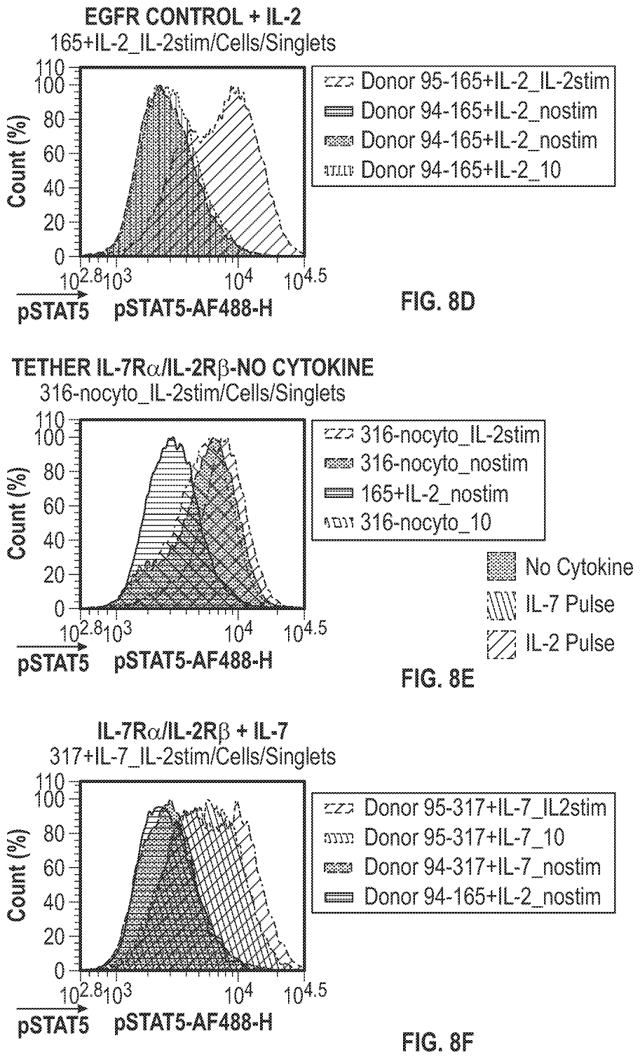

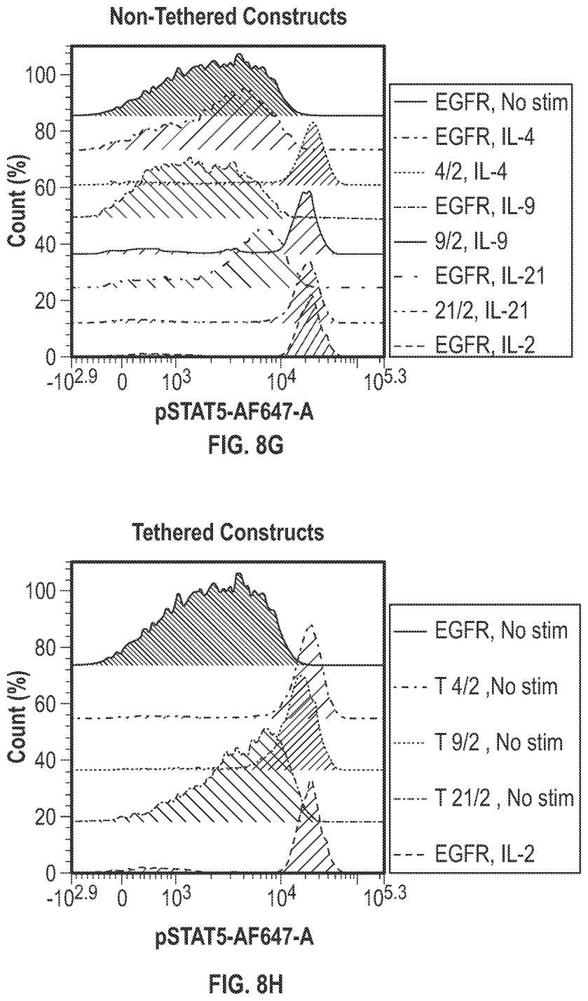

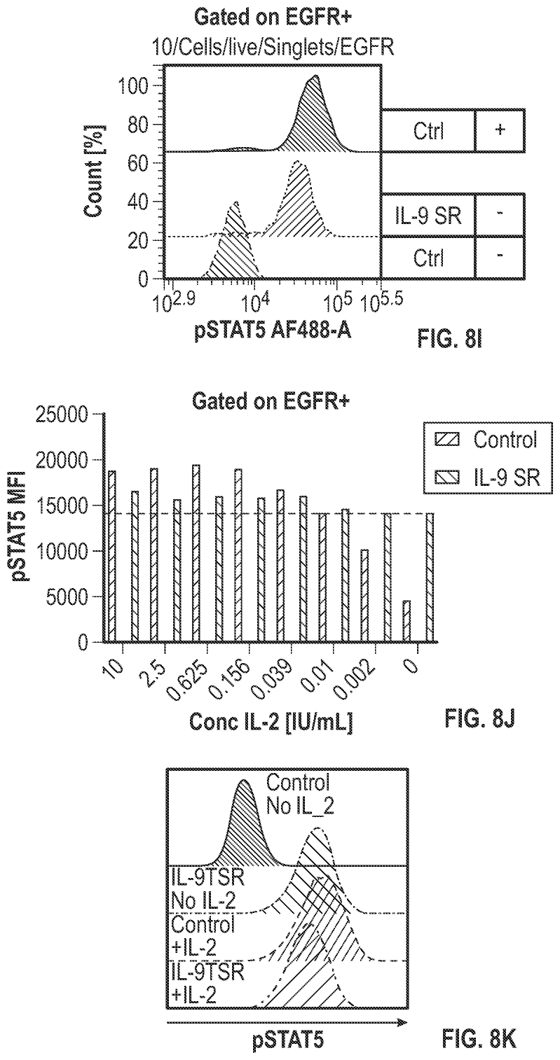

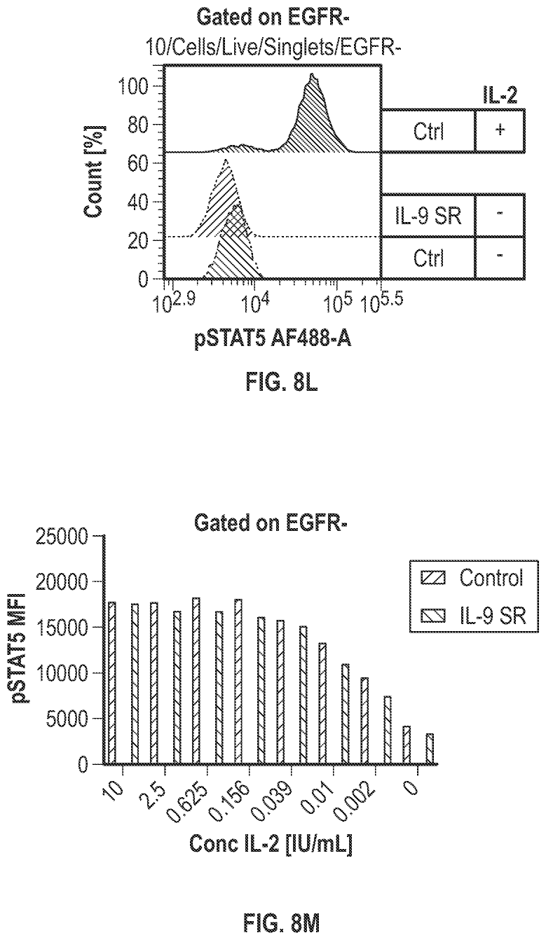

Example 8 demonstrates that not only do the recombinant cytokine receptors activate IL-2 signaling within an IL-2 reporter cell line, but these recombinant cytokine receptors also activate canonical IL-2 signaling upon expression in Treg cells. STAT5 signaling is downstream of IL-2 signal transduction in Treg cells. Upon IL-2 signal activation, STAT5 is phosphorylated and then translocated to the nucleus to initiate transcription of target genes. This STAT5 phosphorylation event can be analyzed using antibodies that bind specifically to the phosphorylated form of STAT5. As a result, relative levels of IL-2 signaling can be compared between Treg populations propagated in different conditions. FIGS. 8A-8M depict the relative IL-2 signaling in Treg cells expressing tethered or untethered recombinant IL-4Rα/IL-2Rβ, IL-7Rα/IL-2Rβ, IL-9Rα/IL-2Rβ, or IL-21Rα/IL-2Rβ receptors compared to control Tregs, as indicated by STAT5 phosphorylation. When these recombinant cytokine receptors are expressed in Tregs, they are able to activate the IL-2 signaling pathway in Tregs in environments that are limited or lacking in IL-2.

Treg cells expressing recombinant IL-4Rα/IL-2Rβ, IL-7Rα/IL-2Rβ, IL-9Rα/IL-2Rβ, or IL-21Rα/IL-2Rβ receptors and control Treg cells were prepared using the same method as Example 4 above. At day 15, Treg cells from each condition were treated for 40 min with IL-2, IL-4, IL-7, IL-9, IL-21, or vehicle, then collected and processed for phosphoflow FACS analysis of pSTAT5 levels.

Phosphoflow FACS assay: Expanded Tregs were washed, and the stimulation beads were removed for 24 hrs before performing the assay. Tregs (5×10+/well) were plated in a 96-well U-bottom plate in 50 μL of complete RPMI with 10% FBS, without IL-2. After 24 hrs, IL-2, IL-4, IL-7, IL-9, IL-21, or control stimuli were prepared at 2× and added 1:1 v/v to wells containing the rested Treg cells for 40 min. Immediately following, Treg cells were fixed, washed with 1× RoboSep Buffer, and then permeabilized. Tregs were subsequently washed twice and stained in 1× RoboSep Buffer for the detection of pSTAT5. Stained cells underwent FACS analysis, and the FACS data were processed as described in Example 3. Representative histograms are overlaid for case of comparison.

FIG. 8A-8C depicts histograms of pSTAT5 signal intensity in recombinant IL-7Rα/IL-2Rβ receptor-expressing Tregs cultured with or without cytokine. Representative data for Tregs expressing tethered or untethered recombinant IL-7Rα/IL-2Rβ cytokine receptors and for control Tregs are overlaid on histograms for each cytokine treatment group (i.e., 40 min treatment with IL-2, IL-7, or vehicle). FIG. 8A depicts pSTAT5 signal intensity in Tregs cultured without cytokine. FIG. 8B depicts pSTAT5 signal intensity in Tregs cultured with IL-7. FIG. 8C depicts pSTAT5 signal intensity in Tregs that were exposed to IL-2 for 40 minutes. High pSTAT5 signal intensity in Tregs expressing tethered recombinant IL-7Rα/IL-2Rβ receptors with or without cytokine signifies a constant activation of the IL-2 signaling pathway within this Treg population. High pSTAT5 signal intensity in Treg cells expressing untethered recombinant IL-7Rα/IL-2Rβ receptors cultured with either IL-7 or IL-2 signifies activation of the IL-2 signaling pathway within this Treg population upon cytokine stimulation

FIG. 8D-8F depicts histograms of pSTAT5 signal intensity in Tregs expressing tethered, untethered, or no recombinant cytokine receptors. Representative data for Treg cells treated for 40 min with IL-7, IL-2, or vehicle are overlaid on histograms for each experimental group. FIG. 8D depicts pSTAT5 signal intensity in control Tregs. FIG. 8E depicts pSTAT5 signal intensity in Tregs expressing tethered recombinant cytokine receptors. FIG. 8F depicts pSTAT5 signal intensity in Tregs expressing untethered recombinant cytokine receptors. High pSTAT5 signal intensity in Tregs expressing tethered recombinant cytokine receptors cultured with or without cytokine signifies constitutive IL-2 signaling within this Treg population. High pSTAT5 signal intensity in Treg cells expressing untethered recombinant cytokine receptors cultured with either IL-7 or IL-2 signifies activation of the IL-2 signaling pathway upon stimulation of either the cognate cytokine, IL-7, or through canonical IL-2 receptor signaling.

FIG. 8G depicts representative histograms of pSTAT5 signal intensity in control Tregs or Tregs expressing untethered recombinant IL-4Rα/IL-2Rβ, IL-9Rα/IL-2Rβ, or IL-21Rα/IL-2Rβ receptors. Tregs were treated for 40 min with vehicle or their cognate cytokine, then collected and analyzed by phosphoflow for pSTAT5 levels. Treatment of recombinant receptor-expressing Tregs with cognate cytokine for 40 min induced an increase in pSTAT5 levels on par with control Tregs that were treated with IL-2, which confirms that each of these untethered recombinant cytokine receptors are capable of transducing IL-2 signaling similar to the native IL-2R in Tregs. FIG. 8H depicts representative histograms of pSTAT5 signal intensity in control Tregs or Tregs expressing tethered recombinant IL-4Rα/IL-2Rβ, IL-9Rα/IL-2Rβ, or IL-21Rα/IL-2Rβ receptors. Tethered recombinant cytokine receptors showed constitutively high pSTAT5 levels that were equivalent to control Tregs treated with IL-2.

FIGS. 8I-8K depict representative histogram (FIGS. 8I and 8K) and graphical representation (FIG. 8J) of Tregs expressing tethered recombinant IL-9Rα/IL-2Rβ receptors that were not treated with IL-2 compared to control Tregs treated with IL-2 or not treated with IL-2. FIGS. 8L-8M depict representative histogram (FIG. 8L) and graphical representation (FIG. 8M) of EGFR− negative Tregs selected from the transfection experiment as a negative control for the tethered recombinant IL-9Rα/IL-2Rβ receptor, compared to control Tregs treated with IL-2 or not treated with IL-2. Results from FIGS. 8I-8M demonstrated that Tregs expressing tethered recombinant IL-9Rα/IL-2Rβ receptors display constitutively activated (i.e., phosphorylated) STAT5 levels, such that treatment with different concentrations of IL-2 have a limited impact on pSTAT5 levels, whereas control Tregs display a dose-dependent pSTAT5 level in response to IL-2 concentration.

### Example 9: Recombinant Cytokine Receptors Activate Cytokine Dose-Dependent IL-2 Signaling in Regulatory T Cells

Example 9 demonstrates that IL-2 signal activation in Tregs expressing one of the recombinant IL-4Rα/IL-2Rβ, IL-7Rα/IL-2Rβ, IL-9Rα/IL-2Rβ, or IL-21Rα/IL-2Rβ receptors happens as a dose-dependent response to exogenous cognate cytokines (i.e., IL-4, IL-7, IL-9, or IL-21, respectively) independent of exogenous IL-2. Activation of this pathway is dose-dependent in a similar manner as canonical IL-2 signaling. The recombinant cytokine receptors still preserve the Treg responsivity and associated signaling dynamics of canonical Treg IL-2 signaling.

Treg cells expressing one of the untethered recombinant IL-4Rα/IL-2Rβ, IL-7Rα/IL-2Rβ. IL-9Rα/IL-2Rβ, or IL-21Rα/IL-2Rβ receptors were prepared using the same method as Example 4 above. At day 15, Treg cells were treated for 40 min with vehicle, 300 IU/mL IL-2, or the following IL-4, IL-7, IL-9, and IL-21 doses: 0.156 ng/ml, 0.625 ng/ml. 2.5 ng/ml. 10 ng/ml, or 100 ng/mL. Then these Treg cells were collected and processed for FACS analysis of STAT5 phosphorylation as described in Example 8 above. Representative histograms are overlaid for ease of comparison.

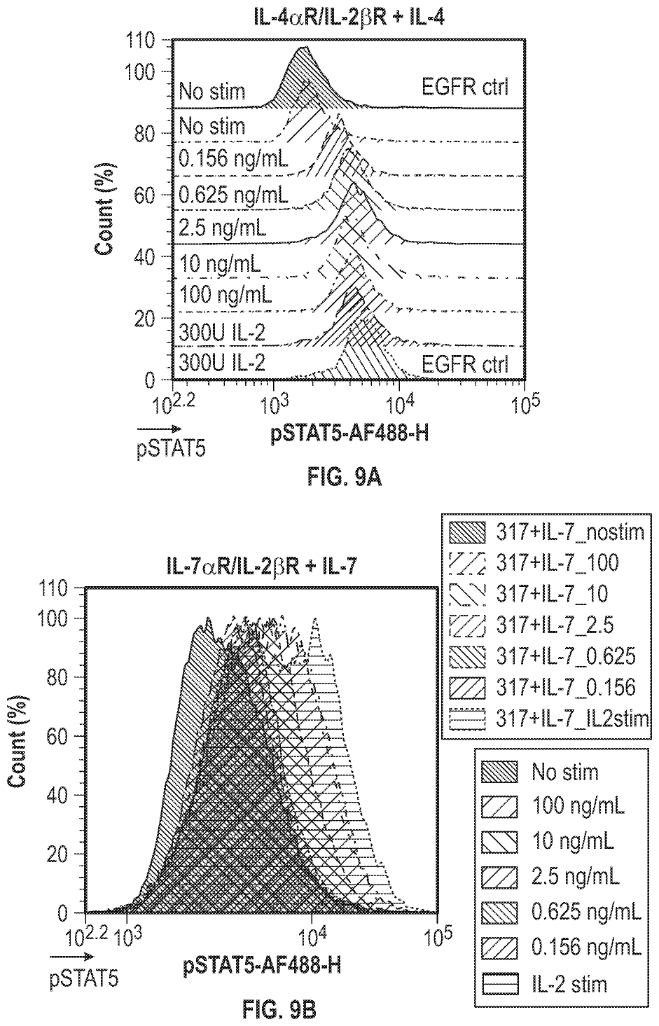

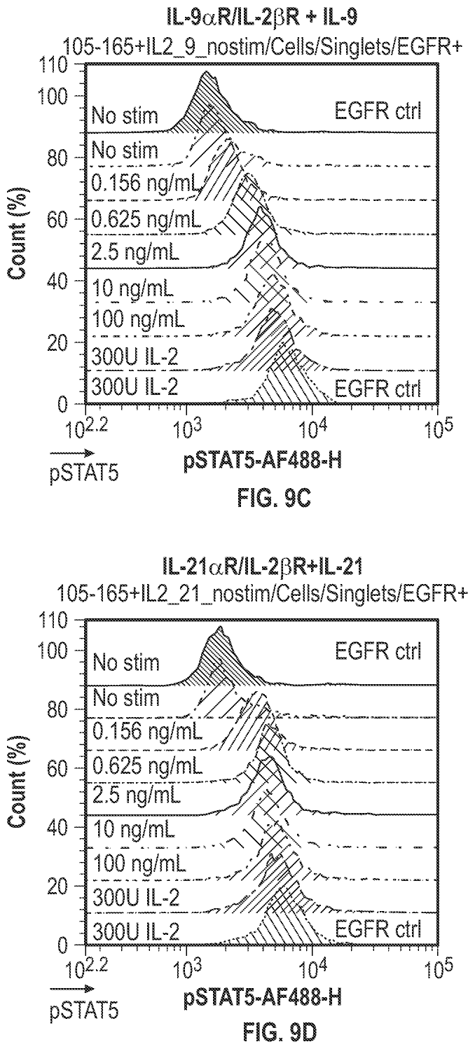

FIGS. 9A-9D depict the pSTAT5 signal intensity dose response to short-duration cytokine treatment in Tregs expressing untethered recombinant IL-4Rα/IL-2Rβ, IL-7Rα/IL-2Rβ. IL-9Rα/IL-2Rβ, or IL-21Rα/IL-2Rβ receptors. FIG. 9A depicts the IL-4 dose-dependent pSTAT5 response of Tregs that expressed recombinant IL-4Rα/IL-2Rβ receptors. FIG. 9B depicts the IL-7 dose-dependent pSTAT5 response of Tregs that expressed recombinant IL-7Rα/IL-2Rβ receptors. FIG. 9C depicts the IL-9 dose-dependent pSTAT5 response of Tregs that expressed recombinant IL-9Rα/IL-2Rβ receptors. FIG. 9D depicts the IL-21 dose-dependent pSTAT5 response of Tregs that expressed recombinant IL-21Rα/IL-2Rβ receptors. Cytokine treatment in each of these recombinant Tregs induced an increase in pSTAT5 signal intensity in a dose-dependent manner such that lower doses induced smaller increases in pSTAT5, and higher doses induced larger increases in pSTAT5. These results show that the IL-2 signaling sensitivity and dynamics are preserved in Tregs that express any of the recombinant IL-4Rα/IL-2Rβ. IL-7Rα/IL-2Rβ. IL-9Rα/IL-2Rβ, or IL-21Rα/IL-2Rβ receptors.

### Example 10: Tethered Recombinant Cytokine Receptors Induce Constitutive IL-2 Signaling in Regulatory T Cells Across an IL-2 Dose Curve

In contrast to the dose-dependency described in Example 9 above, Example 10 below describes constitutive IL-2 signal transduction in Tregs expressing tethered recombinant IL-7Rα/IL-2Rβ receptors. These Tregs display constitutive IL-2 signaling even without treatment of any exogenous cytokine.

Treg cells expressing control vectors or tethered recombinant IL-7Rα/IL-2Rβ receptors were prepared using the same method as Example 4 above. At day 8. Treg cells were treated for 40 min with vehicle or the following IL-2 doses: 0.156 U. 2.5 U, or 10 U, then collected and processed for FACS analysis of pSTAT5 as described in Example 8.

Representative histograms are overlaid for case of comparison.

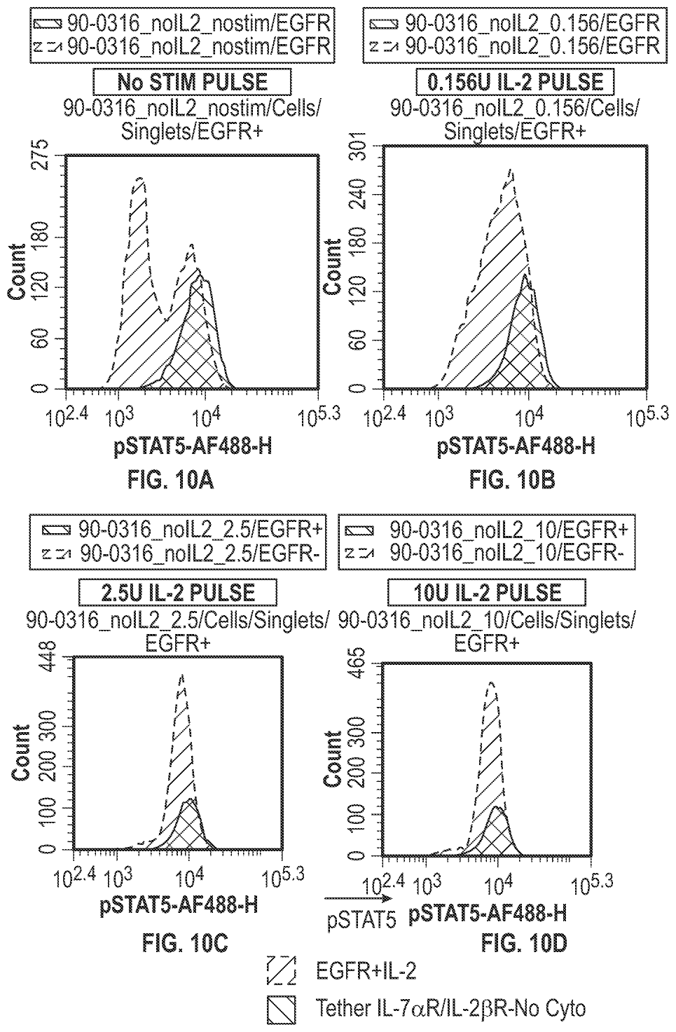

FIGS. 10A-10D depicts pSTAT5 signal intensity as a readout of IL-2 pathway activation in control Tregs and in Tregs expressing tethered recombinant IL-7Rα/IL-2Rβ receptors with or without IL-2 treatment at various doses. FIG. 10A depicts pSTAT5 signal intensity in Tregs without IL-2 treatment. FIG. 10B depicts pSTAT5 signal intensity in Tregs treated with 0.156 U IL-2. FIG. 10C depicts pSTAT5 signal intensity in Tregs treated with 2.5 U IL-2. FIG. 10D depicts pSTAT5 signal intensity in Tregs treated with 10 U IL-2. Across all treatment conditions, pSTAT5 signal intensity in Tregs expressing tethered recombinant IL-7Rα/IL-2Rβ receptors remained constant and high, whereas pSTAT5 signal intensity in control Tregs remained low in the absence of IL-2 and shifted up with intensity in an IL-2 dose-dependent manner. These results demonstrate high constitutive IL-2 signaling in Tregs expressing tethered recombinant IL-7Rα/IL-2Rβ receptors.

Control Tregs and Tregs expressing tethered recombinant IL-4Rα/IL-2Rβ, IL-7Rα/IL-2Rβ. IL-9Rα/IL-2Rβ, and IL-21Rα/IL-2Rβ receptors are cultured for 8 days post-transduction as described herein, then treated for 40 min with the following IL-2 doses: 0 U. 0.156 U. 2.5 U, or 10 U IL-2. These samples are assessed by FACS analysis for pSTAT5 signal intensity.

### Example 11: Regulatory T Cells Expressing Tethered Recombinant Cytokine Receptors Suppress Effector T Cells

Treg cells limit and suppress effector CD4+ and CD8+ T cell activity (e.g., proliferation, cytolysis) to prevent indiscriminate destruction of healthy tissues. As shown in Example 11. Tregs expressing tethered recombinant IL-7Rα/IL-2Rβ receptors displayed equivalent suppressive behavior towards effector CD4+ and CD8+ T cells without cytokine stimulus as the control Tregs display when cultured with IL-2. Therefore, when expressed in Tregs, the recombinant cytokine receptors are able to support and promote Treg suppressive functions in environments that are limited or lacking in IL-2 on par with biologically relevant control Treg suppression levels in environments rich in exogenous IL-2.

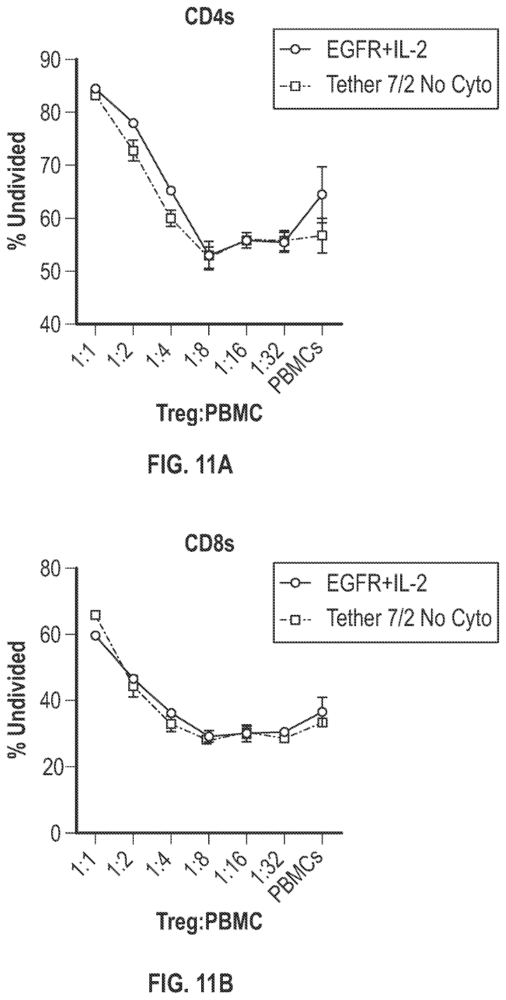

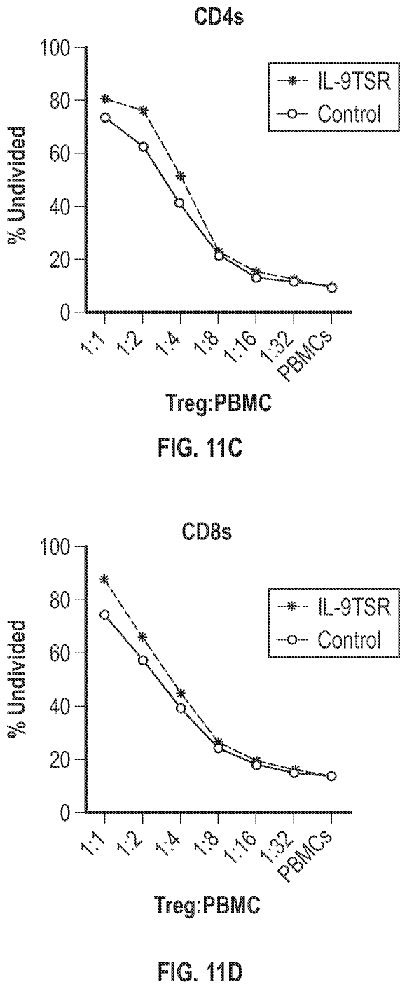

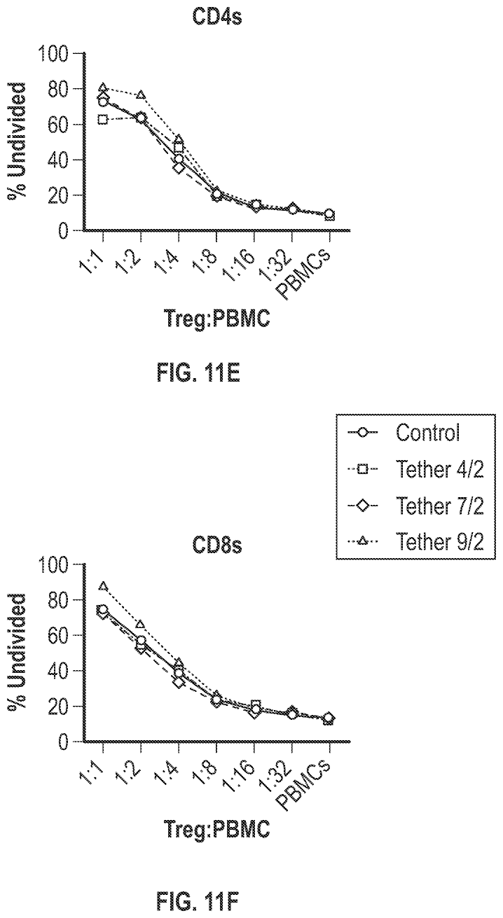

Treg suppression assay: Treg cells expressing control vectors or tethered recombinant IL-7Rα/IL-2Rβ receptors were prepared using the same method as described in Example 3 above for 14 days from fresh human leukopaks in a 96-well U-bottom plate. Tregs expressing tethered recombinant IL-7Rα/IL-2Rβ receptors (FIGS. 11A-11B) or IL-9Rα/IL-2Rβ receptors (FIGS. 11C-11D) were cultured without cytokine, whereas control Tregs were cultured with IL-2. PBMCs were used as Treg responder cells in co-culture with Tregs. PBMCs were isolated as described in Example 3 and then labelled with Cell Trace Violet (CTV) as a cell proliferation dye. PBMCs (5×10⁴/well) were co-cultured with the following Treg effector-to-PBMC target ratios: 1:1, 1:2, 1:4, 1:8, 1:16, and 1:32. The co-culture was activated for 72 hrs using soluble anti-CD3 and anti-CD28 activation beads. The suppressive function of Treg cells was determined by measuring undivided CD4+ and CD8+ T cells after 72 hours.

FIG. 11A depicts the suppressive activity of Tregs expressing tethered recombinant IL-7Rα/IL-2Rβ receptors on CD4+ T cells. FIG. 11B depicts the suppressive activity of Tregs expressing tethered recombinant IL-7Rα/IL-2Rβ receptors on CD8+ T cells. Tregs expressing tethered recombinant IL-7Rα/IL-2Rβ receptors cultured without cytokine behaved in the same capacity as control Tregs cultured with IL-2: both Treg groups suppressed CD4+ and CD8+ T cell division in a ratio-dependent response, such that a higher number of CD4+ or CD8+ T cells to each Treg cell diminished the Treg suppressive impact and resulted in a lower percentage of undivided CD4+ or CD8+ T cells. In contrast, at a one-to-one ratio of Tregs and CD4+ or CD8+ T cells, nearly none of the CD4+ T cells and only about 40% of the CD8+ T cells were able to divide. Therefore, Tregs expressing tethered recombinant IL-7Rα/IL-2Rβ receptors demonstrate constitutive suppressive action similar to the scope of results from Examples 5-8 and 10.

FIG. 11C depicts the suppressive activity of Tregs expressing tethered recombinant IL-9Rα/IL-2Rβ receptors on CD4+ T cells. FIG. 11D depicts the suppressive activity of Tregs expressing tethered recombinant IL-9Rα/IL-2Rβ receptors on CD8+ T cells. Tregs expressing tethered recombinant IL-9Rα/IL-2Rβ receptors cultured without cytokine behaved in the same capacity as control Tregs cultured with IL-2: both Treg groups suppressed CD4+ and CD8+ T cell division in a ratio-dependent response. At a one-to-one ratio of Tregs expressing tethered recombinant IL-9Rα/IL-2Rβ receptors and CD4+ or CD8+ T cells, only about 20% of the CD4+ T cells and CD8+ T cells were able to divide.

FIG. 11E provides a comparison of the suppressive activity of Tregs expressing tethered IL-4Rα/IL-2Rβ, IL-7Rα/IL-2Rβ, or IL-9Rα/IL-2Rβ recombinant cytokine receptors on CD4+ T cells. FIG. 11F depicts the suppressive activity of Tregs expressing tethered IL-4Rα/IL-2Rβ, IL-7Rα/IL-2Rβ, or IL-9Rα/IL-2Rβ recombinant cytokine receptors on CD8+ T cells. Tregs expressing tethered recombinant cytokine receptors cultured without cytokine behaved in the same capacity as control Tregs cultured with IL-2: all Treg groups suppressed CD4+ and CD8+ T cell division in a ratio-dependent response. At a one-to-one ratio of Tregs expressing tethered recombinant cytokine receptors and CD4+ or CD8+ T cells, only about 20% of the CD4+ T cells and CD8+ T cells were able to divide. Of note, Tregs expressing tethered IL-9Rα/IL-2Rβ recombinant cytokine receptors demonstrated a higher degree of CD4+ and CD8+ T cell suppression at 1:1, 1:2, and 1:4 Treg to PBMC ratios.

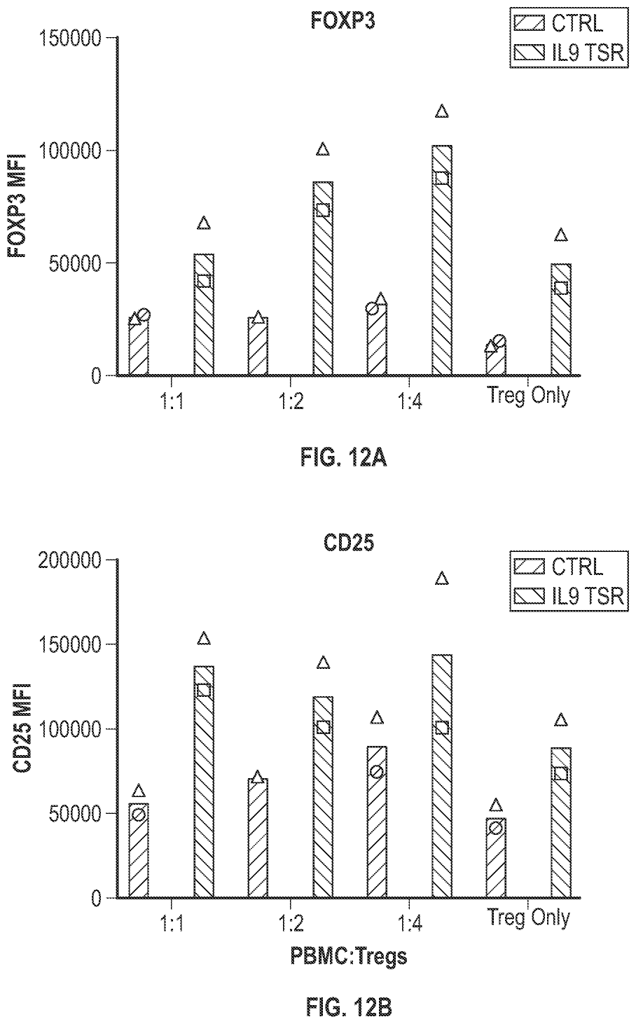

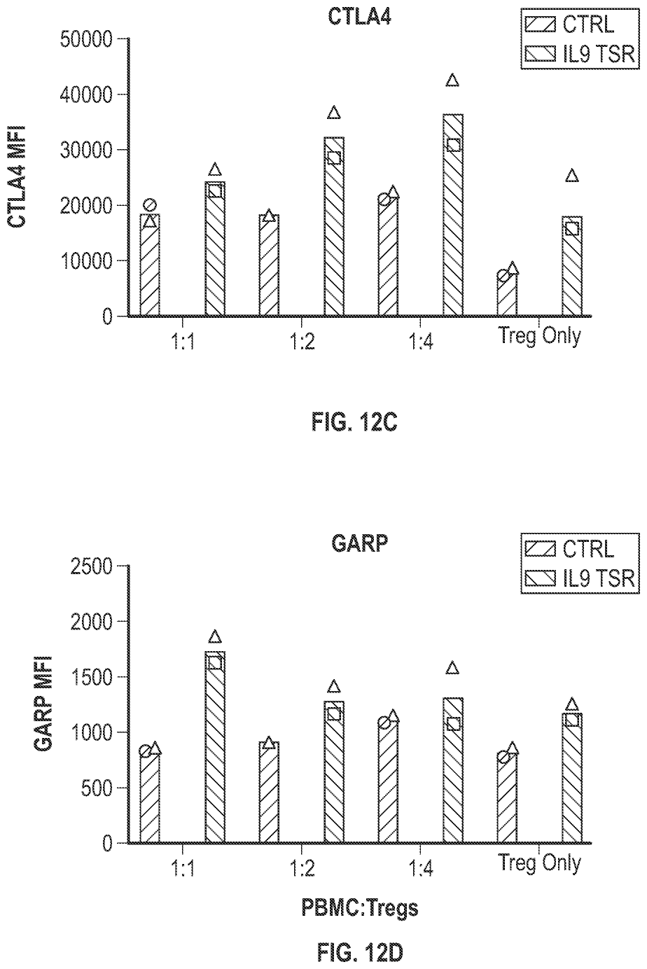

Furthermore, the MFI of FOXP3, CD25, CTLA4, and GARP were analyzed as described above. Tregs expressing tethered IL-9Rα/IL-2Rβ recombinant cytokine receptors were cultured alone or co-cultured with PBMCs at a 1:1, 1:2, and 1:4 ratio of PBMCs to Tregs. Cells were then harvested, stained for and analyzed by flow cytometry, as described above, and the MFI quantified on Treg cells across all four groups. Treg cell levels of FOXP3 (FIG. 12A), CD25 (FIG. 12B), CTLA4 (FIG. 12C), and GARP (FIG. 12D) on co-cultured Tregs expressing tethered recombinant IL-9Rα/IL-2Rβ receptors and PBMCs demonstrated significantly higher levels of each Treg marker (i.e., FOXP3 and CD25) and Treg activation marker (i.e., CTLA4 and GARP) compared to control Tregs at each tested target-to-effector ratio. These results suggest an increase in suppressive function of the Tregs expressing tethered recombinant IL-9Rα/IL-2Rβ receptors over control Tregs.

### Example 12: Regulatory T Cells Expressing IL-21Rα/IL-2Rβ Tethered Recombinant Cytokine Receptors Suppress Effector T Cells

Control Tregs and Tregs expressing tethered IL-21Rα/IL-2Rβ recombinant cytokine receptors are cultured as described herein, then are co-cultured with CD4+ or CD8+ T cells in the same manner as described in Example 11 above. These samples are analyzed for the percentage of CD4+ or CD8+ T cell division to assess the suppressive capacity of Tregs expressing recombinant cytokine receptors as compared to control Tregs. Levels of FOXP3. CD25. CTLA4, and GARP are analyzed using flow cytometry as described above.

Control Tregs and Tregs expressing untethered IL-21Rα/IL-2Rβ recombinant cytokine receptors are cultured as described herein in the presence of their cognate cytokine, then are co-cultured with CD4+ or CD8+ T cells in the same manner as described above. These samples are analyzed for the percentage of CD4+ or CD8+ T cell division to assess the suppressive capacity of Tregs expressing recombinant cytokine receptors as compared to control Tregs. Levels of FOXP3, CD25, CTLA4, and GARP are analyzed using flow cytometry as described above.

### Example 13: Regulatory T Cells Expressing Tethered Recombinant Cytokine Receptors Display Treg Profile After Multiple Rounds of Antigen Stimulation

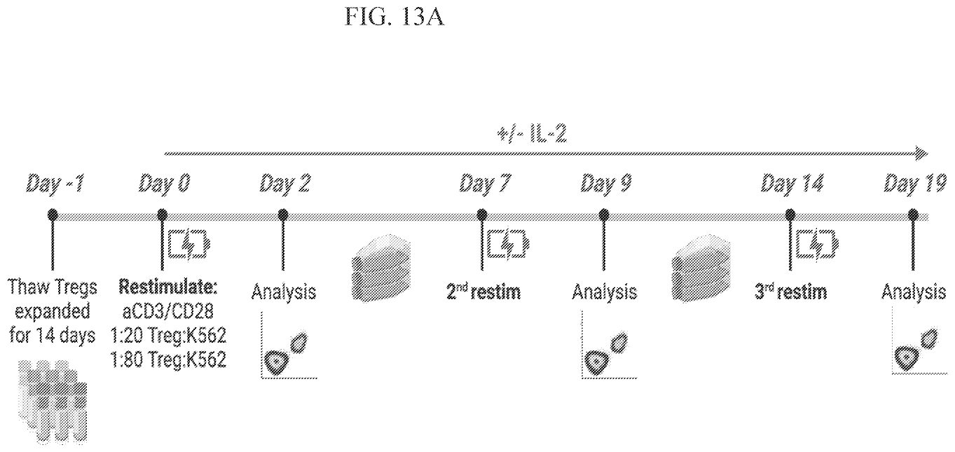

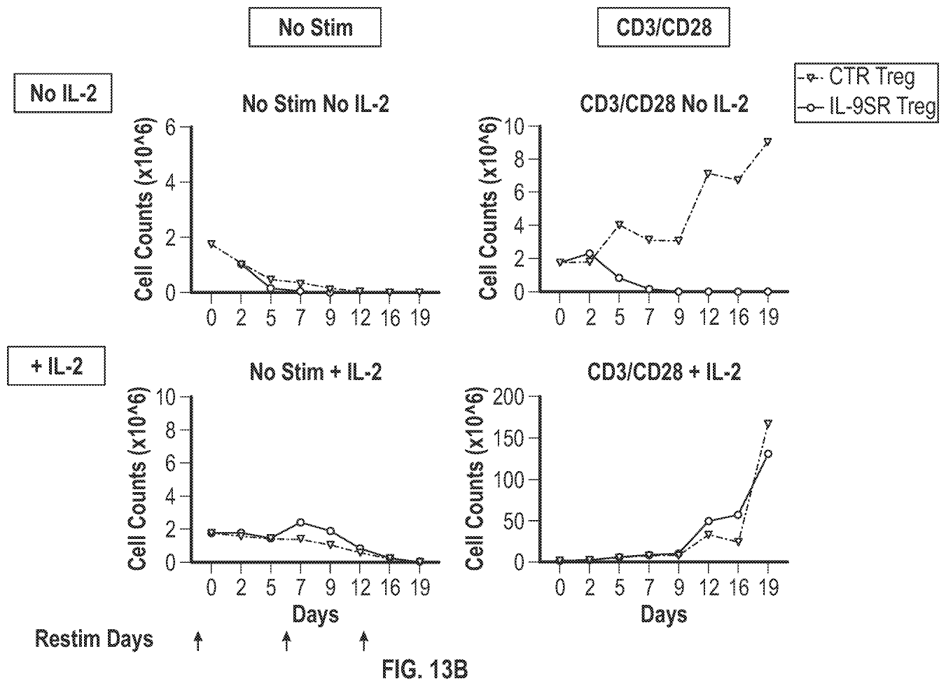

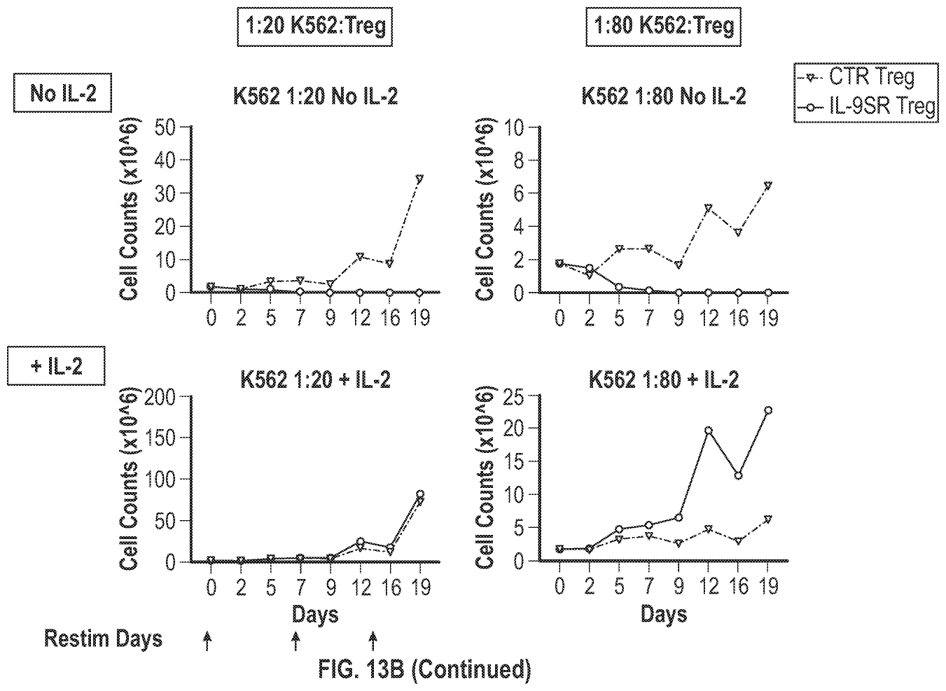

Tregs were cultured in vitro alone, with anti-CD3/anti-CD28 antibody, or co-cultured with CD19-presenting K562 at 1:20 or 1:80 Treg: K562 ratios as shown in FIG. 13A. In brief. Treg cells were thawed and allowed to recover for one day, then activated by anti-CD3/anti-CD28 antibody stimulation or by co-culture with K562 cells that express the CD19 antigen on Day 0. On Day 2, some cells were harvested and analyzed by flow cytometry for Treg phenotype and cytokine production. The remaining cells continued to grow and were re-stimulated on Day 7. Then on Day 9, some cells were harvested and analyzed by flow cytometry for Treg phenotype and cytokine production. The remaining cells continued to grow in vitro, and on Day 14, the Tregs were re-stimulated one file time and the cells harvested for flow cytometric analyses on Day 19.

As shown in FIG. 13B. Tregs expressing tethered recombinant IL-9Rα/IL-2Rβ receptors proliferated after each round of cell stimulation, including both anti-CD3/anti-CD28 antibody stimulation and antigen-specific TCR activation, in both the absence and presence of IL-2. However, control Tregs only proliferated upon combined stimulation conditions and IL-2 treatment.

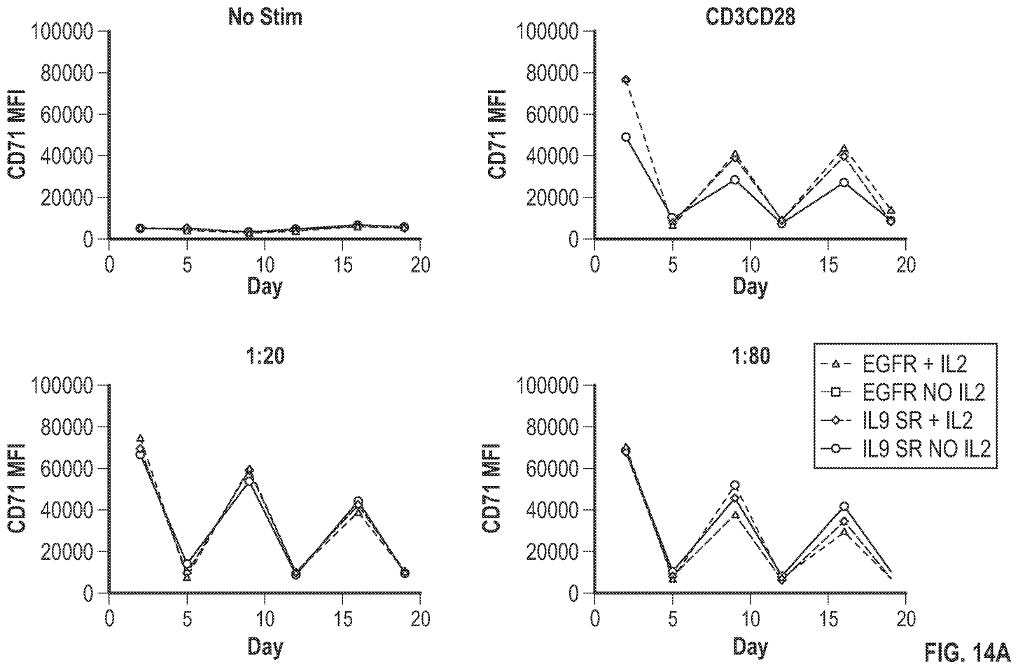

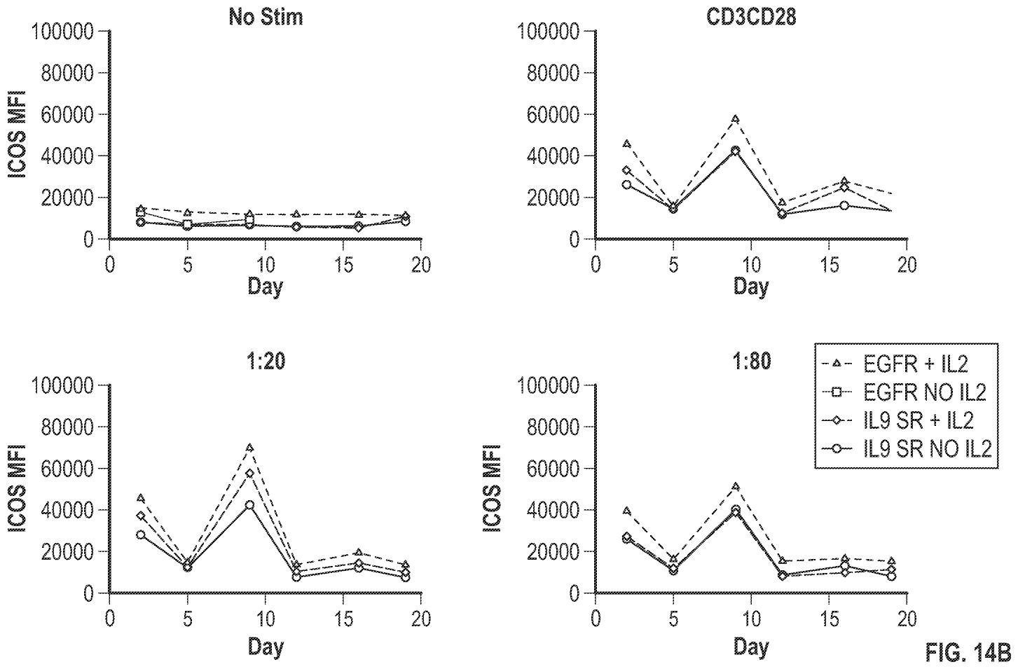

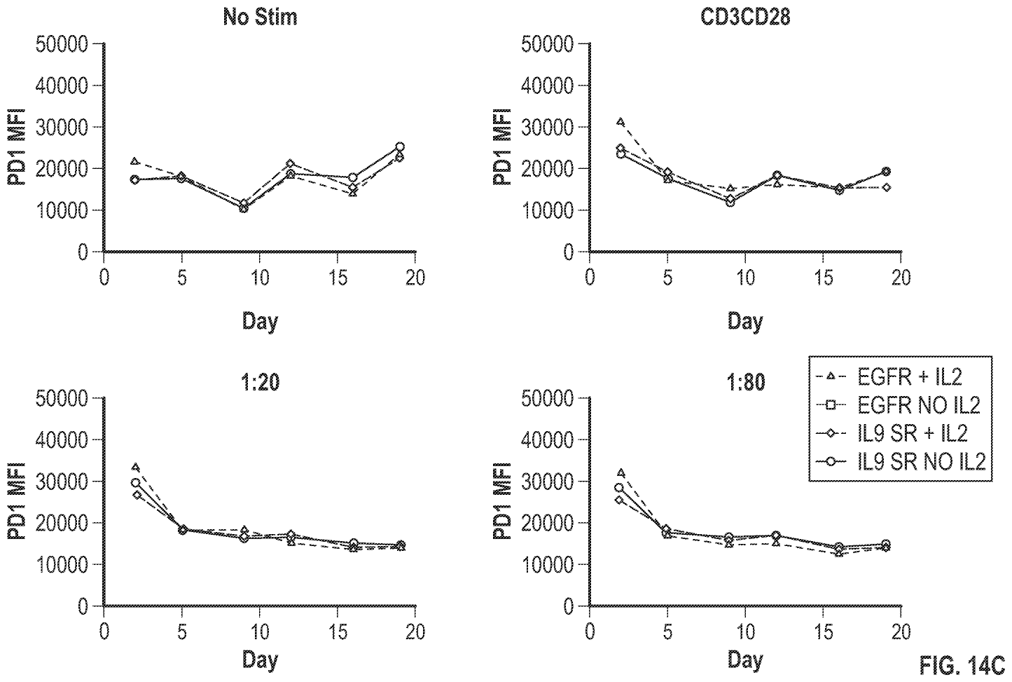

Markers of Tregs, CD71 (FIG. 14A) and ICOS (FIG. 14B), peaked after each round of stimulation but were not present in unstimulated Treg cells. PD-1 levels (FIG. 14C) generally remained low and did not peak or otherwise respond to Treg stimulation. Treg expression of each marker was similar across all conditions, such that there were no observable differences between control Tregs and Tregs expressing tethered recombinant IL-9Rα/IL-2Rβ receptors.

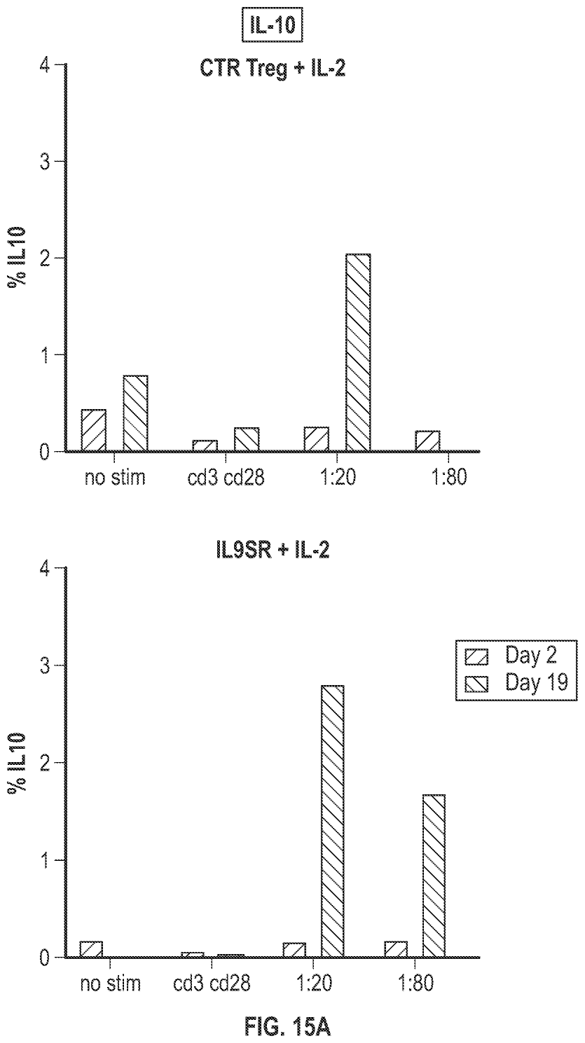

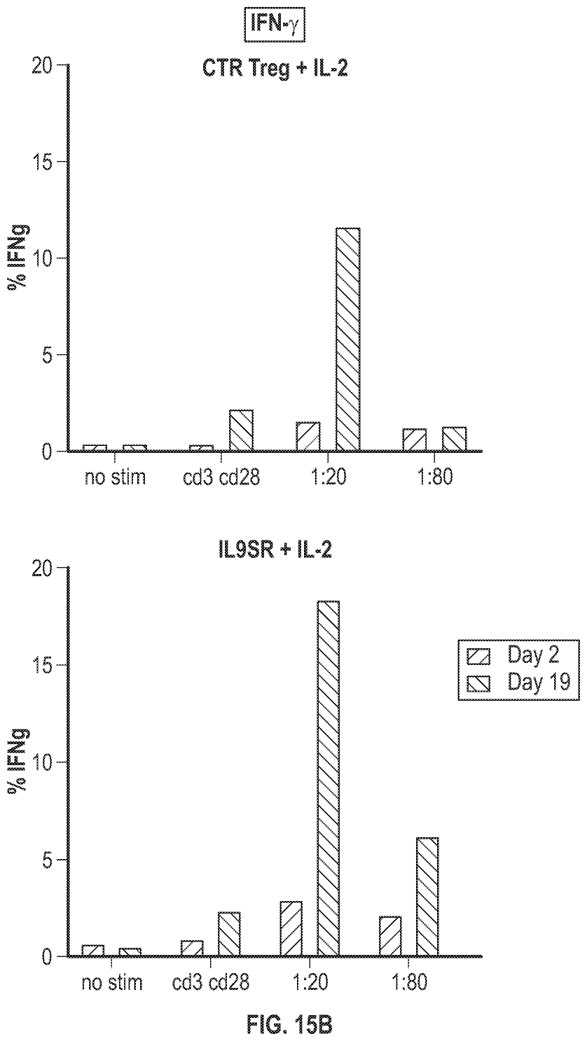

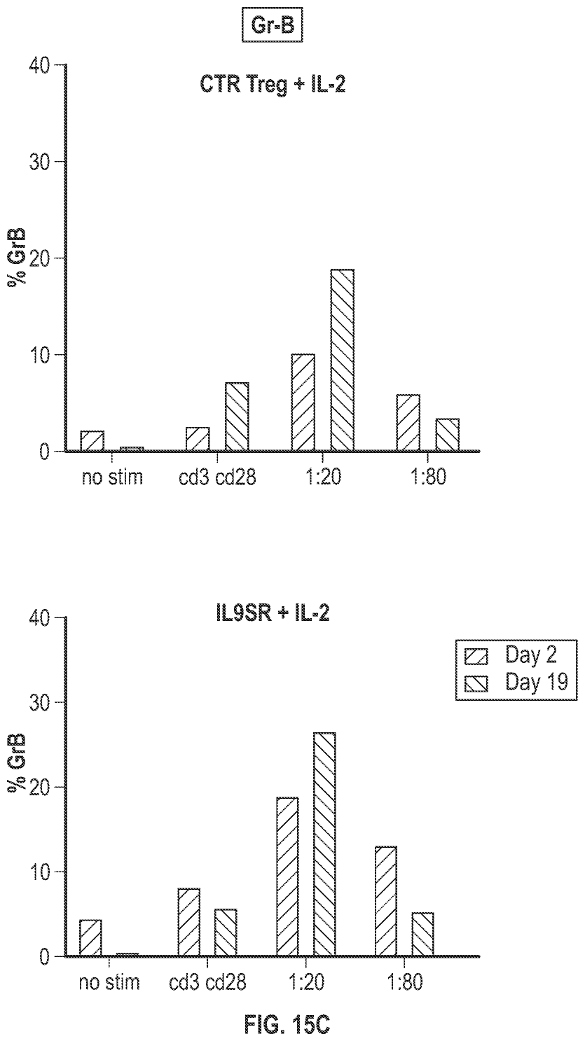

Furthermore, FIGS. 15A-15C show the production levels of IL-10, IFN-γ, and Granzyme B (GrB), respectively, in Tregs expressing tethered recombinant IL-9Rα/IL-2Rβ receptors by Day 2 and Day 19 in co-culture with K562-CD19 cells in the presence of IL-2. These results demonstrate that Tregs expressing tethered recombinant IL-9Rα/IL-2Rβ receptors are able to functionally re-activate upon multiple rounds of restimulation.

### Example 14: Engraftment and Expansion of Regulatory T Cells Expressing Tethered Recombinant Cytokine Receptors In Vivo

Immunocompromised NOD scid gamma (NSG) mice were injected intravenously with Tregs expressing IL-4 tethered IL-4Rα/IL-2Rβ. Tregs expressing IL-7 tethered IL-7Rα/IL-2Rβ. Tregs expressing IL-9 tethered IL-9Rα/IL-2Rβ, or control Tregs expressing the EGFR tag protein. On Day 13, mice were euthanized, and blood, spleen, lung, and liver were harvested to assess for EGFR+ cell count (i.e., how many cells were the adoptively transferred Tregs) to test for Treg engraftment, expansion, and persistence in vivo.

FIG. 16A shows the cell count of Tregs within the spleen, lung, and liver after adoptive cellular transfer into immunocompromised mice. Across all three tissues. Tregs expressing IL-9 tethered IL-9Rα/IL-2Rβ recombinant cytokine receptors showed the highest degree of cell engraftment, expansion, and persistence in vivo. Tregs expressing IL-4 tethered IL-4Rα/IL-2Rβ recombinant cytokine receptors showed about two-fold higher engraftment, expansion, and persistence in vivo compared to control Tregs. The engraftment, expansion, and persistence in vivo of Tregs expressing IL-7 tethered IL-7Rα/IL-2Rβ recombinant cytokine receptors was approximately the same as that of control Tregs.

These results demonstrate that, while Tregs expressing each recombinant cytokine receptor is capable of surviving, proliferating, and persisting in vitro as shown in FIGS. 5A-5H, these receptors display variable degrees of engraftment and expansion in vivo. Of note. Tregs expressing IL-4 tethered IL-4Rα/IL-2Rβ, IL-7 tethered IL-7Rα/IL-2Rβ, and IL-9 tethered IL-9Rα/IL-2Rβ recombinant cytokine receptor were each capable of persisting in vivo, even as the degree of such persistence differed.

As shown in FIG. 16B, across all four tissues, NSG mice injected with Tregs expressing tethered recombinant IL-9Rα/IL-2Rβ receptors showed significant EGFR+ cell count compared to NSG mice injected with control Tregs, thereby confirming the in vitro data as well as the data in FIG. 16A and demonstrating Treg engraftment and expansion in the absence of exogenous IL-2 in an in vivo murine model.

FIG. 16C depicts the expansion of control and tethered recombinant IL-9Rα/IL-2Rβ receptor-expressing Tregs over time. In brief, control or tethered recombinant IL-9Rα/IL-2Rβ receptor-expressing Tregs were unstimulated, anti-CD3/anti-CD28 stimulated, or CAR-activated at Days 0, 7, and 14. Tregs were cultured in either the absence or presence of IL-2, and then the cell count was calculated over time. Without IL-2, only cells that were antibody-stimulated or CAR-activated showed prolonged Treg proliferation.

Furthermore, results from FIG. 16D (graphical representation) and FIG. 16E (FACS plots) assessing the total number of human immune cells and percent that were FOXP3+/HELIOS+ Tregs circulating in the blood of the immunocompromised mice at day 15 after intravenous injection, showed that Tregs that expressed the IL-9 tethered IL-9Rα/IL-2Rβ recombinant cytokine receptor persist in vivo and have a stable phenotype both with and without IL-2 treatment compared to control Tregs without IL-2 treatment.

### Example 15: Functional Activity of Regulatory T Cells Expressing Recombinant Cytokine Receptors In Vivo

After confirming the in vivo capacity of the Tregs expressing a recombinant cytokine receptor to engraft, survive, and proliferate, including across various tissues that contain a robust immunological presence, the suppressive activity of the Tregs was analyzed.

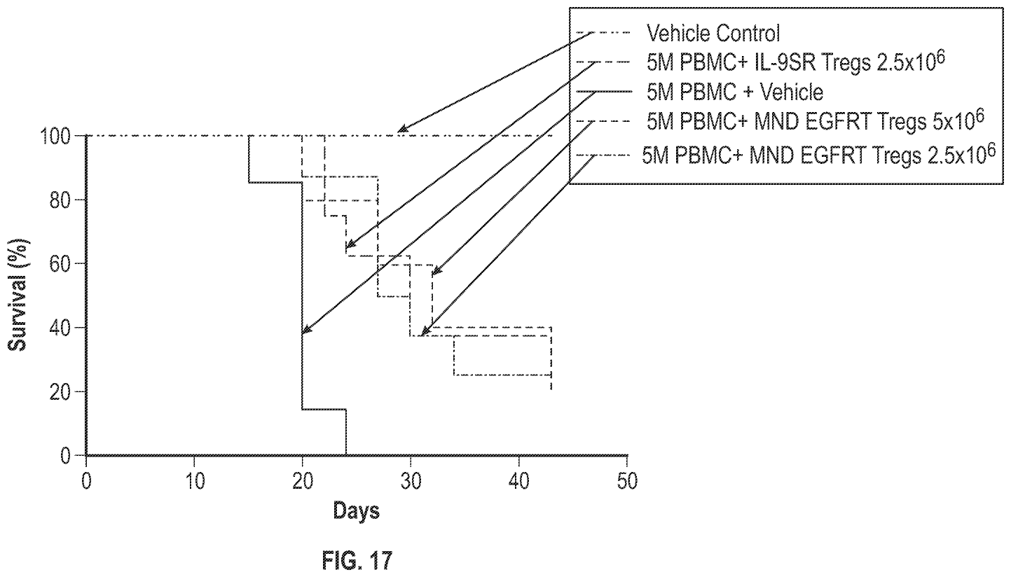

Treg suppressive activity was then tested to compare the ability of control Tregs and Tregs expressing tethered IL-9Rα/IL-2Rβ recombinant cytokine receptors to suppress conventional T cells in an immunocompromised mouse model, i.e., to prevent the development of graft versus host disease (GVHD) within the mice. In short, NSG mice were intravenously injected with vehicle, 5×10⁶ PBMC and vehicle, 5×10⁶ PBMC and 2.5×10⁶ control Tregs, 5×10⁶ PBMC and 5×10⁶ control Tregs, or 5×10⁶ PBMC and 2.5×10⁶ Tregs expressing tethered IL-9Rα/IL-2Rβ recombinant cytokine receptors and then observed over time for survival post-injection. FIG. 17 provides a survival curve of the mice in each group. Mice that were given 5×10⁶ PBMC and 2.5×10⁶ Tregs expressing tethered IL-9Rα/IL-2Rβ recombinant cytokine receptors showed a similar survival curve to mice that were given 5×10⁶ PBMC and 5×10⁶ control Tregs (twice as many control Tregs as experimental Tregs), showing the increased functional efficacy of the experimental Tregs expressing tethered IL-9Rα/IL-2Rβ recombinant cytokine receptors in vivo.

### Example 16: Alternative IL-2 Independent Strategies

A number of alternative strategies for achieving IL-2 independent Tregs were tested. As further explained below, none of these alternatives produced suitable IL-2 independent Treg cells.

Constitutively active STAT5 Treg model: The activation of STAT5 via its phosphorylation is downstream of IL-2 receptor binding its ligand. As a result, JAK1 and/or JAK3 bind to and phosphorylate STAT5, thereby activating STAT5 for nuclear translocation and binding to its target DNA sequences for gene regulation. In order to test for the efficacy of targeting STAT5 activation status to promote Treg proliferation and downstream function (e.g., effector T cell suppression), a FoxP3^{Cre-ERT2} mouse was crossed to the Rosa^{Stat5bCA} mouse in order to generate the Rosa^{Stat5bCA}FoxP3^{Cre-ERT2} mouse with inducible STAT5 constitutive activity (CA-STAT5 or CA-STAT5b). These mice were then administered a myelin basic protein peptide (MBP) fragment that induces an autoimmune response directed to the myelin sheath surrounding motor neurons as a model of multiple sclerosis (known as experimental autoimmune encephalomyelitis, or EAE). Mice were scored on the standard EAE scale of 1-5 (see Table 3 below) after immunization, up until Day 21.

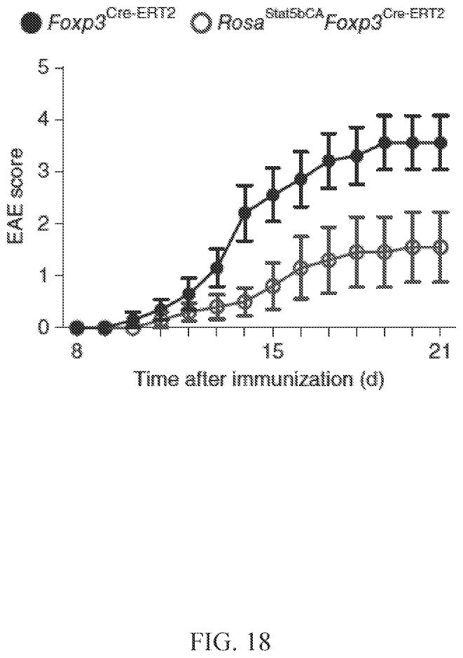

Mice with constitutively active STAT5 showed significant reduction in EAE score, indicating that STAT5 activity was protective against a neurological autoimmune disorder, possibly via its role in Treg activation, as shown in FIG. 18.

TABLE 3
EAE scores

| Clinical Score | Symptoms |
|---|---|
| 0 | No clinical signs; normal activity |
| 1 | Limp tail or hind limb weakness, but not both |
| 2 | Limp tail and hind limb weakness |
| 3 | Partial hind limb paralysis |
| 4 | Complete hind limb paralysis |
| 5 | Moribund state; death by EAE |

CA-STAT5 Tregs were then harvested from the mice and compared to wildtype (WT) Tregs that were treated or untreated with IL-2 in vitro. Cells were then counted to assess proliferation/expansion, assessed by flow cytometric methods described in the Examples above to analyze the Treg profile, co-cultured with CD4+ or CD8+ T cells to analyze suppressive activity, or injected into a mouse model of graft versus host disease (GVHD) to test the impact of the CA-STAT5 Tregs on effector T cells in vivo.

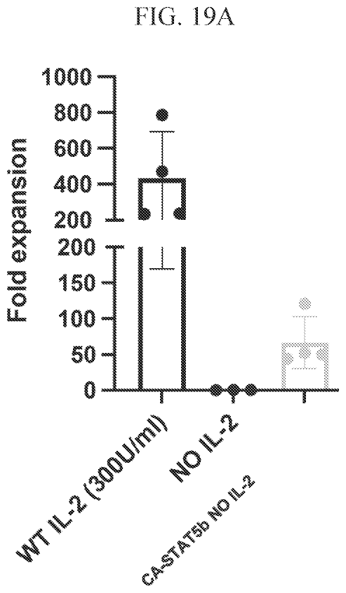

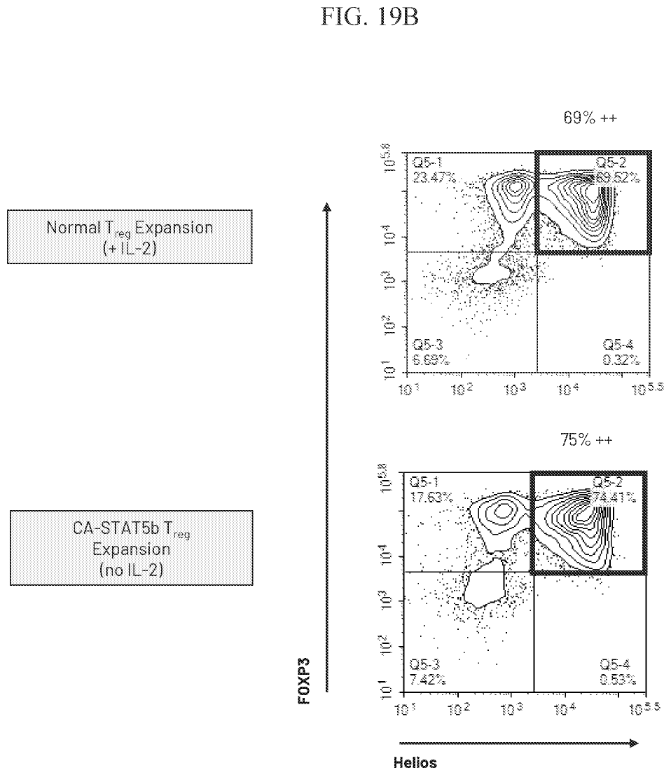

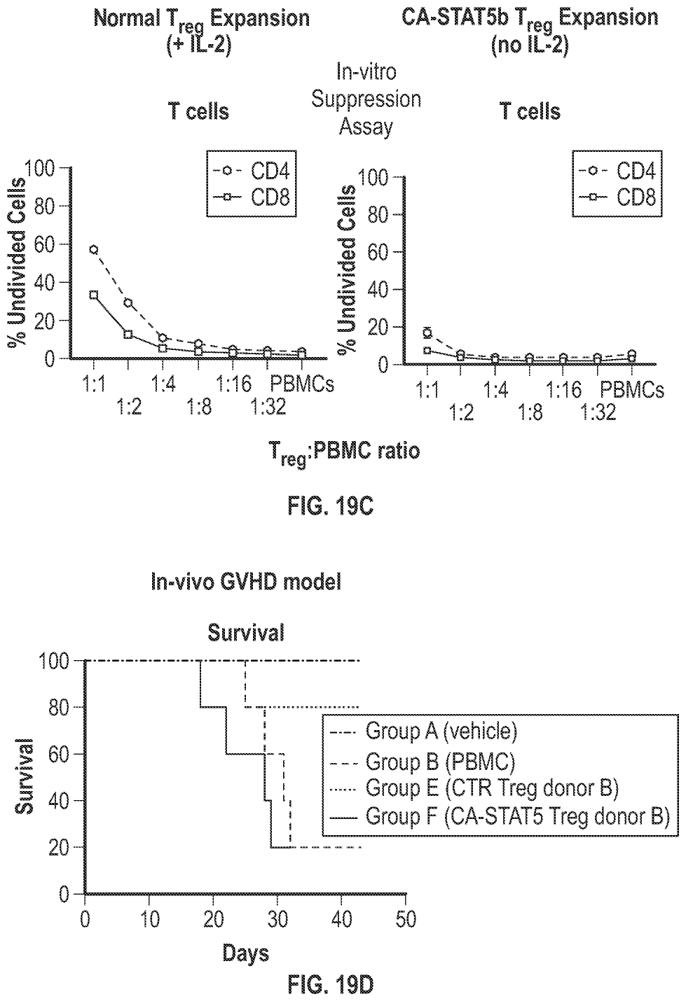

FIG. 19A depicts the expansion of CA-STAT5 Tregs in vitro, albeit at a much-reduced level compared to WT Tregs treated with IL-2. FIG. 19B demonstrates that CA-STAT5 Tregs display a normal Treg phenotype using FOXP3 and HELIOS as Treg markers, both with and without IL-2 treatment. However, FIG. 19C shows a decreased level of CD4+ or CD8+ T cell suppression in co-culture compared to control Tregs. This reduced suppressive activity was confirmed in vivo in FIG. 19D, where the mice receiving an injection of CA-STAT5 Tregs showed a lower percent of overall survival compared to control Tregs by 30-40 days.

Furthermore, RNA sequencing was performed on RNA samples that were isolated from wildtype Tregs and CA-STAT5 Tregs to compare gene signatures. Standard bioinformatic analysis was performed on the RNA sequencing results in order to identify any statistically significant differentially expressed genes (DEGs) between the two conditions.

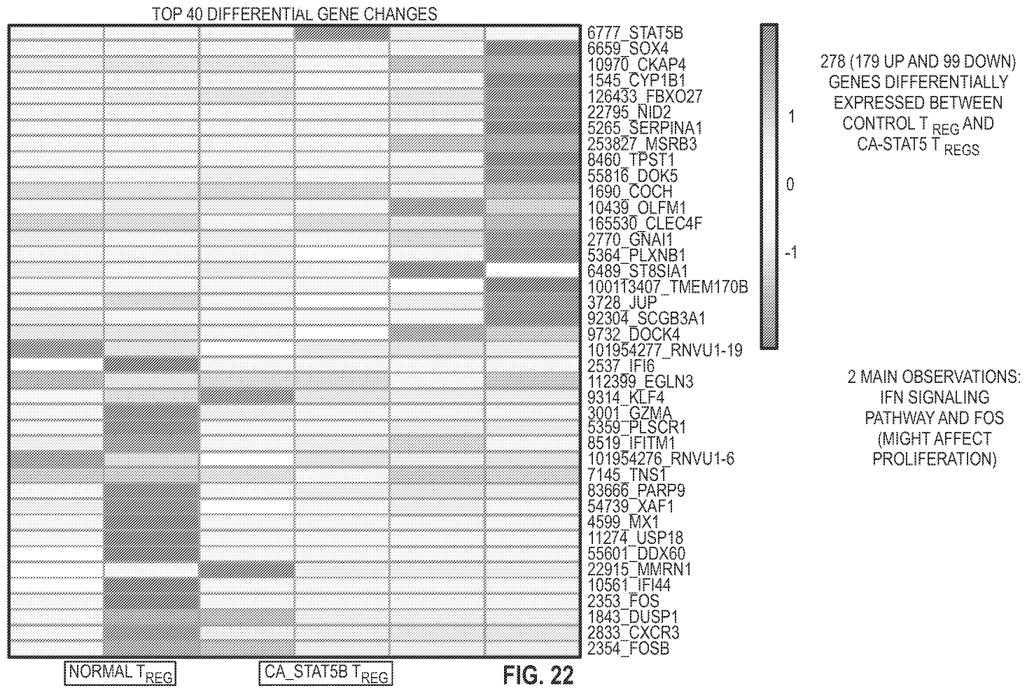

278 DEGs were identified in CA-STAT5 Tregs compared to wildtype Tregs, including 99 downregulated and 179 upregulated DEGS in CA-STAT5 Tregs compared to wildtype Tregs. FIG. 22 shows a heatmap of the top 40 DEGs analyzed by RNA sequencing analysis. Of these DEGS, the main pathways identified from these analyses include interferon pathway family members and FOS pathway family members.

The results from this model demonstrate that the CA-STAT5 cells expanded in the absence of IL-2, albeit not to the same level as wild-type cells treated with IL-2, and the CA-STAT5 cells exhibited lower levels of CD4+ and CD8+ T cell suppression compared to control Tregs. Accordingly, this model demonstrates that the results presented in FIGS. 1A-17 and Examples 1-15 above are unique and surprising.

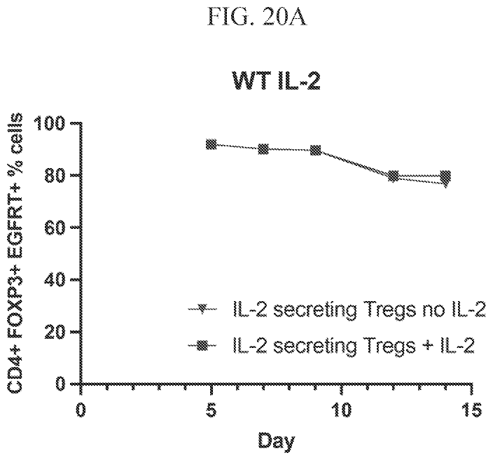

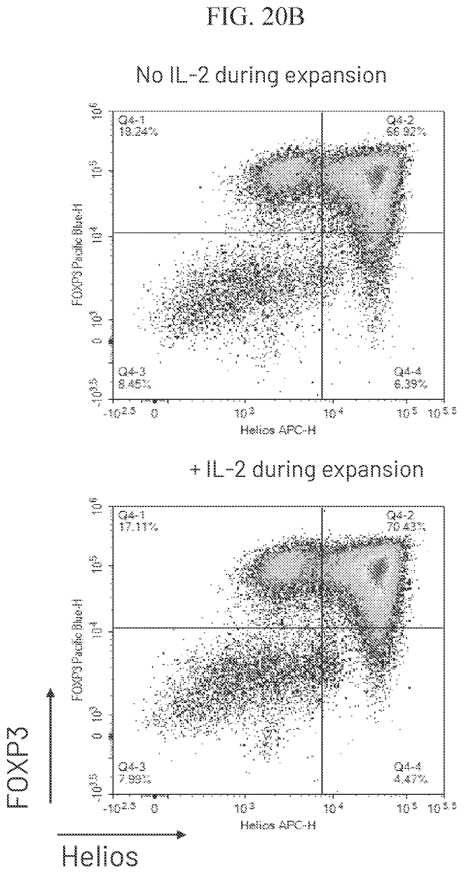

IL-2 secreting Treg model: Tregs were generated that produced and secreted IL-2 to provide IL-2 in an autocrine loop with the goal of self-supporting Treg proliferation. However, when tested in vitro, IL-2 secreting Tregs showed limited expansion (Data not shown). FIG. 20A shows that the IL-2 secreting Tregs display the same percentage of IL-2 secreting Tregs over time when cultured with versus without exogenous IL-2, including the same 10% decrease in Tregs over time. FIG. 20B demonstrates that IL-2 secreting Tregs display a normal Treg phenotype using FOXP3 and HELIOS as Treg markers, both with and without IL-2 treatment. However, in a co-culture experiment with PBMCs, the IL-2 secreting Tregs failed to suppress effector T cells, similarly to the CA-STAT5 Tregs (Data not shown). In line with the CA-STAT5 Treg model, this model also demonstrates that the results presented in FIGS. 1A-17 and Examples 1-15 above are unique and surprising.

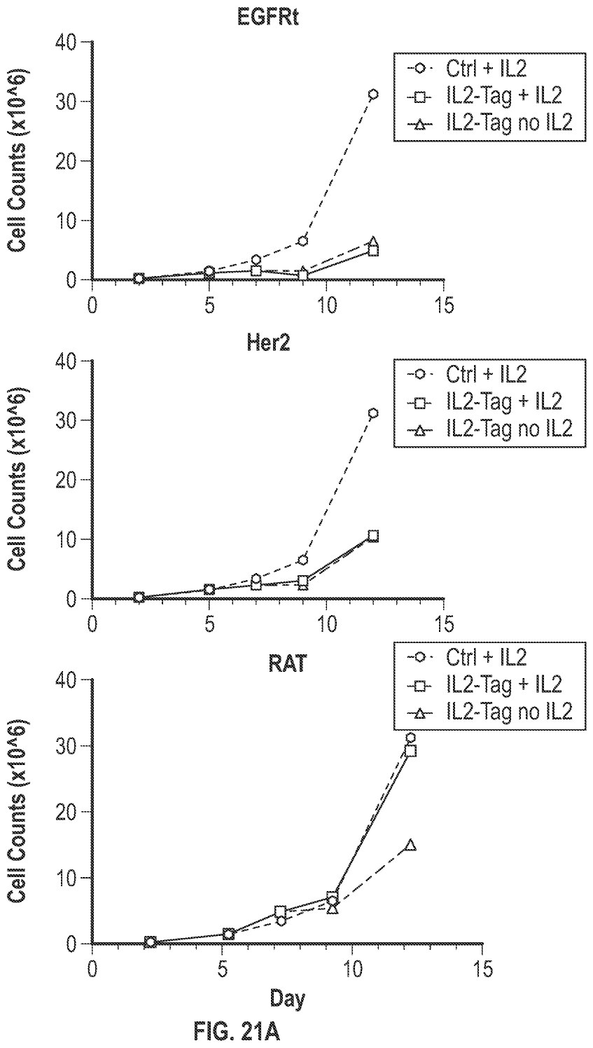

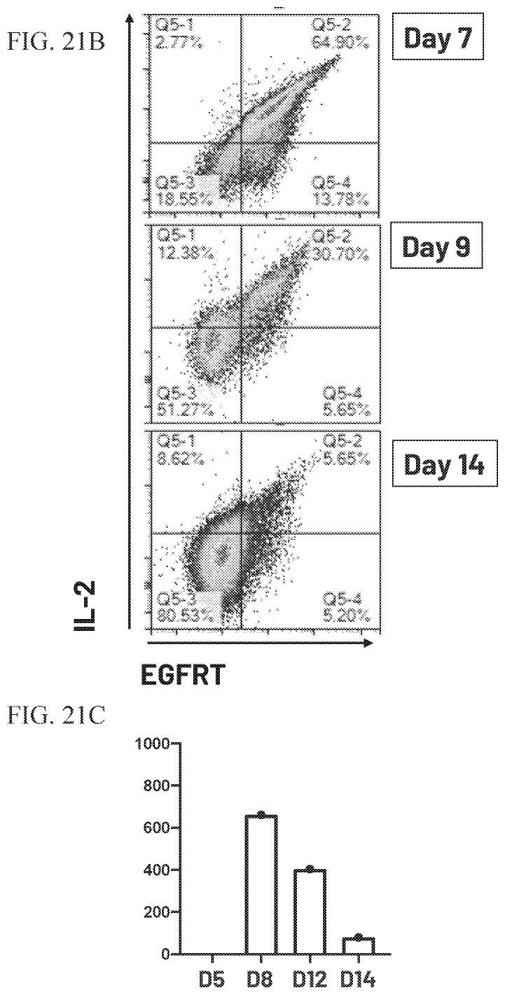

IL-2/Protein-tag tethered Treg model: Tregs were generated where IL-2 was tethered to a protein tag, such as EGFRt. Her2, or IL5Rα tags. This construction method allowed IL-2 to be anchored to the cell membrane to recruit the IL-2 receptor complex and activate the receptor pathway, including STAT5 activation. These Tregs were then tested in vitro for proliferation via cell count (FIG. 21A). Treg persistence over time (FIG. 21B), and IL-2 production (FIG. 21C). As is shown in FIG. 21A. IL-2 tethered Tregs, particularly Tregs where IL-2 was tethered to the EGFRt or Her2 tags, displayed limited expansion over time in the absence of IL-2. Furthermore, these IL-2 tethered Tregs showed a lack of persistence compared to wildtype control Tregs over time, as shown by FIG. 21B. Upon assessing the level of soluble IL-2 in the Treg media over time, it was found that the protein tag (e.g., the Her2 tag tethered to IL-2 as shown in FIG. 21C) released its IL-2 as soluble IL-2 into the media between Days 8-12, thereby reducing the bioavailability of IL-2 directly at the Treg membrane to activate the Tregs long term. As with the CA-STAT5 and IL-2 secreting Treg models described above, this model also demonstrates that the results presented in FIGS. 1A-17 and Examples 1-15 above are unique and surprising.

### Example 17: Recombinant Cytokine Receptors Engineered with IL-2Rβ Transmembrane Domain Promote Regulatory T Cell Survival and Proliferation

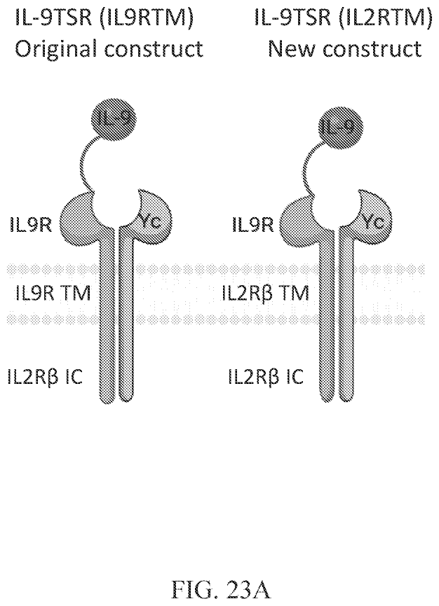

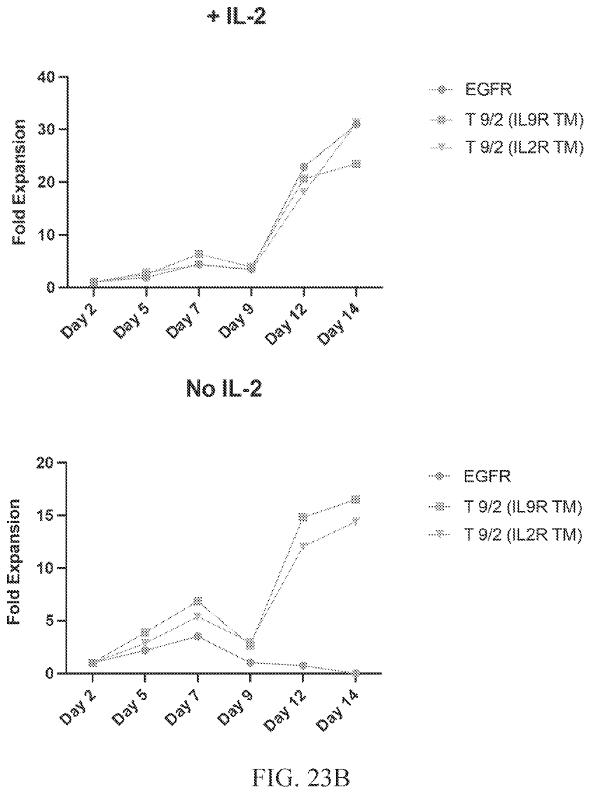

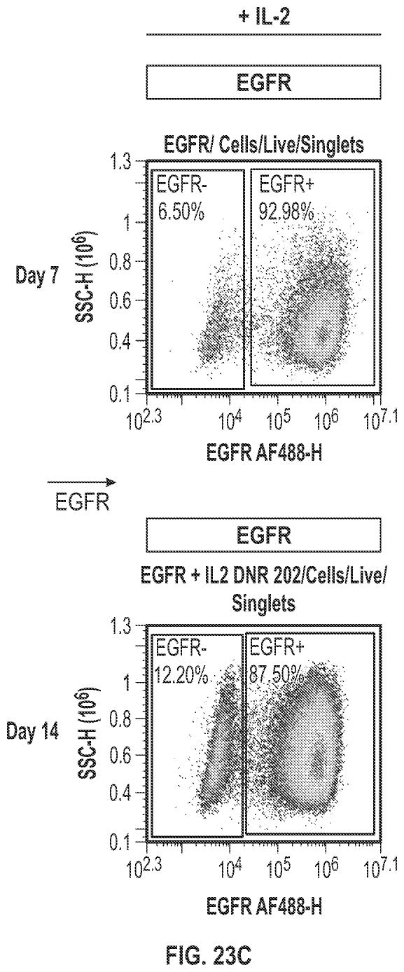

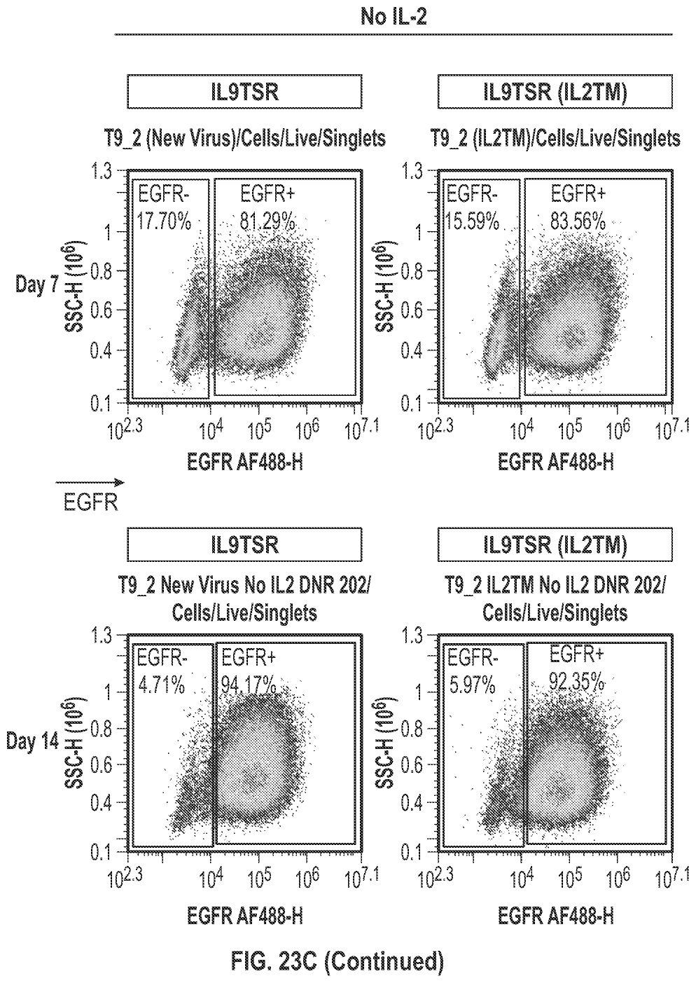

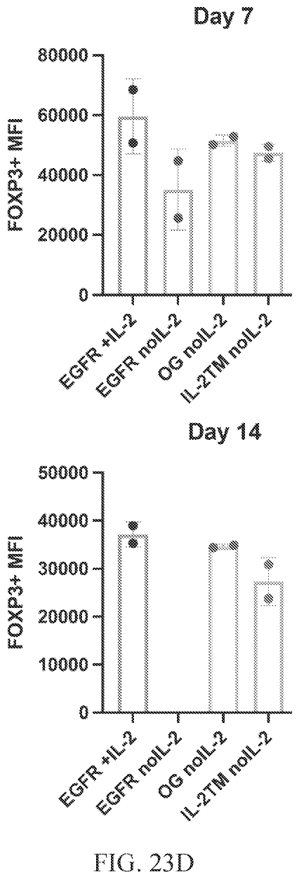

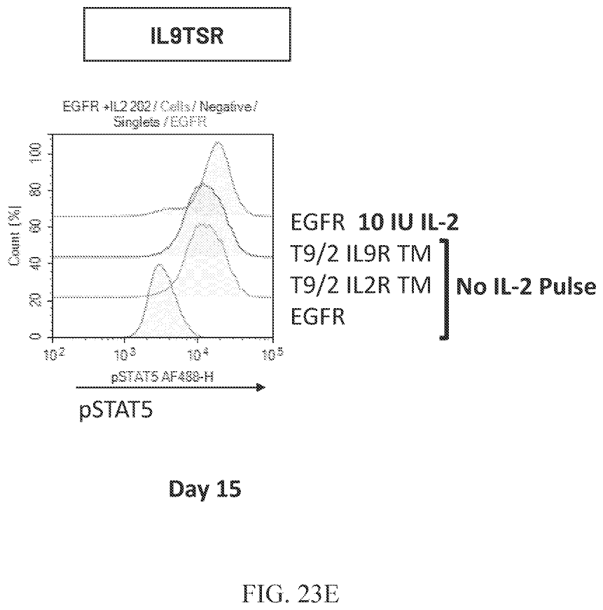

IL-9 tethered IL-9Rα/IL-2Rβ recombinant cytokine receptors were generated wherein the transmembrane domain was an IL-2Rβ transmembrane domain (called “IL9TSR (IL2RTM)” hereafter) as shown in FIG. 23A, and then transduced into Treg cells as described in the Examples above. The IL-9 tethered IL-9Rα/IL-2Rβ recombinant cytokine receptor construct described in Examples 1-15 above, wherein the transmembrane domain was an IL-9Rα transmembrane domain (also called “IL9TSR (IL9RTM)”), was used as a positive control.

Survival and Proliferation: The IL9TSR (IL2RTM) transduced Tregs, as well as the EGFR control Tregs and Tregs transduced with IL9TSR (IL9RTM) (also called “OG”, or original), were cultured for up to 14 days, as described in the Examples above. EGFR control Tregs were cultured with exogenous IL-2, and Tregs expressing either IL9TSR (IL2RTM) or IL9TSR (IL9RTM) constructs were cultured without exogenous IL-2.

Tregs transduced with IL9TSR (IL2RTM) (or “T9/2 (IL2RTM)”) proliferated in the absence of exogenous IL-2 similarly to the Tregs transduced with IL9TSR (IL9RTM) (or “T9/2 (IL9RTM)”), as shown in FIG. 23B. Additionally, Tregs transduced with IL9TSR (IL2RTM) (or “IL9TSR (IL2TM)”) survived in the absence of exogenous IL-2 at both day 7 and day 14 to approximately the same degree as Tregs transduced with IL9TSR (IL9RTM) (or “IL9TSR”), as shown in FIG. 23C.

Treg phenotype: EGFR Treg controls, Tregs transduced with IL9TSR (IL2RTM), and Tregs transduced with IL9TSR (IL9RTM) were cultured to day 7 and to day 14. EGFR Treg controls were cultured with or without exogenous IL-2, and Tregs transduced with IL9TSR (IL2RTM) or IL9TSR (IL9RTM) were cultured without exogenous IL-2. Cells were then harvested at days 7 and 14 and analyzed for FoxP3 protein levels using flow cytometry, as described in the Examples above. At both day 7 and day 14, Tregs transduced with IL9TSR (IL2RTM) (or “IL-2TM”) showed approximately the same FoxP3 MFI as Tregs transduced with IL9TSR (IL9RTM) (or “OG”), as demonstrated in FIG. 23D.

Intracellular IL-2 signaling: EGFR Treg controls, Tregs transduced with IL9TSR (IL2RTM), and Tregs transduced with IL9TSR (IL9RTM) were cultured to day 15 and then harvested and analyzed by phosphoflow analysis for the number of cells expressing phosphorylated STAT5, as described in the Examples above. EGFR Treg controls cultured with exogenous IL-2. Tregs transduced with IL9TSR (IL2RTM) (or “T9/2 IL2RTM”) cultured without exogenous IL-2, and Tregs transduced with IL9TSR (IL9RTM) (or “T9/2 IL9RTM”) cultured without exogenous IL-2 all showed similar levels and distribution of cells expressing pSTAT5 as shown in FIG. 23E, indicative of similar levels of constitutive intracellular IL-2 signaling.

Further, Tregs transduced with IL9TSR (IL2RTM) displayed similar suppressive activity on co-cultured effector T cells (results not shown) as Tregs transduced with IL9TSR (IL9RTM).

Taken together, these results demonstrate that Tregs expressing recombinant cytokine receptors comprising the IL-2Rβ transmembrane domain are effective for conferring IL-2 independence on Treg cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES
(Untethered recombinant cytokine receptor IL-4Rα/
IL-2Rβ, human)
SEQ ID NO: 1
MKVLQEPTCVSDYMSISTCEWKMNGPTNCSTELRLLYQLVFLLSEAHTCI
PENNGGAGCVCHLLMDDVVSADNYTLDLWAGQQLLWKGSFKPSEHVKPRA
PGNLTVHTNVSDTLLLTWSNPYPPDNYLYNHLTYAVNIWSENDPADFRIY
NVTYLEPSLRIAASTLKSGISYRARVRAWAQCYNTTWSEWSPSTKWHNSY
REPFEQHLLLGVSVSCIVILAVCLLCYVSITNCRNTGPWLKKVLKCNTPD
PSKFFSQLSSEHGGDVQKWLSSPFPSSSFSPGGLAPEISPLEVLERDKVT
QLLLQQDKVPEPASLSSNHSLTSCFTNQGYFFFHLPDALEIEACQVYFTY
DPYSEEDPDEGVAGAPTGSSPQPLQPLSGEDDAYCTFPSRDDLLLFSPSL
LGGPSPPSTAPGGSGAGEERMPPSLQERVPRDWDPQPLGPPTPGVPDLVD
FQPPPELVLREAGEEVPDAGPREGVSFPWSRPPGQGEFRALNARLPLNTD
AYLSLQELQGQDPTHLV
(Untethered recombinant cytokine receptor IL-7Rα/
IL-2Rβ, human)
SEQ ID NO: 2
ESGYAQNGDLEDAELDDYSFSCYSQLEVNGSQHSLTCAFEDPDVNITNLE
FEICGALVEVKCLNFRKLQEIYFIETKKFLLIGKSNICVKVGEKSLTCKK
IDLTTIVKPEAPFDLSVVYREGANDFVVTFNTSHLQKKYVKVLMHDVAYR
QEKDENKWTHVNLSSTKLTLLQRKLQPAAMYEIKVRSIPDHYFKGFWSEW
SPSYYFRTPEINNSSGEMDPILLTISILSFFSVALLVILACVLWNCRNTG
PWLKKVLKCNTPDPSKFFSQLSSEHGGDVQKWLSSPFPSSSFSPGGLAPE
ISPLEVLERDKVTQLLLQQDKVPEPASLSSNHSLTSCFTNQGYFFFHLPD
ALEIEACQVYFTYDPYSEEDPDEGVAGAPTGSSPQPLQPLSGEDDAYCTF
PSRDDLLLFSPSLLGGPSPPSTAPGGSGAGEERMPPSLQERVPRDWDPQP
LGPPTPGVPDLVDFQPPPELVLREAGEEVPDAGPREGVSFPWSRPPGQGE
FRALNARLPLNTDAYLSLQELQGQDPTHLV
(Untethered recombinant cytokine receptor IL-9Rα/
IL-2Rβ, human)
SEQ ID NO: 3
SVTGEGQGPRSRTFTCLTNNILRIDCHWSAPELGQGSSPWLLFTSNQAPG
GTHKCILRGSECTVVLPPEAVLVPSDNFTITFHHCMSGREQVSLVDPEYL
PRRHVKLDPPSDLQSNISSGHCILTWSISPALEPMTTLLSYELAFKKQEE
AWEQAQHRDHIVGVTWLILEAFELDPGFIHEARLRVQMATLEDDVVEEER
YTGQWSEWSQPVCFQAPQRQGPLIPPWGWPGNTLVAVSIFLLLTGPTYLL
FNCRNTGPWLKKVLKCNTPDPSKFFSQLSSEHGGDVQKWLSSPFPSSSFS
PGGLAPEISPLEVLERDKVTQLLLQQDKVPEPASLSSNHSLTSCFTNQGY
FFFHLPDALEIEACQVYFTYDPYSEEDPDEGVAGAPTGSSPQPLQPLSGE
DDAYCTFPSRDDLLLFSPSLLGGPSPPSTAPGGSGAGEERMPPSLQERVP
RDWDPQPLGPPTPGVPDLVDFQPPPELVLREAGEEVPDAGPREGVSFPWS
RPPGQGEFRALNARLPLNTDAYLSLQELQGQDPTHLV
(Untethered recombinant cytokine receptor IL-21Rα/
IL-2Rβ, human)
SEQ ID NO: 4
CPDLVCYTDYLQTVICILEMWNLHPSTLTLTWQDQYEELKDEATSCSLHR
SAHNATHATYTCHMDVFHFMADDIFSVNITDQSGNYSQECGSFLLAESIK
PAPPFNVTVTFSGQYNISWRSDYEDPAFYMLKGKLQYELQYRNRGDPWAV
SPRRKLISVDSRSVSLLPLEFRKDSSYELQVRAGPMPGSSYQGTWSEWSD
PVIFQTQSEELKEGWNPHLLLLLLLVIVFIPAFWNCRNTGPWLKKVLKCN
TPDPSKFFSQLSSEHGGDVQKWLSSPFPSSSFSPGGLAPEISPLEVLERD
KVTQLLLQQDKVPEPASLSSNHSLTSCFTNQGYFFFHLPDALEIEACQVY
FTYDPYSEEDPDEGVAGAPTGSSPQPLQPLSGEDDAYCTFPSRDDLLLFS
PSLLGGPSPPSTAPGGSGAGEERMPPSLQERVPRDWDPQPLGPPTPGVPD
LVDFQPPPELVLREAGEEVPDAGPREGVSFPWSRPPGQGEFRALNARLPL
NTDAYLSLQELQGQDPTHLV
(Untethered recombinant cytokine receptor
IL-9RαED/IL-2RβTD + ID, human)
SEQ ID NO: 5
SVTGEGQGPRSRTFTCLTNNILRIDCHWSAPELGQGSSPWLLFTSNQAPG
GTHKCILRGSECTVVLPPEAVLVPSDNFTITFHHCMSGREQVSLVDPEYL
PRRHVKLDPPSDLQSNISSGHCILTWSISPALEPMTTLLSYELAFKKQEE
AWEQAQHRDHIVGVTWLILEAFELDPGFIHEARLRVQMATLEDDVVEEER
YTGQWSEWSQPVCFQAPQRQGPLIPPWGWPIPWLGHLLVGLSGAFGFIIL
VYLLINCRNTGPWLKKVLKCNTPDPSKFFSQLSSEHGGDVQKWLSSPFPS
SSFSPGGLAPEISPLEVLERDKVTQLLLQQDKVPEPASLSSNHSLTSCFT
NQGYFFFHLPDALEIEACQVYFTYDPYSEEDPDEGVAGAPTGSSPQPLQP
LSGEDDAYCTFPSRDDLLLFSPSLLGGPSPPSTAPGGSGAGEERMPPSLQ
ERVPRDWDPQPLGPPTPGVPDLVDFQPPPELVLREAGEEVPDAGPREGVS
FPWSRPPGQGEFRALNARLPLNTDAYLSLQELQGQDPTHLV
(Tethered recombinant cytokine receptor IL-4 +
IL-4Rα/IL-2Rβ, human)
SEQ ID NO: 6
HKCDITLQEIIKTLNSLTEQKTLCTELTVTDIFAASKNTTEKETFCRAAT
VLRQFYSHHEKDTRCLGATAQQFHRHKQLIRFLKRLDRNLWGLAGLNSCP
VKEANQSTLENFLERLKTIMREKYSKCSSGGGGSGGGGSMKVLQEPTCVS
DYMSISTCEWKMNGPTNCSTELRLLYQLVFLLSEAHTCIPENNGGAGCVC
HLLMDDVVSADNYTLDLWAGQQLLWKGSFKPSEHVKPRAPGNLTVHTNVS
DTLLLTWSNPYPPDNYLYNHLTYAVNIWSENDPADFRIYNVTYLEPSLRI
AASTLKSGISYRARVRAWAQCYNTTWSEWSPSTKWHNSYREPFEQHLLLG
VSVSCIVILAVCLLCYVSITNCRNTGPWLKKVLKCNTPDPSKFFSQLSSE
HGGDVQKWLSSPFPSSSFSPGGLAPEISPLEVLERDKVTQLLLQQDKVPE
PASLSSNHSLTSCFTNQGYFFFHLPDALEIEACQVYFTYDPYSEEDPDEG
VAGAPTGSSPQPLQPLSGEDDAYCTFPSRDDLLLFSPSLLGGPSPPSTAP
GGSGAGEERMPPSLQERVPRDWDPQPLGPPTPGVPDLVDFQPPPELVLRE
AGEEVPDAGPREGVSFPWSRPPGQGEFRALNARLPLNTDAYLSLQELQGQ
DPTHLV
(Tethered recombinant cytokine receptor IL-7 +
IL-7Rα/IL-2Rβ, human)
SEQ ID NO: 7
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHGGGGSGGGGSESGYAQNGDLEDAELDDYSFSCYSQLEVNGSQHSLTCA
FEDPDVNITNLEFEICGALVEVKCLNFRKLQEIYFIETKKFLLIGKSNIC
VKVGEKSLTCKKIDLTTIVKPEAPFDLSVVYREGANDFVVTENTSHLQKK
YVKVLMHDVAYRQEKDENKWTHVNLSSTKLTLLQRKLQPAAMYEIKVRSI
PDHYFKGFWSEWSPSYYFRTPEINNSSGEMDPILLTISILSFFSVALLVI
LACVLWNCRNTGPWLKKVLKCNTPDPSKFFSQLSSEHGGDVQKWLSSPFP
SSSFSPGGLAPEISPLEVLERDKVTQLLLQQDKVPEPASLSSNHSLTSCF
TNQGYFFFHLPDALEIEACQVYFTYDPYSEEDPDEGVAGAPTGSSPQPLQ
PLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGV
SFPWSRPPGQGEFRALNARLPLNTDAYLSLQELQGQDPTHLV
(Tethered recombinant cytokine receptor IL-9 +
IL-9Rα/IL-2Rβ, human)
SEQ ID NO: 8
QGCPTLAGILDINFLINKMQEDPASKCHCSANVTSCLCLGIPSDNCTRPC
FSERLSQMTNTTMQTRYPLIFSRVKKSVEVLKNNKCPYFSCEQPCNQTTA
GNALTFLKSLLEIFQKEKMRGMRGKIGGGGSGGGGSSVTGEGQGPRSRTF
TCLTNNILRIDCHWSAPELGQGSSPWLLFTSNQAPGGTHKCILRGSECTV
VLPPEAVLVPSDNFTITFHHCMSGREQVSLVDPEYLPRRHVKLDPPSDLQ
SNISSGHCILTWSISPALEPMTTLLSYELAFKKQEEAWEQAQHRDHIVGV
TWLILEAFELDPGFIHEARLRVQMATLEDDVVEEERYTGQWSEWSQPVCF
QAPQRQGPLIPPWGWPGNTLVAVSIFLLLTGPTYLLFNCRNTGPWLKKVL
KCNTPDPSKFFSQLSSEHGGDVQKWLSSPFPSSSFSPGGLAPEISPLEVL
ERDKVTQLLLQQDKVPEPASLSSNHSLTSCFTNQGYFFFHLPDALEIEAC
QVYFTYDPYSEEDPDEGVAGAPTGSSPQPLQPLSGEDDAYCTFPSRDDLL
LESPSLLGGPSPPSTAPGGSGAGEERMPPSLQERVPRDWDPQPLGPPTPG
VPDLVDFQPPPELVLREAGEEVPDAGPREGVSFPWSRPPGQGEFRALNAR
LPLNTDAYLSLQELQGQDPTHLV
(Tethered recombinant cytokine receptor IL-21 +
IL-21Rα/IL-2Rβ, human)
SEQ ID NO: 9
HKSSSQGQDRHMIRMRQLIDIVDQLKNYVNDLVPEFLPAPEDVETNCEWS
AFSCFQKAQLKSANTGNNERIINVSIKKLKRKPPSTNAGRRQKHRLTCPS
CDSYEKKPPKEFLERFKSLLQKMIHQHLSSRTHGSEDSGGGGSGGGGSCP
DLVCYTDYLQTVICILEMWNLHPSTLTLTWQDQYEELKDEATSCSLHRSA
HNATHATYTCHMDVFHFMADDIFSVNITDQSGNYSQECGSFLLAESIKPA
PPFNVTVTFSGQYNISWRSDYEDPAFYMLKGKLQYELQYRNRGDPWAVSP
RRKLISVDSRSVSLLPLEFRKDSSYELQVRAGPMPGSSYQGTWSEWSDPV
IFQTQSEELKEGWNPHLLLLLLLVIVFIPAFWNCRNTGPWLKKVLKCNTP
DPSKFFSQLSSEHGGDVQKWLSSPFPSSSFSPGGLAPEISPLEVLERDKV
TQLLLQQDKVPEPASLSSNHSLTSCFTNQGYFFFHLPDALEIEACQVYFT
YDPYSEEDPDEGVAGAPTGSSPQPLQPLSGEDDAYCTFPSRDDLLLESPS
LLGGPSPPSTAPGGSGAGEERMPPSLQERVPRDWDPQPLGPPTPGVPDLV
DFQPPPELVLREAGEEVPDAGPREGVSFPWSRPPGQGEFRALNARLPLNT
DAYLSLQELQGQDPTHLV
(P2A peptide, artificial)
SEQ ID NO: 10
SGATNFSLLKQAGDVEENPGP
(IL-4Rα extracellular domain, human)
SEQ ID NO: 11
MKVLQEPTCVSDYMSISTCEWKMNGPTNCSTELRLLYQLVFLLSEAHTCI
PENNGGAGCVCHLLMDDVVSADNYTLDLWAGQQLLWKGSFKPSEHVKPRA
PGNLTVHTNVSDTLLLTWSNPYPPDNYLYNHLTYAVNIWSENDPADFRIY
NVTYLEPSLRIAASTLKSGISYRARVRAWAQCYNTTWSEWSPSTKWHNSY
REPFEQH
(IL-7Rα extracellular domain, human)
SEQ ID NO: 12
ESGYAQNGDLEDAELDDYSFSCYSQLEVNGSQHSLTCAFEDPDVNITNLE
FEICGALVEVKCLNFRKLQEIYFIETKKFLLIGKSNICVKVGEKSLTCKK
IDLTTIVKPEAPFDLSVVYREGANDFVVTFNTSHLQKKYVKVLMHDVAYR
QEKDENKWTHVNLSSTKLTLLQRKLQPAAMYEIKVRSIPDHYFKGFWSEW
SPSYYFRTPEINNSSGEMD
(IL-9Rα extracellular domain, human)
SEQ ID NO: 13
SVTGEGQGPRSRTFTCLTNNILRIDCHWSAPELGQGSSPWLLFTSNQAPG
GTHKCILRGSECTVVLPPEAVLVPSDNFTITFHHCMSGREQVSLVDPEYL
PRRHVKLDPPSDLQSNISSGHCILTWSISPALEPMTTLLSYELAFKKQEE
AWEQAQHRDHIVGVTWLILEAFELDPGFIHEARLRVQMATLEDDVVEEER
YTGQWSEWSQPVCFQAPQRQGPLIPPWGWP
(IL-21Rα extracellular domain, human)
SEQ ID NO: 14
CPDLVCYTDYLQTVICILEMWNLHPSTLTLTWQDQYEELKDEATSCSLHR
SAHNATHATYTCHMDVFHFMADDIFSVNITDQSGNYSQECGSFLLAESIK
PAPPFNVTVTFSGQYNISWRSDYEDPAFYMLKGKLQYELQYRNRGDPWAV
SPRRKLISVDSRSVSLLPLEFRKDSSYELQVRAGPMPGSSYQGTWSEWSD
PVIFQTQSEELKE
(Tethered recombinant cytokine receptor IL-9 +
IL-9RαED/IL-2RβTD + ID, human)
SEQ ID NO: 15
QGCPTLAGILDINFLINKMQEDPASKCHCSANVTSCLCLGIPSDNCTRPC
FSERLSQMTNTTMQTRYPLIFSRVKKSVEVLKNNKCPYFSCEQPCNQTTA
GNALTFLKSLLEIFQKEKMRGMRGKIGGGGSGGGGSSVTGEGQGPRSRTF
TCLTNNILRIDCHWSAPELGQGSSPWLLFTSNQAPGGTHKCILRGSECTV
VLPPEAVLVPSDNFTITFHHCMSGREQVSLVDPEYLPRRHVKLDPPSDLQ
SNISSGHCILTWSISPALEPMTTLLSYELAFKKQEEAWEQAQHRDHIVGV
TWLILEAFELDPGFIHEARLRVQMATLEDDVVEEERYTGQWSEWSQPVCF
QAPQRQGPLIPPWGWPIPWLGHLLVGLSGAFGFIILVYLLINCRNTGPWL
KKVLKCNTPDPSKFFSQLSSEHGGDVQKWLSSPFPSSSFSPGGLAPEISP
LEVLERDKVTQLLLQQDKVPEPASLSSNHSLTSCFTNQGYFFFHLPDALE
IEACQVYFTYDPYSEEDPDEGVAGAPTGSSPQPLQPLSGEDDAYCTFPSR
DDLLLFSPSLLGGPSPPSTAPGGSGAGEERMPPSLQERVPRDWDPQPLGP
PTPGVPDLVDFQPPPELVLREAGEEVPDAGPREGVSFPWSRPPGQGEFRA
LNARLPLNTDAYLSLQELQGQDPTHLV
(IL-4Rα transmembrane domain, human)
SEQ ID NO: 16
LLLGVSVSCIVILAVCLLCYVSIT
(IL-7Rα transmembrane domain, human)
SEQ ID NO: 17
PILLTISILSFFSVALLVILACVLW
(IL-9Rα transmembrane domain, human)
SEQ ID NO: 18
GNTLVAVSIFLLLTGPTYLLF
(IL-21Rα transmembrane domain, human)
SEQ ID NO: 19
GWNPHLLLLLLLVIVFIPAFW
(IL-2Rβ intracellular domain, human)
SEQ ID NO: 20
NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV
(IL-2Rβ transmembrane domain, human)
SEQ ID NO: 21
IPWLGHLLVGLSGAFGFIILVYLLI
(IL-4 cytokine, human)
SEQ ID NO: 22
HKCDITLQEIIKTLNSLTEQKTLCTELTVTDIFAASKNTTEKETFCRAAT
VLRQFYSHHEKDTRCLGATAQQFHRHKQLIRFLKRLDRNLWGLAGLNSCP
VKEANQSTLENFLERLKTIMREKYSKCSS
(IL-7 cytokine, human)
SEQ ID NO: 23
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EH
(IL-9 cytokine, human)
SEQ ID NO: 24
QGCPTLAGILDINFLINKMQEDPASKCHCSANVTSCLCLGIPSDNCTRPC
FSERLSQMTNTTMQTRYPLIFSRVKKSVEVLKNNKCPYFSCEQPCNQTTA
GNALTFLKSLLEIFQKEKMRGMRGKI
(IL-21 cytokine, human)
SEQ ID NO: 25
HKSSSQGQDRHMIRMRQLIDIVDQLKNYVNDLVPEFLPAPEDVETNCEWS
AFSCFQKAQLKSANTGNNERIINVSIKKLKRKPPSTNAGRRQKHRLTCPS
CDSYEKKPPKEFLERFKSLLQKMIHQHLSSRTHGSEDS
(Leader/signal sequence, human)
SEQ ID NO: 26
MLLLVTSLLLCELPHPAFLLIP
(Tether, artificial)
SEQ ID NO: 27
GGGGSGGGGS
(2A linker, artificial)
SEQ ID NO: 28
GSGPR
(Tether, artificial)
SEQ ID NO: 29
(GGGGS)n
(Tether, artificial)
SEQ ID NO: 30
GGGGS
(Tether, artificial)
SEQ ID NO: 31
GGGGSGGGGSGGGGS
(Tether, artificial)
SEQ ID NO: 32
SGGGSGGGGSGGGGSGGGGSGGGSLQ

1. A regulatory T cell (Treg) comprising a recombinant cytokine receptor, wherein the recombinant cytokine receptor comprises
an extracellular cytokine receptor domain, a transmembrane domain, and an intracellular IL-2 receptor beta chain domain, wherein the extracellular cytokine receptor domain binds to a cytokine other than IL-2.

2. The Treg of claim 1, wherein the extracellular cytokine receptor domain is tethered to the cytokine.

3. The Treg of claim 1 or 2, wherein the extracellular cytokine receptor domain is selected from the group consisting of an IL-4 extracellular domain, an IL-7 extracellular domain, an IL-9 extracellular domain, and an IL-21 extracellular domain.

4. The Treg of any one of claims 2-3, wherein the extracellular cytokine receptor domain is tethered to the cytokine by a polypeptide linker, optionally wherein the polypeptide linker comprises glycine and serine residues.

5. The Treg of claim 1, wherein the extracellular cytokine receptor domain is not tethered to the cytokine.

6. The Treg of any one of claims 1-5, wherein the recombinant cytokine receptor engages in IL-2 signaling in the absence of IL-2.

7. The Treg of any one of claims 1-6, wherein the transmembrane domain is a transmembrane domain of a cytokine receptor.

8. The Treg of claim 7, wherein the transmembrane domain is a transmembrane domain of an IL-9 receptor, an IL-2 receptor, an IL-4 receptor, an IL-7 receptor, or an IL-21 receptor.

9. The Treg of any one of claims 1-8, wherein the TM and the ED are from the same cytokine receptor.

10. The Treg of any one of claims 1-9, wherein
the intracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:20; and/or
the extracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO: 11-14; and/or
the transmembrane domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO: 16-19 or 21.

11. The Treg of any one of claims 1-10, wherein the recombinant cytokine receptor comprises
a) an IL-4Rα extracellular domain, an IL-4Rα transmembrane domain, and an IL-2Rβ intracellular domain;
b) an IL-4Rα extracellular domain, an IL-2Rβ transmembrane domain, and an IL-2Rβ intracellular domain;
c) an IL-7Rα extracellular domain, an IL-7Rα transmembrane domain, and an IL-2Rβ intracellular domain;
d) an IL-7Rα extracellular domain, an IL-2Rβ transmembrane domain, and an IL-2Rβ intracellular domain;
e) an IL-9Rα extracellular domain, an IL-9Rα transmembrane domain, and an IL-2Rβ intracellular domain;
f) an IL-9Rα extracellular domain, an IL-2Rβ transmembrane domain, and an IL-2Rβ intracellular domain;
g) an IL-21Rα extracellular domain, an IL-21Rα transmembrane domain, and an IL-2Rβ intracellular domain; or
h) an IL-21Rα extracellular domain, an IL-2Rβ transmembrane domain, and an IL-2Rβ intracellular domain.

12. The Treg of any one of claims 1-11, wherein:
a) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 16, and the extracellular domain comprises an amino acid sequence comprising at least 80%, at least 90%, at least 95%, at least 99%, or 100% sequence identity with the amino acid sequence set forth in SEQ ID NO:11;
b) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 17, and the extracellular domain comprises an amino acid sequence comprising at least 80%, at least 90%, at least 95%, at least 99%, or 100% sequence identity with the amino acid sequence set forth in SEQ ID NO:12;
c) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 18, and the extracellular domain comprises an amino acid sequence comprising at least 80%, at least 90%, at least 95%, at least 99%, or 100% sequence identity with the amino acid sequence set forth in SEQ ID NO:13;
d) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO:13;
e) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO:14;
f) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 16, and the extracellular domain comprises an amino acid sequence comprising at least 80%, at least 90%, at least 95%, at least 99%, or 100% sequence identity with the amino acid sequence set forth in SEQ ID NO: 11, and the cytokine comprises an amino acid sequence comprising at least 80%, at least 90%, at least 95%, at least 99%, or 100% sequence identity with the amino acid sequence set forth in SEQ ID NO: 22;
g) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:17, and the extracellular domain comprises an amino acid sequence comprising at least 80%, at least 90%, at least 95%, at least 99%, or 100% sequence identity with the amino acid sequence set forth in SEQ ID NO:12, and the cytokine comprises an amino acid sequence comprising at least 80%, at least 90%, at least 95%, at least 99%, or 100% sequence identity with the amino acid sequence set forth in SEQ ID NO: 23;
h) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 18, and the extracellular domain comprises an amino acid sequence comprising at least 80%, at least 90%, at least 95%, at least 99%, or 100% sequence identity with the amino acid sequence set forth in SEQ ID NO: 13, and the cytokine comprises an amino acid sequence comprising at least 80%, at least 90%, at least 95%, at least 99%, or 100% sequence identity with the amino acid sequence set forth in SEQ ID NO: 24;
i) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO: 13, and the cytokine comprises an amino acid sequence comprising at least 80%, at least 90%, at least 95%, at least 99%, or 100% sequence identity with the amino acid sequence set forth in SEQ ID NO: 24; or
j) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO:14, and the cytokine comprises an amino acid sequence comprising at least 80%, at least 90%, at least 95%, at least 99%, or 100% sequence identity with the amino acid sequence set forth in SEQ ID NO: 25.

13. The Treg of any one of claims 1, 3, and 5-12, wherein the recombinant cytokine receptor comprises an amino acid sequence comprising at least 80%, at least 85%, at least 90%, at least 95%, at least 96%, at least 97%, at least 98%, at least 99%, or 100% sequence identity with an amino acid sequence selected from the group consisting of SEQ ID NOs: 1-5.

14. The Treg of any one of claims 1-4 and 6-13, wherein the recombinant cytokine receptor comprises an amino acid sequence comprising at least 80%, at least 85%, at least 90%, at least 95%, at least 96%, at least 97%, at least 98%, at least 99%, or 100% sequence identity with an amino acid sequence selected from the group consisting of SEQ ID NOs: 6-9 and 15.

15. The Treg of any one of claims 1-14, wherein the Treg is CD4+, CD25+, and CD127lo, optionally, wherein the Treg expresses FOX3P and/or HELIOS.

16. The Treg of any one of claims 1-15, further comprising a chimeric antigen receptor (CAR).

17. The Treg of any one of claims 1-16 for use in treating an immune-related disorder in an individual.

18. The Treg for use of claim 17, wherein the Treg cells are autologous to the individual, optionally wherein the individual is human.

19. The Treg for use of claim 17 or claim 18, wherein the Treg cells prevent, ameliorate, or cure an immune-related disorder.

20. A recombinant cytokine receptor comprising an extracellular cytokine receptor domain, a transmembrane domain, and an intracellular IL-2 receptor beta chain domain, wherein the extracellular cytokine receptor domain is able to bind to a cytokine other than IL-2.

21. A recombinant cytokine receptor comprising an extracellular cytokine receptor domain, a transmembrane domain, and an intracellular IL-2 receptor beta domain, wherein the extracellular cytokine receptor domain is able to bind to and is tethered to a cytokine other than IL-2.

22. The recombinant cytokine receptor of claim 20 or 21, wherein the extracellular cytokine receptor domain is selected from the group consisting of an IL-4 extracellular domain, an IL-7 extracellular domain, an IL-9 extracellular domain, and an IL-21 extracellular domain.

23. The recombinant cytokine receptor of claim 21 or claim 22, wherein the extracellular cytokine receptor domain is tethered to the cytokine by a polypeptide linker, optionally wherein the polypeptide linker comprises glycine and serine residues.

24. The recombinant cytokine receptor of claim 20 or claim 22, wherein the extracellular cytokine receptor domain is not tethered to the cytokine.

25. The recombinant cytokine receptor of any one of claims 20-24, wherein the recombinant cytokine receptor engages in IL-2 signaling in the absence of IL-2.

26. The recombinant cytokine receptor of any one of claims 20-25, wherein the transmembrane domain is a transmembrane domain of a cytokine receptor, optionally wherein the transmembrane domain is a transmembrane domain of an IL-9 receptor, an IL-2 receptor, an IL-4 receptor, an IL-7 receptor, or an IL-21 receptor.

27. The recombinant cytokine receptor of any one of claims 20-26, wherein the TM and the ED are from the same cytokine receptor.

28. The recombinant cytokine receptor of any one of claims 20-27, wherein
the intracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:20;
the extracellular domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO:11-14; and/or
the transmembrane domain comprises an amino acid sequence comprising at least 80%, at least 85%, at least 90%, at least 95%, at least 96%, at least 97%, at least 98%, at least 99%, or 100% sequence identity with the amino acid sequence set forth in SEQ ID NO: 16-19 or 21.

29. The recombinant cytokine receptor of any one of claims 20-28, wherein the recombinant cytokine receptor comprises
a) an IL-4Rα extracellular domain, an IL-4Rα transmembrane domain, and an IL-2Rβ intracellular domain;
b) an IL-4Rα extracellular domain, an IL-2Rβ transmembrane domain, and an IL-2Rβ intracellular domain;
c) an IL-7Rα extracellular domain, an IL-7Rα transmembrane domain, and an IL-2Rβ intracellular domain;
d) an IL-7Rα extracellular domain, an IL-2Rβ transmembrane domain, and an IL-2Rβ intracellular domain;
e) an IL-9Rα extracellular domain, an IL-9Rα transmembrane domain, and an IL-2Rβ intracellular domain;
f) an IL-9Rα extracellular domain, an IL-2Rβ transmembrane domain, and an IL-2Rβ intracellular domain;
g) an IL-21Rα extracellular domain, an IL-21Rα transmembrane domain, and an IL-2Rβ intracellular domain; or
h) an IL-21Rα extracellular domain, an IL-2Rβ transmembrane domain, and an IL-2Rβ intracellular domain.

30. The recombinant cytokine receptor of any one of claims 20-29, wherein:
a) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 16, and the extracellular domain comprises an amino acid sequence comprising at least 80%, at least 90%, at least 95%, at least 99%, or 100% sequence identity with the amino acid sequence set forth in SEQ ID NO:11;
b) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 17, and the extracellular domain comprises an amino acid sequence comprising at least 80%, at least 90%, at least 95%, at least 99%, or 100% sequence identity with the amino acid sequence set forth in SEQ ID NO:12;
c) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 18, and the extracellular domain comprises an amino acid sequence comprising at least 80%, at least 90%, at least 95%, at least 99%, or 100% sequence identity with the amino acid sequence set forth in SEQ ID NO:13;
d) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO:13;
e) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO:14;
f) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 16, and the extracellular domain comprises an amino acid sequence comprising at least 80%, at least 90%, at least 95%, at least 99%, or 100% sequence identity with the amino acid sequence set forth in SEQ ID NO: 11, and the cytokine comprises an amino acid sequence comprising at least 80%, at least 90%, at least 95%, at least 99%, or 100% sequence identity with the amino acid sequence set forth in SEQ ID NO: 22;
g) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 17, and the extracellular domain comprises an amino acid sequence comprising at least 80%, at least 90%, at least 95%, at least 99%, or 100% sequence identity with the amino acid sequence set forth in SEQ ID NO: 12, and the cytokine comprises an amino acid sequence comprising at least 80%, at least 90%, at least 95%, at least 99%, or 100% sequence identity with the amino acid sequence set forth in SEQ ID NO: 23;
h) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:18, and the extracellular domain comprises an amino acid sequence comprising at least 80%, at least 90%, at least 95%, at least 99%, or 100% sequence identity with the amino acid sequence set forth in SEQ ID NO:13, and the cytokine comprises an amino acid sequence comprising at least 80%, at least 90%, at least 95%, at least 99%, or 100% sequence identity with the amino acid sequence set forth in SEQ ID NO: 24;
i) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO:21, and the extracellular domain comprises an amino acid sequence comprising at least 80%, at least 90%, at least 95%, at least 99%, or 100% sequence identity with the amino acid sequence set forth in SEQ ID NO: 13, and the cytokine comprises an amino acid sequence comprising at least 80%, at least 90%, at least 95%, at least 99%, or 100% sequence identity with the amino acid sequence set forth in SEQ ID NO: 24; or
j) the intracellular domain comprises an amino acid sequence comprising at least 80%, at least 90%, at least 95%, at least 99%, or 100% sequence identity with the amino acid sequence set forth in SEQ ID NO:20, the transmembrane domain comprises an amino acid sequence comprising at least 80%, at least 90%, at least 95%, at least 99%, or 100% sequence identity with the amino acid sequence set forth in SEQ ID NO: 19, and the extracellular domain comprises an amino acid sequence comprising at least 80%, at least 90%, at least 95%, at least 99%, or 100% sequence identity with the amino acid sequence set forth in SEQ ID NO: 14, and the cytokine comprises an amino acid sequence comprising at least 80%, at least 90%, at least 95%, at least 99%, or 100% sequence identity with the amino acid sequence set forth in SEQ ID NO: 25.

31. The recombinant cytokine receptor of any one of claims 20, 22, and 24-30, wherein the recombinant cytokine receptor comprises an amino acid sequence comprising at least 80%, at least 85%, at least 90%, at least 95%, at least 96%, at least 97%, at least 98%, at least 99%, or 100% sequence identity with an amino acid sequence selected from the group consisting of SEQ ID NOs: 1-5.

32. The recombinant cytokine receptor of any one of claims 20-23 and 25-31, wherein the recombinant cytokine receptor comprises an amino acid sequence comprising at least 80%, at least 85%, at least 90%, at least 95%, at least 96%, at least 97%, at least 98%, at least 99%, or 100% sequence identity with an amino acid sequence selected from the group consisting of SEQ ID NOs: 6-9 and 15.

33. A nucleic acid encoding the recombinant cytokine receptor of any one of claims 20-32.

34. A vector comprising the nucleic acid of claim 33.

35. A method of expanding a Treg cell in the absence of IL-2 comprising introducing the nucleic acid of claim 33, or the vector of claim 34 to the Treg cell and culturing the cell in the absence of IL-2.

36. The method of claim 35, further comprising detecting at least one Treg marker selected from the group consisting of CD4+, CD25+, and CD127, optionally further comprising detecting FOXP3 and/or HELIOS.

37. The method of claim 35 or claim 36, wherein the Treg cell is able to proliferate in the absence of IL-2.

38. The method of any one of claims 35-37, wherein one or more markers of endogenous IL-2 signaling is detected, optionally wherein the one or more markers of endogenous IL-2 signaling comprises phosphorylated STAT-5.

39. The method of any one of claims 35-38, wherein the method generates Treg cells comprising a recombinant cytokine receptor, wherein:
i, the in vitro and/or in vivo suppressive activity of the Treg on CD8+ and/or CD4+ T cells is increased compared to a control Treg;
ii, the rate of division of CD4+ and/or CD8+ T cells is decreased when cultured in the presence of the Treg compared to the rate of division of CD4+ and/or CD8+ T cells when cultured without the Treg;
iii, the relative amount of Treg cells in a composition comprising a population of Treg cells transduced with a recombinant cytokine receptor increases over time;
iv, a composition comprising a population of Treg cells transduced with the recombinant cytokine receptor contains more viable cells compared to a composition comprising a population of the same Treg cells not transduced with the recombinant cytokine receptor;
v, at least 60% of the cells in the population of Treg cells transduced with the recombinant cytokine receptor remain viable in vitro 7 days after transduction;
vi, the level of Treg proliferation increases from re-stimulation in vitro and/or in vivo one or more times;
vii, the IL-10 cytokine levels produced by the Treg in vitro and/or in vivo are increased compared to a control Treg;
viii, the IFN-γ cytokine levels produced by the Treg in vitro and/or in vivo are increased compared to a control Treg; and/or
ix, the Gr-B cytokine levels produced by the Treg in vitro and/or in vivo are increased compared to a control Treg.

40. The method of any one of claims 35-39, further comprising culturing the Treg cell in a composition comprising the cytokine, optionally wherein the method further comprises culturing the Treg cell in a composition comprising one or more additional cytokines other than IL-2.

41. The method of any one of claims 35-40, wherein the population of Treg cells transduced with the recombinant cytokine receptor expands at least two-fold more than a population of the same Treg cells not transduced with the recombinant cytokine receptor, optionally wherein the population of Treg cells transduced with the recombinant cytokine receptor expands at least two-fold, and wherein the population of Treg cells transduced with the recombinant cytokine receptor maintains expression of least one Treg marker selected from the group consisting of CD4+, CD25+, and CD127 lo.

42. The Treg for use of any one of claims 17-19, wherein the individual expresses IL-2.
